SEQUENCE LISTING (390 sequences)

SEQ ID NO: 1 - protein, 13 aa, synthetic construct
  source 1..13: note = TRG CDR3 E57 G1
CATWDGFYYKKLF

SEQ ID NO: 2 - protein, 18 aa, synthetic construct
  source 1..18: note = TRD CDR3 E57 D3
CAASSPIRGYTGSDKLIF

SEQ ID NO: 3 - DNA, 921 bp, synthetic construct
  source 1..921: mol_type = other DNA; note = TRG codon optimised E57 G1
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacggcttctactacaagaagctgttcggcagcggcaccaccctggtggtgacagacaagcagctggacgccgacgtgtcccccaagcctaccatcttcctgccctcaattgctgagacaaagctgcagaaggccggcacctacctgtgcctgctggaaaagttcttcccagacgtgatcaagatccactgggaggaaaagaagtccaacaccatcctgggcagccaagaaggcaacaccatgaagaccaacgacacctacatgaagttcagctggctgaccgtgcccgagaagtccctggacaaagaacaccggtgcatcgtgcggcacgagaacaacaagaacggcgtggaccaagaaatcatcttcccacccatcaagaccgacgtgatcacaatggaccccaaggacaactgcagcaaggacgccaacgataccctgctgctgcagctgacaaacaccagcgcctactacatgtacttgttgctgctgctgaagtccgtggtgtacttcgccatcatcacatgctgcctgctgcggcggaccgccttctgctgcaacggcgagaagtcc

SEQ ID NO: 4 - protein, 307 aa, Homo sapiens
  source 1..307: note = TRG full length E57 G1
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 5 - DNA, 891 bp, synthetic construct
  source 1..891: mol_type = other DNA; note = TRD codon optimised E57 D3
atggccatgctgctgggagccagcgtgctgatcctgtggctgcagcccgattgggtcaacagccagcagaagaacgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcagaggctacaccggcagcgacaagctgatcttcggcaagggcaccagagtgaccgtggaacccagaagccagccccacaccaagcccagcgtgttcgtgatgaagaacggcaccaacgtggcctgcctcgtgaaagagttctaccccaaggacatccggatcaacctggtgtccagcaagaagatcaccgagttcgaccccgccatcgtgatcagcccctccggcaagtacaacgccgtgaagctggggaagtacgaggacagcaacagcgtgacctgctccgtgcagcacgacaacaagaccgtgcacagcaccgatttcgaagtgaaaaccgactccaccgaccacgtgaagcccaaagagacagagaacaccaagcagcccagcaagagctgccacaagcccaaggccatcgtgcacaccgagaaagtgaacatgatgagcctgaccgtgctgggcctgcggatgctgttcgccaaaaccgtggccgtgaacttcctgctgaccgccaagctgttctttctg

SEQ ID NO: 6 - protein, 297 aa, Homo sapiens
  source 1..297: note = TRD full length E57 D3
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 7 - protein, 11 aa, synthetic construct
  source 1..11: note = TRG CDR3 E57 G2
CATWDGYKKLF

SEQ ID NO: 8 - protein, 12 aa, synthetic construct
  source 1..12: note = TRG CDR3 E57 G3
CATWDGYYKKLF

SEQ ID NO: 9 - protein, 12 aa, synthetic construct
  source 1..12: note = TRG CDR3 E57 G4
CATWDGFYKKLF

SEQ ID NO: 10 - protein, 13 aa, synthetic construct
  source 1..13: note = TRG CDR3 E57 G5
CATWDAFYYKKLF

SEQ ID NO: 11 - protein, 13 aa, synthetic construct
  source 1..13: note = TRG CDR3 E57 G6
CATWEGFYYKKLF

SEQ ID NO: 12 - protein, 13 aa, synthetic construct
  source 1..13: note = TRG CDR3 E57 G7
CATWEAFYYKKLF

SEQ ID NO: 13 - protein, 13 aa, synthetic construct
  source 1..13: note = TRG CDR3 E57 G8
CATWDGFFYKKLF

SEQ ID NO: 14 - protein, 13 aa, synthetic construct
  source 1..13: note = TRG CDR3 E57 G9
CATWDGYFYKKLF

SEQ ID NO: 15 - protein, 13 aa, synthetic construct
  source 1..13: note = TRG CDR3 E57 G10
CATWDGYYYKKLF

SEQ ID NO: 16 - protein, 13 aa, synthetic construct
  source 1..13: note = TRG CDR3 E57 G11
CATWDGAYYKKLF

SEQ ID NO: 17 - protein, 13 aa, synthetic construct
  source 1..13: note = TRG CDR3 E57 G12
CATWDGSYYKKLF

SEQ ID NO: 18 - protein, 13 aa, synthetic construct
  source 1..13: note = TRG CDR3 E57 G13
CATWDGHYYKKLF

SEQ ID NO: 19 - protein, 19 aa, synthetic construct
  source 1..19: note = TRD CDR3 E57 D1
CAASSPIRGYTSGSDKLIF

SEQ ID NO: 20 - protein, 18 aa, synthetic construct
  source 1..18: note = TRD CDR3 E57 D2
CAASSPIRAYTGSDKLIF

SEQ ID NO: 21 - protein, 18 aa, synthetic construct
  source 1..18: note = TRD CDR3 E57 D4
CAASSPIRGFTGSDKLIF

SEQ ID NO: 22 - protein, 18 aa, synthetic construct
  source 1..18: note = TRD CDR3 E57 D5
CAASSPIKGYTGSDKLIF

SEQ ID NO: 23 - protein, 18 aa, synthetic construct
  source 1..18: note = TRD CDR3 E57 D6
CAASSPIKGFTGSDKLIF

SEQ ID NO: 24 - protein, 18 aa, synthetic construct
  source 1..18: note = TRD CDR3 E57 D7
CAASSPLRGFTGSDKLIF

SEQ ID NO: 25 - protein, 18 aa, synthetic construct
  source 1..18: note = TRD CDR3 E57 D8
CAASSPLRGYTGSDKLIF

SEQ ID NO: 26 - protein, 18 aa, synthetic construct
  source 1..18: note = TRD CDR3 E57 D10
CAASSPLKGYTGSDKLIF

SEQ ID NO: 27 - protein, 18 aa, synthetic construct
  source 1..18: note = TRD CDR3 E57 D11
CAASSPIRAFTGSDKLIF

SEQ ID NO: 28 - DNA, 921 bp, synthetic construct
  source 1..921: mol_type = other DNA; note = sE57 G1
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacggcttctactacaagaagctgttcggcagcggcaccaccctggtggtgacagataaacaacttgatgcagatgtttcccccaagcccactatttttcttccttcaattgctgagacaaagctgcagaaggccggcacctacctgtgcctgctggaaaagttcttcccagacgtgatcaagatccactggcaggaaaagaagtccaacaccatcctgggcagccaggaagggaacaccatgaagaccaacgatacctacatgaagttcagctggctgaccgtgcccgaggaaagcctggacaaagaacacagatgcatcgtgcggcacgagaacaacaagaacggcgtggaccaggaaatcatcttcccacccatcaagaccgatgtgatcaccatggaccccaaggacaacgccagcggcctggtgcctagaggctctggctctggcagaatcgcccggctggaagagaaagtgaaaaccctgaaggcccagaacagcgagctggcctccaccgccaacatgctgagagaacaggtggcccagctgaagcagaaagtgatgaactattga

SEQ ID NO: 29 - DNA, 921 bp, synthetic construct
  source 1..921: mol_type = other DNA; note = sE57cys G1
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacggcttctactacaagaagctgttcggcagcggcaccaccctggtggtgacagataaacaacttgatgcagatgtttcccccaagcccactatttttcttccttcaattgctgagacaaagctgcagaaggccggcacctacctgtgcctgctggaaaagttcttcccagacgtgatcaagatccactggcaggaaaagaagtccaacaccatcctgggcagccaggaagggaacaccatgaagaccaacgatacctacatgaagttcagctggctgaccgtgcccgaggaaagcctggacaaagaacacagatgcatcgtgcggcacgagaacaacaagaacggcgtggaccaggaaatcatcttcccacccatcaagaccgatgtgatcaccatggaccccaaggacaactgcagcggcctggtgcctagaggctctggctctggcagaatcgcccggctggaagagaaagtgaaaaccctgaaggcccagaacagcgagctggcctccaccgccaacatgctgagagaacaggtggcccagctgaagcagaaagtgatgaactattga

SEQ ID NO: 30 - DNA, 987 bp, synthetic construct
  source 1..987: mol_type = other DNA; note = sE57 D3
atggccatgctgctgggagccagcgtgctgatcctgtggctgcagcccgattgggtcaacagccagcagaagaacgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcagaggctacaccggcagcgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagcgcctctggcctggtgcctagaggctctggcagcggcctgaccgatacactgcaggccgagacagaccagctggaagataagaagtccgccctgcagaccgagatcgccaacctgctgaaagagaaagaaaagctggagttcatcctggccgcctacggctccggcctgaacgacattttcgaggcccagaaaatcgagtggcacgagcaccaccaccatcaccactaa

SEQ ID NO: 31 - DNA, 987 bp, synthetic construct
  source 1..987: mol_type = other DNA; note = sE57cys D3
atggccatgctgctgggagccagcgtgctgatcctgtggctgcagcccgattgggtcaacagccagcagaagaacgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcagaggctacaccggcagcgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgctctggcctggtgcctagaggctctggcagcggcctgaccgatacactgcaggccgagacagaccagctggaagataagaagtccgccctgcagaccgagatcgccaacctgctgaaagagaaagaaaagctggagttcatcctggccgcctacggctccggcctgaacgacattttcgaggcccagaaaatcgagtggcacgagcaccaccaccatcaccactaa

SEQ ID NO: 32 - DNA, 921 bp, synthetic construct
  source 1..921: mol_type = other DNA; note = sE57 G5
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacgcgttctactacaagaagctgttcggcagcggcaccaccctggtggtgacagataaacaacttgatgcagatgtttcccccaagcccactatttttcttccttcaattgctgagacaaagctgcagaaggccggcacctacctgtgcctgctggaaaagttcttcccagacgtgatcaagatccactggcaggaaaagaagtccaacaccatcctgggcagccaggaagggaacaccatgaagaccaacgatacctacatgaagttcagctggctgaccgtgcccgaggaaagcctggacaaagaacacagatgcatcgtgcggcacgagaacaacaagaacggcgtggaccaggaaatcatcttcccacccatcaagaccgatgtgatcaccatggaccccaaggacaactgcagcggcctggtgcctagaggctctggctctggcagaatcgcccggctggaagagaaagtgaaaaccctgaaggcccagaacagcgagctggcctccaccgccaacatgctgagagaacaggtggcccagctgaagcagaaagtgatgaactattga

SEQ ID NO: 33 - DNA, 921 bp, synthetic construct; identical to SEQ ID NO: 32
  source 1..921: mol_type = other DNA; note = sE57cys G5

SEQ ID NO: 34 - DNA, 987 bp, synthetic construct
  source 1..987: mol_type = other DNA; note = sE57 D6
atggccatgctgctgggagccagcgtgctgatcctgtggctgcagcccgattgggtcaacagccagcagaagaacgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcaagggcttcaccggcagcgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgctctggcctggtgcctagaggctctggcagcggcctgaccgatacactgcaggccgagacagaccagctggaagataagaagtccgccctgcagaccgagatcgccaacctgctgaaagagaaagaaaagctggagttcatcctggccgcctacggctccggcctgaacgacattttcgaggcccagaaaatcgagtggcacgagcaccaccaccatcaccactaa

SEQ ID NO: 35 - DNA, 987 bp, synthetic construct; identical to SEQ ID NO: 34
  source 1..987: mol_type = other DNA; note = sE57cys D6

SEQ ID NO: 36 - DNA, 127 bp, synthetic construct
  source 1..127: mol_type = other DNA; note = c-Fos dimerization motif
tctggcagaatcgcccggctggaagagaaagtgaaaaccctgaaggcccagaacagcgagctggcctccaccgccaacatgctgagagaacaggtggcccagctgaagcagaaagtgatgaactatt

SEQ ID NO: 37 - DNA, 126 bp, synthetic construct
  source 1..126: mol_type = other DNA; note = c-Jun dimerization motif
agcggcctgaccgatacactgcaggccgagacagaccagctggaagataagaagtccgccctgcagaccgagatcgccaacctgctgaaagagaaagaaaagctggagttcatcctggccgcctac

SEQ ID NO: 38 - DNA, 45 bp, synthetic construct
  source 1..45: mol_type = other DNA; note = Avi-tag
ggcctgaacgacattttcgaggcccagaaaatcgagtggcacgag

SEQ ID NO: 39 - DNA, 18 bp, synthetic construct
  source 1..18: mol_type = other DNA; note = His-tag
caccaccaccatcaccac

SEQ ID NO: 40 - DNA, 1020 bp, synthetic construct
  source 1..1020: mol_type = other DNA; note = BirA sequence
atgggctggtcctgcatcatcctgtttctggtggccaccgccacaggcgtccactctaaggacaacaccgtgcctctgaagctgatcgccctgctggccaatggcgagtttcactctggcgagcagctgggcgaaacactgggaatgagcagagccgccatcaacaagcacatccagacactgagagactggggcgtcgacgtgttcacagtgcctggcaagggctactctctgcccgagcctatccagctgctgaacgccaagcagatcctgggacaactggatggcggatctgtggctgtgctgcccgtgatcgatagcaccaaccagtacctgctggaccggatcggcgagctgaaaagcggagatgcctgtatcgccgagtatcagcaggccggcagaggtagaagaggccggaagtggtttagccccttcggcgccaatctgtacctgagcatgttctggcggctggaacagggacctgccgctgctattggactgtctctggtcatcggcatcgtgatggccgaggtgctgagaaagctgggcgccgacaaagtgcgcgtgaagtggcccaatgacctgtatctgcaggaccggaagctggccggaatcctggttgagctgacaggcaaaacaggcgacgccgctcagatcgtgattggcgctggaatcaatatggccatgcggagagtggaagagagcgtggtcaatcaaggctggatcaccctgcaagaagccggcatcaacctggacagaaataccctggccgccatgctgatcagagagctgagagctgccctggaactgttcgagcaagaaggactggccccttacctgtccagatgggagaagctggacaacttcatcaacagacccgtcaagctgatcatcggcgacaaagagatcttcggcatcagccggggcatcgataagcaaggcgctctgctgctggaacaagacggcatcatcaagccttggatgggcggcgagatcagcctgagatctgccgagaaatga

SEQ ID NO: 41 - DNA, 48 bp, synthetic construct
  source 1..48: mol_type = other DNA; note = E57 fw
tcgtcggcagcgtcagatgtgtataagagacagcggcgtgtactactg

SEQ ID NO: 42 - DNA, 60 bp, synthetic construct
  source 1..60: mol_type = other DNA; note = E57 rv barcode 1
gtctcgtgggctcggagatgtgtataagagacagtcagtgtggtggggctggcttctggg

SEQ ID NO: 43 - DNA, 60 bp, synthetic construct
  source 1..60: mol_type = other DNA; note = E57 rv barcode 2
gtctcgtgggctcggagatgtgtataagagacagtcatgcacgtggggctggcttctggg

SEQ ID NO: 44 - DNA, 60 bp, synthetic construct
  source 1..60: mol_type = other DNA; note = E57 rv barcode 3
gtctcgtgggctcggagatgtgtataagagacagtggtggctgtggggctggcttctggg

SEQ ID NO: 45 - DNA, 60 bp, synthetic construct
  source 1..60: mol_type = other DNA; note = E57 rv barcode 4
gtctcgtgggctcggagatgtgtataagagacaggataagtggtggggctggcttctggg

SEQ ID NO: 46 - DNA, 60 bp, synthetic construct
  source 1..60: mol_type = other DNA; note = E57 rv barcode 5
gtctcgtgggctcggagatgtgtataagagacagattagacggtggggctggcttctggg

SEQ ID NO: 47 - DNA, 60 bp, synthetic construct
  source 1..60: mol_type = other DNA; note = E57 rv barcode 6
gtctcgtgggctcggagatgtgtataagagacagcacttccggtggggctggcttctggg

SEQ ID NO: 48 - DNA, 60 bp, synthetic construct
  source 1..60: mol_type = other DNA; note = E57 rv barcode 7
gtctcgtgggctcggagatgtgtataagagacaggtctgtatgtggggctggcttctggg

SEQ ID NO: 49 - DNA, 60 bp, synthetic construct
  source 1..60: mol_type = other DNA; note = E57 rv barcode 8
gtctcgtgggctcggagatgtgtataagagacagtgtcaaaggtggggctggcttctggg

SEQ ID NO: 50 - DNA, 60 bp, synthetic construct
  source 1..60: mol_type = other DNA; note = E57 rv barcode 9
gtctcgtgggctcggagatgtgtataagagacaggtgtgagagtggggctggcttctggg

SEQ ID NO: 51 - DNA, 60 bp, synthetic construct
  source 1..60: mol_type = other DNA; note = E57 rv barcode 10
gtctcgtgggctcggagatgtgtataagagacagacctcgttgtggggctggcttctggg

SEQ ID NO: 52 - DNA, 60 bp, synthetic construct
  source 1..60: mol_type = other DNA; note = E57 rv barcode 11
gtctcgtgggctcggagatgtgtataagagacagaacccatggtggggctggcttctggg

SEQ ID NO: 53 - DNA, 60 bp, synthetic construct
  source 1..60: mol_type = other DNA; note = E57 rv barcode 12
gtctcgtgggctcggagatgtgtataagagacagtcgacatcgtggggctggcttctggg

SEQ ID NO: 54 - DNA, 60 bp, synthetic construct
  source 1..60: mol_type = other DNA; note = E57 rv barcode 13
gtctcgtgggctcggagatgtgtataagagacagatgtgcttgtggggctggcttctggg

SEQ ID NO: 55 - DNA, 60 bp, synthetic construct
  source 1..60: mol_type = other DNA; note = E57 rv barcode 14
gtctcgtgggctcggagatgtgtataagagacagaatgacaggtggggctggcttctggg

SEQ ID NO: 56 - DNA, 60 bp, synthetic construct
  source 1..60: mol_type = other DNA; note = E57 rv barcode 15
gtctcgtgggctcggagatgtgtataagagacaggcttaacagtggggctggcttctggg

SEQ ID NO: 57 - DNA, 1659 bp, synthetic construct
  source 1..1659: mol_type = other DNA; note = TCR-CD3 E57 G1
atggcctgtcctggatttctgtgggccctcgtgatcagcacctgtctggaattcagcatggcccggacatccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacgggttctactacaagaagctgttcggcagcggcaccaccctggtggtgacagacaagcagctggacgccgacgtgtcccccaagcctaccatcttcctgccctcaattgctgagacaaagctgcagaaggccggcacctacctgtgcctgctggaaaagttcttcccagacgtgatcaagatccactgggaggaaaagaagtccaacaccatcctgggcagccaagaaggcaacaccatgaagaccaacgacacctacatgaagttcagctggctgaccgtgcccgagaagtccctggacaaagaacaccggtgcatcgtgcggcacgagaacaacaagaacggcgtggaccaagaaatcatcttcccacccatcaagaccgacgtgatcacaatggaccccaaggacaacagaacaggtggcggaggatctggcggaggtggaagcggcggaggcggatctcaagttcagctggttcaatctggcggcggagtggtgcagcctggcagatctcttagactgagctgcaaggccagcggctacaccttcaccagatacaccatgcactgggtccgacaggcccctggcaaaggacttgagtggatcggctacatcaaccccagccggggctacaccaactacaaccagaagttcaaggaccggttcaccatcagccgggacaacagcaagaataccgccttcctgcagatggacagcctgcggcctgaagataccggcgtgtacttctgtgcccggtactacgacgaccactactgcctggattattggggccagggcacccctgtgacagtctcttcaggcggtggtggttcaggcggcggaggaagcggaggcggaggttctgatatccagatgacacagagccccagcagcctgtctgcctctgtgggagacagagtgaccatcacctgtagcgccagcagcagcgtgtcctacatgaactggtatcagcagaccccaggcaaggcccctaagcggtggatctacgatacaagcaagctggcctctggcgtgcccagcagattttctggctctggcagcggcaccgactacacctttacaatatccagcctgcagccagaggatatcgccacctactactgccagcagtggtccagcaatcccttcacctttggacagggcaccaagctgcagatcaccagagtggatggctcaggctccggcggactgaacgatatcttcgaggcccagaaaattgagtggcacgaaggttctggtggtggcggacaccaccaccatcatcatcattga

SEQ ID NO: 58 - DNA, 756 bp, synthetic construct
  source 1..756: mol_type = other DNA; note = TCR-CD3 E57 D3
atggcctgtcctggatttctgtgggccctcgtgatcagcacctgtctggaattcagcatggccagcgccgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcagaggctacaccggcagcgacaagctgatcttcggcaagggcaccagggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctga

SEQ ID NO: 59 - DNA, 1659 bp, synthetic construct
  source 1..1659: mol_type = other DNA; note = TCR-CD3 E57 G5
atggcctgtcctggatttctgtgggccctcgtgatcagcacctgtctggaattcagcatggcccggacatccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacgcgttctactacaagaagctgttcggcagcggcaccaccctggtggtgacagacaagcagctggacgccgacgtgtcccccaagcctaccatcttcctgccctcaattgctgagacaaagctgcagaaggccggcacctacctgtgcctgctggaaaagttcttcccagacgtgatcaagatccactgggaggaaaagaagtccaacaccatcctgggcagccaagaaggcaacaccatgaagaccaacgacacctacatgaagttcagctggctgaccgtgcccgagaagtccctggacaaagaacaccggtgcatcgtgcggcacgagaacaacaagaacggcgtggaccaagaaatcatcttcccacccatcaagaccgacgtgatcacaatggaccccaaggacaacagaacaggtggcggaggatctggcggaggtggaagcggcggaggcggatctcaagttcagctggttcaatctggcggcggagtggtgcagcctggcagatctcttagactgagctgcaaggccagcggctacaccttcaccagatacaccatgcactgggtccgacaggcccctggcaaaggacttgagtggatcggctacatcaaccccagccggggctacaccaactacaaccagaagttcaaggaccggttcaccatcagccgggacaacagcaagaataccgccttcctgcagatggacagcctgcggcctgaagataccggcgtgtacttctgtgcccggtactacgacgaccactactgcctggattattggggccagggcacccctgtgacagtctcttcaggcggtggtggttcaggcggcggaggaagcggaggcggaggttctgatatccagatgacacagagccccagcagcctgtctgcctctgtgggagacagagtgaccatcacctgtagcgccagcagcagcgtgtcctacatgaactggtatcagcagaccccaggcaaggcccctaagcggtggatctacgatacaagcaagctggcctctggcgtgcccagcagattttctggctctggcagcggcaccgactacacctttacaatatccagcctgcagccagaggatatcgccacctactactgccagcagtggtccagcaatcccttcacctttggacagggcaccaagctgcagatcaccagagtggatggctcaggctccggcggactgaacgatatcttcgaggcccagaaaattgagtggcacgaaggttctggtggtggcggacaccaccaccatcatcatcattga

SEQ ID NO: 60 - DNA, 756 bp, synthetic construct
  source 1..756: mol_type = other DNA; note = TCR-CD3 E57 D6
atggcctgtcctggatttctgtgggccctcgtgatcagcacctgtctggaattcagcatggccagcgccgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcaagggcttcaccggcagcgacaagctgatcttcggcaagggcaccagggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctga

SEQ ID NO: 61 - DNA, 1653 bp, synthetic construct
  source 1..1653: mol_type = other DNA; note = TCR-CD3 E57 G2
atggcctgtcctggatttctgtgggccctcgtgatcagcacctgtctggaattcagcatggcccggacatccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacggctacaagaagctgttcggcagcggcaccaccctggtggtgacagacaagcagctggacgccgacgtgtcccccaagcctaccatcttcctgccctcaattgctgagacaaagctgcagaaggccggcacctacctgtgcctgctggaaaagttcttcccagacgtgatcaagatccactgggaggaaaagaagtccaacaccatcctgggcagccaagaaggcaacaccatgaagaccaacgacacctacatgaagttcagctggctgaccgtgcccgagaagtccctggacaaagaacaccggtgcatcgtgcggcacgagaacaacaagaacggcgtggaccaagaaatcatcttcccacccatcaagaccgacgtgatcacaatggaccccaaggacaacagaacaggtggcggaggatctggcggaggtggaagcggcggaggcggatctcaagttcagctggttcaatctggcggcggagtggtgcagcctggcagatctcttagactgagctgcaaggccagcggctacaccttcaccagatacaccatgcactgggtccgacaggcccctggcaaaggacttgagtggatcggctacatcaaccccagccggggctacaccaactacaaccagaagttcaaggaccggttcaccatcagccgggacaacagcaagaataccgccttcctgcagatggacagcctgcggcctgaagataccggcgtgtacttctgtgcccggtactacgacgaccactactgcctggattattggggccagggcacccctgtgacagtctcttcaggcggtggtggttcaggcggcggaggaagcggaggcggaggttctgatatccagatgacacagagccccagcagcctgtctgcctctgtgggagacagagtgaccatcacctgtagcgccagcagcagcgtgtcctacatgaactggtatcagcagaccccaggcaaggcccctaagcggtggatctacgatacaagcaagctggcctctggcgtgcccagcagattttctggctctggcagcggcaccgactacacctttacaatatccagcctgcagccagaggatatcgccacctactactgccagcagtggtccagcaatcccttcacctttggacagggcaccaagctgcagatcaccagagtggatggctcaggctccggcggactgaacgatatcttcgaggcccagaaaattgagtggcacgaaggttctggtggtggcggacaccaccaccatcatcatcattga

SEQ ID NO: 62 - DNA, 759 bp, synthetic construct
  source 1..759: mol_type = other DNA; note = TCR-CD3 E57 D1
atggcctgtcctggatttctgtgggccctcgtgatcagcacctgtctggaattcagcatggccagcgccgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcagaggctacaccagcggcagcgacaagctgatcttcggcaagggcaccagggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctga

SEQ ID NO: 63 - protein, 4 aa, synthetic construct
  source 1..4: note = Flexible linker
TSGS

SEQ ID NO: 64 - protein, 5 aa, synthetic construct
  source 1..5: note = Flexible linker
GGGGS

SEQ ID NO: 65 - protein, 4 aa, synthetic construct
  source 1..4: note = Flexible linker
GGGS

SEQ ID NO: 66 - protein, 18 aa, synthetic construct
  source 1..18: note = Flexible linker
KESGSVSSEQLAQFRSLD

SEQ ID NO: 67 - protein, 14 aa, synthetic construct
  source 1..14: note = Flexible linker
EGKSSGSGSESKST

SEQ ID NO: 68 - protein, 12 aa, synthetic construct
  source 1..12: note = Flexible linker
GSAGSAAGSGEF

SEQ ID NO: 69 - protein, 5 aa, synthetic construct
  source 1..5: note = Rigid linker
EAAAK

SEQ ID NO: 70 - protein, 5 aa, synthetic construct
  source 1..5: note = Rigid linker
EAAAR

SEQ ID NO: 71 - protein, 5 aa, synthetic construct
  source 1..5: note = Rigid linker
PAPAP

SEQ ID NO: 72 - protein, 12 aa, synthetic construct
  source 1..12: note = Rigid linker
AEAAAKEAAAKA

SEQ ID NO: 73 - protein, 51 aa, synthetic construct
  source 1..51: note = Rigid linker
ILTHDSSIRYLQEIYNSNNQKIVNLKEKVAQLEAQCQEPCKDTVQIHDITG

SEQ ID NO: 74 - protein, 30 aa, synthetic construct
  source 1..30: note = Flexible linker
SLNGGGGSGGGGSGGGGSGGGGSGGGGSTS

SEQ ID NO: 75 - protein, 20 aa, synthetic construct
  source 1..20: note = Flexible linker
SGGSGGGGSGGGSGGGGSLQ

SEQ ID NO: 76 - protein, 26 aa, synthetic construct
  source 1..26: note = Flexible linker
SGGGSGGGGSGGGGSGGGGSGGGSLQ

SEQ ID NO: 77 - protein, 15 aa, synthetic construct
  source 1..15: note = Flexible linker
GGGGSGGGGSGGGGS

SEQ ID NO: 78 - protein, 17 aa, synthetic construct
  source 1..17: note = linker
RTGGGGSGGGGSGGGGS

SEQ ID NO: 79 - protein, 7 aa, synthetic construct
  source 1..7: note = TRG CDR3 E57 G1
WDGFYYK

SEQ ID NO: 80 - protein, 5 aa, synthetic construct
  source 1..5: note = TRG CDR3 E57 G2
WDGYK

SEQ ID NO: 81 - protein, 6 aa, synthetic construct
  source 1..6: note = TRG CDR3 E57 G3
WDGYYK

SEQ ID NO: 82 - protein, 6 aa, synthetic construct
  source 1..6: note = TRG CDR3 E57 G4
WDGFYK

SEQ ID NO: 83 - protein, 7 aa, synthetic construct
  source 1..7: note = TRG CDR3 E57 G5
WDAFYYK

SEQ ID NO: 84 - protein, 7 aa, synthetic construct
  source 1..7: note = TRG CDR3 E57 G6
WEGFYYK

SEQ ID NO: 85 - protein, 7 aa, synthetic construct
  source 1..7: note = TRG CDR3 E57 G7
WEAFYYK

SEQ ID NO: 86 - protein, 7 aa, synthetic construct
  source 1..7: note = TRG CDR3 E57 G8
WDGFFYK

SEQ ID NO: 87 - protein, 7 aa, synthetic construct
  source 1..7: note = TRG CDR3 E57 G9
WDGYFYK

SEQ ID NO: 88 - protein, 7 aa, synthetic construct
  source 1..7: note = TRG CDR3 E57 G10
WDGYYYK

SEQ ID NO: 89 - protein, 7 aa, synthetic construct
  source 1..7: note = TRG CDR3 E57 G11
WDGAYYK

SEQ ID NO: 90 - protein, 7 aa, synthetic construct
  source 1..7: note = TRG CDR3 E57 G12
WDGSYYK

SEQ ID NO: 91 - protein, 7 aa, synthetic construct
  source 1..7: note = TRG CDR3 E57 G13
WDGHYYK

SEQ ID NO: 92 - protein, 6 aa, synthetic construct
  source 1..6: note = TRD CDR3 E57 D1
IRGYTS

SEQ ID NO: 93 - protein, 6 aa, synthetic construct
  source 1..6: note = TRD CDR3 E57 D2
IRAYTG

SEQ ID NO: 94 - protein, 6 aa, synthetic construct
  source 1..6: note = TRD CDR3 E57 D3
IRGYTG

SEQ ID NO: 95 - protein, 6 aa, synthetic construct
  source 1..6: note = TRD CDR3 E57 D4
IRGFTG

SEQ ID NO: 96 - protein, 6 aa, synthetic construct
  source 1..6: note = TRD CDR3 E57 D5
IKGYTG

SEQ ID NO: 97 - protein, 6 aa, synthetic construct
  source 1..6: note = TRD CDR3 E57 D6
IKGFTG

SEQ ID NO: 98 - protein, 6 aa, synthetic construct
  source 1..6: note = TRD CDR3 E57 D7
LRGFTG

SEQ ID NO: 99 - protein, 6 aa, synthetic construct
  source 1..6: note = TRD CDR3 E57 D8
LRGYTG

SEQ ID NO: 100 - protein, 6 aa, synthetic construct
  source 1..6: note = TRD CDR3 E57 D10
LKGYTG

SEQ ID NO: 101 - protein, 6 aa, synthetic construct
  source 1..6: note = TRD CDR3 E57 D11
IRAFTG

SEQ ID NO: 102 - protein, 23 aa, synthetic construct
  source 1..23: note = TRGC1 TM domain
YYMYLLLLLKSVVYFAIITCCLL

SEQ ID NO: 103 - protein, 23 aa, synthetic construct
  source 1..23: note = TRGC2 TM domain
YYTYLLLLLKSVVYFAIITCCLL

SEQ ID NO: 104 - protein, 23 aa, synthetic construct
  source 1..23: note = TRDC TM domain
LGLRMLFAKTVAVNFLLTAKLFF

SEQ ID NO: 105 - protein, 245 aa, synthetic construct
  source 1..245: note = OKT3 scFV
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRVDGS

SEQ ID NO: 106 - protein, 25 aa, synthetic construct
  source 1..25: note = signal pept gamma
MACPGFLWALVISTCLEFSMARTSS

SEQ ID NO: 107 - protein, 25 aa, synthetic construct
  source 1..25: note = signal pept delta
MACPGFLWALVISTCLEFSMASAAQ

SEQ ID NO: 108 - DNA, 367 bp, synthetic construct
  source 1..367: mol_type = other DNA; note = MSCV promoter
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcact

SEQ ID NO: 109 - DNA, 6539 bp, synthetic construct
  source 1..6539: mol_type = other DNA; note = pLenti 6.3
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatgcatgctgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcactagcggccctaatacgactcactatagcccggctcgaggctaggcgcgaattcgccacccggatccacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 110 - protein, 18 aa, synthetic construct
  source 1..18: note = TRD CDR3 E57 D9
CAASSPLKGFTGSDKLIF

SEQ ID NO: 111 - protein, 6 aa, synthetic construct
  source 1..6: note = TRD CDR3 E57 D9
LKGFTG

SEQ ID NO: 112 - protein, 173 aa, synthetic construct
  source 1..173: note = Cg1
DKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 113 - DNA, 13120 bp, synthetic construct
  source 1..13120: mol_type = other DNA; note = pHCAG-L2EOP
ggggaggagcggggggaggagcggggggaggagcggggggaggagcggccagaccccgaaaacgggccccccccaaaacacaccccccgggggtcgcgcgcggccctttaaagcggcggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacgactctaggaattaattcttgaagacgaaagggcctcgtgatacgcctatttttataggttaatgtcatgataataatggtttcttagggtcgaccggtcgaccggtcgaccggtcgaccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggtcgaccggtcgaccggtcgaccggtcgaccggtcgaccatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcatgaattattccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacaggcagggggcggggcccgggccccgacttcccggttcggcggtaatgagatacgagccccgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgataagctgatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacttgtttattgcagcttataatggttacaaataaagcaatagcatcacaaatttcacaaataaagcatttttttcactgcattctagttgtggtttgtccaaactcatcaatgtatcttatcatgtctggatcggtttgaagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagcgtttaaacttaagcttggtaccgagctcggatccactagtccagtgtggtggaattctgcagatatccagcacagtggcggccgctcgagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggtgaaacagactttgaattttgaccttctcaagttggcgggagacgtcgagtccaaccctgggcccatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaagggctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggaacttgtttattgcagcttataatggttacaaataaagcaatagcatcacaaatttcacaaataaagcatttttttcactgcattctagttgtggtttgtccaaactcatcaatgtatcttatcatgtctggatctctgacctgagattggcggcactgaggtagagatgcccgaacccccccgagggagcgcgggacgcggccggggagggctggggccggggagggctggggccggggagggctggggccggggagggctggggccggggagggctggggccggggagggctggggctggggaggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcaggtgtggtggatgacgtgacgtgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcgg

SEQ ID NO: 114 - protein, 21 aa, synthetic construct
  source 1..21: note = Avi-tag
GLNDIFEAQKIEWHEGSGGGG

SEQ ID NO: 115 - protein, 7 aa, synthetic construct
  source 1..7: note = His-tag
HHHHHHH

SEQ ID NO: 116 - protein, 239 aa, synthetic construct
  source 1..239: note = eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 117 - protein, 550 aa, synthetic construct
  source 1..550: note = luciferase
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 118 - protein, 545 aa, synthetic construct [sequence omitted]

SEQ ID NO: 119 - protein, 548 aa, synthetic construct
  source 1..548: note = luciferase
MEMEKEENVVYGPLPFYPIEEGSAGIQLHKYMHQYAKLGAIAFSNALTGVDISYQEYFDITCRLAEAMKNFGMKPEEHIALCSENCEEFFIPVLAGLYIGVAVAPTNEIYTLRELNHSLGIAQPTIVFSSRKGLPKVLEVQKTVTCIKKIVILDSKVNFGGHDCMETFIKKHVELGFQPSSFVPIDVKNRKQHVALLMNSSGSTGLPKGVRITHEGAVTRFSHAKDPIYGNQVSPGTAILTVVPFHHGFGMFTTLGYFACGYRVVMLTKFDEELFLRTLQDYKCTSVILVPTLFAILNKSELIDKFDLSNLTEIASGGAPLAKEVGEAVARRFNLPGVRQGYGLTETTSAFIITPEGDDKPGASGKVVPLFKVKVIDLDTKKTLGVNRRGEICVKGPSLMLGYSNNPEATRETIDEEGWLHTGDIGYYDEDEHFFIVDRLKSLIKYKGYQVPPAELESVLLQHPNIFDAGVAGVPDPDAGELPGAVVVMEKGKTMTEKEIVDYVNSQVVNHKRLRGGVRFVDEVPKGLTGKIDAKVIREILKKPQAKM

SEQ ID NO: 120 - protein, 116 aa, synthetic construct
  source 1..116: note = Variable CDR3 G1
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVT

SEQ ID NO: 121 - protein, 114 aa, synthetic construct
  source 1..114: note = Variable CDR3 G2
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVT

SEQ ID NO: 122 - protein, 115 aa, synthetic construct
  source 1..115: note = Variable CDR3 G3
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVT

SEQ ID NO: 123 - protein, 115 aa, synthetic construct
  source 1..115: note = Variable CDR3 G4
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVT

SEQ ID NO: 124 - protein, 116 aa, synthetic construct
  source 1..116: note = Variable CDR3 G5
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVT

SEQ ID NO: 125 - protein, 116 aa, synthetic construct
  source 1..116: note = Variable CDR3 G6
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVT

SEQ ID NO: 126 - protein, 116 aa, synthetic construct
  source 1..116: note = Variable CDR3 G7
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVT

SEQ ID NO: 127 - protein, 116 aa, synthetic construct
  source 1..116: note = Variable CDR3 G8
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVT

SEQ ID NO: 128 - protein, 116 aa, synthetic construct
  source 1..116: note = Variable CDR3 G9
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVT

SEQ ID NO: 129 - protein, 116 aa, synthetic construct
  source 1..116: note = Variable CDR3 G10
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVT

SEQ ID NO: 130 - protein, 116 aa, synthetic construct
  source 1..116: note = Variable CDR3 G11
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVT

SEQ ID NO: 131 - protein, 116 aa, synthetic construct
  source 1..116: note = Variable CDR3 G12
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVT

SEQ ID NO: 132 - protein, 116 aa, synthetic construct
  source 1..116: note = Variable CDR3 G13
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVT

SEQ ID NO: 133 - protein, 119 aa, synthetic construct
  source 1..119: note = Variable CDR3 D1
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPR

SEQ ID NO: 134 - protein, 118 aa, synthetic construct
  source 1..118: note = Variable CDR3 D2
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPR

SEQ ID NO: 135 - protein, 118 aa, synthetic construct
  source 1..118: note = Variable CDR3 D3
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPR

SEQ ID NO: 136 - protein, 118 aa, synthetic construct
  source 1..118: note = Variable CDR3 D4
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPR

SEQ ID NO: 137 - protein, 118 aa, synthetic construct
  source 1..118: note = Variable CDR3 D5
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPR

SEQ ID NO: 138 - protein, 118 aa, synthetic construct
  source 1..118: note = Variable CDR3 D6
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPR

SEQ ID NO: 139 - protein, 118 aa, synthetic construct
  source 1..118: note = Variable CDR3 D7
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPR

SEQ ID NO: 140 - protein, 118 aa, synthetic construct
  source 1..118: note = Variable CDR3 D8
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPR

SEQ ID NO: 141 - protein, 118 aa, synthetic construct
  source 1..118: note = Variable CDR3 D9
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPR

SEQ ID NO: 142 - protein, 118 aa, synthetic construct
  source 1..118: note = Variable CDR3 D10
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPR

SEQ ID NO: 143 - protein, 118 aa, synthetic construct
  source 1..118: note = Variable CDR3 D11
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPR

SEQ ID NO: 144 - protein, 134 aa, synthetic construct
  source 1..134: note = SP +Variable CDR3 G1
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVT

SEQ ID NO: 145 - protein, 132 aa, synthetic construct
  source 1..132: note = SP +Variable CDR3 G2
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVT

SEQ ID NO: 146 - protein, 133 aa, synthetic construct
  source 1..133: note = SP +Variable CDR3 G3
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVT

SEQ ID NO: 147 - protein, 133 aa, synthetic construct
  source 1..133: note = SP +Variable CDR3 G4
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVT

SEQ ID NO: 148 - protein, 134 aa, synthetic construct
  source 1..134: note = SP +Variable CDR3 G5
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVT

SEQ ID NO: 149 - protein, 134 aa, synthetic construct
  source 1..134: note = SP +Variable CDR3 G6
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVT

SEQ ID NO: 150 - protein, 134 aa, synthetic construct
  source 1..134: note = SP +Variable CDR3 G7
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVT

SEQ ID NO: 151 - protein, 134 aa, synthetic construct
  source 1..134: note = SP +Variable CDR3 G8
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVT

SEQ ID NO: 152 - protein, 134 aa, synthetic construct
  source 1..134: note = SP +Variable CDR3 G9
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVT

SEQ ID NO: 153 - protein, 134 aa, synthetic construct
  source 1..134: note = SP +Variable CDR3 G10
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVT

SEQ ID NO: 154 - protein, 134 aa, synthetic construct
  source 1..134: note = SP +Variable CDR3 G11
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVT

SEQ ID NO: 155 - protein, 134 aa, synthetic construct
  source 1..134: note = SP +Variable CDR3 G12
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVT

SEQ ID NO: 156 - protein, 134 aa, synthetic construct
  source 1..134: note = SP +Variable CDR3 G13
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVT

SEQ ID NO: 157 - protein, 145 aa, synthetic construct
  source 1..145: note = SP +Variable CDR3 D1
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPR

SEQ ID NO: 158 - protein, 144 aa, synthetic construct
  source 1..144: note = SP +Variable CDR3 D2
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPR

SEQ ID NO: 159 - protein, 144 aa, synthetic construct
  source 1..144: note = SP +Variable CDR3 D3
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPR

SEQ ID NO: 160 - protein, 144 aa, synthetic construct
  source 1..144: note = SP +Variable CDR3 D4
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPR

SEQ ID NO: 161 - protein, 144 aa, synthetic construct
  source 1..144: note = SP +Variable CDR3 D5
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPR

SEQ ID NO: 162 - protein, 144 aa, synthetic construct
  source 1..144: note = SP +Variable CDR3 D6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPR

SEQ ID NO: 163 - protein, 144 aa, synthetic construct
  source 1..144: note = SP +Variable CDR3 D7
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPR

SEQ ID NO: 164 - protein, 144 aa, synthetic construct
  source 1..144: note = SP +Variable CDR3 D8
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPR

SEQ ID NO: 165 - protein, 144 aa, synthetic construct
  source 1..144: note = SP +Variable CDR3 D9
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPR

SEQ ID NO: 166 - protein, 144 aa, synthetic construct
  source 1..144: note = SP +Variable CDR3 D10
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPR

SEQ ID NO: 167 - protein, 144 aa, synthetic construct
  source 1..144: note = SP +Variable CDR3 D11
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPR

SEQ ID NO: 168 - protein, 236 aa, synthetic construct
  source 1..236: note = Variable + Constant (sTCR) G1
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 169 - protein, 234 aa, synthetic construct
  source 1..234: note = Variable + Constant (sTCR) G2
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 170 - protein, 235 aa, synthetic construct
  source 1..235: note = Variable + Constant (sTCR) G3
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 171 - protein, 235 aa, synthetic construct
  source 1..235: note = Variable + Constant (sTCR) G4
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 172 - protein, 236 aa, synthetic construct
  source 1..236: note = Variable + Constant (sTCR) G5
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 173 - protein, 236 aa, synthetic construct
  source 1..236: note = Variable + Constant (sTCR) G6
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 174 - protein, 236 aa, synthetic construct
  source 1..236: note = Variable + Constant (sTCR) G7
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 175 - protein, 236 aa, synthetic construct
  source 1..236: note = Variable + Constant (sTCR) G8
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 176 - protein, 236 aa, synthetic construct
  source 1..236: note = Variable + Constant (sTCR) G9
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 177 - protein, 236 aa, synthetic construct
  source 1..236: note = Variable + Constant (sTCR) G10
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 178 - protein, 236 aa, synthetic construct
  source 1..236: note = Variable + Constant (sTCR) G11
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 179 - protein, 236 aa, synthetic construct
  source 1..236: note = Variable + Constant (sTCR) G12
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 180 - protein, 236 aa, synthetic construct
  source 1..236: note = Variable + Constant (sTCR) G13
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 181 - protein, 228 aa, synthetic construct
  source 1..228: note = Variable +Constant (sTCR) D1
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 182 - protein, 227 aa, synthetic construct
  source 1..227: note = Variable +Constant (sTCR) D2
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 183 - protein, 227 aa, synthetic construct
  source 1..227: note = Variable +Constant (sTCR) D3
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 184 - protein, 227 aa, synthetic construct
  source 1..227: note = Variable +Constant (sTCR) D4
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 185 - protein, 227 aa, synthetic construct
  source 1..227: note = Variable +Constant (sTCR) D5
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 186 - protein, 227 aa, synthetic construct
  source 1..227: note = Variable +Constant (sTCR) D6
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 187 - protein, 227 aa, synthetic construct
  source 1..227: note = Variable +Constant (sTCR) D7
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 188 - protein, 227 aa, synthetic construct
  source 1..227: note = Variable +Constant (sTCR) D8
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 189 - protein, 227 aa, synthetic construct
  source 1..227: note = Variable +Constant (sTCR) D9
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 190 - protein, 227 aa, synthetic construct
  source 1..227: note = Variable +Constant (sTCR) D10
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 191 - protein, 227 aa, synthetic construct
  source 1..227: note = Variable +Constant (sTCR) D11
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 192 - protein, 238 aa, synthetic construct
  source 1..238: note = Variable + Constant (sTCR) cystein G1
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 193 - protein, 236 aa, synthetic construct
  source 1..236: note = Variable + Constant (sTCR) cystein G2
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 194 - protein, 237 aa, synthetic construct
  source 1..237: note = Variable + Constant (sTCR) cystein G3
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 195 - protein, 237 aa, synthetic construct
  source 1..237: note = Variable + Constant (sTCR) cystein G4
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 196 - protein, 238 aa, synthetic construct
  source 1..238: note = Variable + Constant (sTCR) cystein G5
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 197 - protein, 238 aa, synthetic construct
  source 1..238: note = Variable + Constant (sTCR) cystein G6
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 198 - protein, 238 aa, synthetic construct
  source 1..238: note = Variable + Constant (sTCR) cystein G7
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 199 - protein, 238 aa, synthetic construct
  source 1..238: note = Variable + Constant (sTCR) cystein G8
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 200 - protein, 238 aa, synthetic construct
  source 1..238: note = Variable + Constant (sTCR) cystein G9
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 201 - protein, 238 aa, synthetic construct
  source 1..238: note = Variable + Constant (sTCR) cystein G10
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 202 - protein, 238 aa, synthetic construct
  source 1..238: note = Variable + Constant (sTCR) cystein G11
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 203 - protein, 238 aa, synthetic construct
  source 1..238: note = Variable + Constant (sTCR) cystein G12
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 204 - protein, 238 aa, synthetic construct
  source 1..238: note = Variable + Constant (sTCR) cystein G13
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 205 - protein, 229 aa, synthetic construct
  source 1..229: note = Variable +Constant (sTCR) cystein D1
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 206 - protein, 228 aa, synthetic construct
  source 1..228: note = Variable +Constant (sTCR) cystein D2
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 207 - protein, 228 aa, synthetic construct
  source 1..228: note = Variable +Constant (sTCR) cystein D3
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 208 - protein, 228 aa, synthetic construct
  source 1..228: note = Variable +Constant (sTCR) cystein D4
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 209 - protein, 228 aa, synthetic construct
  source 1..228: note = Variable +Constant (sTCR) cystein D5
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 210 - protein, 228 aa, synthetic construct
  source 1..228: note = Variable +Constant (sTCR) cystein D6
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 211 - protein, 228 aa, synthetic construct
  source 1..228: note = Variable +Constant (sTCR) cystein D7
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 212 - protein, 228 aa, synthetic construct
  source 1..228: note = Variable +Constant (sTCR) cystein D8
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 213 - protein, 228 aa, synthetic construct
  source 1..228: note = Variable +Constant (sTCR) cystein D9
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 214 - protein, 228 aa, synthetic construct
  source 1..228: note = Variable +Constant (sTCR) cystein D10
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 215 - protein, 228 aa, synthetic construct
  source 1..228: note = Variable +Constant (sTCR) cystein D11
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 216 - protein, 254 aa, synthetic construct
  source 1..254: note = Extracellular (sTCR) G1
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 217 - protein, 252 aa, synthetic construct
  source 1..252: note = Extracellular (sTCR) G2
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 218 - protein, 253 aa, synthetic construct
  source 1..253: note = Extracellular (sTCR) G3
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 219 - protein, 253 aa, synthetic construct
  source 1..253: note = Extracellular (sTCR) G4
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 220 - protein, 254 aa, synthetic construct
  source 1..254: note = Extracellular (sTCR) G5
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 221 - protein, 254 aa, synthetic construct
  source 1..254: note = Extracellular (sTCR) G6
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 222 - protein, 254 aa, synthetic construct
  source 1..254: note = Extracellular (sTCR) G7
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 223 - protein, 254 aa, synthetic construct
  source 1..254: note = Extracellular (sTCR) G8
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 224 - protein, 254 aa, synthetic construct
  source 1..254: note = Extracellular (sTCR) G9
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 225 - protein, 254 aa, synthetic construct
  source 1..254: note = Extracellular (sTCR) G10
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 226 - protein, 254 aa, synthetic construct
  source 1..254: note = Extracellular (sTCR) G11
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 227 - protein, 254 aa, synthetic construct
  source 1..254: note = Extracellular (sTCR) G12
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 228 - protein, 254 aa, synthetic construct
  source 1..254: note = Extracellular (sTCR) G13
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 229 - protein, 254 aa, synthetic construct
  source 1..254: note = Extracellular (sTCR) D1
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 230 - protein, 253 aa, synthetic construct
  source 1..253: note = Extracellular (sTCR) D2
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 231 - protein, 253 aa, synthetic construct
  source 1..253: note = Extracellular (sTCR) D3
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 232 - protein, 253 aa, synthetic construct
  source 1..253: note = Extracellular (sTCR) D4
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 233 - protein, 253 aa, synthetic construct
  source 1..253: note = Extracellular (sTCR) D5
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 234 - protein, 253 aa, synthetic construct
  source 1..253: note = Extracellular (sTCR) D6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 235 - protein, 253 aa, synthetic construct
  source 1..253: note = Extracellular (sTCR) D7
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 236 - protein, 253 aa, synthetic construct
  source 1..253: note = Extracellular (sTCR) D8
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 237 - protein, 253 aa, synthetic construct
  source 1..253: note = Extracellular (sTCR) D9
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 238 - protein, 253 aa, synthetic construct
  source 1..253: note = Extracellular (sTCR) D10
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 239 - protein, 253 aa, synthetic construct
  source 1..253: note = Extracellular (sTCR) D11
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 240 - protein, 256 aa, synthetic construct
  source 1..256: note = Extracellular (sTCR) cystein G1
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 241 - protein, 254 aa, synthetic construct
  source 1..254: note = Extracellular (sTCR) cystein G2
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 242 - protein, 255 aa, synthetic construct
  source 1..255: note = Extracellular (sTCR) cystein G3
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 243 - protein, 255 aa, synthetic construct
  source 1..255: note = Extracellular (sTCR) cystein G4
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 244 - protein, 256 aa, synthetic construct
  source 1..256: note = Extracellular (sTCR) cystein G5
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 245 - protein, 256 aa, synthetic construct
  source 1..256: note = Extracellular (sTCR) cystein G6
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 246 - protein, 256 aa, synthetic construct
  source 1..256: note = Extracellular (sTCR) cystein G7
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 247 - protein, 256 aa, synthetic construct
  source 1..256: note = Extracellular (sTCR) cystein G8
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 248 - protein, 256 aa, synthetic construct
  source 1..256: note = Extracellular (sTCR) cystein G9
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 249 - protein, 256 aa, synthetic construct
  source 1..256: note = Extracellular (sTCR) cystein G10
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 250 - protein, 256 aa, synthetic construct
  source 1..256: note = Extracellular (sTCR) cystein G11
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 251 - protein, 256 aa, synthetic construct
  source 1..256: note = Extracellular (sTCR) cystein G12
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 252 - protein, 256 aa, synthetic construct
  source 1..256: note = Extracellular (sTCR) cystein G13
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCS

SEQ ID NO: 253 - protein, 255 aa, synthetic construct
  source 1..255: note = Extracellular (sTCR) cystein D1
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 254 - protein, 254 aa, synthetic construct
  source 1..254: note = Extracellular (sTCR) cystein D2
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 255 - protein, 254 aa, synthetic construct
  source 1..254: note = Extracellular (sTCR) cystein D3
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 256 - protein, 254 aa, synthetic construct
  source 1..254: note = Extracellular (sTCR) cystein D4
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 257 - protein, 254 aa, synthetic construct
  source 1..254: note = Extracellular (sTCR) cystein D5
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 258 - protein, 254 aa, synthetic construct
  source 1..254: note = Extracellular (sTCR) cystein D6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 259 - protein, 254 aa, synthetic construct
  source 1..254: note = Extracellular (sTCR) cystein D7
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 260 - protein, 254 aa, synthetic construct
  source 1..254: note = Extracellular (sTCR) cystein D8
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 261 - protein, 254 aa, synthetic construct
  source 1..254: note = Extracellular (sTCR) cystein D9
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 262 - protein, 254 aa, synthetic construct
  source 1..254: note = Extracellular (sTCR) cystein D10
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 263 - protein, 254 aa, synthetic construct
  source 1..254: note = Extracellular (sTCR) cystein D11
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSC

SEQ ID NO: 264 - protein, 236 aa, synthetic construct; identical to SEQ ID NO: 168
  source 1..236: note = Variable +Constant CD3-TCR engager G1

SEQ ID NO: 265 - protein, 234 aa, synthetic construct; identical to SEQ ID NO: 169
  source 1..234: note = Variable +Constant CD3-TCR engager G2

SEQ ID NO: 266 - protein, 235 aa, synthetic construct; identical to SEQ ID NO: 170
  source 1..235: note = Variable +Constant CD3-TCR engager G3

SEQ ID NO: 267 - protein, 235 aa, synthetic construct; identical to SEQ ID NO: 171
  source 1..235: note = Variable +Constant CD3-TCR engager G4

SEQ ID NO: 268 - protein, 236 aa, synthetic construct; identical to SEQ ID NO: 172
  source 1..236: note = Variable +Constant CD3-TCR engager G5

SEQ ID NO: 269 - protein, 236 aa, synthetic construct; identical to SEQ ID NO: 173
  source 1..236: note = Variable +Constant CD3-TCR engager G6

SEQ ID NO: 270 - protein, 236 aa, synthetic construct; identical to SEQ ID NO: 174
  source 1..236: note = Variable +Constant CD3-TCR engager G7

SEQ ID NO: 271 - protein, 236 aa, synthetic construct; identical to SEQ ID NO: 175
  source 1..236: note = Variable +Constant CD3-TCR engager G8

SEQ ID NO: 272 - protein, 236 aa, synthetic construct; identical to SEQ ID NO: 176
  source 1..236: note = Variable +Constant CD3-TCR engager G9

SEQ ID NO: 273 - protein, 236 aa, synthetic construct; identical to SEQ ID NO: 177
  source 1..236: note = Variable +Constant CD3-TCR engager G10

SEQ ID NO: 274 - protein, 236 aa, synthetic construct; identical to SEQ ID NO: 178
  source 1..236: note = Variable +Constant CD3-TCR engager G11

SEQ ID NO: 275 - protein, 236 aa, synthetic construct; identical to SEQ ID NO: 179
  source 1..236: note = Variable +Constant CD3-TCR engager G12

SEQ ID NO: 276 - protein, 236 aa, synthetic construct; identical to SEQ ID NO: 180
  source 1..236: note = Variable +Constant CD3-TCR engager G13

SEQ ID NO: 277 - protein, 228 aa, synthetic construct; identical to SEQ ID NO: 181
  source 1..228: note = Variable +Constant CD3-TCR engager D1

SEQ ID NO: 278 - protein, 227 aa, synthetic construct; identical to SEQ ID NO: 182
  source 1..227: note = Variable +Constant CD3-TCR engager D2

SEQ ID NO: 279 - protein, 227 aa, synthetic construct; identical to SEQ ID NO: 183
  source 1..227: note = Variable +Constant CD3-TCR engager D3

SEQ ID NO: 280 - protein, 227 aa, synthetic construct; identical to SEQ ID NO: 184
  source 1..227: note = Variable +Constant CD3-TCR engager D4

SEQ ID NO: 281 - protein, 227 aa, synthetic construct; identical to SEQ ID NO: 185
  source 1..227: note = Variable +Constant CD3-TCR engager D5

SEQ ID NO: 282 - protein, 227 aa, synthetic construct; identical to SEQ ID NO: 186
  source 1..227: note = Variable +Constant CD3-TCR engager D6

SEQ ID NO: 283 - protein, 227 aa, synthetic construct; identical to SEQ ID NO: 187
  source 1..227: note = Variable +Constant CD3-TCR engager D7

SEQ ID NO: 284 - protein, 227 aa, synthetic construct; identical to SEQ ID NO: 188
  source 1..227: note = Variable +Constant CD3-TCR engager D8

SEQ ID NO: 285 - protein, 227 aa, synthetic construct; identical to SEQ ID NO: 189
  source 1..227: note = Variable +Constant CD3-TCR engager D9

SEQ ID NO: 286 - protein, 227 aa, synthetic construct; identical to SEQ ID NO: 190
  source 1..227: note = Variable +Constant CD3-TCR engager D10

SEQ ID NO: 287 - protein, 227 aa, synthetic construct; identical to SEQ ID NO: 191
  source 1..227: note = Variable +Constant CD3-TCR engager D11

SEQ ID NO: 288 - protein, 259 aa, synthetic construct
  source 1..259: note = Extracellular CD3-TCR engager G1
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 289 - protein, 257 aa, synthetic construct
  source 1..257: note = Extracellular CD3-TCR engager G2
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 290 - protein, 258 aa, synthetic construct
  source 1..258: note = Extracellular CD3-TCR engager G3
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 291 - protein, 258 aa, synthetic construct
  source 1..258: note = Extracellular CD3-TCR engager G4
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 292 - protein, 259 aa, synthetic construct
  source 1..259: note = Extracellular CD3-TCR engager G5
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 293 - protein, 259 aa, synthetic construct
  source 1..259: note = Extracellular CD3-TCR engager G6
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 294 - protein, 259 aa, synthetic construct
  source 1..259: note = Extracellular CD3-TCR engager G7
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 295 - protein, 259 aa, synthetic construct
  source 1..259: note = Extracellular CD3-TCR engager G8
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 296 - protein, 259 aa, synthetic construct
  source 1..259: note = Extracellular CD3-TCR engager G9
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 297 - protein, 259 aa, synthetic construct
  source 1..259: note = Extracellular CD3-TCR engager G10
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 298 - protein, 259 aa, synthetic construct
  source 1..259: note = Extracellular CD3-TCR engager G11
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 299 - protein, 259 aa, synthetic construct
  source 1..259: note = Extracellular CD3-TCR engager G12
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 300 - protein, 259 aa, synthetic construct
  source 1..259: note = Extracellular CD3-TCR engager G13
MACPGFLWALVISTCLEFSMARTSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 301 - protein, 252 aa, synthetic construct
  source 1..252: note = Extracellular CD3-TCR engager D1
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 302 - protein, 251 aa, synthetic construct
  source 1..251: note = Extracellular CD3-TCR engager D2
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 303 - protein, 251 aa, synthetic construct
  source 1..251: note = Extracellular CD3-TCR engager D3
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 304 - protein, 251 aa, synthetic construct
  source 1..251: note = Extracellular CD3-TCR engager D4
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 305 - protein, 251 aa, synthetic construct
  source 1..251: note = Extracellular CD3-TCR engager D5
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 306 - protein, 251 aa, synthetic construct
  source 1..251: note = Extracellular CD3-TCR engager D6
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 307 - protein, 251 aa, synthetic construct
  source 1..251: note = Extracellular CD3-TCR engager D7
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 308 - protein, 251 aa, synthetic construct
  source 1..251: note = Extracellular CD3-TCR engager D8
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 309 - protein, 251 aa, synthetic construct
  source 1..251: note = Extracellular CD3-TCR engager D9
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 310 - protein, 251 aa, synthetic construct
  source 1..251: note = Extracellular CD3-TCR engager D10
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 311 - protein, 251 aa, synthetic construct
  source 1..251: note = Extracellular CD3-TCR engager D11
MACPGFLWALVISTCLEFSMASADDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 312 - protein, 258 aa, synthetic construct
  source 1..258: note = Extracellular E57 G1
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 313 - protein, 256 aa, synthetic construct
  source 1..256: note = Extracellular E57 G2
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 314 - protein, 257 aa, synthetic construct
  source 1..257: note = Extracellular E57 G3
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 315 - protein, 257 aa, synthetic construct
  source 1..257: note = Extracellular E57 G4
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 316 - protein, 258 aa, synthetic construct
  source 1..258: note = Extracellular E57 G5
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 317 - protein, 258 aa, synthetic construct
  source 1..258: note = Extracellular E57 G6
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 318 - protein, 258 aa, synthetic construct
  source 1..258: note = Extracellular E57 G7
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 319 - protein, 258 aa, synthetic construct
  source 1..258: note = Extracellular E57 G8
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 320 - protein, 258 aa, synthetic construct
  source 1..258: note = Extracellular E57 G9
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 321 - protein, 258 aa, synthetic construct
  source 1..258: note = Extracellular E57 G10
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 322 - protein, 258 aa, synthetic construct
  source 1..258: note = Extracellular E57 G11
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 323 - protein, 258 aa, synthetic construct
  source 1..258: note = Extracellular E57 G12
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 324 - protein, 258 aa, synthetic construct
  source 1..258: note = Extracellular E57 G13
SSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMY

SEQ ID NO: 325 - protein, 248 aa, synthetic construct
  source 1..248: note = Extracellular E57 D1
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 326 - protein, 247 aa, synthetic construct
  source 1..247: note = Extracellular E57 D2
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 327 - protein, 247 aa, synthetic construct
  source 1..247: note = Extracellular E57 D3
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 328 - protein, 247 aa, synthetic construct
  source 1..247: note = Extracellular E57 D4
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 329 - protein, 247 aa, synthetic construct
  source 1..247: note = Extracellular E57 D5
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 330 - protein, 247 aa, synthetic construct
  source 1..247: note = Extracellular E57 D6
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 331 - protein, 247 aa, synthetic construct
  source 1..247: note = Extracellular E57 D7
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 332 - protein, 247 aa, synthetic construct
  source 1..247: note = Extracellular E57 D8
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 333 - protein, 247 aa, synthetic construct
  source 1..247: note = Extracellular E57 D9
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 334 - protein, 247 aa, synthetic construct
  source 1..247: note = Extracellular E57 D10
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 335 - protein, 247 aa, synthetic construct
  source 1..247: note = Extracellular E57 D11
DQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 336 - protein, 305 aa, synthetic construct
  source 1..305: note = Complete E57 G2
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 337 - protein, 306 aa, synthetic construct
  source 1..306: note = Complete E57 G3
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 338 - protein, 306 aa, synthetic construct
  source 1..306: note = Complete E57 G4
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 339 - protein, 307 aa, synthetic construct
  source 1..307: note = Complete E57 G5
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 340 - protein, 307 aa, synthetic construct
  source 1..307: note = Complete E57 G6
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 341 - protein, 307 aa, synthetic construct
  source 1..307: note = Complete E57 G7
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWEAFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 342 - protein, 307 aa, synthetic construct
  source 1..307: note = Complete E57 G8
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 343 - protein, 307 aa, synthetic construct
  source 1..307: note = Complete E57 G9
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYFYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 344 - protein, 307 aa, synthetic construct
  source 1..307: note = Complete E57 G10
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGYYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 345 - protein, 307 aa, synthetic construct
  source 1..307: note = Complete E57 G11
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGAYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 346 - protein, 307 aa, synthetic construct
  source 1..307: note = Complete E57 G12
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGSYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 347 - protein, 307 aa, synthetic construct
  source 1..307: note = Complete E57 G13
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGHYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 348 - protein, 298 aa, synthetic construct
  source 1..298: note = Complete E57 D1
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTSGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 349 - protein, 297 aa, synthetic construct
  source 1..297: note = Complete E57 D2
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 350 - protein, 297 aa, synthetic construct
  source 1..297: note = Complete E57 D4
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 351 - protein, 297 aa, synthetic construct
  source 1..297: note = Complete E57 D5
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 352 - protein, 297 aa, synthetic construct
  source 1..297: note = Complete E57 D6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 353 - protein, 297 aa, synthetic construct
  source 1..297: note = Complete E57 D7
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 354 - protein, 297 aa, synthetic construct
  source 1..297: note = Complete E57 D8
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 355 - protein, 297 aa, synthetic construct
  source 1..297: note = Complete E57 D9
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 356 - protein, 297 aa, synthetic construct
  source 1..297: note = Complete E57 D10
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPLKGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 357 - protein, 297 aa, synthetic construct
  source 1..297: note = Complete E57 D11
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRAFTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 358 - protein, 6 aa, synthetic construct
  source 1..6: note = linker
GSGGGG

SEQ ID NO: 359 - protein, 12 aa, synthetic construct
  source 1..12: note = linker
ASGLVPRGSGSG

SEQ ID NO: 360 - protein, 10 aa, synthetic construct
  source 1..10: note = linker
GLVPRGSGSG

SEQ ID NO: 361 - protein, 11 aa, synthetic construct
  source 1..11: note = linker
SGLVPRGSGSG

SEQ ID NO: 362 - protein, 40 aa, synthetic construct
  source 1..40: note = c-Jun dimerization motif
RIARLEEKVKTLKAQNSELASTANMLREQVAQLKQKVMNY

SEQ ID NO: 363 - protein, 39 aa, synthetic construct
  source 1..39: note = c-Fos dimerization motif
LTDTLQAETDQLEDKKSALQTEIANLLKEKEKLEFILAA

SEQ ID NO: 364 - protein, 18 aa, synthetic construct
  source 1..18: note = t2A sequence
EGRGSLLTCGDVEENPGP

SEQ ID NO: 365 - protein, 15 aa, synthetic construct
  source 1..15: note = Avi-tag
GLNDIFEAQKIEWHE

SEQ ID NO: 366 - protein, 6 aa, synthetic construct
  source 1..6: note = 6x His-tag
HHHHHH

SEQ ID NO: 367 - protein, 7 aa, synthetic construct
  source 1..7: note = 7x His-tag
HHHHHHH

SEQ ID NO: 368 - protein, 339 aa, synthetic construct
  source 1..339: note = BirA sequence
MGWSCIILFLVATATGVHSKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 369 - protein, 5 aa, synthetic construct
  source 1..5: note = E57 G5
DAFYY

SEQ ID NO: 370 - protein, 5 aa, synthetic construct
  source 1..5: note = E57 G7
EAFYY

SEQ ID NO: 371 - protein, 5 aa, synthetic construct
  source 1..5: note = E57 G9
DGYFY

SEQ ID NO: 372 - protein, 5 aa, synthetic construct
  source 1..5: note = E57 G10
DGYYY

SEQ ID NO: 373 - protein, 5 aa, synthetic construct
  source 1..5: note = E57 G11
DGAYY

SEQ ID NO: 374 - protein, 5 aa, synthetic construct
  source 1..5: note = E57 G12
DGSYY

SEQ ID NO: 375 - protein, 6 aa, synthetic construct
  source 1..6: note = E57 gamma CDR1
EGSTGY

SEQ ID NO: 376 - protein, 8 aa, synthetic construct
  source 1..8: note = E57 gamma CDR2
YDSYTSSV

SEQ ID NO: 377 - protein, 6 aa, synthetic construct
  source 1..6: note = E57 delta CDR1
NSMFDY

SEQ ID NO: 378 - protein, 7 aa, synthetic construct
  source 1..7: note = E57 delta CDR2
ISSIKDK

SEQ ID NO: 379 - protein, 11 aa, synthetic construct
  source 1..11: note = gamma CDR3
CATWEGYKKLF

SEQ ID NO: 380 - protein, 18 aa, synthetic construct
  source 1..18: note = delta CDR3
CAASSPIEGSKWSDKLIF

SEQ ID NO: 381 - protein, 189 aa, synthetic construct
  source 1..189: note = Cg2
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 382 - protein, 189 aa, synthetic construct
  source 1..189: note = Cg2
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 383 - protein, 153 aa, synthetic construct
  source 1..153: note = Cd
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 384 - protein, 5 aa, synthetic construct
  source 1..5: note = linker
GGGSG

SEQ ID NO: 385 - protein, 5 aa, synthetic construct
  source 1..5: note = linker
GGSGG

SEQ ID NO: 386 - protein, 5 aa, synthetic construct
  source 1..5: note = linker
GSGGG

SEQ ID NO: 387 - protein, 6 aa, synthetic construct
  source 1..6: note = linker
GSGGGP

SEQ ID NO: 388 - protein, 5 aa, synthetic construct
  source 1..5: note = linker
GGEPS

SEQ ID NO: 389 - protein, 7 aa, synthetic construct
  source 1..7: note = linker
GGEGGGP

SEQ ID NO: 390 - protein, 12 aa, synthetic construct
  source 1..12: note = linker
GGEGGGSEGGGS

(19) United States
(12) Patent Application Publication
(10) Pub. No.: US 2024/0075068 A1
(43) Pub. Date: Mar. 7, 2024

(54) Novel soluble gamma T-cell (or soluble delta T-cell) receptor chains (or soluble gammadelta T-cell receptors) or fragments thereof that mediate an anti-tumour or an anti-infective response

(71) Applicant: Gadeta B.V., Utrecht (NL)

(72) Inventors: Thijs Van Montfort, Utrecht (NL); Haakan Norell, Vallentuna (SE)

(21) Appl. No.: 18/353,565

(22) Filed: Jul. 17, 2023

(30) Foreign Application Priority Data
Jul. 15, 2022 (EP) EP22185217.1

(51) Int. Cl. A61K 35/17; A61K 39/00; A61P 35/00; C07K 16/28

(52) U.S. Cl. CPC A61K 35/17; A61K 39/4611; A61K 39/4632; A61K 39/464429; A61P 35/00; C07K 16/2809; C07K 16/2896; A61K 2239/13

(57) ABSTRACT

Novel soluble γT-cell receptor chains, soluble δT-cell receptor chains, soluble γδTCRs, or fragments thereof, mediating anti-tumour responses or anti-infective responses are provided.

### CROSS-REFERENCE TO RELATED APPLICATION

This application claims the benefit of EP Provisional Application No. EP22185217.1 filed Jul. 15, 2022, the disclosure of which is hereby incorporated by reference in its entirety.

### INCORPORATION BY REFERENCE OF SEQUENCE LISTING

This application contains a Sequence listing that has been submitted in computer readable (XML) format via EFS-Web and is hereby incorporated by reference in its entirety. The XML copy, created on Jul. 11, 2023, is named “P6109415US_Sequences_20230711.xml” and is 533,000 bytes in size.

### FIELD OF THE INVENTION

The present invention relates to soluble γT-cell receptor chains, soluble δT-cell receptor chains, soluble γδT-cell receptors, fragments thereof, and cells expressing them. The invention is useful for anti-tumour and anti-infective therapeutics.

### BACKGROUND OF THE INVENTION

Our immune system utilizes different lines of defence to protect us from infections as well as cancer. In order to cover the magnitude of potential invaders and internal threats our adaptive immune system has the possibility to raise up to 10¹⁶ αβTCR combinations as well as 10¹¹ variations in immunoglobulins (Chien Y H, et al, 2014. Annu. Rev. Immunol.).

Among all immune receptor chains, TCRδs have even the highest potential diversity in the CDR3 loop (approximately 10¹⁶ combinations for murine TCRδ) owing to the presence of multiple D gene segments (two in mice, three in human, and up to five in cattle) that can join together. Each D gene segment can be read in all three open reading frames, and N nucleotides can be inserted into the junctions of the joining segments. Thus, despite the limited diversity at the VJ junctions of TCR γ-chains, the potential diversity generated at the combined CDR3 junctions (approximately 10¹⁸ combinations) is still higher than that of αβTCRs (˜10¹⁶) and immunoglobulins (˜10¹¹). (Chien Y H et al, 2014. Annu Rev. Immunol.). TCRδ and TCRγ chains may be particularly useful for immunotherapeutics against cancer and infections. Accordingly, there is still a need for identifying new γT- and δT-cell receptor chains, and γδT-cell receptors, that will mediate an anti-tumour response. There is still a need for identifying new γT- and δT-cell receptor chains, and γδT-cell receptors, that will mediate an anti-infective response. There is still a need for improved treatments utilizing γT- and δT-cell receptor chains, and γδT-cell receptors.

### SUMMARY OF THE INVENTION

In a first aspect, there is provided a soluble γT-cell receptor chain or a fragment thereof comprising a γCDR3 region, wherein said γCDR3 region is represented by an amino acid sequence comprising at least 60% sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 4-10 of SEQ ID NO: 1. In some embodiments, the soluble γT-cell receptor chain or fragment thereof comprises a modification in the γCDR3 region relative to SEQ ID NO: 1 at an amino acid position corresponding to a position selected from one or more of positions 5-9 of SEQ ID NO: 1. In some embodiments, the modification is an amino acid substitution.

In some embodiments, the soluble γT-cell receptor chain or fragment thereof comprises an amino acid substitution in the γCDR3 region relative to SEQ ID NO: 1 at the amino acid position corresponding to position 5 of SEQ ID NO: 1, preferably a substitution of an aspartic acid by a glutamic acid.

In some embodiments, the soluble γT-cell receptor chain or fragment thereof comprises an amino acid substitution in the γCDR3 region relative to SEQ ID NO: 1 at the amino acid position corresponding to position 6 of SEQ ID NO: 1, preferably a substitution of a glycine by an alanine.

In some embodiments, the soluble γT-cell receptor chain or fragment thereof comprises an amino acid substitution in the γCDR3 region relative to SEQ ID NO: 1 at the amino acid position corresponding to position 7 of SEQ ID NO: 1, preferably a substitution of a phenylalanine by an alanine, serine, or tyrosine.

In some embodiments, the soluble γT-cell receptor chain or fragment thereof comprises an amino acid substitution in the γCDR3 region relative to SEQ ID NO: 1 at the amino acid position corresponding to position 8 of SEQ ID NO: 1, preferably a substitution of a tyrosine by a phenylalanine.

In some embodiments, the soluble γT-cell receptor chain or fragment thereof comprises an amino acid substitution in the γCDR3 region relative to SEQ ID NO: 1 at the amino acid position corresponding to position 9 of SEQ ID NO: 1.

In some embodiments, the γCDR3 region comprises an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374) at the amino acid positions corresponding to positions 5-9 of SEQ ID NO: 1.

In some embodiments, the γCDR3 region comprises an amino acid sequence selected from the group consisting of WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), and WDGSYYK (SEQ ID NO: 90) at the amino acid positions corresponding to positions 4-10 of SEQ ID NO: 1.

In some embodiments, the soluble γT-cell receptor chain or fragment thereof further comprises a γCDR1 region represented by an amino acid sequence comprising at least 70% sequence identity or similarity with SEQ ID NO: 375, and a γCDR2 region represented by an amino acid sequence comprising at least 70% sequence identity or similarity with SEQ ID NO: 376.

In some embodiments, the soluble γT-cell receptor chain or fragment thereof is a γ4T-cell receptor chain or fragment thereof.

In some embodiments, the soluble γT-cell receptor chain or fragment thereof further comprises a Cγ1 or Cγ2 constant region or fragment thereof.

In some embodiments, the Cγ1 constant region or fragment thereof comprises an amino acid sequence comprising at least 70% identity or similarity with SEQ ID NO: 112, or the Cγ2 constant region or fragment thereof comprises an amino acid sequence comprising at least 70% identity or similarity with SEQ ID NO: 381 or SEQ ID NO: 382.

In some embodiments, the soluble γT-cell receptor chain or fragment thereof comprises an amino acid sequence comprising at least 70% identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172,196, 220, 244, 268, 292, or 316.

In some embodiments, the soluble γT-cell receptor chain or fragment thereof further comprises a T-cell- and/or NK-cell binding domain, preferably a CD3-binding domain.

In some embodiments, the soluble γT-cell receptor chain or fragment thereof mediates an anti-tumor or anti-infective response, preferably against a target cell expressing endothelial protein C receptor (EPCR).

In a second aspect, there is provided a soluble δT-cell receptor chain or a fragment thereof comprising a δCDR3 region, wherein said δCDR3 region is represented by an amino acid sequence comprising at least 60% sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 7-12 of SEQ ID NO: 2. In some embodiments, the modification is an amino acid substitution.

In some embodiments, the soluble δT-cell receptor chain or fragment thereof comprises an amino acid substitution in the δCDR3 region relative to SEQ ID NO: 2 at the amino acid position corresponding to position 7 of SEQ ID NO: 2, preferably a substitution of an isoleucine by a leucine.

In some embodiments, the soluble δT-cell receptor chain or fragment thereof comprises an amino acid substitution in the δCDR3 region relative to SEQ ID NO: 2 at the amino acid position corresponding to position 8 of SEQ ID NO: 2, preferably a substitution of an arginine by a lysine.

In some embodiments, the soluble δT-cell receptor chain or fragment thereof comprises an amino acid substitution in the δCDR3 region relative to SEQ ID NO: 2 at the amino acid position corresponding position 9 of SEQ ID NO: 2.

In some embodiments, the soluble δT-cell receptor chain or fragment thereof comprises an amino acid substitution in the δCDR3 region relative to SEQ ID NO: 2 at the amino acid position corresponding to position 10 of SEQ ID NO: 2, preferably a substitution of a tyrosine by a phenylalanine.

In some embodiments, the soluble δT-cell receptor chain or fragment thereof comprises an amino acid substitution in the δCDR3 region relative to SEQ ID NO: 2 at the amino acid position corresponding to position 11 of SEQ ID NO: 2.

In some embodiments, the soluble δT-cell receptor chain or fragment thereof comprises an amino acid substitution in the δCDR3 region relative to SEQ ID NO: 2 at the amino acid position corresponding to position 12 of SEQ ID NO: 2.

In some embodiments, the δCDR3 region comprises an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100) at the amino acid positions corresponding to positions 7-12 of SEQ ID NO: 2.

In some embodiments, the soluble δT-cell receptor chain or fragment thereof further comprises a δCDR1 region represented by an amino acid sequence comprising at least 70% sequence identity or similarity with SEQ ID NO: 377, and a δCDR2 region represented by an amino acid sequence comprising at least 70% sequence identity or similarity with SEQ ID NO: 378.

In some embodiments, the receptor chain or fragment thereof is a δ5T-cell receptor chain or fragment thereof.

In some embodiments, the soluble δT-cell chain or fragment thereof further comprises a Cδ constant region or fragment thereof.

In some embodiments, the Cδ constant region or fragment thereof comprises an amino acid sequence comprising at least 70% identity or similarity with SEQ ID NO: 383.

In some embodiments, the soluble δT-cell chain or fragment thereof comprises an amino acid sequence comprising at least 70% identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330.

In some embodiments, the soluble δT-cell receptor chain or fragment thereof further comprises a T-cell- and/or NK-cell binding domain, preferably a CD3-binding domain.

In some embodiments, the soluble δT-cell receptor chain or fragment thereof mediates an anti-tumor or anti-infective response, preferably against a target cell expressing endothelial protein C receptor (EPCR).

In a third aspect, there is provided a soluble γδT-cell receptor or fragment thereof comprising a γCDR3 and a δCDR3 region, wherein the γδT-cell receptor or fragment thereof comprises a soluble γT-cell receptor chain or fragment thereof of the first aspect and/or a soluble δT-cell receptor chain or fragment thereof of the second aspect.

In some embodiments, the soluble γδT-cell receptor or fragment thereof comprises:
a soluble γT-cell receptor chain or fragment thereof comprising a γCDR3 region comprising the amino acid sequence DAFYY (SEQ ID NO: 369) at the amino acid positions corresponding to positions 5-9 of SEQ ID NO: 1, preferably comprising a γCDR3 region comprising the amino acid sequence SEQ ID NO: 10, and/or,
a soluble δT-cell receptor chain or fragment thereof comprising a δCDR3 region comprising the amino acid sequence IKGFTG (SEQ ID NO: 97) at the amino acid positions corresponding to positions 7-12 of SEQ ID NO: 2, preferably comprising a δCDR3 region comprising amino acid sequence SEQ ID NO: 23.

In some embodiments, the soluble γδT-cell receptor or fragment thereof comprises:
a soluble γT-cell receptor chain or fragment thereof comprising a γCDR3 region comprising the amino acid sequence WDAFYYK (SEQ ID NO: 83) at the amino acid positions corresponding to positions 4-10 of SEQ ID NO: 1, preferably comprising a γCDR3 region comprising the amino acid sequence SEQ ID NO: 10, and/or,
a soluble δT-cell receptor chain or fragment thereof comprising a δCDR3 region comprising the amino acid sequence IKGFTG (SEQ ID NO: 97) at the amino acid positions corresponding to positions 7-12 of SEQ ID NO: 2, preferably comprising a δCDR3 region comprising amino acid sequence SEQ ID NO: 23.

In a fourth aspect, there is provided a nucleic acid molecule encoding a soluble γT-cell receptor chain or fragment thereof of the first aspect, a soluble δT-cell receptor chain or fragment thereof of the second aspect, or a soluble γδT-cell receptor or fragment thereof of the third aspect. In a fifth aspect, there is provided a nucleic acid construct comprising a nucleic acid molecule of the fourth aspect.

In a sixth aspect, there is provided a cell expressing a soluble γT-cell receptor chain or fragment thereof of the first aspect, a soluble δT-cell receptor chain or fragment thereof of the second aspect, a soluble γδT-cell receptor or fragment thereof of the third aspect, or comprising a nucleic acid molecule of the fourth aspect or a nucleic acid construct of the fifth aspect.

In a seventh aspect there is provided a composition, preferably a pharmaceutical composition, comprising a soluble γT-cell receptor chain or fragment thereof of the first aspect, a soluble δT-cell receptor chain or fragment thereof of the second aspect, a soluble γδT-cell receptor or fragment thereof of the third aspect, a nucleic acid molecule of the fourth aspect, a nucleic acid construct of the fifth aspect, or a cell of the sixth aspect.

In an eighth aspect, there is provided a soluble γT-cell receptor chain or fragment thereof of the first aspect, a soluble δT-cell receptor chain or fragment thereof of the second aspect, a soluble γδT-cell receptor or fragment thereof of the third aspect, a nucleic acid molecule of the fourth aspect, a nucleic acid construct of the fifth aspect, a cell of the sixth aspect, or a composition of the seventh aspect, for use as a medicament. In some embodiments, the provided soluble γT-cell receptor chain or fragment thereof, soluble δT-cell receptor chain or fragment thereof, soluble γδT-cell receptor or fragment thereof, nucleic acid molecule, nucleic acid construct, cell, or composition is for use in treating, regressing, curing, and/or delaying a cancer or an infection in a subject, preferably wherein the subject is a human being.

### DESCRIPTION OF THE INVENTION

### Soluble γT-Cell Receptor Chains, δT-Cell Receptor Chains, γδT-Cell Receptors, or Fragments Thereof

Provided herein are soluble γT-cell receptor chains or fragments thereof comprising a γCDR3 region, soluble δT-cell receptor chains or fragments thereof comprising a δCDR3 region, and soluble γδT-cell receptors or fragments thereof comprising a γCDR3 and a δCDR3 region. A “soluble” γT-cell receptor chain, δT-cell receptor chain, γδT-cell receptor, or fragment thereof as used herein refers to a polypeptide that may be in solution, i.e., that is not embedded in a cellular membrane. A soluble γT-cell receptor chain, soluble δT-cell receptor chain, soluble γδT-cell receptor, or fragment thereof may be a chimeric polypeptide comprising additional sequences, as described later herein. A soluble γT-cell receptor chain or fragment thereof, a soluble δT-cell receptor chain or fragment thereof, and/or a soluble γδT-cell receptor or fragment thereof is preferably able to mediate an anti-tumour or anti-infective response. Preferably, it is able to mediate an anti-tumour or anti-infective response against a target cell expressing endothelial protein C receptor (EPCR).

In the context of the disclosure the term “fragment” of a γT-cell or δT-cell receptor chain or γδT-cell receptor may be replaced by the term “part”, the two terms being interchangeable. As used herein, the term “T-cell receptor” may be abbreviated as “TCR”. The term “γT-cell receptor chain” (or γCDR3 region) may be alternatively referred to as “gamma T-cell receptor chain” (or gamma CDR3 region) or “gT-cell receptor chain” (or gCDR3 region). The term “δT-cell receptor chain” (or δCDR3 region) may be alternatively referred to as “delta T-cell receptor chain” (or delta CDR3 region) or “dT-cell receptor chain” (or dCDR3 region).

A fragment or part of a polypeptide may correspond to at least 1%, at least 2%, at least 3%, at least 4%, at least 5%, at least 10%, at least 20%, at least 30%, at least 40% of the length of a polypeptide, for example as represented by an amino acid sequence with a specific SEQ ID NO, or at least 50%, or at least 60%, or at least 70%, or at least 80%, or at least 90% of the length of the polypeptide. A fragment or part of a polypeptide may correspond to an extracellular domain of a polypeptide, such as a γT-cell receptor chain, a δT-cell receptor chain, or a γδT-cell receptor, or a fragment of said extracellular domain, as discussed later herein. A fragment or part of a polypeptide may correspond to a complete variable region and/or a fragment or part of a constant region of a γT-cell receptor chain, a δT-cell receptor chain, or a γδT-cell receptor. A fragment or part of a polypeptide may correspond to a fragment or part of a variable region and/or a fragment or part of a constant region of a γT-cell receptor chain, a δT-cell receptor chain, or a γδT-cell receptor. A fragment or part of a polypeptide may correspond to, or comprise, a CDR3 region of a γT-cell receptor chain, a δT-cell receptor chain, or a γδT-cell receptor or a fragment or part thereof.

A fragment or part of a polypeptide is preferably a functional fragment or part thereof. It may mean that this fragment or part exhibits a similar activity as the polypeptide it is derived from. In the context of the disclosure, an activity may for example be the mediation of an anti-tumour or an anti-infective response as explained later herein. A similar anti-tumour or anti-infective response may mean that the fragment or part of the polypeptide mediates at least 50% of said anti-tumour or anti-infective response, or at least 60%, or at least 70%, or at least 80%, or at least 90%, or at least 110%, or at least 120%, or more, as compared to the polypeptide it is derived from. In some embodiments, a fragment or part of a soluble γT-cell receptor chain, soluble δT-cell receptor chain, or soluble γδT-cell receptor corresponds to an extracellular domain or fragment or part thereof, as described later herein.

An “amino acid modification” as described herein may refer to a modification resulting in an amino acid sequence being modified (altered). Accordingly, an amino acid modification is to be understood as also encompassing modifications to the nucleotide sequence which encodes an amino acid sequence to be modified.

Such a modification may, for example, be an amino acid substitution, insertion, deletion, or a combination thereof. In some embodiments, an amino acid modification is an amino acid substitution. In some embodiments, an amino acid modification is an amino acid insertion. In some embodiments, an amino acid modification is an amino acid deletion. In some embodiments, an amino acid substitution is a substitution of a hydrophobic, charged, or polar amino acid.

In some embodiments, an amino acid deletion is a deletion of a hydrophobic, charged, or polar amino acid.

In some embodiments, an amino acid deletion is a deletion of a hydrophobic amino acid. In some embodiments, an amino acid deletion is a deletion of a charged amino acid. In some embodiments, an amino acid deletion is a deletion of a polar amino acid.

An amino acid modification in a parent (reference) amino acid sequence may result in a variant amino acid sequence (alternatively referred to herein as mutant or derivative amino acid sequence). Accordingly, a soluble γT-cell receptor chain, soluble δT-cell receptor chain, soluble γδT-cell receptor, or a fragment thereof, comprising a variant amino acid sequence may be called a variant or mutant or derivative soluble γT-cell receptor chain, soluble δT-cell receptor chain, soluble γδT-cell receptor, or a fragment thereof.

An amino acid substitution refers to a sequence modification that replaces an amino acid residue in a parent (reference) by another amino acid (or a nucleotide in a nucleotide sequence comprised by a nucleic acid molecule encoding the amino acid sequence) which results in a variant (mutant or derivative) sequence that has the same number of amino acids. An amino acid substitution may correspond to a substitution by any other amino acid. An amino acid substitution may correspond to a substitution of a hydrophobic, charged, or polar amino acid by any other amino acid. An amino acid substitution may correspond to a substitution of a hydrophobic, charged or polar amino acid by a hydrophobic, charged, or polar amino acid. An amino acid substitution may correspond to a substitution of a hydrophobic amino acid. An amino acid substitution may correspond to a substitution of a polar amino acid. An amino acid substitution may correspond to a substitution of a charged amino acid. An amino acid substitution may correspond to a substitution of an L-amino acid by a D-amino acid. An amino acid substitution may correspond to a substitution by a non-natural amino acid. An amino acid substitution may be conservative. A definition of “conservative” amino acid substitutions is provided later herein. In embodiments wherein multiple amino acids are substituted, they may correspond to consecutive positions, to positions that are not consecutive, or to positions that are spatially apart in the amino acid sequence. The skilled person understands that amino acid modifications in the context of the disclosure may be combined, e.g., an amino acid sequence may comprise an amino acid substitution and an amino acid insertion and/or deletion relative to an amino acid sequence, for example an amino acid sequence having a SEQ ID NO as described herein.

A soluble γT-cell receptor chain or fragment thereof described herein may, for example, be a γ1, γ2, γ3, γ4, γ5, γ8, γ9, γ10, or γ11 chain or fragment thereof, for example a γ2, γ3, γ4, γ5, γ8, γ9, or γ11 chain or fragment thereof. A γCDR3 region comprised in a soluble γT-cell receptor chain fragment may, for example, be comprised in a γ1, γ2, γ3, γ4, γ5, γ8, γ9, γ10, or γ11 chain fragment, for example in a γ2, γ3, γ4, γ5, γ8, γ9, or γ11 chain fragment. A preferred soluble γT-cell receptor chain is a γ4T-cell receptor chain. A preferred γCDR3 region is comprised in a soluble γ4T-cell receptor chain fragment.

A soluble δT-cell receptor chain or fragment thereof described herein may, for example, be a δ1, δ2, δ3, δ4, δ5, δ6, δ7, or δ8 chain or fragment thereof, for example a δ1, δ2, δ3, or δ5 chain or fragment thereof. A δCDR3 region comprised in a soluble δT-cell receptor chain fragment may be comprised in a δ1, δ2, δ3, δ4, δ5, δ6, δ7, or δ8 chain fragment, for example a δ1, δ2, δ3, or δ5 chain fragment. A preferred soluble δT-cell receptor chain is a δ5T-cell receptor chain. A preferred δCDR3 region is comprised in a soluble δ5T-cell receptor chain fragment.

A soluble γδT-cell receptor or fragment thereof described herein may, for example, comprise:
(a) a soluble γT-cell receptor chain or fragment thereof that is a γ1, γ2, γ3, γ4, γ5, γ8, γ9, γ10, or γ11 chain or fragment thereof, for example a γ2, γ3, γ4, γ5, γ8, γ9, or γ11 chain or fragment thereof,
(b) a soluble δT-cell receptor chain or fragment thereof that is a δ1, δ2, δ3, δ4, δ5, δ6, δ7, or δ8 chain or fragment thereof, for example a δ1, δ2, δ3, or δ5 chain or fragment thereof, or
(c) a combination of any soluble γT-cell receptor chain or fragment thereof of (a) and any soluble δT-cell receptor chain or fragment thereof of (b).

A preferred soluble γδT-cell receptor is a soluble γ4δ5T-cell receptor. A preferred fragment of a soluble γδT-cell receptor comprising a γCDR3 and a δCDR3 region comprises a γCDR3 region of a γ4T-cell receptor and a δCDR3 region of a δ5T-cell receptor.

Preferably, the soluble γT-cell receptor chains or fragments thereof comprising a γCDR3 region, the δT-cell receptor chains or fragments thereof comprising a δCDR3 region, and/or the γδT-cell receptors or fragments thereof comprising a γCDR3 and a δCDR3 region described herein are of mammalian, more preferably of human origin.

Each polypeptide, such as a soluble γT-cell receptor chain, variant, or fragment thereof described herein may also be represented by its encoding nucleic acid molecule (and the nucleotide sequence it comprises) instead of the amino acid sequence it comprises. The same holds for each soluble δT-cell receptor chain, variant, or fragment thereof and for each γδT-cell receptor chain, variant, or fragment thereof described herein.

In some embodiments, a soluble γT-cell receptor chain or fragment thereof and/or a soluble δT-cell receptor chain or fragment thereof described herein comprises a modification selected from an amino acid substitution, deletion, insertion, and combinations thereof as compared to a reference (starting) sequence. Preferably, the amino acid substitution is a substitution of a hydrophobic, charged or polar amino acid. Substitution of hydrophobic, charged, or polar amino acids is further discussed in the section “general definitions” later herein. Preferably, the modification is comprised in the γCDR3 region and/or δCDR3 region.

A reference (starting) sequence as used herein is to be understood as the original amino acid sequence (or the nucleotide sequence encoding the amino acid sequence) to which the modification is introduced.

In some embodiments, a soluble γT-cell receptor chain or fragment thereof and/or a soluble δT-cell receptor chain or fragment thereof described herein comprises at least one, at least two, at least three, at least four, at least five, at least six, or at least seven amino acid modifications as compared to a reference (starting) sequence. In some embodiments, a soluble γT-cell receptor chain or fragment thereof and/or a soluble δT-cell receptor chain or fragment thereof comprises from 1 to 7, from 1 to 6, from 1 to 5, from 1 to 4, from 1 to 3, or from 1 to 2 amino acid modifications as compared to a reference (starting) sequence. Preferably, the amino acid modifications are comprised in the γCDR3 and/or δCDR3 regions.

In some embodiments, a soluble γT-cell receptor chain or fragment thereof and/or a soluble δT-cell receptor chain or fragment thereof comprises at least one, at least two, at least three, at least four, at least five, at least six, or at least seven amino acid substitutions as compared to a reference (starting) sequence. In some embodiments, a soluble γT-cell receptor chain or fragment thereof and/or a soluble δT-cell receptor chain or fragment thereof comprises from 1 to 7, from 1 to 6, from 1 to 5, from 1 to 4, from 1 to 3, or from 1 to 2 amino acid substitutions as compared to a reference (starting) sequence. Preferably, the amino acid substitutions are comprised in the γCDR3 and/or δCDR3 regions.

In some embodiments, a soluble γT-cell receptor chain or fragment thereof and/or a soluble δT-cell receptor chain or fragment thereof comprises a deletion of at least one, at least two, at least three, at least four, at least five, at least six, or at least seven amino acids as compared to a reference (starting) sequence. In some embodiments, a soluble γT-cell receptor chain or fragment thereof and/or a soluble δT-cell receptor chain or fragment thereof comprises from 1 to 7, from 1 to 6, from 1 to 5, from 1 to 4, from 1 to 3, or from 1 to 2 amino acid deletions as compared to a reference (starting) sequence. Preferably, the amino acid deletions are comprised in the γCDR3 and/or δCDR3 regions.

In some embodiments, a soluble γT-cell receptor chain or fragment thereof and/or a soluble δT-cell receptor chain or fragment thereof comprises an insertion of at least one, at least two, at least three, at least four, at least five, at least six, or at least seven amino acids as compared to a reference (starting) sequence. In some embodiments, a soluble γT-cell receptor chain or fragment thereof and/or a soluble δT-cell receptor chain or fragment thereof comprises from 1 to 7, from 1 to 6, from 1 to 5, from 1 to 4, from 1 to 3, or from 1 to 2 amino acid insertions as compared to a reference (starting) sequence. Preferably, the amino acid insertions are comprised in the γCDR3 and/or δCDR3 regions.

A preferred reference γT-cell receptor chain amino acid sequence is represented by or comprises SEQ ID NO: 4. A preferred reference γCDR3 region amino acid sequence is represented by SEQ ID NO: 1. SEQ ID NO: 1 represents the γCDR3 region of a γT-cell receptor chain amino acid sequence represented by or comprising SEQ ID NO: 4. A preferred reference δT-cell receptor chain amino acid sequence is represented by or comprises SEQ ID NO: 6. A preferred reference δCDR3 region amino acid sequence is represented by SEQ ID NO: 2. SEQ ID NO: 2 represents the δCDR3 region of a δT-cell receptor chain amino acid sequence represented by or comprising SEQ ID NO: 6.

In some embodiments, the amino acid modifications may be introduced at specific positions of a reference amino acid sequence (or nucleotide sequence encoding the amino acid sequence). Preferred specific positions in the case of a soluble γT-cell receptor chain or fragment thereof described herein may be located in the γCDR3 region. Preferred specific positions in the case of a soluble δT-cell receptor chain or fragment thereof described herein may be located in the δCDR3 region.

The skilled person is aware of how to locate an amino acid sequence corresponding to the CDR3 region (or a nucleotide sequence encoding a CDR3 region) of a γT-cell- or δT-cell receptor chain (or fragments thereof) in a reference sequence, as well as specific positions of a CDR3 region, using standardized nomenclature such as the one provided by the International Immunogenetics Information System (IMGT, Lefranc et al., 2005 (Nucl Acids Res 33: D593-D597) and Lefranc et al., 2014 (Front Immunol 5:22), both of which are incorporated herein in their entireties, further described in the public database available at imgt.org).

According to IMGT nomenclature, in both γT-cell- and δT-cell receptor chains, the CDR3 region is delimited by (but does not include) the anchor positions C104 and F118 or (or W118). Using this information, the skilled person can locate the amino acid sequence encoding a γCDR3 region or a δCDR3 region in any reference amino acid sequence (or a nucleotide sequence encoding it) of a γT-cell receptor or δT-cell receptor (or fragment thereof), as well as corresponding positions in other reference sequences, and subsequently introduce one or more amino acid modifications as described herein.

As a non-limiting example, the γCDR3 region in SEQ ID NO: 4 is located between C113 (corresponding to C104 according to IMGT) and F125 (corresponding to F118 according to IMGT).

As another non-limiting example, the δCDR3 region in SEQ ID NO: 6 is located between C116 (corresponding to C104 according to IMGT) and F133 (corresponding to F118 according to IMGT).

The location of the corresponding γCDR3 or δCDR3 regions, as well as specific corresponding amino acid positions therein, in other reference sequences as well as in the soluble γT-cell receptor chains, soluble δT-cell receptor chains, or fragments thereof described herein may be similarly identified.

A polypeptide, such as a soluble γT-cell receptor chain, a soluble δT-cell receptor chain, or fragments thereof, or a soluble polypeptide comprising them, may be obtained using any molecular toolbox technique known to the skilled person. Variants may, for example, be generated by introducing predetermined modifications to a reference nucleotide sequence encoding a polypeptide. Predetermined modifications may, for example, be introduced via site-directed mutagenesis. Alternatively, nucleic acid molecules comprising predetermined modifications may be synthesized and supplied by commercial vendors, for example by Integrated DNA Technologies (IA, USA), and others. Variants may alternatively (or in addition) be generated by random mutagenesis of a reference nucleotide sequence encoding a polypeptide, for example using error-prone PCR, PCR with mismatched primers, ambiguous base analogs, mutagenic agents, and the like.

As an example, starting from a reference nucleic acid molecule encoding a soluble γT-cell receptor chain and/or a soluble δT-cell receptor chain (or fragments thereof comprising a CDR3 region) site-directed mutagenesis or random mutagenesis may be applied to generate a plurality of nucleic acid molecules encoding soluble γT-cell receptor chains and/or soluble δT-cell receptor chains (or fragments thereof comprising a CDR3 region) having distinct modifications as described herein. Such nucleic acid molecules may alternatively be synthesized and supplied by commercial vendors as discussed above.

The nucleic acid molecules may be comprised in a nucleic acid construct or a vector, as described later herein. Preferred vectors are plasmids and viral vectors, with retroviral and lentiviral vectors being more preferred and lentiviral vectors being most preferred.

Nucleic acid molecules, constructs, and vectors described herein may comprise additional nucleotide sequences. Exemplary sequences are regulatory sequences, sequences encoding signal peptides, sequences encoding linker peptides, sequences facilitating the co-expression of a soluble γT-cell receptor chain (or fragment thereof comprising a γCDR3 region) and a soluble δT-cell receptor chain (or fragment thereof comprising a δCDR3 region) (in embodiments wherein they are encoded by a single nucleic acid molecule, construct, or vector), and the like. A further description of additional nucleotide sequences is provided later herein.

Nucleic acid molecules, constructs, and vectors described herein may be comprised in a library of vectors, preferably of plasmids or viral vectors, more preferably of retroviral or lentiviral vectors, most preferably of lentiviral vectors, or a library of host cells. A library of vectors or of host cells refers to a population of vectors or of host cells, each of which comprises one or more, preferably one, nucleic acid molecule of a plurality of molecules, with nucleic acid molecule preferably being distinct. A library of viral vectors may be constructed directly, for example from assembling nucleic acid molecules encoding soluble γT-cell receptor chains, soluble δT-cell receptor chains, soluble γδT-cell receptors, or fragments thereof together with vector backbones (the backbones optionally being linear). Alternatively, a library of plasmids may first be constructed, out of which the assembled nucleic acids can be obtained (e.g. via PCR) and used for viral vector library construction. Each vector in a library may encode a soluble γT-cell receptor chain or fragment thereof comprising a γCDR3 region, a soluble δT-cell receptor chain or fragment thereof comprising a δCDR3 region, or a soluble γδT-cell receptor or fragment thereof comprising a γCDR3 and a δCDR3 region (bicistronic vector). Examples of library construction are provided in the experimental section herein.

Host cell libraries may, for example, be constructed by transformation of the host cells with the nucleic acid molecules (or with the nucleic acid constructs or vectors comprising them) followed by cell culturing to obtain a population of transformed cells. Suitable host cells, transformation methods, and culturing methods are known to the skilled person and discussed in standard handbooks available in the art. Preferred host cells may be selected from bacteria (e.g., E. coli), yeasts (e.g., P. pastoris, S. cerevisiae), insect cells (e.g. Sf9 cells), mammalian cells (such as CHO cells), and human cells, for example human cell lines (e.g., HEK293 or HEK293F or derivatives thereof), or immunoresponsive cells, preferably T-cells. Nucleic acid molecules may be obtained from the library of host cells using any DNA isolation method known to the skilled person.

The ability of a soluble γT-cell receptor chain, soluble δT-cell receptor chain, soluble γδT-cell receptor, or fragment thereof, or of soluble polypeptides comprising them, to mediate an anti-tumor or anti-infective response may be assessed using T-cells and/or NK-cells, preferably human T-cells and/or NK-cells.

T-cells, alternatively called T-lymphocytes, belong to a group of white blood cells named lymphocytes, which play a role in cell-mediated immunity. T-cells originate from hematopoietic stem cells in the bone marrow, mature in the thymus, and gain their full function in peripheral lymphoid tissues. During T-cell development, CD4−CD8− T-cells (negative for both the CD4 and CD8 co-receptor) are committed either to an αβ or γδ fate as a result of an initial βTCR or δTCR gene rearrangement. Cells that undergo early β-chain rearrangement express a pre-TCR structure composed of a complete β-chain and a pre-TCRα chain on the cell surface. Such cells switch to a CD4+CD8+ state, rearrange the TCRα-chain locus, and express a mature αβTCR on the surface. CD4−CD8− T-cells that successfully complete the γ gene rearrangement before the β-gene rearrangement express a functional γδTCR and remain CD4−CD8−. The T-cell receptor associates with the CD3 protein complex. Mature T-cells, i.e., expressing an αβTCR or a γδTCR, express the T-cell receptor complex on the cell surface. The γδT-cells, which constitute about 1-5% of the total population of T-cells, can be divided in further subpopulations which, in humans, is based on TCRδ-chain expression. Within the extracellular domain of a T-cell receptor three complementarity determining regions (CDR1, CDR2, CDR3) are located. CDR regions are composed during the development of a T-cell where so-called variable-(V), diverse-(D), and joining-(J)-gene segments are randomly combined to generate diverse TCRs. Of the three CDR regions CDR3, for both αβT-cells and γδT-cells, is the most variable one, and is therefore the key player in antigen/ligand recognition.

AβT-cells may be defined with respect to function as T lymphocytes that express an αβTCR, which recognize peptides bound to MHC molecules (major histocompatibility complex), which are expressed on the surface of various cells. MHC molecules present peptides derived from the proteins of a cell. When for example a cell is infected with a virus, the MHC will present viral peptides, and the interaction between the αβTCR on the T-cell and the MHC-complex on the target cell (i.e., the virus infected cell) activates specific types of T-cells which initiate and immune responses to eliminate the infected cell. Hence, αβT-cells may be functionally defined as being cells capable of recognizing peptides bound to MHC molecules. APT-cells may be selected from peripheral blood for example via the CD3 antigen. APT-cells may also be selected with an antibody specific for the αβTCR, many of which are commercially available such as the ones offered by ThermoFisher Scientific (Waltham, MA, USA). From such selected cells, the nucleic acid (or amino acid) sequence corresponding to the αT-cell receptor chain and/or the PT-cell receptor chain may be determined by sequencing using standard methods available in the art. Hence, αβT cells may also be defined as being cells naturally comprising a nucleic acid (or amino acid) sequence corresponding to a functional αT-cell receptor chain and/or a functional PT-cell receptor chain. γδT-cells may be functionally defined in that they are specifically and rapidly activated by e.g., (but not limited to) a set of non-peptidic phosphorylated isoprenoid precursors, collectively named phosphoantigens or stress signals medicated by non-classical HLA molecules like CD1 (this is for example the case for the Vγ9δ2 T-cell subset). Phosphoantigens are produced by virtually all living cells, though the levels are usually very low in healthy cells, and increased in transformed/malignant cells or cells infected by e.g., Mycobacterium tuberculosis, which deliver a derivate of phosphoantigens. Activation of γδT-cells comprises clonal expansion, cytotoxic activity and expression and release of cytokines. γδT-cells are also defined by expression of the γδT-cell receptor. For example, cells may be selected using an antibody specific for the γδT-cell receptor, many of which are commercially available such as the ones offered by ThermoFisher Scientific (MA, USA). Hence, γδT-cells may also be defined as being cells naturally comprising a nucleic acid (or amino acid) sequence corresponding to a functional γT-cell receptor chain and/or a functional δT-cell receptor chain.

The person skilled in the art is well capable of selecting and/or identifying cell populations characterized by expression of an antigen or receptor on the surface of the cell such as described throughout herein. It is understood that with regard to expression on the surface of cells, such as expression of CD3, CD4, CD8, αβTCR, and γδTCR, and fragments thereof, this typically involves a population of cells of which a portion of cells have a much higher level of expression of the respective polypeptide when compared to cells having a lower level of expression. Hence, the terms positive or negative are to be understood as being relative, i.e., positive cells have a much higher expression level as compared to cells being negative. Cells being negative in this sense may thus still have an expression level which may be detectable.

Expression on the surface of cells may be analyzed using, for example, fluorescence activated cell sorting (FACS), and many specific antibodies are commercially available, e.g., targeting CD3, CD4, CD8, αβTCR, γδTCR, that are suitable for such FACS analysis, such as the ones offered by ThermoFisher Scientific (MA, USA). As an example, αβT cells can also be defined and selected as being positive for αβTCR expression in FACS. The same holds for γδT cells and γδTCR expression. Conditions that allow the selection of negative and/or positive cells may be according to the manufacturer's protocols. Optionally, additional techniques such as magnetic bead separation may be utilized.

Further examples of antibodies that may be suitable for selection of T-cells are available from BD Pharmingen (BD, Franklin Lakes, NJ USA) such as Vδ2-FITC (clone B6, #555738), or from Thermofisher Scientific (Waltham, MA, USA) such as Vγ1-PE-Cy7 (clone TS8.2, #25-5679-42), or from Biolegend (San Diego, CA, USA) such as αβTCR-BV785 (clone IP26, #306742), or from Beckman Coulter (Brea, CA, USA) such as pan-γδTCR-PE (clone IMMU510, #IM1418U), or from Miltenyi Biotec (Bergisch Gladbach, Germany) such as CD3-VioGreen (clone REA613, #130-113-142, or from Biolegend (San Diego, CA, USA) such as anti-biotin αβTCR (clone IP26, #306704), with many others being commercially available.

The mediation of an anti-tumor or anti-infective response by soluble γT-cell receptor chains, soluble δT-cell receptor chains, soluble γδT-cell receptors, or fragments thereof, or by soluble polypeptides comprising them, may be assessed directly by bringing them into contact with T-cells and/or NK-cells (and preferably a target antigen), as described later herein.

Alternatively, the γT-cell receptor chains, δT-cell receptor chains, γδT-cell receptors, or fragments thereof may first be expressed in a non-soluble form in T-cells and/or NK-cells (i.e., in a form where they are embedded in the cellular membrane), and the T-cells and/or NK-cells expressing them may then be assessed for their anti-tumor or anti-infective response. The skilled person is aware of how to generate non-soluble versions of the soluble polypeptides described herein. As a non-limiting example, transmembrane regions (TM) may be included in the polypeptides to facilitate their embedding in the cellular membrane, such as the ones represented by SEQ ID NOs: 102-104 (or fragments thereof), or any other suitable region known to the skilled person such as appropriate signal peptides. Nucleic acid molecules encoding the polypetides may be transduced to the T-cells according to standard molecular toolbox techniques known to the skilled person, examples of which are described elsewhere herein.

As demonstrated in the experimental section herein, the capacity of soluble γT-cell receptor chains, soluble δT-cell receptor chains, soluble γδT-cell receptors, or fragments thereof described herein to mediate an anti-tumor or anti-infective response can be reliably predicted by assessing the anti-tumor or anti-infective response of T-cells expressing them in a non-soluble form in the assays described herein.

With respect to the assays described herein, the use of any T-cell type, being a primary cell or any cell line can suffice, as long as the cell population, or a substantial part thereof, is able to express a γδT-cell receptor or fragment thereof and/or be activated by a soluble γT-cell receptor chain, soluble δT-cell receptor chain, soluble γδT-cell receptor, or fragment thereof (e.g., capable of exerting a cytotoxic response and/or cytokine production as later defined herein). The cell may be a progenitor cell, preferably a blood progenitor cell such as a thymocyte or a blood stem cell, which, after it has been provided with the right stimuli, can develop into a T-cell.

A cell as used herein is “engineered” when it has been transformed, modified or transduced to comprise a heterologous or exogenous nucleic acid molecule, and when it preferably expresses the polypeptide encoding by the nucleic acid molecule. As used herein, the term “engineered cell” may be replaced by “modified cell” or “transformed cell”.

In cases wherein the mediation of an anti-tumor or anti-infective response of a polypeptide described herein is assessed using T-cells, the cells may stimulated by contacting them with an antigen or epitope specific for a γδT-cell receptor or fragment thereof. In cases wherein soluble polypeptides are assayed, the soluble polypeptides and the T-cells are preferably together brought into contact with the antigens or epitopes.

In some embodiments, the T-cells are stimulated by contacting them with an antigen or epitope specific for a γδT-cell receptor or fragment thereof which is a multimer, for example a dimer, trimer, tetramer, and the like. In some embodiments, the antigen is EPCR (Endothelial protein C receptor), preferably human EPCR. In some embodiments, the T-cells are stimulated by contacting them with a target cell, preferably a target cell expressing EPCR. A target cell may natively express an antigen, for example EPCR, and/or antigen expression may be introduced and/or enhanced in a target cell, for example via ectopic gene expression, gene overexpression, or any other genomic toolbox technique known to the skilled person. Examples of target cells are tumour cells, infected cells, or infectious agents as described later herein.

In some embodiments, the contacting step has a duration of at least 1 hour, at least 2 hours, at least 4 hours, at least 8 hours, at least 16 hours, at least 18 hours, at least 20 hours, at least 1 day, at least 2 days, at least 3 days, at least 4 days, or at least 5 days.

In some embodiments wherein the T-cells are contacted with target cells, the contacting may involve any suitable effector:target (E:T) ratio suitable for stimulation to occur. Non-limiting examples of suitable E:T ratios are 3:1, 2:1, 1:1, 1:2, 1:3, 1:4, 1:5, 1:6, 1:7, 1:8, 1:9, and others.

The stimulation step may involve culturing of the engineered T-cells in order for proliferation, activation, and/or degranulation to occur. In some embodiments, culturing of the engineered T-cells involves co-culturing with target cells. Suitable growth media and culturing conditions will depend on the engineered cells used and will be known to the skilled person, with many media and protocols being commercially available, for example the TEXMACS™ medium (Miltenyi Biotec, Bergisch Gladbach, Germany). A further example is provided in the experimental section herein. The abovementioned description of the stimulation step also holds for assays using soluble γT-cell receptor chains, soluble δT-cell receptor chains, soluble γδT-cell receptors, or fragments thereof, in which the soluble γT-cell receptor chains, δT-cell receptor chains, γδT-cell receptors, or fragments thereof are brought into contact with the T-cells and the antigens and/or epitopes.

The anti-tumour or anti-infective response that is mediated may be improved (increased) relative to a control γT-cell receptor chain, δT-cell receptor chain, γδT-cell receptor, or fragment thereof. In some embodiments wherein the γT-cell receptor chain, δT-cell receptor chain, γδT-cell receptor, or fragment thereof comprises an amino acid modification compared to a reference sequence, the polypeptide comprising the reference sequence may be used as a control. Alternatively, a polypeptide comprising a different sequence may be used as a control.

The control polypeptide may be in a soluble or a non-soluble form, depending on the type of assays that is used. Multiple combinations of γT- and δT-cell receptor chains or fragments thereof may be assessed, by e.g., pairing a specific γT-cell receptor chain or fragment thereof with multiple different δT-cell receptor chains or fragments thereof, and vice versa.

In some embodiments, a control γT-cell receptor chain comprises a γCDR3 region represented by SEQ ID NO: 1 or SEQ ID NO: 379. In some embodiments, a control γT-cell receptor chain is represented by or comprises SEQ ID NO: 4. In some embodiments, a control δT-cell receptor chain comprises a δCDR3 region represented by SEQ ID NO: 2 or SEQ ID NO: 380. In some embodiments, a control δT-cell receptor chain is represented by or comprises SEQ ID NO: 6. In some embodiments, a control γδT-cell receptor comprises a γCDR3 region represented by SEQ ID NO: 1 or SEQ ID NO: 379 and a δCDR3 region represented by SEQ ID NO: 2 or SEQ ID NO: 380. In some embodiments, a control γδT-cell receptor comprises a γT-cell receptor chain represented by or comprising SEQ ID NO: 4 and a δT-cell receptor chain represented by or comprising SEQ ID NO: 6.

In some embodiments, a control γT-cell receptor chain comprises a γCDR3 region represented by SEQ ID NO: 7. In some embodiments, a control γT-cell receptor chain is represented by or comprises SEQ ID NO: 336. In some embodiments, a control δT-cell receptor chain comprises a δCDR3 region represented by SEQ ID NO: 19. In some embodiments, a control δT-cell receptor chain is represented by or comprises SEQ ID NO: 348. In some embodiments, a control γδT-cell receptor comprises a γCDR3 region represented by SEQ ID NO: 7 and a δCDR3 region represented by SEQ ID NO: 19. In some embodiments, a control γδT-cell receptor comprises a γT-cell receptor chain represented by or comprising SEQ ID NO: 336 and a δT-cell receptor chain represented by or comprising SEQ ID NO: 348.

In some embodiments, a soluble γT-cell receptor chain or a fragment thereof mediates an improved anti-tumour or anti-infective response compared to a soluble γT-cell receptor chain comprising a γCDR3 region represented by SEQ ID NO: 1. In some embodiments, a soluble δT-cell receptor chain or a fragment thereof mediates an improved anti-tumour or anti-infective response compared to a soluble δT-cell receptor chain comprising a δCDR3 region represented by SEQ ID NO: 2. In some embodiments, a soluble γδT-cell receptor or a fragment thereof mediates an improved anti-tumour or anti-infective response compared to a soluble γδT-cell receptor comprising a γCDR3 region represented by SEQ ID NO: 1 and a δCDR3 region represented by SEQ ID NO: 2.

In some embodiments, a soluble γT-cell receptor chain or a fragment thereof mediates an improved anti-tumour or anti-infective response compared to a soluble γT-cell receptor chain comprising a γCDR3 region comprising SEQ ID NO: 379. In some embodiments, a soluble δT-cell receptor chain or a fragment thereof mediates an improved anti-tumour or anti-infective response compared to a soluble δT-cell receptor chain comprising a δCDR3 region comprising SEQ ID NO: 380. In some embodiments, a soluble γδT-cell receptor or a fragment thereof mediates an improved anti-tumour or anti-infective response compared to a soluble γδT-cell receptor comprising a γCDR3 region comprising SEQ ID NO: 379 and a δCDR3 region comprising SEQ ID NO: 2. In some embodiments, a soluble γδT-cell receptor or a fragment thereof mediates an improved anti-tumour or anti-infective response compared to a soluble γδT-cell receptor comprising a γCDR3 region comprising SEQ ID NO: 1 and a δCDR3 region comprising SEQ ID NO: 380.

An anti-tumour or anti-infective response may be improved by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, or at least 10-fold relative to a control, preferably soluble, γT-cell receptor chain, δT-cell receptor chain, or γδT-cell receptor.

In some embodiments, assessing (measuring) the anti-tumour and/or anti-infective activity/response comprises contacting engineered T-cells expressing polypeptides as described herein with tumour cells, tumour cell lines, infected cells, or infectious agents such as e.g., fungal cells. Assessing an anti-tumour or anti-infective activity may include any assay in which an anti-tumour or anti-infective effect may be determined, such as having an effect on tumour cell or infected cell or infectious agent division rate, i.e., the speed with which the tumour or infected cells or infectious agents divide, cell death, cytolysis/cytotoxicity of the tumour or infected cell or infectious agent, binding to the tumour or infected cells or infected agents, etc.

Tumour cells may be any kind of tumour cells. As a non-limiting example, they may be primary tumour cells from a patient. The tumour cells may be tumour cells from cell lines, such as (but not limited to) the cell lines listed hereafter: HT-29, RKO, T84, LS174T, SW480, KM12, LS180, HT55, MDST-8, MDA-MB-231, and others, which are well known in the art. Tumour cell lines may be obtained from the American Type Culture Collection (ATCC, Manassas, Virginia) and the like.

Infected cells may, for example, be cells that have been infected by a bacterium or a virus. The infection may result in the infected cell displaying an antigen or epitope that is a target of a γT-cell receptor chain, a δT-cell receptor chain, a γδT-cell receptor, or a fragment thereof as described herein. Non-limiting examples are Plasmodium falciparum, Mycobacterium (M.) tuberculosis and M. leprae. Infectious agents may, for example be, bacteria or fungal cells.

In some embodiments, a target cell expresses EPCR (Endothelial protein C receptor), preferably human EPCR.

In some embodiments, assessing (measuring) an anti-tumour or anti-infective response includes contacting an engineered T-cell expressing a polypeptide as described herein with a tumour or infected cell or infectious agent and measuring its ability to lyse the tumour or infected cell or infectious agent. The contacting step may, for example, have a duration (incubation period) from 10 hours to 1, 2, 3, 4, 5 days. The measurement of the ability to lyse the tumour or infected cell or infectious agent may include initially providing a fixed number of tumour or infected cells or infectious agents with which the T-cell is contacted and, after an incubation period, counting the number of the viable tumour or infected cells or infectious agents. An anti-tumour or anti-infective response may be considered to be present when the number of viable tumour or infected cells or infectious agents at the end of the incubation step is less than 95%, less than 90%, less than 80%, less than 70%, less than 60%, less than 50%, less than 40%, less than 30%, less than 20%, or less than 10% of the initial number of tumour or infected cells or infectious agents at the onset of the incubation step.

Alternatively, an anti-tumour or anti-infective response may be considered to be present when the number of viable tumour or infected cells or infectious agents at the end of the incubation step with the engineered T-cell is lower than the number of tumour or infected cells or infectious agents at the end of a similar incubation/contacting step with control (comparable) T-cells not expressing a γT-cell receptor chain, δT-cell receptor chain, γδT-cell receptor, or fragment thereof as described herein or expressing a control γT-cell receptor chain, δT-cell receptor chain, γδT-cell receptor, or fragment thereof.

Lower in this context may mean at least 5% lower, at least 10% lower, at least 20% lower, at least 30% lower, at least 40% lower, at least 50% lower, at least 60% lower, at least 70% lower, at least 80% lower, at least 90% lower. Such cells may be considered to exhibit an improved anti-tumour or anti-infective response compared to the control cells.

In addition, or as alternative to the counting of the number of viable tumour or infected cells or infectious agents at the end of the incubation/contacting step, one may also perform a ⁵¹Chromium-release assay which is known to the skilled person. The amount of ⁵¹Chromium release is a measure of the number of cells that have been lysed.

In some embodiments, one may assess the cytotoxicity of the engineered T-cells by incubating them with tumour or infected cells or infectious agents at E:T (Effector:Target) ratio of 3:1, 2:1, 1:1, 1:2, 1:3, 1:4, 1:5, 1:6, 1:7, 1:8, 1:9, or any other suitable E:T ratio. The incubation may have a duration of from 10 hours to 1, 2, 3, 4, 5 days. In some embodiments, the duration is 1 or 2 days. Control T-cells (not expressing a γT-cell receptor chain, δT-cell receptor chain, γδT-cell receptor, or fragment thereof as described herein) or expressing control γT-cell receptor chains, δT-cell receptor chains, or γδT-cell receptors, may also be used. Cytotoxicity may be measured using e.g., an xCELLigence assay (Agilent, CA, USA) and plotted as percentage of cytolysis relative to maximum cytolysis induced by treatment of the target cells with the detergent Triton-X-100.

In some embodiments, the percentage of target cell cytolysis obtained by engineered T-cells is higher (preferably at least 5%, at least 10%, at least 20%, at least 30%, at least 40%, at least 50%, at least 60%, at least 70%, at least 80%, at least 90%, at least 100%, or more) than the percentage of cytolysis obtained when the same target cells are contacted with control (comparable) T-cells not expressing a γT-cell receptor chain, δT-cell receptor chain, γδT-cell receptor, or fragment thereof as described herein or expressing a control γT-cell receptor chain, δT-cell receptor chain, γδT-cell receptor, or fragment thereof. Such cells may be considered to exhibit an improved anti-tumour or anti-infective response compared to the control cells.

Cytotoxicity may alternatively be measured using e.g., an LDH release assay from target cells, by calculating the LDH release fold-change when the target cells are incubated with engineered T-cells relative to when the same target cells are incubated with control T-cells. In some embodiments, the LDH release when the target cells are incubated with engineered T-cells is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, or at least 10-fold relative to when the same target cells are incubated with control T-cells.

Alternatively, cytotoxicity may be measured using e.g., a luciferase-based cytotoxicity assay, in which the target cells are pre-transduced to express luciferase and cytotoxicity is measured by measuring decreased luciferase activity relative to target cells cultured alone or incubated with control T-cells as discussed above. Such assays may be performed according to commercial protocols, for example by adding D-luciferine substrate (e.g., from ThermoFisher Scientific, Waltham, MA, USA) to target cells incubated with the T-cells and reading the luminescence in culture endpoint mode using a Glomax luminometer according to the manufacturer's instructions (Promega, WI, USA).

Further examples of cytotoxicity measurements are provided in the experimental section herein.

An anti-tumour response may also be assessed by assessing (measuring) the binding of engineered T-cells expressing a γT-cell receptor chain, a δT-cell receptor chain, a γδT-cell receptor, or a fragment thereof described herein to a tumour or infected cell after contacting both cells together. Such a contacting step may have a duration of from 10 hours to 1, 2, 3, 4, 5 days. When binding of said T-cell to the tumour cell or infected cell or infectious agent is detected at the end of the contacting step, said T-cell may be considered to exhibit an anti-tumour or anti-infective response.

Alternatively and preferably, the binding of said T-cell to said tumour or infected cell or infectious agent at the end of the contacting step is higher (preferably at least 10%, at least 20%, at least 30%, at least 40%, at least 50%, at least 60%, at least 70%, at least 80%, at least 90%, at least 100% or more) than the binding displayed by control (comparable) T-cells not expressing the γT-cell receptor chain, δT-cell receptor chain, γδT-cell receptor, or fragment thereof, or expressing a control γT-cell receptor chain, δT-cell receptor chain, γδT-cell receptor, or fragment thereof, to the same tumour or infected cell. Such cells may be considered to exhibit an improved anti-tumour or anti-infective response compared to the control cells.

As soon as an effect can be seen in any of the assays described above, or any other similar assay available to the skilled person, one can conclude that the γT-cell receptor chain, δT-cell receptor chain, γδT-cell receptor, or fragment thereof mediates an anti-tumour or anti-infective response or activity. The skilled person may then obtain the γT-cell receptor chains, δT-cell receptor chains, γδT-cell receptors, or fragments thereof in a soluble form.

In these assays, the control cells may, for example, be T-cells that are untransduced or that are transduced with an empty viral vector or that are transduced with a control γT-cell receptor chain, δT-cell receptor chain, γδT-cell receptor, or fragment thereof.

In some embodiments, assessing (measuring) an anti-tumour or anti-infective response includes assessing the expression of a T-cell activation and/or degranulation marker. The expression of a T-cell activation and/or degranulation marker may be linked to the activation of a cell by a γδTCR. Thus, assessment of the expression of a T-cell activation and/or degranulation marker can be used to assess the capacity of a γδTCR to transmit an activation signal to a T-cell.

Assessing the expression of a T-cell activation and/or degranulation marker after stimulation of the engineered T-cells as an alternative to the assays described above has an added benefit of further increasing the throughput with which soluble γT-cell receptor chains, δT-cell receptor chains, γδT-cell receptors, or fragments thereof, that mediate an anti-tumour or anti-infective response may be identified.

In some embodiments, a T-cell activation marker is a cytokine such as IFN-γ, IL-2 or TNFα. Cytokine production may be determined, e.g. via antibody staining, ELISA and/or quantitative PCR for the expressed mRNA. Assays for determining the production of a cytokine such as IFN-γ, IL-2 or TNFα are commercially widely available (for example from &D Systems, Minneapolis, MN, US).

When production of a cytokine such as IL-2, TNFα, or IFN-γ is detected during or at the end of a contacting step with a tumour or infected cell or infectious agent such as described earlier herein, the T-cell may be considered to exhibit an anti-tumour or anti-infective response.

Alternatively and preferably, when the amount of IFN-γ, IL-2 or TNFα produced during or at the end of the contacting step with the T-cell is higher (preferably at least 10%, at least 20%, at least 30%, at least 40%, at least 50%, at least 60%, at least 70%, at least 80%, at least 90%, at least 100% or more) than the amount of IFN-γ, IL-2 or TNFα produced when the tumour or infected cell or infectious agent is contacted with control (comparable) T-cells, the cells may be considered to exhibit an improved anti-tumour or anti-infective response compared to the control cells.

In some embodiments, the T-cell activation and/or degranulation marker is a surface expressed protein. In some embodiments, the T-cell activation and/or degranulation marker is selected from CD25 (for example Uniprot Ref: P01589), 41BB (for example Uniprot Ref: Q07011), CD62L (for example Uniprot Ref: P14151), Nur77 (for example Uniprot Ref: P22736), NOR1 (for example Uniprot Ref: Q92570), EGR2 (for example Uniprot Ref: P11161), LAG3 (for example Uniprot Ref: P18627), CD40L (for example Uniprot Ref: P29965), CD38 (for example Uniprot Ref: P28907), HLA-DR (for example Uniprot Ref: P01903), FASL (for example Uniprot Ref: P48023), CD63 (for example Uniprot Ref: P08962), CD69 (for example Uniprot Ref: Q07108), and/or CD107 (LAMP1, for example Uniprot Ref: P11279). Preferably, the T-cell activation and/or degranulation maker is CD69 (for example Uniprot Ref: Q07108) and/or CD107a (LAMP1, for example Uniprot Ref: P11279). Assessment of expression of a surface-expressed protein may be done using flow cytometry, for example using FACS as discussed elsewhere herein. As a non-limiting example, in the case of CD69 and/or CD107a, the T-cells may be stained with anti-CD69 (e.g., CD69-APC (Clone REA824, Miltenyi Biotec, Gladback Germany)) and/or anti-CD107a (e.g., CD107a-BV421 (H4A3, Biolegend, CA, USA)) antibodies, allowing assessment (measurement) of their expression using flow cytometry. An additional example is provided in the experimental section herein.

When expression of a surface-expressed T-cell activation and/or degranulation marker is detected during or at the end of a contacting step with a tumour or infected cell or infectious agent such as described earlier herein, the T-cell may be considered to exhibit an anti-tumour or anti-infective response.

Alternatively and preferably, when the expression of a surface-expressed T-cell activation and/or degranulation marker in an engineered T-cell during or at the end of the contacting step with a tumour or infected cell or infectious agent is higher (preferably at least 10%, at least 20%, at least 30%, at least 40%, at least 50%, at least 60%, at least 70%, at least 80%, at least 90%, at least 100% or more) than its expression in a control (comparable) T-cell, the cells may be considered to exhibit an improved anti-tumour or anti-infective response compared to the control cells.

In some embodiments, the T-cells may express a reporter construct. A “reporter construct” as used herein refers to a nucleic acid construct comprising a promoter sequence which can drive transcription of a nucleic acid molecule following the activation of the T-cell by a γδTCR (e.g., a promoter which is activated following the transmittal of a signal via the γδTCR complex by a change in its conformation and/or position following its activation), and a nucleic acid molecule encoding a polypeptide the activity of which can be detected (reporter polypeptide), for example using flow cytometry. Such reporter constructs can be used to assess the activation by a γδTCR and convert it to a detectable signal via the expression of the reporter polypeptide. Non-limiting examples of promoter sequences that can be comprised in a reporter construct are promoters from or derived from a response element protein selected from nuclear factor of activated T-cells (NFAT), Nuclear Factor kappa-light-chain-enhancer of activated B cells (NF-KB), Activator protein 1 (AP-I), Nur response element (NurRE), Interferon gamma (IFN-γ), CD69, Early growth response protein 1 (EGR1), Early growth response protein 2 (EGR2), IL2, and any combination thereof.

In some embodiments, the promoter comprised in a reporter construct comprises, or is, an NFAT response element or a variant thereof. A NFAT response element may have a nucleotide sequence of WGGAAA, wherein “W” stands for A or T. A promoter may comprise one or more of NFAT response elements or variants thereof, preferably one or more response elements having a nucleotide sequence of WGGAAA. A promoter comprising a variant of an NFAT response element may have at least 40%, 50%, 60%, 70%, 80%, 90%, 95%, 99% or 100% or 110%, 120%, 130%, 140%, 150% or more of the promoter activity of the original counterpart as measured under the same experimental conditions, for example using qPCR or any other suitable method known to the skilled person.

Non-limiting examples of a polypeptide the activity of which can be detected is a fluorescent or luminescent protein, for example green fluorescent protein (GFP), enhanced green fluorescent protein (eGFP), yellow fluorescent protein (YFP), red fluorescent protein (RFP), Blue fluorescent protein (BFP), cyan fluorescent protein (CFP), violet-excitable green fluorescent (Sapphire), or luciferase. In some embodiments, the polypeptide the activity of which can be detected is GFP. An exemplary GFP sequence comprises SEQ ID NO: 116. In some embodiments, the polypeptide the activity of which can be detected is luciferase. Exemplary luciferase sequences comprise SEQ ID NO: 117, 118, or 119. Detection of activity of a fluorescent or luminescent protein (such as luciferase) is discussed later herein.

Stimulation of T-cells may be performed as described earlier herein, for example by bringing the engineered T-cells into contact with a tumour or infected cell or infectious agent.

In such embodiments, when the expression of the reporter polypeptide during or at the end of the contacting step with the T-cell is higher (preferably at least 10%, at least 20%, at least 30%, at least 40%, at least 50%, at least 60%, at least 70%, at least 80%, at least 90%, at least 100% or more) than its expression when the tumour or infected cell or infectious agent is contacted with control (comparable) T-cells, the cells may be considered to exhibit an improved anti-tumour or anti-infective response compared to the control cells.

In some embodiments, a soluble γT-cell receptor chain, a soluble δT-cell receptor chain, a soluble γδT-cell receptor, or a fragment thereof described herein demonstrates a similar or improved specificity and/or affinity towards a target as compared to a control γT-cell receptor chain, δT-cell receptor chain, γδT-cell receptor, or a fragment thereof. A target may be any target described herein, for example a target cell, antigen, epitope, or another molecule.

In some embodiments, the target is EPCR or a cell expressing EPCR. In some embodiments, a control γT-cell receptor chain amino acid sequence is represented by (or comprises) SEQ ID NO: 4 and/or comprises a γCDR3 region represented by SEQ ID NO: 1 or SEQ ID NO: 379. In some embodiments, a control δT-cell receptor chain amino acid sequence is represented by (or comprises) SEQ ID NO: 6 and/or comprises a δCDR3 region represented by SEQ ID NO: 2 or SEQ ID NO: 380. In some embodiments, a control γδT-cell receptor comprises a γT-cell receptor chain amino acid sequence represented by (or comprising) SEQ ID NO: 4 and a δT-cell receptor chain amino acid sequence represented by (or comprising) SEQ ID NO: 6, and/or comprises a γCDR3 region represented by SEQ ID NO: 1 or SEQ ID NO: 379 and a δCDR3 region represented by SEQ ID NO: 2 or SEQ ID NO: 380.

In some embodiments, a control γT-cell receptor chain amino acid sequence is represented by (or comprises) SEQ ID NO: 336 and/or comprises a γCDR3 region represented by SEQ ID NO: 7. In some embodiments, a control δT-cell receptor chain amino acid sequence is represented by (or comprises) SEQ ID NO: 348 and/or comprises a δCDR3 region represented by SEQ ID NO: 19. In some embodiments, a control γδT-cell receptor comprises a γT-cell receptor chain amino acid sequence represented by (or comprising) SEQ ID NO: 336 and a δT-cell receptor chain amino acid sequence represented by (or comprising) SEQ ID NO: 348, and/or comprises a γCDR3 region represented by SEQ ID NO: 7 and a δCDR3 region represented by SEQ ID NO: 19.

Specificity and/or affinity towards a target may be “similar” when the soluble γT-cell receptor chain, soluble δT-cell receptor chain, soluble γδT-cell receptor, or fragment thereof demonstrates at least 50%, at least 60%, at least 70%, at least 80%, at least 90%, or 100% of the specificity and/or affinity demonstrated by the control γT-cell receptor chain, δT-cell receptor chain, γδT-cell receptor, or fragment thereof towards the same target.

Specificity and/or affinity towards a target may be “improved” when the soluble γT-cell receptor chain, soluble δT-cell receptor chain, soluble γδT-cell receptor, or fragment thereof demonstrates a specificity and/or affinity that is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, at least 10-fold, or at least 100-fold, relative to the specificity and/or affinity demonstrated by the control γT-cell receptor chain, δT-cell receptor chain, γδT-cell receptor, or fragment thereof towards the same target.

Specificity and/or affinity of a soluble γT-cell receptor chain, soluble δT-cell receptor chain, soluble γδT-cell receptor, or a fragment thereof towards a target may be measured using standard methods, for example by assessing (measuring) their binding to a target.

Binding of the soluble γT-cell receptor chains, soluble δT-cell receptor chains, soluble γδT-cell receptors, or fragments thereof to a target may be assessed by any suitable method known to the skilled person, such as the methods described herein. Additional examples of such methods are described in WO2018/234319A1 and US20210355188A1, incorporated herein by reference in their entireties. Briefly, target binding analysis of soluble γT-cell receptor chains, soluble δT-cell receptor chains, soluble γδT-cell receptors, or fragments thereof (as well as of soluble, such as chimeric, polypeptides comprising them as described later herein) may be carried out by surface plasmon resonance, e.g., using a BIAcore 3000 or BIAcore T200 instrument (Cytiva Lifesciences, MA, USA), or by biolayer interferometry, e.g., using a ForteBio Octet instrument (Sartorius, DE). As an example, biotinylated target monomers may be immobilized on to streptavidin-coupled CM-5 sensor chips. Equilibrium binding constants may be determined using serial dilutions of soluble γT-cell receptor chains, soluble δT-cell receptor chains, soluble γδT-cell receptors, or fragments thereof injected at a constant flow rate of 30 μl min⁻¹ over a flow cell coated with ˜200 response units (RU) of the target. Equilibrium responses may be normalised for each soluble TCR (or fragment thereof) concentration by subtracting the bulk buffer response on a control flow cell containing an irrelevant target. The K_{D} value may be obtained by non-linear curve fitting using Prism software (Dotmatics, MA, USA) and the Langmuir binding isotherm, bound=C*Max/(C+KD), where “bound” is the equilibrium binding in RU at injected soluble TCR (or fragment thereof) concentration C and Max is the maximum binding.

For high affinity interactions, binding parameters may, for example, be determined by single cycle kinetics analysis. Preferably, five different concentrations of soluble TCR (or fragment thereof) are injected over a flow cell coated with ˜100-200 RU of target molecule using a flow rate of 50-60 μl min⁻¹. Preferably, 60-120 μl of soluble TCR are injected at a top concentration of between 50-100 nM, with successive 2 fold dilutions used for the other four injections. The lowest concentration is preferably injected first. To measure the dissociation phase buffer is preferably injected until ≥10% dissociation occurs, typically after 1-3 hours. Kinetic parameters are calculated using a suitable software, e.g., using BIAevaluation® software (GE Healthcare, IL, USA). The dissociation phase is preferably fitted to a single exponential decay equation enabling calculation of half-life. The equilibrium constant K_{D} is preferably calculated from k_{off}/kₒₙ.

A soluble γT-cell receptor chain, soluble δT-cell receptor chain, soluble γδT-cell receptor, or fragment thereof, may be an isolated polypeptide. A soluble γT-cell receptor chain, soluble δT-cell receptor chain, soluble γδT-cell receptor, or fragment thereof, may be synthetically made. A soluble γT-cell receptor chain, soluble δT-cell receptor chain, soluble γδT-cell receptor, or fragment thereof may be a variant of a reference sequence.

Preferably, a soluble γT-cell receptor chain or fragment thereof is a soluble γ1T-, γ2T-, γ3T-, γ4T-, γ5T-, γ8T-, γ9T-, γ10T-, or γ11T-cell receptor chain or fragment thereof, more preferably is a soluble γ4T-cell receptor chain or fragment thereof. Preferably, a soluble δT-cell receptor chain or fragment thereof is a soluble δ1T-, δ2T-, δ3T-, δ4T-, δ5T-, δ6T-, δ7T-, or δ8T-cell receptor chain or fragment thereof, more preferably is a soluble δ5T-cell receptor chain or fragment thereof.

A soluble γT-cell receptor chain, soluble δT-cell receptor chain, soluble γδT-cell receptor, or fragment thereof, may be expressed by a cell, as described later herein.

In embodiments wherein the soluble γT-cell receptor chain, soluble δT-cell receptor chain, soluble γδT-cell receptor, or fragment thereof is expressed by a cell, said γT-cell receptor chain, δT-cell receptor chain, γδT-cell receptor, or fragment thereof is preferably exogenous (heterologous) to said cell.

In an aspect, there is provided a soluble γT-cell receptor chain or fragment thereof comprising a γCDR3 region, said receptor chain or fragment thereof being represented by an amino acid sequence comprising an amino acid modification relative to a reference sequence, preferably relative to SEQ ID NO: 1 or SEQ ID NO: 4. The amino acid modification may be at a position corresponding to a position selected from one or more positions in the reference sequence.

In an aspect, there is provided a soluble γT-cell receptor chain or fragment thereof comprising a γCDR3 region, said receptor chain or fragment thereof comprising a modification in the γCDR3 region at an amino acid position corresponding to a position selected from one or more of positions 116-122 of SEQ ID NO: 4.

In some embodiments, a soluble γT-cell receptor chain or fragment thereof comprising a γCDR3 region comprises a modification in the γCDR3 region at an amino acid position corresponding to a position selected from one or more of positions 117-121 of SEQ ID NO: 4.

In some embodiments, a soluble γT-cell receptor chain or fragment thereof comprising a γCDR3 region comprises a modification in the γCDR3 region at an amino acid position corresponding to a position selected from one or more of positions 4-10 of SEQ ID NO: 1.

In some embodiments, a soluble γT-cell receptor chain or fragment thereof comprising a γCDR3 region comprises a modification in the γCDR3 region at an amino acid position corresponding to a position selected from one or more of positions 5-9 of SEQ ID NO: 1.

The skilled person understands that amino acid position 1 (first position) of SEQ ID NO: 1 corresponds to a cysteine (C), and that amino acid position 1 (first position) of SEQ ID NO: 4 corresponds to a methionine (M). Likewise, the skilled person understands that amino acid position 1 (first position) of SEQ ID NO: 2 corresponds to a cysteine (C), and that amino acid position 1 (first position) of SEQ ID NO: 6 corresponds to a methionine (M). The remaining corresponding positions are to be numbered accordingly.

In an aspect, there is provided a soluble γT-cell receptor chain or a fragment thereof comprising a γCDR3 region, wherein said γCDR3 region is represented by an amino acid sequence comprising at least 60%, at least 70%, at least 80%, at least 85%, or at least 90% sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 116-122 of SEQ ID NO: 4, or from one or more of positions 4-10 of SEQ ID NO: 1, preferably from one or more of positions 4-10 of SEQ ID NO: 1. In some embodiments, it comprises a modification in the γCDR3 region relative to SEQ ID NO: 1 at an amino acid position corresponding to a position selected from one or more of positions 117-121 of SEQ ID NO: 4, or from one or more of positions 5-9 of SEQ ID NO: 1, preferably from one or more of positions 5-9 of SEQ ID NO: 1.

In some embodiments, a soluble γT-cell receptor chain or fragment thereof comprising a γCDR3 region comprises at least one, at least two, at least three, at least four, at least five, at least six, or at least seven amino acid modifications in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

Modifications are discussed earlier herein. For example, a modification may be an amino acid substitution, insertion, deletion, or a combination thereof, preferably it is an amino acid substitution.

In some embodiments, a soluble γT-cell receptor chain or fragment thereof comprising a γCDR3 region comprises from 1 to 7, from 1 to 6, from 1 to 5, from 1 to 4, from 1 to 3, or from 1 to 2 amino acid modifications in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

In some embodiments, a soluble γT-cell receptor chain or fragment thereof comprising a γCDR3 region comprises at least one, at least two, at least three, at least four, at least five, at least six, or at least seven amino acid substitutions in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

In some embodiments, a soluble γT-cell receptor chain or fragment thereof comprising a γCDR3 region comprises from 1 to 7, from 1 to 6, from 1 to 5, from 1 to 4, from 1 to 3, or from 1 to 2 amino acid substitutions in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1. Preferably, an amino acid substitution is a substitution of a hydrophobic, charged, or polar amino acid.

In some embodiments, a soluble γT-cell receptor chain or fragment thereof comprising a γCDR3 region comprises at least one, at least two, at least three, at least four, at least five, at least six, or at least seven amino acid deletions in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

In some embodiments, a soluble γT-cell receptor chain or fragment thereof comprising a γCDR3 region comprises from 1 to 7, from 1 to 6, from 1 to 5, from 1 to 4, from 1 to 3, or from 1 to 2 amino acid deletions in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

In some embodiments, a soluble γT-cell receptor chain or fragment thereof comprising a γCDR3 region comprises at least one, at least two, at least three, at least four, at least five, at least six, or at least seven amino acid insertions in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

In some embodiments, a soluble γT-cell receptor chain or fragment thereof comprising a γCDR3 region comprises from 1 to 7, from 1 to 6, from 1 to 5, from 1 to 4, from 1 to 3, or from 1 to 2 amino acid insertions in the γCDR3 region relative to SEQ ID NO: 1 at amino acid positions corresponding to positions 116-122, preferably positions 117-121, of SEQ ID NO: 4 or to positions 4-10, preferably positions 5-9, of SEQ ID NO: 1.

In some embodiments, a soluble γT-cell receptor chain or fragment thereof comprising a γCDR3 region comprises a modification in the γCDR3 region relative to SEQ ID NO: 1 at an amino acid position corresponding to position 116 of SEQ ID NO: 4 or to position 4 of SEQ ID NO: 1.

In some embodiments, a soluble γT-cell receptor chain or fragment thereof comprising a γCDR3 region comprises a modification in the γCDR3 region relative to SEQ ID NO: 1 at an amino acid position corresponding to position 117 of SEQ ID NO: 4 or to position 5 of SEQ ID NO: 1. Preferably, said modification is an amino acid substitution (e.g., a substitution of an aspartic acid), more preferably an amino acid substitution by a glutamic acid, most preferably an amino acid substitution of an aspartic acid by a glutamic acid.

In some embodiments, a soluble γT-cell receptor chain or fragment thereof comprising a γCDR3 region comprises a modification in the γCDR3 region relative to SEQ ID NO: 1 at an amino acid position corresponding to position 118 of SEQ ID NO: 4 or to position 6 of SEQ ID NO: 1. Preferably, said modification is an amino acid substitution (e.g., a substitution of a glycine), more preferably an amino acid substitution by an alanine, most preferably an amino acid substitution of a glycine by an alanine.

In some embodiments, a soluble γT-cell receptor chain or fragment thereof comprising a γCDR3 region comprises a modification in the γCDR3 region relative to SEQ ID NO: 1 at an amino acid position corresponding to position 119 of SEQ ID NO: 4 or to position 7 of SEQ ID NO: 1. Preferably, said modification is an amino acid substitution (e.g., a substitution of a phenylalanine), more preferably an amino acid substitution by an alanine, serine, or tyrosine, most preferably an amino acid substitution of a phenylalanine by an alanine, serine, or tyrosine.

In some embodiments, a soluble γT-cell receptor chain or fragment thereof comprising a γCDR3 region comprises a modification in the γCDR3 region relative to SEQ ID NO: 1 at an amino acid position corresponding to position 120 of SEQ ID NO: 4 or to position 8 of SEQ ID NO: 1. Preferably, said modification is an amino acid substitution (e.g., a substitution of a tyrosine), more preferably an amino acid substitution by a phenylalanine, most preferably an amino acid substitution of a tyrosine by a phenylalanine.

In some embodiments, a soluble γT-cell receptor chain or fragment thereof comprising a γCDR3 region comprises a modification in the γCDR3 region relative to SEQ ID NO: 1 at an amino acid position corresponding to position 121 of SEQ ID NO: 4 or to position 9 of SEQ ID NO: 1.

In some embodiments, a soluble γT-cell receptor chain or fragment thereof comprising a γCDR3 region comprises a modification in the γCDR3 region relative to SEQ ID NO: 1 at an amino acid position corresponding to position 122 of SEQ ID NO: 4 or to position 10 of SEQ ID NO: 1.

Preferably, the γCDR3 region is represented by an amino acid sequence comprising at least 60%, at least 70%, at least 80%, at least 85%, or at least 90%, sequence identity or similarity with SEQ ID NO: 1.

In some embodiments, a preferred soluble γT-cell receptor chain or fragment thereof comprising a γCDR3 region comprises a modification in the γCDR3 region relative to SEQ ID NO: 1 at an amino acid position corresponding to a position selected from one or more of positions 117-121 of SEQ ID NO: 4 or from one or more of positions 5-9 of SEQ ID NO: 1, preferably from one or more of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369).

Accordingly, in some embodiments, the γCDR3 region is represented by an amino acid sequence comprising at least 60%, at least 70%, at least 80%, at least 85%, or at least 90%, sequence identity or similarity with SEQ ID NO: 1, and comprises an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374), preferably selected from the group consisting of DAFYY (SEQ ID NO: 369), DGYYY (SEQ ID NO: 372), DGAYY (SEQ ID NO: 373), and DGSYY (SEQ ID NO: 374), more preferably comprises DAFYY (SEQ ID NO: 369), relative to SEQ ID NO: 1 at the amino acid positions corresponding to positions 117-121 of SEQ ID NO: 4 or to positions 5-9 of SEQ ID NO: 1, preferably to positions 5-9 of SEQ ID NO: 1.

In some embodiments, a preferred soluble γT-cell receptor chain or fragment thereof comprising a γCDR3 region comprises a modification in the γCDR3 region relative to SEQ ID NO: 1 at an amino acid position corresponding to a position selected from one or more of positions 116-122 of SEQ ID NO: 4, said modification selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGYYYK (SEQ ID NO: 88), WDGAYYK (SEQ ID NO: 89), or WDGSYYK (SEQ ID NO: 90), more preferably is WDAFYYK (SEQ ID NO: 83).

In some embodiments, a preferred soluble γT-cell receptor chain or fragment thereof comprising a γCDR3 region comprises a modification in the γCDR3 region relative to SEQ ID NO: 1 at an amino acid position corresponding to a position selected from one or more of positions 4-10 of SEQ ID NO: 1, said modification selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGYYYK (SEQ ID NO: 88), WDGAYYK (SEQ ID NO: 89), or WDGSYYK (SEQ ID NO: 90), more preferably is WDAFYYK (SEQ ID NO: 83).

Accordingly, in some embodiments, the γCDR3 region is represented by an amino acid sequence comprising at least 60%, at least 70%, at least 80%, at least 85%, or at least 90%, sequence identity or similarity with SEQ ID NO: 1, and comprises an amino acid sequence selected from the group consisting of WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), and WDGSYYK (SEQ ID NO: 90), preferably selected from the group consisting of WDAFYYK (SEQ ID NO: 83), WDGYYYK (SEQ ID NO: 88), WDGAYYK (SEQ ID NO: 89), and WDGSYYK (SEQ ID NO: 90), more preferably comprises WDAFYYK (SEQ ID NO: 83), at the amino acid positions corresponding to positions 116-122 of SEQ ID NO: 4 or to positions 4-10 of SEQ ID NO: 1, preferably to positions 4-10 of SEQ ID NO: 1.

In some embodiments, a soluble γT-cell receptor chain or fragment thereof comprising a γCDR3 region comprises, consists essentially of, or consists of, preferably comprises, an amino acid sequence having at least 60%, at least 70%, at least 80%, at least 90%, at least 95%, at least 96%, or at least 97%, sequence identity or similarity with the amino acid sequence SEQ ID NO: 4 and comprises a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, preferably of positions 4-10 of SEQ ID NO: 1, said modification preferably selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), more preferably selected from WDAFYYK (SEQ ID NO: 83), WDGYYYK (SEQ ID NO: 88), WDGAYYK (SEQ ID NO: 89), or WDGSYYK (SEQ ID NO: 90), most preferably is WDAFYYK (SEQ ID NO: 83).

In some embodiments, a soluble γT-cell receptor chain or fragment thereof comprises a γCDR3 region represented by an amino acid sequence having at least 60%, at least 70%, at least 80%, at least 90%, at least 95%, or 100% sequence identity or similarity with an amino acid sequence selected from SEQ ID NO: 8-18, preferably selected from SEQ ID NO: 10, 12, 14, 15, 16, or 17, more preferably selected from SEQ ID NO: 10, 15, 16, or 17, most preferably with SEQ ID NO: 10.

In some embodiments, a soluble γT-cell receptor chain or fragment thereof comprising a γCDR3 region comprises, consists essentially of, or consists of, preferably comprises, an amino acid sequence comprising at least 60%, at least 70%, at least 80%, at least 85%, at least 90%, at least 95%, at least 96%, at least 97%, at least 98%, at least 99%, or 100%, identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172, 196, 220, 244, 268, 292, or 316.

In preferred embodiments, the soluble γT-cell receptor chain or fragment thereof described herein further comprises a γCDR1 region represented by an amino acid sequence comprising at least 60%, at least 70%, at least 80%, at least 85%, at least 90%, at least 95%, or 100%, sequence identity or similarity with SEQ ID NO: 375, and a γCDR2 region represented by an amino acid sequence comprising at least 60%, at least 70%, at least 80%, at least 85%, at least 90%, at least 95%, or 100%, sequence identity or similarity with SEQ ID NO: 376.

In some embodiments, the γCDR3 region does not comprise SEQ ID NO: 379.

In some embodiments, the soluble γT-cell receptor chain or fragment thereof described herein further comprises a Cγ1 or Cγ2 constant region or fragment thereof. A Cγ2 constant region as used herein refers to a constant region encoded by a TRGC2 gene or variant thereof. Such a gene and region are known to the skilled person, for example see Uniprot Ref: P03986, SEQ ID NO: 381, and SEQ ID NO: 382 provided herein. A Cγ1 region as used herein refers to a constant region encoded by a TRGC1 gene or variant thereof. Such a gene and region are known to the skilled person, for example see Uniprot Ref: P0CF51 and SEQ ID NO: 112 provided herein. The TRGC genes encode the extracellular region of typically 110 amino acids (C-region), the connecting region (CO), the transmembrane region (TM), and the cytoplasmic region (CY). The TRGC1 gene comprises three exons and typically encodes a C-region of 173 AA (Cγ1), whereas the TRGC2 gene comprises four or five exons, owing to the duplication or triplication of a region that includes Exon 2 (EX2, EX2T and/or EX2R) and typically encodes a C-region (Cγ2) of 189 or 205 AA, respectively. Accordingly, a TRGC2 (Cγ2) region typically differs from a TRGC1 (Cγ1) region by having 16-32 extra amino acids in the connecting peptide. Additionally, Exon 2 of the TRGC1 gene has a cysteine involved in the interchain disulfide bridge, whereas the cysteine is not conserved in Exon 2 of the human TRGC2 gene. The frequency of γδTCR comprising Cγ1 or Cγ2 regions differs among the different γδT-cell subsets. For example, the constant gamma region of the Vγ9Vδ2 TCR expressed by the most abundant γ6T lymphocytes in human adult blood is exclusively encoded by TRGC1 gene, while the non-Vγ9Vδ2 TCRs tend to express a Cγ2 domain encoded by the TRGC2 gene.

In some embodiments, the Cγ1 constant region or fragment thereof comprises an amino acid sequence comprising at least 60%, at least 70%, at least 80%, at least 85%, at least 90%, at least 95%, or 100% identity or similarity with SEQ ID NO: 112. In some embodiments, the Cγ2 constant region or fragment thereof comprises an amino acid sequence comprising at least 60%, at least 70%, at least 80%, at least 85%, at least 90%, at least 95%, or 100% identity or similarity with SEQ ID NO: 381 or SEQ ID NO: 382.

In some embodiments, the Cγ1 constant region, the Cγ2 constant region, or the fragment there of lacks the transmembrane and/or cytoplasmic region or a fragment thereof. In some embodiments, a Cγ1 region or a fragment thereof does not comprise SEQ ID NO: 102 or a fragment thereof. In some embodiments, a Cγ2 region or a fragment thereof does not comprise SEQ ID NO: 103 or a fragment thereof.

Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In some embodiments, a soluble γT-cell receptor chain or fragment thereof comprising a γCDR3 region provided herein mediates an anti-tumour or anti-infective response that is improved relative to a control γT-cell receptor chain, preferably a γT-cell receptor chain comprising SEQ ID NO: 4 and/or a γT-cell receptor chain comprising a γCDR3 region represented by SEQ ID NO: 1 or SEQ ID NO: 379. In some embodiments, the improvement is of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, or at least 10-fold relative to a control γT-cell receptor chain, preferably a γT-cell receptor chain comprising SEQ ID NO: 4 and/or a γT-cell receptor chain comprising a γCDR3 region represented by SEQ ID NO: 1 or SEQ ID NO: 379.

In some embodiments, a soluble γT-cell receptor chain or fragment thereof comprising a γCDR3 region provided herein has a target specificity and/or affinity that is improved relative to a control γT-cell receptor chain, preferably a γT-cell receptor chain comprising SEQ ID NO: 4 and/or a γT-cell receptor chain comprising a γCDR3 region represented by SEQ ID NO: 1 or SEQ ID NO: 379. In some embodiments, the improvement is of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, at least 10-fold, or at least 100-fold relative to a control γT-cell receptor chain, preferably a γT-cell receptor chain comprising SEQ ID NO: 4 and/or a γT-cell receptor chain comprising a γCDR3 region represented by SEQ ID NO: 1 or SEQ ID NO: 379. Preferably, the target is endothelial protein C receptor (EPCR) or a cell expressing EPCR, for example a cancer cell expressing EPCR.

Another example of a control soluble γT-cell receptor chain comprises SEQ ID NO: 336 and/or comprises a γCDR3 region represented by SEQ ID NO: 7.

In an aspect, there is provided a soluble δT-cell receptor chain or fragment thereof comprising a δCDR3 region, said receptor chain or fragment thereof being represented by an amino acid sequence comprising an amino acid modification relative to a reference sequence, preferably relative to SEQ ID NO: 2 or SEQ ID NO: 6. The amino acid modification may be at a position corresponding to a position selected from one or more positions in the reference sequence.

In an aspect, there is provided a soluble δT-cell receptor chain or fragment thereof comprising a δCDR3 region, said receptor chain or fragment thereof comprising a modification in the δCDR3 region at an amino acid position corresponding to a position selected from one or more of positions 122-127 of SEQ ID NO: 6. In some embodiments, a soluble δT-cell receptor chain or fragment thereof comprising a δCDR3 region comprises a modification in the δCDR3 region at an amino acid position corresponding to a position selected from one or more of positions 7-12 of SEQ ID NO: 2.

In an aspect, there is provided a soluble δT-cell receptor chain or a fragment thereof comprising a δCDR3 region, wherein said δCDR3 region is represented by an amino acid sequence comprising at least 60%, at least 70%, at least 80%, at least 85%, or at least 90%, sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 122-127 of SEQ ID NO: 6, or from one or more of positions 7-12 of SEQ ID NO: 2, preferably from one or more of positions 7-12 of SEQ ID NO: 2.

Modifications are discussed earlier herein. For example, a modification may be an amino acid substitution, insertion, deletion, or a combination thereof, preferably it is an amino acid substitution.

In some embodiments, a soluble δT-cell receptor chain or fragment thereof comprising a δCDR3 region comprises at least one, at least two, at least three, at least four, at least five, or at least six amino acid modifications in the δCDR3 region relative to SEQ ID NO: 2 at amino acid positions corresponding to positions 122-127 of SEQ ID NO: 6 or positions 7-12 of SEQ ID NO: 2. In some embodiments, a soluble δT-cell receptor chain or fragment thereof comprising a δCDR3 region comprises from 1 to 6, from 1 to 5, from 1 to 4, from 1 to 3, or from 1 to 2 amino acid modifications in the δCDR3 region relative to SEQ ID NO: 2 at amino acid positions corresponding to positions 122-127 of SEQ ID NO: 6 or positions 7-12 of SEQ ID NO: 2.

In some embodiments, a soluble δT-cell receptor chain or fragment thereof comprising a δCDR3 region comprises at least one, at least two, at least three, at least four, at least five, or at least six amino acid substitutions in the δCDR3 region relative to SEQ ID NO: 2 at amino acid positions corresponding to positions 122-127 of SEQ ID NO: 6 or positions 7-12 of SEQ ID NO: 2. In some embodiments, a soluble δT-cell receptor chain or fragment thereof comprising a δCDR3 region comprises from 1 to 6, from 1 to 5, from 1 to 4, from 1 to 3, or from 1 to 2 amino acid substitutions in the δCDR3 region relative to SEQ ID NO: 2 at amino acid positions corresponding to positions 122-127 of SEQ ID NO: 6 or positions 7-12 of SEQ ID NO: 2. Preferably, an amino acid substitution is a substitution of a hydrophobic, charged, or polar amino acid.

In some embodiments, a soluble δT-cell receptor chain or fragment thereof comprising a δCDR3 region comprises at least one, at least two, at least three, at least four, at least five, or at least six amino acid deletions in the δCDR3 region relative to SEQ ID NO: 2 at amino acid positions corresponding to positions 122-127 of SEQ ID NO: 6 or positions 7-12 of SEQ ID NO: 2. In some embodiments, a soluble δT-cell receptor chain or fragment thereof comprising a δCDR3 region comprises from 1 to 6, from 1 to 5, from 1 to 4, from 1 to 3, or from 1 to 2 amino acid deletions in the δCDR3 region relative to SEQ ID NO: 2 at amino acid positions corresponding to positions 122-127 of SEQ ID NO: 6 or positions 7-12 of SEQ ID NO: 2.

In some embodiments, a soluble δT-cell receptor chain or fragment thereof comprising a δCDR3 region comprises at least one, at least two, at least three, at least four, at least five, or at least six amino acid insertions in the δCDR3 region relative to SEQ ID NO: 2 at amino acid positions corresponding to positions 122-127 of SEQ ID NO: 6 or positions 7-12 of SEQ ID NO: 2. In some embodiments, a soluble δT-cell receptor chain or fragment thereof comprising a δCDR3 region comprises from 1 to 6, from 1 to 5, from 1 to 4, from 1 to 3, or from 1 to 2 amino acid insertions in the γCDR3 region relative to SEQ ID NO: 2 at amino acid positions corresponding to positions 122-127 of SEQ ID NO: 6 or positions 7-12 of SEQ ID NO: 2.

In some embodiments, a soluble δT-cell receptor chain or fragment thereof comprising a δCDR3 region comprises a modification in the δCDR3 region relative to SEQ ID NO: 2 at an amino acid position corresponding to position 122 of SEQ ID NO: 6 or to position 7 of SEQ ID NO: 2. Preferably, said modification is an amino acid substitution (e.g., a substitution of an isoleucine), more preferably an amino acid substitution by a leucine, most preferably an amino acid substitution of an isoleucine by a leucine.

In some embodiments, a soluble δT-cell receptor chain or fragment thereof comprising a δCDR3 region comprises a modification in the δCDR3 region relative to SEQ ID NO: 2 at an amino acid position corresponding to position 123 of SEQ ID NO: 6 or to position 8 of SEQ ID NO: 2. Preferably, said modification is an amino acid substitution (e.g., a substitution of an arginine), more preferably an amino acid substitution by a lysine, most preferably an amino acid substitution of an arginine by a lysine.

In some embodiments, a soluble δT-cell receptor chain or fragment thereof comprising a δCDR3 region comprises a modification in the δCDR3 region relative to SEQ ID NO: 2 at an amino acid position corresponding to position 124 of SEQ ID NO: 6 or to position 9 of SEQ ID NO: 2.

In some embodiments, a soluble δT-cell receptor chain or fragment thereof comprising a δCDR3 region comprises a modification in the δCDR3 region relative to SEQ ID NO: 2 at an amino acid position corresponding to position 125 of SEQ ID NO: 6 or to position 10 of SEQ ID NO: 2. Preferably, said modification is an amino acid substitution (e.g., a substitution of a tyrosine), more preferably an amino acid substitution by a phenylalanine. most preferably an amino acid substitution of a tyrosine by a phenylalanine.

In some embodiments, a soluble δT-cell receptor chain or fragment thereof comprising a δCDR3 region comprises a modification in the δCDR3 region relative to SEQ ID NO: 2 at an amino acid position corresponding to position 126 of SEQ ID NO: 6 or to position 11 of SEQ ID NO: 2.

In some embodiments, a soluble δT-cell receptor chain or fragment thereof comprising a δCDR3 region comprises a modification in the δCDR3 region relative to SEQ ID NO: 2 at an amino acid position corresponding to position 127 of SEQ ID NO: 6 or to position 12 of SEQ ID NO: 2.

Preferably, the δCDR3 region is represented by an amino acid sequence comprising at least 60%, at least 70%, at least 80%, at least 85%, or at least 90%, sequence identity or similarity with SEQ ID NO: 2.

In some embodiments, a preferred soluble δT-cell receptor chain or fragment thereof comprising a δCDR3 region comprises a modification in the δCDR3 region relative to SEQ ID NO: 2 at an amino acid position corresponding to a position selected from one or more of positions 122-127 of SEQ ID NO: 6, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, a preferred soluble δT-cell receptor chain or fragment thereof comprising a δCDR3 region comprises a modification in the δCDR3 region relative to SEQ ID NO: 2 at an amino acid position corresponding to a position selected from one or more of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

Accordingly, in some embodiments, the δCDR3 region is represented by an amino acid sequence comprising at least 60%, at least 70%, at least 80%, at least 85%, or at least 90%, sequence identity or similarity with SEQ ID NO: 2, and comprises an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprises IKGFTG (SEQ ID NO: 97), at the amino acid positions corresponding to positions 122-127 of SEQ ID NO: 6 or to positions 7-12 of SEQ ID NO: 2, preferably to positions 7-12 of SEQ ID NO: 2.

In some embodiments, a soluble δT-cell receptor chain or fragment thereof comprising a δCDR3 region comprises, consists essentially of, or consists of, preferably comprises, an amino acid sequence having at least 60%, at least 70%, at least 80%, at least 90%, at least 95%, at least 96%, or at least 97%, sequence identity or similarity with the amino acid sequence SEQ ID NO: 6 and comprises a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, preferably from one or more of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, a soluble δT-cell receptor chain or fragment thereof comprises a δCDR3 region represented by an amino acid sequence having at least 60%, at least 70%, at least 80%, at least 90%, at least 95%, or 100% sequence identity or similarity with an amino acid sequence selected from SEQ ID NO: 20-27 or 110, preferably selected from SEQ ID NO: 21, 22, 23, 24, 26, or 110, more preferably selected from SEQ ID NO: 21, 22 or 23, most preferably with SEQ ID NO: 23.

In some embodiments, a soluble δT-cell receptor chain or fragment thereof comprising a δCDR3 region comprises, consists essentially of, or consists of, preferably comprises, an amino acid sequence comprising at least 60%, at least 70%, at least 80%, at least 85%, at least 90%, at least 95%, at least 96%, at least 97%, at least 98%, at least 99%, or 100%, identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330.

In preferred embodiments, the soluble δT-cell receptor chain or fragment thereof described herein further comprises a δCDR1 region represented by an amino acid sequence comprising at least 60%, at least 70%, at least 80%, at least 85%, at least 90%, at least 95%, or 100%, sequence identity or similarity with SEQ ID NO: 377, and a δCDR2 region represented by an amino acid sequence comprising at least 60%, at least 70%, at least 80%, at least 85%, at least 90%, at least 95%, or 100%, sequence identity or similarity with SEQ ID NO: 378.

In some embodiments, the soluble δT-cell receptor chain or fragment thereof described herein further comprises a Cδ constant region or fragment thereof. A Cδ constant region as used herein refers to a constant region encoded by a TRDC gene or variant thereof. Such a gene and region are known to the skilled person, for example see Uniprot Ref: B7Z8K6, and SEQ ID NO: 383 provided herein.

In some embodiments, the Cδ constant region or fragment thereof comprises an amino acid sequence comprising at least 60%, at least 70%, at least 80%, at least 85%, at least 90%, at least 95%, or 100% identity or similarity with SEQ ID NO: 383.

In some embodiments, the Cδ constant region or the fragment thereof lacks the transmembrane and/or cytoplasmic region or a fragment thereof. In some embodiments, the Cδ region or fragment thereof does not comprise SEQ ID NO: 104 or a fragment thereof.

In some embodiments, the δCDR3 region does not comprise SEQ ID NO: 380.

Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In some embodiments, a soluble δT-cell receptor chain or fragment thereof comprising a δCDR3 region provided herein mediates an anti-tumour or anti-infective response that is improved relative to a control δT-cell receptor chain, preferably a δT-cell receptor chain comprising SEQ ID NO: 6 and/or a δT-cell receptor chain comprising a δCDR3 region represented by SEQ ID NO: 2 or SEQ ID NO: 380. In some embodiments, the improvement is of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, or at least 10-fold relative to a control δT-cell receptor chain, preferably a δT-cell receptor chain comprising SEQ ID NO: 6 and/or a δT-cell receptor chain comprising a δCDR3 region represented by SEQ ID NO: 2 or SEQ ID NO: 380.

In some embodiments, a soluble δT-cell receptor chain or fragment thereof comprising a δCDR3 region provided herein has a target specificity and/or affinity that is improved relative to a control δT-cell receptor chain, preferably a δT-cell receptor chain comprising SEQ ID NO: 6 and/or a δT-cell receptor chain comprising a δCDR3 region represented by SEQ ID NO: 2 or SEQ ID NO: 380. In some embodiments, the improvement is of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, at least 10-fold, or at least 100-fold relative to a control δT-cell receptor chain, preferably a δT-cell receptor chain comprising SEQ ID NO: 6 and/or a δT-cell receptor chain comprising a δCDR3 region represented by SEQ ID NO: 2 or SEQ ID NO: 380. Preferably, the target is endothelial protectin C receptor (EPCR) or a cell expressing EPCR, for example a cancer cell expressing EPCR.

Another example of a control soluble δT-cell receptor chain comprises SEQ ID NO: 348 and/or comprises a δCDR3 region represented by SEQ ID NO: 19.

It is understood that a soluble γT-cell receptor chain or fragment thereof described herein may be paired with a soluble δT-cell receptor chain or fragment thereof. Pairing with any soluble δT-cell receptor chain or fragment thereof may be contemplated, as long as they are able to form a soluble γδTCR that can mediate an anti-tumour or anti-infective response.

It is also understood that a soluble δT-cell receptor chain or fragment thereof described herein may be paired with a soluble γT-cell receptor chain or fragment thereof. Pairing with any soluble γT-cell receptor chain or fragment thereof may be contemplated, as long as they are able to form a soluble γδTCR that can mediate an anti-tumour or anti-infective response.

In an aspect, there is provided a soluble γδT-cell receptor or fragment thereof comprising a γCDR3 and a δCDR3 region, wherein the γδT-cell receptor or fragment thereof comprises a soluble γT-cell receptor chain or fragment thereof as described herein, and/or, preferably and, a soluble δT-cell receptor chain or fragment thereof as described herein.

In some embodiments, a soluble γδT-cell receptor or fragment thereof comprising a γCDR3 and a δCDR3 region comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 116-122, preferably positions 117-121, of SEQ ID NO: 4, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6.

In some embodiments, a soluble γδT-cell receptor or fragment thereof comprising a γCDR3 and a δCDR3 region comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 4-10, preferably positions 5-9, of SEQ ID NO: 1, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 7-12 of SEQ ID NO: 2.

In some embodiments, a soluble γδT-cell receptor or fragment thereof comprising a γCDR3 and a δCDR3 region comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 116-122, preferably positions 117-121, of SEQ ID NO: 4, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 4-10, preferably positions 5-9, of SEQ ID NO: 1, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 7-12 of SEQ ID NO: 2.

In some embodiments, a soluble γδT-cell receptor or fragment thereof comprising a γCDR3 and a δCDR3 region comprises:
a soluble γT-cell receptor chain or a fragment thereof comprising a γCDR3 region, wherein said γCDR3 region is represented by an amino acid sequence comprising at least 60%, at least 70%, at least 80%, at least 85%, or at least 90%, sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 116-122 of SEQ ID NO: 4, or from one or more of positions 4-10 of SEQ ID NO: 1, and/or, preferably and;
a soluble δT-cell receptor chain or a fragment thereof comprising a δCDR3 region, wherein said δCDR3 region is represented by an amino acid sequence comprising at least 60%, at least 70%, at least 80%, at least 85%, or at least 90% sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 122-127 of SEQ ID NO: 6, or from one or more of positions 7-12 of SEQ ID NO: 2.

Suitable amino acid modifications for soluble γT-cell receptor chains, δT-cell receptor chains, or fragments thereof have been described earlier herein. Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In some embodiments, a soluble γδT-cell receptor or fragment thereof comprising a γCDR3 and a δCDR3 region comprises:
a soluble γT-cell receptor chain or fragment thereof comprising a γCDR3 region, said receptor or fragment thereof comprising a modification in the γCDR3 region relative to SEQ ID NO: 1 at an amino acid position corresponding to a position selected from one or more of positions 117-121 of SEQ ID NO: 4 or of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369), and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, a soluble γδT-cell receptor or fragment thereof comprising a γCDR3 and a δCDR3 region comprises:
a soluble γT-cell receptor chain or fragment thereof comprising a γCDR3 region, said receptor or fragment thereof comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, said modification selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGYYYK (SEQ ID NO: 88), WDGAYYK (SEQ ID NO: 89), or WDGSYYK (SEQ ID NO: 90), more preferably is WDAFYYK (SEQ ID NO: 83), and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, a soluble γδT-cell receptor or fragment thereof comprising a γCDR3 and a δCDR3 region comprises:
a soluble γT-cell receptor chain or fragment thereof comprising a γCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 (or with an amino acid sequence encoded by SEQ ID NO: 3) and comprising a modification in the γCDR3 region relative to SEQ ID NO: 1 at an amino acid position corresponding to a position selected from one or more of positions 117-121 of SEQ ID NO: 4 or of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369), and/or, preferably and;
a soluble δT-cell receptor chain or fragment thereof comprising a δCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 (or with an amino acid sequence encoded by SEQ ID NO: 5) and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, a soluble γδT-cell receptor or fragment thereof comprising a γCDR3 and a δCDR3 region comprises:
a soluble γT-cell receptor chain or fragment thereof comprising a γCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 (or with an amino acid sequence encoded by SEQ ID NO: 3) and comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, said modification preferably selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), more preferably selected from WDAFYYK (SEQ ID NO: 83), WDGYYYK (SEQ ID NO: 88), WDGAYYK (SEQ ID NO: 89), or WDGSYYK (SEQ ID NO: 90), most preferably is WDAFYYK (SEQ ID NO: 83), and/or, preferably and;
a soluble δT-cell receptor chain or fragment thereof comprising a δCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 (or with an amino acid sequence encoded by SEQ ID NO: 5) and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, a soluble γδT-cell receptor or fragment thereof comprising a γCDR3 and a δCDR3 region comprises:
a γCDR3 region represented by an amino acid sequence comprising at least 60%, at least 70%, at least 80%, at least 85%, or at least 90%, sequence identity or similarity with SEQ ID NO: 1, and comprising an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374), preferably selected from the group consisting of DAFYY (SEQ ID NO: 369), DGYYY (SEQ ID NO: 372), DGAYY (SEQ ID NO: 373), and DGSYY (SEQ ID NO: 374), more preferably comprising DAFYY (SEQ ID NO: 369), at the amino acid positions corresponding to positions 117-121 of SEQ ID NO: 4 or to positions 5-9 of SEQ ID NO: 1, and/or, preferably and;
a δCDR3 region represented by an amino acid sequence comprising at least 60%, at least 70%, at least 80%, at least 85%, or at least 90%, sequence identity or similarity with SEQ ID NO: 2, and comprising an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprising IKGFTG (SEQ ID NO: 97), at the amino acid positions corresponding to positions 122-127 of SEQ ID NO: 6 or to positions 7-12 of SEQ ID NO: 2.

In some embodiments, a soluble γδT-cell receptor or fragment thereof comprising a γCDR3 and a δCDR3 region comprises:
a γCDR3 region represented by an amino acid sequence comprising at least 60%, at least 70%, at least 80%, at least 85%, or at least 90%, sequence identity or similarity with SEQ ID NO: 1, and comprising an amino acid sequence selected from the group consisting of WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), and WDGSYYK (SEQ ID NO: 90), preferably selected from the group consisting of WDAFYYK (SEQ ID NO: 83), WDGYYYK (SEQ ID NO: 88), WDGAYYK (SEQ ID NO: 89), and WDGSYYK (SEQ ID NO: 90), more preferably comprising WDAFYYK (SEQ ID NO: 83), at the amino acid positions corresponding to positions 116-122 of SEQ ID NO: 4 or to positions 4-10 of SEQ ID NO: 1, and/or, preferably and;
a δCDR3 region represented by an amino acid sequence comprising at least 60%, at least 70%, at least 80%, at least 85%, or at least 90%, sequence identity or similarity with SEQ ID NO: 2, and comprising an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprising IKGFTG (SEQ ID NO: 97), at the amino acid positions corresponding to positions 122-127 of SEQ ID NO: 6 or to positions 7-12 of SEQ ID NO: 2.

In some embodiments, a soluble γδT-cell receptor or fragment thereof comprising a γCDR3 and a δCDR3 region comprises:
a γCDR3 region represented by an amino acid sequence having at least 60%, at least 70%, at least 80%, at least 90%, at least 95%, or 100% sequence identity or similarity with an amino acid sequence selected from SEQ ID NO: 8-18, preferably selected from SEQ ID NO: 10, 12, 14, 15, 16, or 17, more preferably selected from SEQ ID NO: 10, 15, 16, or 17, most preferably with SEQ ID NO: 10, and/or, preferably and;
a δCDR3 region represented by an amino acid sequence having at least 60%, at least 70%, at least 80%, at least 90%, at least 95%, or 100% sequence identity or similarity with an amino acid sequence selected from SEQ ID NO: 20-27 or 110, preferably selected from SEQ ID NO: 21, 22, 23, 24, 26, or 110, more preferably selected from SEQ ID NO: 21, 22 or 23, most preferably with SEQ ID NO: 23.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence comprising at least 60%, at least 70%, at least 80%, at least 85%, at least 90%, at least 95%, at least 96%, at least 97%, at least 98%, at least 99%, or 100%, identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172, 196, 220, 244, 268, 292, or 316, and/or, preferably and;
a soluble δT-cell receptor chain or fragment thereof comprising a δCDR3 region comprising, consisting essentially of, or consisting of, preferably comprising, an amino acid sequence comprising at least 60%, at least 70%, at least 80%, at least 85%, at least 90%, at least 95%, at least 96%, at least 97%, at least 98%, at least 99%, or 100%, identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330.

In preferred embodiments, the soluble γδT-cell receptor or fragment thereof further comprises:
a γCDR1 region represented by an amino acid sequence comprising at least 60%, at least 70%, at least 80%, at least 85%, at least 90%, at least 95%, or 100%, sequence identity or similarity with SEQ ID NO: 375, and a γCDR2 region represented by an amino acid sequence comprising at least 70%, at least 80%, at least 85%, at least 90%, at least 95%, or 100%, sequence identity or similarity with SEQ ID NO: 376, and/or, preferably and;
a δCDR1 region represented by an amino acid sequence comprising at least 60%, at least 70%, at least 80%, at least 85%, at least 90%, at least 95%, or 100%, sequence identity or similarity with SEQ ID NO: 377, and a δCDR2 region represented by an amino acid sequence comprising at least 70%, at least 80%, at least 85%, at least 90%, at least 95%, or 100%, sequence identity or similarity with SEQ ID NO: 378.

In some embodiments, the soluble γδT-cell receptor or fragment thereof further comprises:
a Cγ1 constant region, a Cγ2 constant region, or a fragment thereof, and/or, preferably and;
a Cδ constant region or fragment thereof.

Preferably, the Cγ1 constant region or fragment thereof comprises an amino acid sequence comprising at least 60%, at least 70%, at least 80%, at least 85%, at least 90%, at least 95%, or 100% identity or similarity with SEQ ID NO: 112. Preferably, the Cγ2 constant region or fragment thereof comprises an amino acid sequence comprising at least 60%, at least 70%, at least 80%, at least 85%, at least 90%, at least 95%, or 100% identity or similarity with SEQ ID NO: 381 or SEQ ID NO: 382. Preferably, the Cδ constant region or fragment thereof comprises an amino acid sequence comprising at least 60%, at least 70%, at least 80%, at least 85%, at least 90%, at least 95%, or 100% identity or similarity with SEQ ID NO: 383.

In some embodiments, the γCDR3 region does not comprise SEQ ID NO: 379 and the δCDR3 region does not comprise SEQ ID NO: 380.

In some embodiments, a preferred soluble γδT-cell receptor or fragment thereof comprising a γCDR3 and a δCDR3 region comprises:
a soluble γT-cell receptor chain or fragment thereof comprising a γCDR3 region comprising the amino acid sequence DAFYY (SEQ ID NO: 369) at the amino acid positions corresponding to positions 117-121 of SEQ ID NO: 4 or to positions 5-9 of SEQ ID NO: 1, preferably comprising a γCDR3 region comprising the amino acid sequence SEQ ID NO: 10, and/or, preferably and;
a soluble δT-cell receptor chain or fragment thereof comprising a δCDR3 region comprising the amino acid sequence IKGFTG (SEQ ID NO: 97) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising WDAFYYK (SEQ ID NO: 83) in the γCDR3 region at amino acid positions corresponding to positions 116-122 of SEQ ID NO: 4, preferably comprising a γCDR3 region comprising amino acid sequence SEQ ID NO: 10, and/or, preferably and;
a soluble δT-cell receptor chain or fragment thereof comprising IKGFTG (SEQ ID NO: 97) in the δCDR3 region at amino acid positions corresponding to positions 122-127 of SEQ ID NO: 6, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising WDAFYYK (SEQ ID NO: 83) in the γCDR3 region at amino acid positions corresponding to positions 4-10 of SEQ ID NO: 1, preferably comprising a γCDR3 region comprising amino acid sequence SEQ ID NO: 10, and/or, preferably and;
a soluble δT-cell receptor chain or fragment thereof comprising IKGFTG (SEQ ID NO: 97) in the δCDR3 region at amino acid positions corresponding to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 124, 148, 172, 196, 220, 244, 268, 292, or 316, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 138, 162,186, 210, 234, 258, 282, 306, or 330.

In some embodiments, a soluble γδT-cell receptor or fragment thereof comprising a γCDR3 and a δCDR3 region comprises:
a soluble γT-cell receptor chain or fragment thereof comprising a γCDR3 region comprising the amino acid sequence DGAYY (SEQ ID NO: 373) at the amino acid positions corresponding to positions 117-121 of SEQ ID NO: 4 or to positions 5-9 of SEQ ID NO: 1, preferably comprising a γCDR3 region comprising the amino acid sequence SEQ ID NO: 16, and/or, preferably and;
a soluble δT-cell receptor chain or fragment thereof comprising a δCDR3 region comprising the amino acid sequence IKGYTG (SEQ ID NO: 96) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 22.

In some embodiments, a soluble γδT-cell receptor or fragment thereof comprising a γCDR3 and a δCDR3 region comprises:
a soluble γT-cell receptor chain or fragment thereof comprising WDGAYYK (SEQ ID NO: 89) in the γCDR3 region at amino acid positions corresponding to positions 116-122 of SEQ ID NO: 4, preferably comprising a γCDR3 region comprising amino acid sequence SEQ ID NO: 16, and/or, preferably and;
a soluble δT-cell receptor chain or fragment thereof comprising IKGYTG (SEQ ID NO: 96) in the δCDR3 region at amino acid positions corresponding to positions 122-127 of SEQ ID NO: 6, preferably comprising a δCDR3 region comprising amino acid sequence SEQ ID NO: 22.

In some embodiments, a soluble γδT-cell receptor or fragment thereof comprising a γCDR3 and a δCDR3 region comprises:
a soluble γT-cell receptor chain or fragment thereof comprising WDGAYYK (SEQ ID NO: 89) in the γCDR3 region at amino acid positions corresponding to positions 4-10 of SEQ ID NO: 1, preferably comprising a γCDR3 region comprising amino acid sequence SEQ ID NO: 16, and/or, preferably and;
a soluble δT-cell receptor chain or fragment thereof comprising IKGYTG (SEQ ID NO: 96) in the δCDR3 region at amino acid positions corresponding to positions 7-12 of SEQ ID NO: 2, preferably comprising a δCDR3 region comprising amino acid sequence SEQ ID NO: 22.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 130, 154, 178, 202, 226, 250, 274, 298, or 322 and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 137, 161, 185, 209, 233, 257, 281, 305, or 329.

In some embodiments, a soluble γδT-cell receptor or fragment thereof comprising a γCDR3 and a δCDR3 region comprises:
a soluble γT-cell receptor chain or fragment thereof comprising a γCDR3 region comprising the amino acid sequence DGSYY (SEQ ID NO: 374) at the amino acid positions corresponding to positions 117-121 of SEQ ID NO: 4 or to positions 5-9 of SEQ ID NO: 1, preferably comprising a γCDR3 region comprising the amino acid sequence SEQ ID NO: 17, and/or, preferably and;
a soluble δT-cell receptor chain or fragment thereof comprising a δCDR3 region comprising the amino acid sequence IKGFTG (SEQ ID NO: 97) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising WDGSYYK (SEQ ID NO: 90) in the γCDR3 region at amino acid positions corresponding to positions 116-122 of SEQ ID NO: 4, preferably comprising a γCDR3 region comprising amino acid sequence SEQ ID NO: 17, and/or, preferably and;
a soluble δT-cell receptor chain or fragment thereof comprising IKGFTG (SEQ ID NO: 97) in the δCDR3 region at amino acid positions corresponding to positions 122-127 of SEQ ID NO: 6, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising WDGSYYK (SEQ ID NO: 90) in the γCDR3 region at amino acid positions corresponding to positions 4-10 of SEQ ID NO: 1, preferably comprising a γCDR3 region comprising amino acid sequence SEQ ID NO: 17, and/or, preferably and;
a soluble δT-cell receptor chain or fragment thereof comprising IKGFTG (SEQ ID NO: 97) in the δCDR3 region at amino acid positions corresponding to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 131, 155, 179, 203, 227, 251, 275, 299, or 323, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 138, 162,186, 210, 234, 258, 282, 306, or 330.

In some embodiments, a soluble γδT-cell receptor or fragment thereof comprising a γCDR3 and a δCDR3 region comprises:
a soluble γT-cell receptor chain or fragment thereof comprising a γCDR3 region comprising the amino acid sequence DGYYY (SEQ ID NO: 372) at the amino acid positions corresponding to positions 117-121 of SEQ ID NO: 4 or to positions 5-9 of SEQ ID NO: 1, preferably comprising a γCDR3 region comprising the amino acid sequence SEQ ID NO: 15, and/or, preferably and;
a soluble δT-cell receptor chain or fragment thereof comprising a δCDR3 region comprising the amino acid sequence IKGFTG (SEQ ID NO: 97) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising WDGYYYK (SEQ ID NO: 88) in the γCDR3 region at amino acid positions corresponding to positions 116-122 of SEQ ID NO: 4, preferably comprising a γCDR3 region comprising amino acid sequence SEQ ID NO: 15, and/or, preferably and;
a soluble δT-cell receptor chain or fragment thereof comprising IKGFTG (SEQ ID NO: 97) in the δCDR3 region at amino acid positions corresponding to positions 122-127 of SEQ ID NO: 6, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising WDGYYYK (SEQ ID NO: 88) in the γCDR3 region at amino acid positions corresponding to positions 4-10 of SEQ ID NO: 1, preferably comprising a γCDR3 region comprising amino acid sequence SEQ ID NO: 15, and/or, preferably and;
a soluble δT-cell receptor chain or fragment thereof comprising IKGFTG (SEQ ID NO: 97) in the δCDR3 region at amino acid positions corresponding to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 129, 153, 177, 201, 225, 249, 273, 297, or 321, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 138, 162,186, 210, 234, 258, 282, 306, or 330.

In some embodiments, a soluble γδT-cell receptor or fragment thereof comprising a γCDR3 and a δCDR3 region comprises:
a soluble γT-cell receptor chain or fragment thereof comprising a γCDR3 region comprising the amino acid sequence DAFYY (SEQ ID NO: 369) at the amino acid positions corresponding to positions 117-121 of SEQ ID NO: 4 or to positions 5-9 of SEQ ID NO: 1, preferably comprising a γCDR3 region comprising the amino acid sequence SEQ ID NO: 10, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising WDAFYYK (SEQ ID NO: 83) in the γCDR3 region at amino acid positions corresponding to positions 116-122 of SEQ ID NO: 4, preferably comprising a γCDR3 region comprising amino acid sequence SEQ ID NO: 10, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising WDAFYYK (SEQ ID NO: 83) in the γCDR3 region at amino acid positions corresponding to positions 4-10 of SEQ ID NO: 1, preferably comprising a γCDR3 region comprising amino acid sequence SEQ ID NO: 10, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 124, 148, 172, 196, 220, 244, 268, 292, or 316, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising a γCDR3 region comprising the amino acid sequence DGYYY (SEQ ID NO: 372) at the amino acid positions corresponding to positions 117-121 of SEQ ID NO: 4 or to positions 5-9 of SEQ ID NO: 1, preferably comprising a γCDR3 region comprising the amino acid sequence SEQ ID NO: 15, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising WDGYYYK (SEQ ID NO: 88) in the γCDR3 region at amino acid positions corresponding to positions 116-122 of SEQ ID NO: 4, preferably comprising a γCDR3 region comprising amino acid sequence SEQ ID NO: 15, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising WDGYYYK (SEQ ID NO: 88) in the γCDR3 region at amino acid positions corresponding to positions 4-10 of SEQ ID NO: 1, preferably comprising a γCDR3 region comprising amino acid sequence SEQ ID NO: 15, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 129, 153, 177, 201, 225, 249, 273, 297, or 321, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, or 327.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising the amino acid sequence IRGFTG (SEQ ID NO: 95) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 21.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising IRGFTG (SEQ ID NO: 95) in the δCDR3 region at amino acid positions corresponding to positions 122-127 of SEQ ID NO: 6, preferably comprising a δCDR3 region comprising amino acid sequence SEQ ID NO: 21.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising IRGFTG (SEQ ID NO: 95) in the δCDR3 region at amino acid positions corresponding to positions 7-12 of SEQ ID NO: 2, preferably comprising a δCDR3 region comprising amino acid sequence SEQ ID NO: 21.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 136, 160,184, 208, 232, 256, 280, 304, or 328.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising the amino acid sequence IKGYTG (SEQ ID NO: 96) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 22.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising IKGYTG (SEQ ID NO: 96) in the δCDR3 region at amino acid positions corresponding to positions 122-127 of SEQ ID NO: 6, preferably comprising a δCDR3 region comprising amino acid sequence SEQ ID NO: 22.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising IKGYTG (SEQ ID NO: 96) in the δCDR3 region at amino acid positions corresponding to positions 7-12 of SEQ ID NO: 2, preferably comprising a δCDR3 region comprising amino acid sequence SEQ ID NO: 22.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 137, 161, 185, 209, 233, 257, 281, 305, or 329.

In some embodiments, a γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising the amino acid sequence IKGFTG (SEQ ID NO: 97) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising IKGFTG (SEQ ID NO: 97) in the δCDR3 region at amino acid positions corresponding to positions 122-127 of SEQ ID NO: 6, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising IKGFTG (SEQ ID NO: 97) in the δCDR3 region at amino acid positions corresponding to positions 7-12 of SEQ ID NO: 2, preferably comprising a δCDR3 region comprising amino acid sequence SEQ ID NO: 23.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising, consisting essentially of, or consisting oft preferably comprising, an amino acid sequence having at least 60%, at least 70%, at least 80%, at least 90%, at least 95%, or 100% sequence identity or similarity with SEQ ID NO: 138, 162,186, 210, 234, 258, 282, 306, or 330.

In some embodiments, a γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a δT-cell receptor chain or fragment thereof comprising a δCDR3 region comprising the amino acid sequence LKGFTG (SEQ ID NO: 111) at the amino acid positions corresponding to positions 122-127 of SEQ ID NO: 6 or to positions 7-12 of SEQ ID NO: 2, preferably comprising a δCDR3 region comprising amino acid sequence SEQ ID NO: 110.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising LKGFTG (SEQ ID NO: 111) in the δCDR3 region at amino acid positions corresponding to positions 122-127 of SEQ ID NO: 6, preferably comprising a δCDR3 region comprising amino acid sequence SEQ ID NO: 110.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising LKGFTG (SEQ ID NO: 111) in the δCDR3 region at amino acid positions corresponding to positions 7-12 of SEQ ID NO: 2, preferably comprising a δCDR3 region comprising amino acid sequence SEQ ID NO: 110.

In some embodiments, a soluble γδT-cell receptor or fragment thereof comprising a γCDR3 and a δCDR3 region comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with SEQ ID NO: 141, 165,189, 210, 237, 261, 285, 309, or 333.

In some embodiments, a soluble γδT-cell receptor or fragment thereof comprising a γCDR3 and a δCDR3 region provided herein mediates an anti-tumour or anti-infective response that is improved relative to a control γδT-cell receptor, preferably a γδT-cell receptor comprising a γT-cell receptor chain comprising SEQ ID NO: 4 and/or a γCDR3 region represented by SEQ ID NO: 1 and a δT-cell receptor chain comprising SEQ ID NO: 6 and/or a δCDR3 region represented by SEQ ID NO: 2. In some embodiments, the improvement is of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, or at least 10-fold relative to a control γδT-cell receptor, preferably a γδT-cell receptor comprising a γT-cell receptor chain comprising SEQ ID NO: 4 and/or a γCDR3 region represented by SEQ ID NO: 1 and a δT-cell receptor chain comprising SEQ ID NO: 6 and/or a δCDR3 region represented by SEQ ID NO: 2.

In some embodiments, a soluble γδT-cell receptor or fragment thereof comprising a γCDR3 and a δCDR3 region provided herein has a target specificity and/or affinity that is improved relative to a control γδT-cell receptor, preferably a γδT-cell receptor comprising a γT-cell receptor chain comprising SEQ ID NO: 4 and/or a γCDR3 region represented by SEQ ID NO: 1 and a δT-cell receptor chain comprising SEQ ID NO: 6 and/or a δCDR3 region represented by SEQ ID NO: 2. In some embodiments, the improvement is of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% (2-fold), at least 3-fold, at least 4-fold, at least 5-fold, at least 6-fold, at least 7-fold, at least 8-fold, at least 9-fold, at least 10-fold, or at least 100-fold relative to a control γδT-cell receptor, preferably a γδT-cell receptor comprising a γT-cell receptor chain comprising SEQ ID NO: 4 and/or a γCDR3 region represented by SEQ ID NO: 1 and a δT-cell receptor chain comprising SEQ ID NO: 6 and/or a δCDR3 region represented by SEQ ID NO: 2. Preferably, the target is endothelial protein C receptor (EPCR) or a cell expressing EPCR, for example a cancer cell expressing EPCR.

Another example of a control γδT-cell receptor comprises a γT-cell receptor chain comprising SEQ ID NO: 336 and/or a γCDR3 region represented by SEQ ID NO: 7 and a δT-cell receptor chain comprising SEQ ID NO: 348 and/or comprising a δCDR3 region represented by SEQ ID NO: 19.

The skilled person is able to identify amino acids positions in other soluble γT-cell receptor chains, δT-cell receptor chains, γδT-cell receptor chains, or fragments thereof, corresponding to specific positions in SEQ ID NOs: 1, 2, 4, and 6 using standardized nomenclature as described earlier herein.

A soluble γT-cell receptor chain or fragment thereof comprising a γCDR3 region, soluble δT-cell receptor chain or fragment thereof comprising a δCDR3 region, or soluble γδT-cell receptor chain or fragment thereof comprising a γCDR3 region and a δCDR3 region as described herein may be comprised in a soluble polypeptide.

Thus, in an aspect, there is provided a soluble polypeptide comprising a γT-cell receptor chain or a fragment thereof comprising a γCDR3 region, wherein said γCDR3 region is represented by an amino acid sequence comprising at least 60%, at least 70%, at least 80%, at least 85%, or at least 90% sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 116-122 of SEQ ID NO: 4, or from one or more of positions 4-10 of SEQ ID NO: 1, preferably from one or more of positions 4-10 of SEQ ID NO: 1. In some embodiments, it comprises a modification in the γCDR3 region relative to SEQ ID NO: 1 at an amino acid position corresponding to a position selected from one or more of positions 117-121 of SEQ ID NO: 4, or from one or more of positions 5-9 of SEQ ID NO: 1, preferably from one or more of positions 5-9 of SEQ ID NO: 1.

In some embodiments, a preferred soluble polypeptide comprises a γT-cell receptor chain or fragment thereof comprising a γCDR3 region comprises a modification in the γCDR3 region relative to SEQ ID NO: 1 at an amino acid position corresponding to a position selected from one or more of positions 117-121 of SEQ ID NO: 4 or from one or more of positions 5-9 of SEQ ID NO: 1, preferably from one or more of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369). In some embodiments, the γCDR3 region is represented by an amino acid sequence comprising at least 60%, at least 70%, at least 80%, at least 85%, or at least 90%, sequence identity or similarity with SEQ ID NO: 1, and comprises an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374), preferably selected from the group consisting of DAFYY (SEQ ID NO: 369), DGYYY (SEQ ID NO: 372), DGAYY (SEQ ID NO: 373), and DGSYY (SEQ ID NO: 374), more preferably comprises DAFYY (SEQ ID NO: 369), relative to SEQ ID NO: 1 at the amino acid positions corresponding to positions 117-121 of SEQ ID NO: 4 or to positions 5-9 of SEQ ID NO: 1, preferably to positions 5-9 of SEQ ID NO: 1.

In some embodiments, a soluble polypeptide comprises a γT-cell receptor chain or fragment thereof comprising a γCDR3 region, wherein said receptor chain or fragment thereof comprises a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, preferably of positions 4-10 of SEQ ID NO: 1, preferably wherein said modification is selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGYYYK (SEQ ID NO: 88), WDGAYYK (SEQ ID NO: 89), or WDGSYYK (SEQ ID NO: 90), more preferably is WDAFYYK (SEQ ID NO: 83).

In some embodiments, a soluble polypeptide comprises a γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 and comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, preferably of positions 4-10 of SEQ ID NO: 1, said modification preferably selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), more preferably selected from WDAFYYK (SEQ ID NO: 83), WDGYYYK (SEQ ID NO: 88), WDGAYYK (SEQ ID NO: 89), or WDGSYYK (SEQ ID NO: 90), most preferably is WDAFYYK (SEQ ID NO: 83).

In some embodiments, a soluble polypeptide comprises a γT-cell receptor chain or fragment thereof comprising a γCDR3 region, wherein the γCDR3 region is represented by an amino acid sequence having at least 60%, at least 70%, at least 80%, at least 90%, at least 95%, or 100% sequence identity or similarity with an amino acid sequence selected from SEQ ID NO: 1 or 8-18, preferably selected from SEQ ID NO: 10, 12, 14, 15, 16, or 17, more preferably selected from SEQ ID NO: 10, 15, 16, or 17, most preferably with SEQ ID NO: 10.

In some embodiments, a soluble polypeptide comprises a γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172, 196, 220, 244, 268, 292, or 316.

In preferred embodiments, the γT-cell receptor chain or fragment thereof comprised in the soluble polypeptide further comprises a γCDR1 region represented by an amino acid sequence comprising at least 60%, at least 70%, at least 80%, at least 85%, at least 90%, at least 95%, or 100%, sequence identity or similarity with SEQ ID NO: 375, and a γCDR2 region represented by an amino acid sequence comprising at least 60%, at least 70%, at least 80%, at least 85%, at least 90%, at least 95%, or 100%, sequence identity or similarity with SEQ ID NO: 376. In some embodiments, the γT-cell receptor chain or fragment thereof comprised in the soluble polypeptide further comprises a Cγ1 or Cγ2 constant region or fragment thereof. In some embodiments, the Cγ1 constant region or fragment thereof comprises an amino acid sequence comprising at least 60%, at least 70%, at least 80%, at least 85%, at least 90%, at least 95%, or 100% identity or similarity with SEQ ID NO: 112. In some embodiments, the Cγ2 constant region or fragment thereof comprises an amino acid sequence comprising at least 60%, at least 70%, at least 80%, at least 85%, at least 90%, at least 95%, or 100% identity or similarity with SEQ ID NO: 381 or SEQ ID NO: 382.

In some embodiments, the Cγ1 constant region, the Cγ2 constant region, or the fragment there of lacks the transmembrane and/or cytoplasmic region or a fragment thereof. In some embodiments, a Cγ1 region or fragment thereof does not comprise SEQ ID NO: 102 or a fragment thereof. In some embodiments, a Cγ2 or a fragment thereof region does not comprise SEQ ID NO: 103 or a fragment thereof.

In some embodiments, a soluble polypeptide does not comprise a γCDR3 region comprising SEQ ID NO: 379.

In an aspect, there is provided a soluble polypeptide comprising a δT-cell receptor chain or a fragment thereof comprising a δCDR3 region, wherein said δCDR3 region is represented by an amino acid sequence comprising at least 60%, at least 70%, at least 80%, at least 85%, or at least 90%, sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 122-127 of SEQ ID NO: 6, or from one or more of positions 7-12 of SEQ ID NO: 2, preferably from one or more of positions 7-12 of SEQ ID NO: 2. In some embodiments, the δCDR3 region is represented by an amino acid sequence comprising at least 60%, at least 70%, at least 80%, at least 85%, or at least 90%, sequence identity or similarity with SEQ ID NO: 2, and comprises an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprises IKGFTG (SEQ ID NO: 97), at the amino acid positions corresponding to positions 122-127 of SEQ ID NO: 6 or to positions 7-12 of SEQ ID NO: 2, preferably to positions 7-12 of SEQ ID NO: 2.

In some embodiments, a soluble polypeptide comprises a δT-cell receptor chain or fragment thereof comprising a δCDR3 region, wherein said receptor chain or fragment thereof comprises a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, preferably of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, a soluble polypeptide comprises a δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, preferably of positions 7-21 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, a soluble polypeptide comprises a δT-cell receptor chain or fragment thereof comprising a δCDR3 region, wherein the δCDR3 region is represented by an amino acid sequence having at least 60%, at least 70%, at least 80%, at least 90%, at least 95%, or 100% sequence identity or similarity with an amino acid sequence selected from SEQ ID NO: 2, 20-27, or 110, preferably selected from SEQ ID NO: 21, 22, 23, 24, 26, or 110, more preferably selected from SEQ ID NO: 21, 22 or 23, most preferably with SEQ ID NO NO: 23.

In some embodiments, a soluble polypeptide comprises a δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330.

In preferred embodiments, the δT-cell receptor chain or fragment thereof comprised in the soluble polypeptide further comprises a δCDR1 region represented by an amino acid sequence comprising at least 60%, at least 70%, at least 80%, at least 85%, at least 90%, at least 95%, or 100%, sequence identity or similarity with SEQ ID NO: 377, and a δCDR2 region represented by an amino acid sequence comprising at least 60%, at least 70%, at least 80%, at least 85%, at least 90%, at least 95%, or 100%, sequence identity or similarity with SEQ ID NO: 378. In some embodiments, the δT-cell receptor chain or fragment thereof comprised in the soluble polypeptide further comprises a Cδ constant region or fragment thereof. In some embodiments, the Cδ constant region or fragment thereof comprises an amino acid sequence comprising at least 60%, at least 70%, at least 80%, at least 85%, at least 90%, at least 95%, or 100% identity or similarity with SEQ ID NO: 383. In some embodiments, the Cδ constant region or the fragment thereof lacks the transmembrane and/or cytoplasmic region or a fragment thereof. In some embodiments, the Cδ region or fragment thereof does not comprise SEQ ID NO: 104 or a fragment thereof.

In some embodiments, a soluble polypeptide does not comprise a δCDR3 region comprising SEQ ID NO: 380.

In an aspect, there is provided a a soluble polypeptide comprising a γδT-cell receptor or fragment thereof comprising a γCDR3 and a δCDR3 region, wherein the γδT-cell receptor or fragment thereof comprises a soluble γT-cell receptor chain or fragment thereof as described herein, and/or, preferably and, a soluble δT-cell receptor chain or fragment thereof as described herein.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 116-122, preferably 117-121, of SEQ ID NO: 4 or of positions 4-10, preferably 5-9, of SEQ ID NO: 1, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 116-122, preferably 117-121, of SEQ ID NO: 4 or of positions 4-10, preferably 5-9, of SEQ ID NO: 1, and/or, preferably and;
a δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with SEQ ID NO: 135, 159, 183, 207, 231, 255, 279, 303, 327.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or a fragment thereof comprising a γCDR3 region, wherein said γCDR3 region is represented by an amino acid sequence comprising at least 60%, at least 70%, at least 80%, at least 85%, or at least 90%, sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 116-122 of SEQ ID NO: 4, or from one or more of positions 4-10 of SEQ ID NO: 1, and/or, preferably and;
a soluble δT-cell receptor chain or a fragment thereof comprising a δCDR3 region, wherein said δCDR3 region is represented by an amino acid sequence comprising at least 60%, at least 70%, at least 80%, at least 85%, or at least 90% sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 122-127 of SEQ ID NO: 6, or from one or more of positions 7-12 of SEQ ID NO: 2.

In some embodiments, a soluble polypeptide comprises a γδT-cell receptor or fragment thereof comprising a γCDR3 and a δCDR3 region comprising:
a γCDR3 region represented by an amino acid sequence having at least 60%, at least 70%, at least 80%, at least 90%, at least 95%, or 100% sequence identity or similarity with an amino acid sequence selected from SEQ ID NO: 1 or 8-18, preferably selected from SEQ ID NO: 10, 12, 14, 15, 16, or 17, more preferably selected from SEQ ID NO: 10, 15, 16, or 17, most preferably with SEQ ID NO: 10, and/or, preferably and;
a δCDR3 region represented by an amino acid sequence having at least 60%, at least 70%, at least 80%, at least 90%, at least 95%, or 100% sequence identity or similarity with an amino acid sequence selected from SEQ ID NO: 2, 20-27, or 110, preferably selected from SEQ ID NO: 21, 22, 23, 24, 26, or 110, more preferably selected from SEQ ID NO: 21, 22 or 23, most preferably with SEQ ID NO: 23.

In some embodiments, a soluble polypeptide comprises a γδT-cell receptor or fragment thereof comprising a γCDR3 and a δCDR3 region comprising:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172, 196, 220, 244, 268, 292, or 316, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330.

Suitable amino acid modifications for γT-cell receptor chains, δT-cell receptor chains, or fragments thereof have been described earlier herein. Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor or fragment thereof comprising a modification in the γCDR3 region relative to SEQ ID NO: 1 at an amino acid position corresponding to a position selected from one or more of positions 117-121 of SEQ ID NO: 4 or of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369), and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, a soluble polypeptide comprises a γδT-cell receptor or fragment thereof comprising a γCDR3 and a δCDR3 region, wherein the γδT-cell receptor or fragment thereof comprises:
a γCDR3 region represented by an amino acid sequence comprising at least 60%, at least 70%, at least 80%, at least 85%, or at least 90%, sequence identity or similarity with SEQ ID NO: 1, and comprising an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374), preferably selected from the group consisting of DAFYY (SEQ ID NO: 369), DGYYY (SEQ ID NO: 372), DGAYY (SEQ ID NO: 373), and DGSYY (SEQ ID NO: 374), more preferably comprising DAFYY (SEQ ID NO: 369), at the amino acid positions corresponding to positions 117-121 of SEQ ID NO: 4 or to positions 5-9 of SEQ ID NO: 1, and/or, preferably and;
a δCDR3 region represented by an amino acid sequence comprising at least 60%, at least 70%, at least 80%, at least 85%, or at least 90%, sequence identity or similarity with SEQ ID NO: 2, and comprising an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprising IKGFTG (SEQ ID NO: 97), at the amino acid positions corresponding to positions 122-127 of SEQ ID NO: 6 or to positions 7-12 of SEQ ID NO: 2.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 (or with an amino acid sequence encoded by SEQ ID NO: 3) and comprising a modification in the γCDR3 region relative to SEQ ID NO: 1 at an amino acid position corresponding to a position selected from one or more of positions 117-121 of SEQ ID NO: 4 or of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369), and/or, preferably and;
a soluble δT-cell receptor chain or fragment thereof comprising a δCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 (or with an amino acid sequence encoded by SEQ ID NO: 5) and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor or fragment thereof comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, said modification selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGYYYK (SEQ ID NO: 88), WDGAYYK (SEQ ID NO: 89), or WDGSYYK (SEQ ID NO: 90), more preferably is WDAFYYK (SEQ ID NO: 83), and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, a soluble polypeptide comprises a γδT-cell receptor or fragment thereof comprising a γCDR3 and a δCDR3 region, wherein the γδT-cell receptor or fragment thereof comprises:
a γCDR3 region represented by an amino acid sequence comprising at least 60%, at least 70%, at least 80%, at least 85%, or at least 90%, sequence identity or similarity with SEQ ID NO: 1, and comprising an amino acid sequence selected from the group consisting of WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), and WDGSYYK (SEQ ID NO: 90), preferably selected from the group consisting of WDAFYYK (SEQ ID NO: 83), WDGYYYK (SEQ ID NO: 88), WDGAYYK (SEQ ID NO: 89), and WDGSYYK (SEQ ID NO: 90), more preferably comprising WDAFYYK (SEQ ID NO: 83), at the amino acid positions corresponding to positions 116-122 of SEQ ID NO: 4 or to positions 4-10 of SEQ ID NO: 1, and/or, preferably and;
a δCDR3 region represented by an amino acid sequence comprising at least 60%, at least 70%, at least 80%, at least 85%, or at least 90%, sequence identity or similarity with SEQ ID NO: 2, and comprising an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprising IKGFTG (SEQ ID NO: 97), at the amino acid positions corresponding to positions 122-127 of SEQ ID NO: 6 or to positions 7-12 of SEQ ID NO: 2.

In some embodiments, a soluble polypeptide comprises a γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 (or with an amino acid sequence encoded by SEQ ID NO: 3) and comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, said modification preferably selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), more preferably selected from WDAFYYK (SEQ ID NO: 83), WDGYYYK (SEQ ID NO: 88), WDGAYYK (SEQ ID NO: 89), or WDGSYYK (SEQ ID NO: 90), most preferably is WDAFYYK (SEQ ID NO: 83), and/or, preferably and;
a soluble δT-cell receptor chain or fragment thereof comprising a δCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 (or with an amino acid sequence encoded by SEQ ID NO: 5) and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In preferred embodiments, the γδT-cell receptor or fragment thereof comprised in the soluble polypeptide further comprises:
a γCDR1 region represented by an amino acid sequence comprising at least 60%, at least 70%, at least 80%, at least 85%, at least 90%, at least 95%, or 100%, sequence identity or similarity with SEQ ID NO: 375, and a γCDR2 region represented by an amino acid sequence comprising at least 70%, at least 80%, at least 85%, at least 90%, at least 95%, or 100%, sequence identity or similarity with SEQ ID NO: 376, and/or, preferably and;
a δCDR1 region represented by an amino acid sequence comprising at least 60%, at least 70%, at least 80%, at least 85%, at least 90%, at least 95%, or 100%, sequence identity or similarity with SEQ ID NO: 377, and a δCDR2 region represented by an amino acid sequence comprising at least 70%, at least 80%, at least 85%, at least 90%, at least 95%, or 100%, sequence identity or similarity with SEQ ID NO: 378.

In some embodiments, the γδT-cell receptor or fragment thereof comprised in the soluble polypeptide further comprises:
a Cγ1 constant region, a Cγ2 constant region, or a fragment thereof, and/or, preferably and;
a Cδ constant region or fragment thereof.

Preferably, the Cγ1 constant region or fragment thereof comprises an amino acid sequence comprising at least 60%, at least 70%, at least 80%, at least 85%, at least 90%, at least 95%, or 100% identity or similarity with SEQ ID NO: 112. Preferably, the Cγ2 constant region or fragment thereof comprises an amino acid sequence comprising at least 60%, at least 70%, at least 80%, at least 85%, at least 90%, at least 95%, or 100% identity or similarity with SEQ ID NO: 381 or SEQ ID NO: 382. Preferably, the Cδ constant region or fragment thereof comprises an amino acid sequence comprising at least 60%, at least 70%, at least 80%, at least 85%, at least 90%, at least 95%, or 100% identity or similarity with SEQ ID NO: 383.

In some embodiments, a soluble polypeptide comprises or consists of the extracellular domain of a γT-cell receptor chain, a δT-cell receptor chain, a γδT-cell receptor, or a fragment thereof, optionally fused to additional domains, as described herein.

The skilled person is able to obtain an extracellular domain of a γT-cell receptor chain, a δT-cell receptor chain, a γδT-cell receptor, or a fragment thereof described herein using standardized nomenclature such as the one provided by the International Immunogenetics Information System (IMGT, Lefranc et al., supra) to pinpoint the exact amino acids corresponding to the domain.

Further, the transmembrane domains of human γT- and δT-cell receptor chains are generally conserved and their sequences are available to the skilled person; see Uniprot Ref: P0CF51 for TRGC1 chains, Uniprot Ref: P03986 for TRGC2 chains, and Uniprot Ref: B7Z8K6 for TRDC chains. Using this information, the skilled person may arrive at γT-cell receptor chains, δT-cell receptor chains, γδT-cell receptors, or fragments thereof, which do not comprise a transmembrane domain. Accordingly, in some embodiments, a soluble polypeptide does not comprise a transmembrane domain of a γT-cell receptor chain, a δT-cell receptor chain, or a γδT-cell receptor, or a fragment thereof. In some embodiments, a soluble polypeptide does not comprise a cytoplasmic domain of a γT-cell receptor chain, a δT-cell receptor chain, or a γδT-cell receptor, or a fragment thereof. In some embodiments, a soluble polypeptide does not comprise SEQ ID NO: 102 or a fragment thereof. In some embodiments, a soluble polypeptide does not comprise SEQ ID NO: 103 or a fragment thereof. In some embodiments, a soluble polypeptide does not comprise SEQ ID NO: 104 or a fragment thereof.

In some embodiments, a soluble polypeptide comprises a γT-cell receptor chain or a fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 1 or 8-18, SEQ ID NO: 120 or 122-132, 144 or 146-156, 312 or 314-324, preferably selected from SEQ ID NO: 10, 12, 14, 15, 16, 17, 124, 126, 128, 129, 130, 131, 148, 150, 152, 153, 154, 155, 316, 318, 320, 321, 322, or 323, more preferably selected from SEQ ID NO: 10, 15, 16, 17, 124, 129, 130, 131, 148, 153, 154, 155, 316, 321, 322, or 323, most preferably selected from SEQ ID NO: 10, 124, 148, or 316.

In some embodiments, a soluble polypeptide comprises a δT-cell receptor chain or a fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 2, 20-27, 110, 134-143, 158-167, or 326-335, preferably selected from SEQ ID NO: 21, 22, 23, 24, 26, 136, 137, 138, 139, 141, 142, 160, 161, 162, 163, 165, 166, 328, 329, 330, 331, 333, or 334, more preferably selected from SEQ ID NO: 21, 22, 23, 136, 137, 138, 160, 161, 162, 328, 329, or 330, most preferably selected from SEQ ID NO: 23, 138, 162, or 330.

In some embodiments, a soluble polypeptide comprises a γδT-cell receptor or a fragment thereof comprising a γCDR3 region and a δCDR3 region, wherein said γδT-cell receptor or fragment thereof comprises:
a soluble γT-cell receptor chain or a fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 1 or 8-18, SEQ ID NO: 120 or 122-132, 144 or 146-156, 312 or 314-324, preferably selected from SEQ ID NO: 10, 12, 14, 15, 16, 17, 124, 126, 128, 129, 130, 131, 148, 150, 152, 153, 154, 155, 316, 318, 320, 321, 322, or 323, more preferably selected from SEQ ID NO: 10, 15, 16, 17, 124, 129, 130, 131, 148, 153, 154, 155, 316, 321, 322, or 323, most preferably selected from SEQ ID NO: 10, 124, 148, or 316, and/or, preferably and;
a soluble δT-cell receptor chain or a fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 2, 20-27, 110, 134-143, 158-167, or 326-335, preferably selected from SEQ ID NO: 21, 22, 23, 24, 26, 136, 137, 138, 139, 141, 142, 160, 161, 162, 163, 165, 166, 328, 329, 330, 331, 333, or 334, more preferably selected from SEQ ID NO: 21, 22, 23, 136, 137, 138, 160, 161, 162, 328, 329, or 330, most preferably selected from SEQ ID NO: 23, 138, 162, or 330.

Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

A soluble polypeptide described herein may in some embodiments be a chimeric polypeptide, i.e., a polypeptide that comprises various forms or parts of binding entities such as, but not limited to, a γT-cell receptor chain, a δT-cell receptor chain, a γδT-cell receptor (or fragments thereof such as extracellular domains or fragments thereof), an antibody, an scFv, a B-cell receptor, a VHH, or any combination thereof.

As a non-limiting example, γδTCR-antibody chimeric polypeptides can be generated and tested before arriving at a desired chimeric polypeptide, by replacing the heavy and light chain variable domains of an antibody by γδTCRs, γδTCR extracellular domains, γδTCR-variable domains, γδTCR-CDR3 regions, or fragments thereof.

In some embodiments, the soluble polypeptide is in single chain format. Suitable single chain formats include, but are not limited to, soluble γδTCR polypeptides of the Vγ-L-Vδ, Vδ-L-Vγ, Vγ-Cγ (Cγ1 or Cγ2)-L-Vδ, Vγ-L-Vδ-Cδ, or Vγ-Cγ (Cγ1 or Cγ2)-L-Vδ-Cδ types, wherein Vγ and Vδ correspond to, respectively, the γT- and δT-cell receptor chain variable regions (or fragments thereof), Cγ (Cγ1 or Cγ2) and Cδ correspond to, respectively, the γT- and δT-cell receptor chain constant regions (or fragments thereof), and L is a linker sequence, such as the linker sequences described herein.

In some embodiments, the soluble polypeptide is a chimeric polypeptide comprising a γT-cell receptor chain, a δT-cell receptor chain, a γδT-cell receptor, or a fragment thereof (such as e.g., an extracellular domain or fragment thereof) as described herein, that is fused (i.e., physically linked) with a T-cell- and/or NK-cell-binding domain. Such a soluble polypeptide may be called a bispecific polypeptide. A γT-cell receptor chain, δT-cell receptor chain, γδT-cell receptor, or fragment thereof may be fused with a T-cell- and/or NK-cell-binding domain directly, or via a linker peptide or additional domain as described later herein. In embodiments wherein the soluble polypeptide comprises a γδT-cell receptor or a fragment thereof fused with a T-cell- and/or NK-cell-binding domain, said polypeptide may be alternatively called a γδTCR bispecific engager.

Such a bispecific polypeptide may be advantageous, as it may first bind to a T- and/or NK-cell and then recruit the cell to a tumour cell, or to an infection site, thus mediating an anti-tumour or an anti-infective response without the requirement for its expression in a cellular membrane of an engineered T- and/or NK-cell.

A T-cell- and/or NK-cell-binding domain is to be understood as a domain that specifically binds to a T-cell and/or NK-cell, for example via binding to a receptor, an antigen, or epitope that is present on or displayed by the T-cell and/or NK-cell. In some embodiments, the T-cell and/or NK-cell is a mammalian cell, preferably a human cell. Preferably, binding of a T-cell- or NK-cell-binding domain to the respective T-cell or NK-cell results in the stimulation and/or activation of the T-cell or NK-cell.

In some embodiments, the T-cell- and/or NK-cell-binding domain is derived from, or is, an antibody, a single heavy chain variable domain antibody (such as for example a camelid VHH), a shark immunoglobulin-derived variable new antigen receptor, an scFv, a tandem scFv, a Fab, an Fc domain of an antibody, an scFab, an antibody mimetic (such as for example a designed ankyrin repeat protein), a binding protein based on a Z domain of protein A, a binding protein based on a fibronectin type III domain, a lipocalin, and combinations thereof. A T-cell- and/or NK-cell-binding domain may also be derived from, is, a minibody, a F(ab′)2 fragment, a dsFv, a nanobody (these constructs, marketed by Ablynx (Belgium), comprise synthetic single immunoglobulin variable heavy domains derived from a camelid (e.g. camel or llama) antibody), a Domain Antibody (Domantis (Belgium), comprising an affinity matured single immunoglobulin variable heavy domain or immunoglobulin variable light domain), or a alternative protein scaffold that exhibits antibody like binding characteristics such as Affibodies (Affibody (Sweden), comprising engineered protein A scaffold) or Anticalins (Pieris (Germany)), comprising engineered anticalins).

In some embodiments, the T-cell- and/or NK-cell-binding domain is of mammalian origin, preferably of human origin.

In some embodiments, the T-cell- and/or NK-cell-binding domain is selected from the group of CD3-, CD4-, CD8-, CD16-, CD56-, CD103-, CD154-, CD314-binding domains, and combinations thereof. In some embodiments, a T-cell-binding domain is a CD3-binding domain, also referred to herein as an “anti-CD3” binding domain.

In preferred embodiments, a soluble polypeptide is a chimeric polypeptide comprising a γT-cell receptor chain, a δT-cell receptor chain, a γδT-cell receptor, or a fragment thereof (such as e.g., an extracellular domain or fragment thereof) as described herein, and an anti-CD3 binding domain.

In embodiments wherein the soluble polypeptide comprises a γδT-cell receptor or a fragment thereof fused with a CD3-binding domain, said polypeptide may be alternatively called a γδTCR-T-cell bispecific engager or a γδTCR-CD3 bispecific engager.

Such binding domains are known to the skilled person, and are further described in e.g., WO2007/062245, Liao et al., 2000 (Gene Ther 7: 339-47), WO2001/051644, Arakawa et al., 1996 (J Biochem 120: 657-62), Adair et al., 1994 (Human Antibodies 5: 41-47), Kipriyanov et al., 1997 (Protein Engin Design Selection 10:445), van Diest et al., 2021 (J Immunother Cancer 2021; 9:e003850), and WO2019/156566, all of which are incorporated herein by reference in their entireties. For example, such binding domains include commercially available binding domains such as the ones offered by Creative Biolabs (Shirley, NY, USA). The anti-CD3 binding domain may, for example, be an OKT3, UCHT-1, BMA-031, or 12F6 binding domain. In some embodiments, a soluble polypeptide is a chimeric polypeptide comprising a γT-cell receptor chain, a δT-cell receptor chain, a γδT-cell receptor, or a fragment thereof (such as e.g., an extracellular domain or fragment thereof) as described herein, and an scFv domain, preferably an anti-CD3 scFv domain. A non-limiting example of a suitable anti-CD3 scFv domain is SEQ ID NO: 105.

Linkage of the soluble γT-cell receptor chain, δT-cell receptor chain, γδT-cell receptor, or fragment thereof may, for example, be via covalent or non-covalent attachment.

Binding of soluble polypeptides comprising T-cell- and/or NK-cell-binding domains to the T-cells and/or NK-cells may be assessed utilizing any assay suitable for measuring an anti-tumour or anti-infective response known to the skilled person, such as the ones described herein, by bringing the soluble polypeptides into contact with the T-cells and/or NK-cells and measuring their anti-tumour or anti-infective response.

As an example, T-cell activation mediated by soluble TCR-anti-CD3 chimeric polypeptides may be determined by measuring IFNγ secretion using an ELISpot assay. Assays can be performed e.g., using a human IFN-γELISPOT kit (BD Biosciences, NJ, USA) according to the manufacturer's instructions. Peripheral blood mononuclear cells (PBMC), isolated from fresh donor blood, isolated T-cells, or other suitable T-cell or NK-cells may be used as effector cells. To further determine suitability for therapeutic use, the TCR-anti-CD3 chimeric polypeptides may be tested for non-specific activation in the presence of normal cells derived from healthy human tissues using the same ELISPOT methodology as described above

In some embodiments, a soluble polypeptide is a chimeric polypeptide comprising a γT-cell receptor chain, a δT-cell receptor chain, a γT-cell receptor, or a fragment thereof (such as e.g., an extracellular domain or fragment thereof) as described herein, and an Fc domain of an antibody.

In some embodiments, the γT-cell receptor chain, δT-cell receptor chain, γδT-cell receptor, or fragment thereof (such as e.g., extracellular domain or fragment thereof) comprised in the soluble, such as chimeric, polypeptides described herein are fused to an extracellular domain of an immune checkpoint-related molecule (or fragment thereof), such as for example an immune checkpoint inhibitor. The term “immune checkpoint inhibitor” as used herein refers to polypeptides, such as, but not limited to, inhibitory receptors, expressed by T- and/or NK-cells.

Accordingly, in some embodiments, a soluble polypeptide is a chimeric polypeptide comprising a γT-cell receptor chain, a δT-cell receptor chain, a γδT-cell receptor, or a fragment thereof (such as e.g., an extracellular domain or fragment thereof) as described herein, an extracellular immune checkpoint inhibitor domain, and a T-cell- and/or NK-cell-binding domain, preferably an anti-CD3 scFv or Fc domain. Such a soluble polypeptide may be called a trispecific polypeptide. Suitable extracellular immune checkpoint inhibitor domains may be derived from, but are not limited to, the group consisting of the adenosine A2A receptor, programmed death 1 (PD1) receptor, T-cell immunoglobulin domain, mucin domain 3, and V-domain Ig suppressor of T cell activation (TIGIT). Among the suitable immune checkpoint inhibitor domains, the extracellular domain of PD1 (or fragment thereof) is preferred. Such trispecific polypeptides may be advantageous in mediating an enhanced anti-tumour or anti-infective response. As a non-limiting example, the presence of the extracellular PD1 domain (or fragment thereof) in a trispecific polypeptide may interact with the PD-L1 ligand in a tumour cell, thereby enhancing the anti-tumour response of the T- and/or NK-cell that is recruited to the tumour cell via the binding domain of the polypeptide. Accordingly, in some embodiments, a soluble polypeptide is a chimeric polypeptide comprising a γT-cell receptor chain, a δT-cell receptor chain, a γδT-cell receptor, or a fragment thereof (such as e.g., an extracellular domain or fragment thereof), an extracellular domain of PD1, and a T-cell- and/or NK-cell-binding domain, preferably an anti-CD3 scFv or Fc domain. In some embodiments, the anti-CD3 scFv domain is represented by SEQ ID NO: 105 or a variant thereof.

In some embodiments, a soluble, such as a chimeric, polypeptide described herein may optionally further comprise a linker peptide between the domains which provides conformational flexibility to the chimeric polypeptide. Suitable linker peptides are known to the skilled person, and may for example be selected from peptides comprising from 1 to 50 amino acid residues, from 5 to 40 amino acid residues, or from 10 to 20 amino acid residues. Examples of suitable linker peptides are described in e.g., WO1999/42077, WO2006/040153, WO2006/122825, WO2011/001152A1, and WO2019/156566, all of which are incorporated herein by reference in their entireties. Additional examples of suitable linker peptides are Gly-Ser linkers. Additional examples of suitable linker peptides comprise or consist of Gly-Gly or Gly-Gly-Ser or Gly-Ser-Gly or Tyr-Gly-Ser. Additional examples of suitable linker peptides are provided in Table 1 below. A linker peptide sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, a linker peptide is at least 1, at least 3, at least 5, at least 7, at least 9, at least 11, or at least 15 amino acids in length. In some embodiments, a linker peptide is at most 5, at most 7, at most 9, at most 11, at most 15, at most 20, at most 25, or at most 50 amino acids in length.

A flexible linker peptide can have a sequence containing stretches of glycine and serine residues. The small size of the glycine and serine residues provides flexibility and allows for mobility of the connected functional domains. The incorporation of serine or threonine can maintain the stability of the linker peptide in aqueous solutions by forming hydrogen bonds with the water molecules, thereby reducing unfavorable interactions between the linker and protein moieties. Flexible linker peptides can also contain additional amino acids such as threonine and alanine to maintain flexibility, as well as polar amino acids such as lysine and glutamine to improve solubility. A rigid linker peptide can have, for example, an alpha helix-structure. An alpha-helical rigid linker peptide can act as a spacer between protein domains.

In some embodiments, a linker peptide may, comprise any of the sequences in Table 1, or repeats thereof (e.g., 2, 3, 4, 5, 6, 7, 8, 9, or 10 repeats of any of SEQ ID NOs: 63-78, 358-361, or 384-390).

TABLE 1
Exemplary linker peptides

| SEQ ID NO: | Name | Sequence |
|---|---|---|
| 63 | Flexible linker | TSGS |
| 64 | Flexible linker | GGGGS |
| 65 | Flexible linker | GGGS |
| 66 | Flexible linker | KESGSVSSEQLAQFRSLD |
| 67 | Flexible linker | EGKSSGSGSESKST |
| 68 | Flexible linker | GSAGSAAGSGEF |
| 69 | Rigid linker | EAAAK |
| 70 | Rigid linker | EAAAR |
| 71 | Rigid linker | PAPAP |
| 72 | Rigid linker | AEAAAKEAAAKA |
| 73 | Rigid linker | ILTHDSSIRYLQEIYNSNNQKIVNLKEKVAQLEAQCQE |
| | | PCKDTVQIHDITG |
| 74 | Flexible linker | SLNGGGGSGGGGSGGGGSGGGGSGGGGSTS |
| 75 | Flexible linker | SGGSGGGGSGGGSGGGGSLQ |
| 76 | Flexible linker | SGGGSGGGGSGGGGSGGGGSGGGSLQ |
| 77 | Flexible linker | GGGGSGGGGSGGGGS |
| 78 | linker | RTGGGGSGGGGSGGGGS |
| 358 | linker | GSGGGG |
| 359 | linker | ASGLVPRGSGSG |
| 360 | linker | GLVPRGSGSG |
| 361 | linker | SGLVPRGSGSG |
| 384 | linker | GGGSG |
| 385 | linker | GGSGG |
| 386 | linker | GSGGG |
| 387 | linker | GSGGGP |
| 388 | linker | GGEPS |
| 389 | linker | GGEGGGP |
| 390 | linker | GGEGGGSEGGGS |

In some embodiments, a soluble, such as a chimeric, polypeptide comprises a linker peptide with at least 1, at least 2, at least 3, at least 4, or at least 5 amino acid insertions, deletions, or substitutions relative to any of SEQ ID NOs: 63-78, 358-361, or 384-390. The insertions, deletions, or substitutions can be at the N-terminus, the C-terminus, within the sequence, or a combination thereof. The insertions, deletions, or substitutions can be contiguous or non-contiguous. In some cases, the substitutions are conservative. In some cases, the substitutions are non-conservative.

In some embodiments, a soluble, such as a chimeric, polypeptide comprises a linker peptide with an amino acid sequence selected from Gly-Gly, Gly-Gly-Ser, Gly-Ser-Gly, Tyr-Gly-Ser, or repeats thereof. In some embodiments, a soluble, such as a chimeric, polypeptide does not contain any linker peptides, for example, the chimeric polypeptide is a direct fusion of amino acid sequences from other proteins with no intervening amino acid sequence.

A soluble, such as a chimeric, polypeptide as described herein may optionally comprise additional domains, for example a domain facilitating polypeptide excretion (in embodiments wherein the soluble polypeptide is produced by a cell, i.e., a signal peptide), and/or polypeptide isolation and/or purification and/or stability. Such domains and their applications are known in the art. Additional examples of signal peptides are represented by SEQ ID NO: 106 (γ chain) and SEQ ID NO: 107 (δ chain).

Examples of suitable domains facilitating polypeptide isolation and/or purification, and/or stability, may be derived from an Avi-tag, His-tag, c-myc domain, hemagglutinin tag, glutathione-S-transferase, maltose-binding protein, FLAG tag peptide, biotin acceptor peptide, streptravidin-binding peptide, calmodulin-binding peptide, bovine serum albumin, and others. Additional examples of suitable domains facilitating polypeptide isolation and/or purification, and/or stability, are encoded by SEQ ID NO: 38 (Avi-tag, suitable for biotinylation purposes) and SEQ ID NO: 39 (His-tag, suitable for purification purposes) or are represented by SEQ ID NO: 365-367.

In soluble, such as chimeric, polypeptides described herein, a T-cell- and/or NK-cell-binding domain, an immune checkpoint inhibitor domain, and/or an additional domain may be fused to a γT-cell receptor chain, a δT-cell receptor chain, a γδT-cell receptor, or a fragment thereof (such as e.g., an extracellular domain or fragment thereof) at the N-terminus or C-terminus of the receptor chain, receptor, or fragment thereof. In cases where multiple domains are comprised in a chimeric polypeptide, the skilled person understands that each additional domain may also be fused at the N-terminus or C-terminus of the domain it is fused to. Optionally, linkers may be comprised between the domains as described earlier herein.

In some embodiments, a soluble, such as chimeric, polypeptide as described herein is a dimer, or a higher multimer such as a trimer. In some embodiments, dimerization or multimerization is facilitated by the inclusion of a dimerization or multimerization domain in the polypeptide, for example a leucine zipper, a Jun-Fos interaction motif (such as for example described in Pack and Pluckthun, 1992, Biochemistry 31, 1579-1584; de Kruif and Logtenberg, 1996. JBC 271: 7630-7634, incorporated herein by reference in their entireties), or any other suitable such domain known to the skilled person. Additional examples of dimerization domains are encoded by SEQ ID NO: 36 (c-Fos dimerization motif) and SEQ ID NO: 37 (c-Jun dimerization motif) or are represented by SEQ ID NO: 363 (c-Fos dimerization motif) and SEQ ID NO: 362 (c-Jun dimerization motif). Optionally, a chimeric polypeptide comprising a γT-cell receptor chain or fragment thereof comprising a γCDR3 region may comprise a c-Jun dimerization motif. Optionally, a chimeric polypeptide comprising a δT-cell receptor chain or fragment thereof comprising a δCDR3 region may comprise a c-Fos dimerization motif.

Alternatively, a bivalent or multivalent polypeptide may be generated via chemical cross-linking using standard methods.

The skilled person understands that a dimer or a higher multimer as described herein may be a dimer or multimer of soluble polypeptides comprising the same or different γT-cell receptor chains, δT-cell receptor chains, γδT-cell receptors, or fragment s thereof (such as e.g., extracellular domains or fragments thereof), and/or T-cell and/or NK-cell-binding domains, said polypeptides and/or domains optionally having different targets.

In embodiments wherein the soluble, such as chimeric, polypeptide comprises a γδT-cell receptor or a fragment thereof, formation of the receptor heterodimer (or fragment thereof) from the γT-cell receptor and δT-cell receptor chain (or fragments thereof) may be promoted by inclusion of any of the dimerization domains, or any other dimerization method discussed above. Alternatively, or in addition, it can be promoted by inclusion of cysteines in the chimeric polypeptide, for example in the constant γ- and/or δ-regions or in any other region, to promote formation of cysteine bonds (cysteine bridges). Additional examples are provided in the experimental section herein.

A soluble, such as chimeric, polypeptide described herein may be subject to post translational modifications. Glycosylation is an example of such a modification, which comprises the covalent attachment of oligosaccharide moieties to defined amino acids in the γδTCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov March; 8(3):226-34, incorporated herein by reference in its entirety). For soluble γδTCRs of the invention glycosylation may be controlled, as an example, by using particular cell lines for their production (including but not limited to mammalian cell lines such as Chinese hamster ovary (CHO) cells or human embryonic kidney (HEK) cells as discussed elsewhere herein), or by chemical modification. Such modifications may be desirable, since glycosylation can improve pharmacokinetics, reduce immunogenicity and more closely mimic a native human protein (Sinclair and Elliott, (2005) Pharm Sci. August; 94(8): 1626-35, incorporated herein by reference in its entirety).

The skilled person may arrive at the soluble, such as chimeric, polypeptides described herein utilizing standard molecular toolbox techniques. A soluble, such as chimeric, polypeptide described herein may be synthetic or may be produced by an engineered cell, as described later herein. Optionally, the polypeptide may be isolated and/or purified after its production. Suitable downstream processing methods for isolation and/or purification of polypeptides from cell cultures are well-known in the art. Examples of suitable isolation and/or purification techniques are chromatographic methods such as high performance liquid chromatography, size exclusion chromatography, ion exchange chromatography, affinity chromatography (such as for example utilizing His-tags or protein A), immunoaffinity chromatography, immunoprecipitation, and the like. As a non-limiting example, a chimeric polypeptide comprising a γT-cell receptor chain, a δT-cell receptor chain, a γδT-cell receptor, or a fragment thereof (such as e.g., an extracellular domain or fragment thereof), and a T- and/or NK-cell-binding domain (for example an anti-CD3 scFv or Fc domain) may be produced by a cell, for example a cell line such as HEK293 or HEK293F cells, and subsequently purified using affinity chromatography (for example via a His-tag peptide). A proper folding of the chimeric polypeptide can be probed using conformational-specific antibodies that can target γ and δ variable domains. Purity can be assessed by standard methods, for example SDS-PAGE and Coomassie staining. Chimeric polypeptides may then be used in antibody dependent cell mediated cytotoxicity (ADCC), complement dependent cytotoxicity (CDC) and antibody drug conjugate (ADC) assays to determine functional efficacy. After performing in vitro assays, functional efficacy of chimeric polypeptides can be tested in vitro and/or in vivo. Accordingly, in some embodiments, a soluble, such as a chimeric, polypeptide described herein is expressed by a cell. In some embodiments, the cell is selected from a bacterium (e.g., E. coli), a yeast (e.g., P. pastoris, S. cerevisiae), an insect cell (e.g. an Sf9 cell), a mammalian cell, or a human cell, for example a human cells line (e.g., HEK293 or HEK293F or derivatives thereof). In some embodiments, the cell is an immunoresponsive cell, preferably selected from T-cells, iPSC-derived T-cells, αβT-cells, γδT-cells, or NK cells, more preferably selected from γδT-cells or αβT-cells, most preferably αβT-cells.

The skilled person understands that the assays measuring mediation of anti-tumour or anti-infective activity or target binding described earlier herein are also applicable to the soluble polypeptides described herein. As a non-limiting example, a chimeric polypeptide comprising a γT-cell receptor chain, a δT-cell receptor chain, a γδT-cell receptor, or a fragment thereof (such as e.g., an extracellular domain or fragment thereof) and a T-cell- and/or NK-cell-binding domain (and optionally any other suitable domain discussed herein) may be provided together with T-cells in the assays described herein, and the anti-tumour or anti-infective response of the T-cells as mediated by the chimeric polypeptide may then be assessed using any of the assays described above. Control T-cells as described above, such as T-cells that have not been provided together with soluble polypeptides of the disclosure, may similarly be used for the comparisons. Optionally, different amounts of soluble polypeptides and/or ratios of soluble polypeptides to T-cells may be tested.

Alternatively, the capacity of a soluble polypeptide to bind to a target cell, after bringing the polypeptide into contact with said cell, may be assessed. In some embodiments, the contacting step has a duration of at least 30 minutes, at least 1 hour, at least 2 hours, at least 4 hours, at least 8 hours, at least 16 hours, at least 18 hours, at least 20 hours, at least 1 day, at least 2 days, at least 3 days, at least 4 days, or at least 5 days. Optionally, varying amounts of soluble polypeptide may be utilized, for example from 0.01 μg to 100 μg, from 0.01 μg to 10 μg, from 0.1 μg to 10 μg, from 1 μg to 10 mg, or any other suitable amount.

In a further aspect, there is provided a conjugate comprising a γT-cell receptor chain, a δT-cell receptor chain, a γδT-cell receptor, or a fragment thereof (such as e.g., an extracellular domain or fragment thereof), as described herein, which is linked to an agent. The skilled person understands that the type of agent used depends on the type of applications envisaged. Such a conjugate may, for example, be linked to a substrate (e.g. chemicals, nanoparticles) and may be used e.g., to administer chemotherapy to a target of interest. As another example, in diagnostics expression of defined ligands may be tested by taking advantage of soluble γδTCRs linked to fluorochromes which are used as staining tool or for the biochemical isolation of the ligand.

In some embodiments, the agent is selected from the group consisting of a diagnostic agent, a therapeutic agent, an anti-cancer agent, a chemical, a nanoparticle, a chemotherapeutic agent, a fluorescent protein, or an enzyme whose catalytic activity could be detected.

Examples of suitable agents include, but are not limited to:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate 21arbour21ate, auristatin E vincristine and doxorubicin;
peptide cytotoxins, i.e. proteins or fragments thereof with the ability to kill mammalian cells. Examples of such agents include ricin, diphtheria toxin, Pseudomonas bacterial exotoxin A, Dnase and Rnase;
radio-nuclides, i.e. unstable isotopes of elements which decay with the concurrent emission of one or more of α or β particles, or γ rays. Examples of such agents include iodine 131, rhenium 186, indium 111, yttrium 90, bismuth 210 and 213, actinium 225 and astatine 213; chelating agents may be used to facilitate the association of these radio-nuclides to the high affinity γδTCRs, or multimers thereof;
immuno-stimulants, i.e., immune effector molecules which stimulate immune response. Examples of such agents include cytokines such as IL-2 and IFN-γ;
superantigens and mutants thereof;
chemokines such as IL-8, platelet factor 4, melanoma growth stimulatory protein, etc;
alternative protein scaffolds with antibody like binding characteristics;
complement activators;
xenogeneic protein domains, allogeneic protein domains, viral/bacterial protein domains, viral/bacterial peptides.

In some embodiments, the conjugate is linked to a liposome containing the agent. This can prevent damaging effects during the transport in the body and can ensure that the agent exerts its activity after binding of the soluble γT-cell receptor chain, δT-cell receptor chain, γδT-cell receptor, or fragment thereof to its target.

In some embodiments, the fluorescent protein is selected from the group consisting of: green fluorescent protein (GFP), yellow fluorescent protein (YFP), red fluorescent protein (RFP), blue fluorescent protein (BFP), cyan fluorescent variant known (CFP), yellow fluorescent protein (YFP), violet-excitable green fluorescent protein known as Sapphire, and cyan-excitable green fluorescent protein known as enhanced green fluorescent protein (eGFP). The presence of a fluorescent protein can be assessed by live cell imaging, flow cytometry, and/or fluorescent spectrophotometry. Fluorescent reporters can be detected using various means including but not limited to microscopy, visual observation, flow cytometry, Luminex, and the like. In some embodiments, a fluorescent reporter is detected using flow cytometry.

In some embodiments, the enzyme whose activity could be detected is selected from the group consisting of luciferase, beta galactosidase, beta-lactamase, catalase, alkaline phosphatase, and the like. As a non-limiting example, luciferase activity can be detected by commercially available assays, e.g., by the Luciferase 1000 Assay System, Nano-Glo or the Bio-Glo (Promega, WI, US). The Luciferase 1000 Assay System contains coenzyme A (CoA) besides luciferin as a substrate, resulting in a strong light intensity lasting for at least one minute when the manufacturer's protocol is followed. Alternatively, D-luciferin can also be utilized. In some cases, prior to an intracellular luciferase assay, it may be helpful to lyse the cells prior to detection. In some embodiments a Luciferase assay is used wherein the luciferase is secreted from the cells. Hence, in some cases, the assay can be performed without lysis of the cells.

The abovementioned assays may also be used for detection of the activity of a reporter polypeptide in the methods described earlier herein.

The disclosure further provides nucleic acid molecules encoding the polypeptides described herein. A nucleic acid molecule described herein may in some cases be a synthetic nucleic acid molecule or be part of a synthetic construct. A nucleic acid molecule described herein may in some cases be a codon optimized molecule, preferably for expression in a mammalian cell, more preferably in a human cell. A definition of codon optimization is provided later herein.

Accordingly, in an aspect, there is provided a nucleic acid molecule encoding a soluble γT-cell receptor chain or fragment thereof comprising a γCDR3 region as described herein.

In some embodiments, the nucleic acid molecule encodes a soluble γT-cell receptor chain or a fragment thereof comprising a γCDR3 region, wherein said γCDR3 region is represented by an amino acid sequence comprising at least 60%, at least 70%, at least 80%, at least 85%, or at least 90% sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 116-122 of SEQ ID NO: 4, or from one or more of positions 4-10 of SEQ ID NO: 1, preferably from one or more of positions 4-10 of SEQ ID NO: 1. In some embodiments, it comprises a modification in the γCDR3 region relative to SEQ ID NO: 1 at an amino acid position corresponding to a position selected from one or more of positions 117-121 of SEQ ID NO: 4, or from one or more of positions 5-9 of SEQ ID NO: 1, preferably from one or more of positions 5-9 of SEQ ID NO: 1.

In some embodiments, a preferred nucleic acid molecule encodes a soluble γT-cell receptor chain or fragment thereof comprising a γCDR3 region comprising a modification in the γCDR3 region relative to SEQ ID NO: 1 at an amino acid position corresponding to a position selected from one or more of positions 117-121 of SEQ ID NO: 4 or from one or more of positions 5-9 of SEQ ID NO: 1, preferably from one or more of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369). In some embodiments, the γCDR3 region is represented by an amino acid sequence comprising at least 60%, at least 70%, at least 80%, at least 85%, or at least 90%, sequence identity or similarity with SEQ ID NO: 1, and comprises an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374), preferably selected from the group consisting of DAFYY (SEQ ID NO: 369), DGYYY (SEQ ID NO: 372), DGAYY (SEQ ID NO: 373), and DGSYY (SEQ ID NO: 374), more preferably comprises DAFYY (SEQ ID NO: 369), relative to SEQ ID NO: 1 at the amino acid positions corresponding to positions 117-121 of SEQ ID NO: 4 or to positions 5-9 of SEQ ID NO: 1, preferably to positions 5-9 of SEQ ID NO: 1.

In some embodiments, the nucleic acid molecule encodes a soluble γT-cell receptor chain or fragment thereof comprising a γCDR3 region, wherein said receptor chain or fragment thereof comprises a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, preferably of positions 4-10 of SEQ ID NO: 1, preferably wherein said modification is selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGAYYK (SEQ ID NO: 89), or WDGSYYK (SEQ ID NO: 90), more preferably is WDAFYYK (SEQ ID NO: 83). In some embodiments, the γCDR3 region is represented by an amino acid sequence comprising at least 60%, at least 70%, at least 80%, at least 85%, or at least 90%, sequence identity or similarity with SEQ ID NO: 1, and comprises an amino acid sequence selected from the group consisting of WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), and WDGSYYK (SEQ ID NO: 90), preferably selected from the group consisting of WDAFYYK (SEQ ID NO: 83), WDGYYYK (SEQ ID NO: 88), WDGAYYK (SEQ ID NO: 89), and WDGSYYK (SEQ ID NO: 90), more preferably comprises WDAFYYK (SEQ ID NO: 83), at the amino acid positions corresponding to positions 116-122 of SEQ ID NO: 4 or to positions 4-10 of SEQ ID NO: 1, preferably to positions 4-10 of SEQ ID NO: 1.

In some embodiments, the nucleic acid molecule encodes a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 and comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, preferably of positions 4-10 of SEQ ID NO: 1, said modification preferably selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), more preferably selected from WDAFYYK (SEQ ID NO: 83), WDGYYYK (SEQ ID NO: 88), WDGAYYK (SEQ ID NO: 89), or WDGSYYK (SEQ ID NO: 90), most preferably is WDAFYYK (SEQ ID NO: 83).

In some embodiments, the nucleic acid molecule encodes a soluble γT-cell receptor chain or fragment thereof comprising a γCDR3 region represented by an amino acid sequence having at least 60%, at least 70%, at least 80%, at least 90%, at least 95%, or 100% sequence identity or similarity with an amino acid sequence selected from SEQ ID NO: 1 or 8-18, preferably selected from SEQ ID NO: 10, 12, 14, 15, 16, or 17, more preferably selected from SEQ ID NO: 10, 15, 16, or 17, most preferably with SEQ ID NO: 10.

In some embodiments, the nucleic acid molecule encodes a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172, 196, 220, 244, 268, 292, or 316. In some embodiments, the soluble γT-cell receptor chain or fragment thereof comprising a γCDR3 region is comprised in a chimeric polypeptide.

In some embodiments, the nucleic acid molecule encodes a soluble γT-cell receptor chain or fragment thereof comprising a γCDR3 region and comprises, consists essentially of, or consists of, preferably comprises, a nucleotide sequence having at least 60%, at least 70%, at least 80%, at least 90%, at least 95%, or 100% sequence identity with a nucleotide sequence selected from SEQ ID NO: 28, 29, 32, 33, 57, or 59, preferably selected from SEQ ID NO: 32, 33, or 59.

In preferred embodiments, the encoded soluble γT-cell receptor chain or fragment thereof further comprises a γCDR1 region represented by an amino acid sequence comprising at least 60%, at least 70%, at least 80%, at least 85%, at least 90%, at least 95%, or 100%, sequence identity or similarity with SEQ ID NO: 375, and a γCDR2 region represented by an amino acid sequence comprising at least 60%, at least 70%, at least 80%, at least 85%, at least 90%, at least 95%, or 100%, sequence identity or similarity with SEQ ID NO: 376. In some embodiments, the encoded soluble γT-cell receptor chain or fragment thereof further comprises a Cγ1 or Cγ2 constant region or fragment thereof, as described herein.

In an aspect, there is provided a nucleic acid molecule encoding a soluble δT-cell receptor chain or a fragment thereof comprising a δCDR3 region as described herein.

In some embodiments, the nucleic acid molecule encodes a soluble δT-cell receptor chain or a fragment thereof comprising a δCDR3 region, wherein said δCDR3 region is represented by an amino acid sequence comprising at least 60%, at least 70%, at least 80%, at least 85%, or at least 90%, sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 122-127 of SEQ ID NO: 6, or from one or more of positions 7-12 of SEQ ID NO: 2, preferably from one or more of positions 7-12 of SEQ ID NO: 2.

In some embodiments, the nucleic acid molecule encodes a soluble δT-cell receptor chain or fragment thereof comprising a δCDR3 region, wherein said receptor chain or fragment thereof comprises a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, preferably of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97). In some embodiments, the δCDR3 region is represented by an amino acid sequence comprising at least 60%, at least 70%, at least 80%, at least 85%, or at least 90%, sequence identity or similarity with SEQ ID NO: 2, and comprises an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprises IKGFTG (SEQ ID NO: 97), at the amino acid positions corresponding to positions 122-127 of SEQ ID NO: 6 or to positions 7-12 of SEQ ID NO: 2, preferably to positions 7-12 of SEQ ID NO: 2.

In some embodiments, the nucleic acid molecule encodes a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, preferably of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96), or IKGFTG (SEQ ID NO: 97), most preferably is IKGFTG (SEQ ID NO: 97).

In some embodiments, the nucleic acid molecule encodes a soluble δT-cell receptor chain or fragment thereof comprising a δCDR3 region represented by an amino acid sequence having at least at least 60%, at least 70%, at least 80%, at least 90%, at least 95%, or 100% sequence identity or similarity with an amino acid sequence selected from SEQ ID NO: 2, 20-27, or 110, preferably selected from SEQ ID NO: 21, 22, 23, 24, 26, or 110, more preferably selected from SEQ ID NO: 21, 22 or 23, most preferably with SEQ ID NO: 23.

In some embodiments, the nucleic acid molecule encodes a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330. In some embodiments, the soluble δT-cell receptor chain or fragment thereof comprising a δCDR3 region is comprised in a chimeric polypeptide.

In some embodiments, the nucleic acid molecule encodes a soluble δT-cell receptor chain or fragment thereof comprising a δCDR3 region and comprises, consists essentially of, or consists of, preferably comprises, a nucleotide sequence having at least 60%, at least 70%, at least 80%, at least 90%, at least 95%, or 100% sequence identity with a nucleotide sequence selected from SEQ ID NO: 30, 31, 34, 35, 58, or 60, preferably selected from SEQ ID NO: 34, 35, or 60.

In preferred embodiments, the encoded soluble δT-cell receptor chain or fragment thereof further comprises a δCDR1 region represented by an amino acid sequence comprising at least 60%, at least 70%, at least 80%, at least 85%, at least 90%, at least 95%, or 100%, sequence identity or similarity with SEQ ID NO: 377, and a δCDR2 region represented by an amino acid sequence comprising at least 60%, at least 70%, at least 80%, at least 85%, at least 90%, at least 95%, or 100%, sequence identity or similarity with SEQ ID NO: 378. In some embodiments, the encoded soluble δT-cell receptor chain or fragment thereof further comprises a Cδ constant region or fragment thereof, as described herein.

In an aspect, there is provided a nucleic acid molecule encoding a soluble γδT-cell receptor or a fragment thereof comprising a γCDR3 region and a δCDR3 region as described herein.

In some embodiments, the nucleic acid molecule encodes a soluble γδT-cell receptor or a fragment thereof comprising a γCDR3 region and a δCDR3 region, wherein the γδT-cell receptor or fragment thereof comprises a soluble γT-cell receptor chain or fragment thereof as described herein, and/or, preferably and, a soluble δT-cell receptor chain or fragment thereof as described herein.

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 116-122, preferably 117-121, of SEQ ID NO: 4 or of positions 4-10, preferably 5-9, of SEQ ID NO: 1, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2.

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain comprising the amino acid sequence SEQ ID NO: 120, 144, 168, 192, 216, 240, 264, 288, or 312, or an amino acid sequence having at least 60%, at least 70%, at least 80%, at least 90%, at least 95%, or 100% sequence identity or similarity with SEQ ID NO: 120, 144, 168, 192, 216, 240, 264, 288, or 312, and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2.

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain or fragment thereof comprising a γCDR3 region, said receptor chain or fragment thereof comprising a modification in the γCDR3 region relative to SEQ ID NO: 1 at an amino acid position corresponding to a position selected from one or more of positions 116-122, preferably 117-121, of SEQ ID NO: 4 or of positions 4-10, preferably 5-9, of SEQ ID NO: 1, and/or, preferably and;
a soluble δT-cell receptor chain comprising the amino acid sequence SEQ ID NO: 135, 159, 183, 207, 231, 255, 279, 303, or 327, or an amino acid sequence having at least 60%, at least 70%, at least 80%, at least 90%, at least 95%, or 100% sequence identity or similarity with an amino acid sequence selected from SEQ ID NOs: 135, 159, 183, 207, 231, 255, 279, 303, or 327.

In some embodiments, the nucleic acid molecule encodes a soluble γδT-cell receptor or fragment thereof comprising a γCDR3 and a δCDR3 region, wherein the γδT-cell receptor or fragment thereof comprises:
a soluble γT-cell receptor chain or a fragment thereof comprising a γCDR3 region, wherein said γCDR3 region is represented by an amino acid sequence comprising at least 60%, at least 70%, at least 80%, at least 85%, or at least 90%, sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 116-122 of SEQ ID NO: 4, or from one or more of positions 4-10 of SEQ ID NO: 1, and/or, preferably and;
a soluble δT-cell receptor chain or a fragment thereof comprising a δCDR3 region, wherein said δCDR3 region is represented by an amino acid sequence comprising at least 60%, at least 70%, at least 80%, at least 85%, or at least 90% sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 122-127 of SEQ ID NO: 6, or from one or more of positions 7-12 of SEQ ID NO: 2.

In some embodiments, the nucleic acid molecule encodes a soluble γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, said receptor or fragment thereof comprising a modification in the γCDR3 region relative to SEQ ID NO: 1 at an amino acid position corresponding to a position selected from one or more of positions 117-121 of SEQ ID NO: 4 or of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369), and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, the nucleic acid molecule encodes a soluble γδT-cell receptor or fragment thereof comprising a γCDR3 and a δCDR3 region, wherein the γδT-cell receptor or fragment thereof comprises:
a γCDR3 region represented by an amino acid sequence comprising at least 60%, at least 70%, at least 80%, at least 85%, or at least 90%, sequence identity or similarity with SEQ ID NO: 1, and comprising an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374), preferably selected from the group consisting of DAFYY (SEQ ID NO: 369), DGYYY (SEQ ID NO: 372), DGAYY (SEQ ID NO: 373), and DGSYY (SEQ ID NO: 374), more preferably comprising DAFYY (SEQ ID NO: 369), at the amino acid positions corresponding to positions 117-121 of SEQ ID NO: 4 or to positions 5-9 of SEQ ID NO: 1, and/or, preferably and;
a δCDR3 region represented by an amino acid sequence comprising at least 60%, at least 70%, at least 80%, at least 85%, or at least 90%, sequence identity or similarity with SEQ ID NO: 2, and comprising an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprising IKGFTG (SEQ ID NO: 97), at the amino acid positions corresponding to positions 122-127 of SEQ ID NO: 6 or to positions 7-12 of SEQ ID NO: 2.

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain or fragment thereof comprising a γCDR3 region, said receptor or fragment thereof comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, said modification selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), preferably selected from WDAFYYK (SEQ ID NO: 83), WDGYYYK (SEQ ID NO: 88), WDGAYYK (SEQ ID NO: 89), or WDGSYYK (SEQ ID NO: 90), more preferably is WDAFYYK (SEQ ID NO: 83), and/or, preferably and;
a soluble δT-cell receptor chain or fragment thereof comprising a δCDR3 region, said receptor chain or fragment thereof comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), preferably selected from IRGFTG (SEQ ID NO: 95), IKGYTG (SEQ ID NO: 96) or IKGFTG (SEQ ID NO: 97), more preferably is IKGFTG (SEQ ID NO: 97).

In some embodiments, the nucleic acid molecule encodes a soluble γδT-cell receptor or a fragment thereof comprising a γCDR3 region and a δCDR3 region, wherein the γδT-cell receptor or fragment thereof comprises:
a γCDR3 region represented by an amino acid sequence comprising at least 60%, at least 70%, at least 80%, at least 85%, or at least 90%, sequence identity or similarity with SEQ ID NO: 1, and comprising an amino acid sequence selected from the group consisting of WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), and WDGSYYK (SEQ ID NO: 90), preferably selected from the group consisting of WDAFYYK (SEQ ID NO: 83), WDGYYYK (SEQ ID NO: 88), WDGAYYK (SEQ ID NO: 89), and WDGSYYK (SEQ ID NO: 90), more preferably comprising WDAFYYK (SEQ ID NO: 83), at the amino acid positions corresponding to positions 116-122 of SEQ ID NO: 4 or to positions 4-10 of SEQ ID NO: 1, and/or, preferably and;
a δCDR3 region represented by an amino acid sequence comprising at least 60%, at least 70%, at least 80%, at least 85%, or at least 90%, sequence identity or similarity with SEQ ID NO: 2, and comprising an amino acid sequence selected from the group consisting of an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100), preferably selected from the group consisting of IRGFTG (SEQ ID NO: 95), IKGYTG (SEQ ID NO: 96), and IKGFTG (SEQ ID NO: 97), more preferably comprising IKGFTG (SEQ ID NO: 97), at the amino acid positions corresponding to positions 122-127 of SEQ ID NO: 6 or to positions 7-12 of SEQ ID NO: 2.

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain or fragment thereof comprising a γCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 (or with an amino acid sequence encoded by SEQ ID NO: 3) and comprising a modification in the γCDR3 region relative to SEQ ID NO: 1 at an amino acid position corresponding to a position selected from one or more of positions 117-121 of SEQ ID NO: 4 or of positions 5-9 of SEQ ID NO: 1, said modification selected from DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), or DGSYY (SEQ ID NO: 374), preferably selected from DAFYY (SEQ ID NO: 369), DGYYY (SEQ ID NO: 372), DGAYY (SEQ ID NO: 373), or DGSYY (SEQ ID NO: 374), more preferably is DAFYY (SEQ ID NO: 369), and/or, preferably and;
a soluble δT-cell receptor chain or fragment thereof comprising a δCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 6 (or with an amino acid sequence encoded by SEQ ID NO: 5) and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain or fragment thereof comprising a γCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, sequence identity or similarity with the amino acid sequence SEQ ID NO: 4 (or with an amino acid sequence encoded by SEQ ID NO: 3) and comprising a modification in the γCDR3 region relative to SEQ ID NO: 1 at an amino acid position corresponding to a position selected from one or more of positions 116-122 of SEQ ID NO: 4 or of positions 4-10 of SEQ ID NO: 1, preferably of positions 4-10 of SEQ ID NO: 1, said modification preferably selected from WDAFYYK (SEQ ID NO: 83), WEAFYYK (SEQ ID NO: 85), WDGYFYK (SEQ ID NO: 87), WDGYYYK (SEQ ID NO: 88), WDGAYYK (SEQ ID NO: 89), or WDGSYYK (SEQ ID NO: 90), more preferably selected from WDAFYYK (SEQ ID NO: 83), WDGYYYK (SEQ ID NO: 88), WDGAYYK (SEQ ID NO: 89), or WDGSYYK (SEQ ID NO: 90), most preferably is WDAFYYK (SEQ ID NO: 83), and/or, preferably and;
a soluble δT-cell receptor chain or fragment thereof comprising a δCDR3 region, said receptor chain or fragment thereof comprising, consisting essentially of, or consisting of, preferably comprising, an amino acid sequence having at least 60%, at least 70%, at least 80%, at least 90%, at least 95%, at least 96%, or at least 97%, identity or similarity with the amino acid sequence SEQ ID NO: 6 (or with an amino acid sequence encoded by SEQ ID NO: 5) and comprising a modification in the δCDR3 region relative to SEQ ID NO: 2 at an amino acid position corresponding to a position selected from one or more of positions 122-127 of SEQ ID NO: 6 or of positions 7-12 of SEQ ID NO: 2, preferably of positions 7-12 of SEQ ID NO: 2, said modification preferably selected from IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), or LKGYTG (SEQ ID NO: 100), more preferably selected from IRGFTG (SEQ ID NO: 95), IKGYTG (SEQ ID NO: 96) or IKGFTG (SEQ ID NO: 97), most preferably is IKGFTG (SEQ ID NO: 97).

In some embodiments, the nucleic acid molecule encodes a soluble γδT-cell receptor or a fragment thereof comprising a γCDR3 region and a δCDR3 region, wherein the γδT-cell receptor or fragment thereof comprises:
a γCDR3 region represented by an amino acid sequence having at least at least 60%, at least 70%, at least 80%, at least 90%, at least 95%, or 100% sequence identity or similarity with an amino acid sequence selected from SEQ ID NO: 1 or 8-18, preferably selected from SEQ ID NO: 10, 12, 14, 15, 16, or 17, more preferably selected from SEQ ID NO: 10, 15, 16, or 17, most preferably with SEQ ID NO: 10, and/or, preferably and;
a δCDR3 region represented by an amino acid sequence having at least at least 60%, at least 70%, at least 80%, at least 90%, at least 95%, or 100% sequence identity or similarity with an amino acid sequence selected from SEQ ID NO: 2, 20-27, or 110, preferably selected from SEQ ID NO: 21, 22, 23, 24, 26, or 110, more preferably selected from SEQ ID NO: 21, 22 or 23, most preferably with SEQ ID NO: 23.

In some embodiments, the nucleic acid molecule encodes a soluble γδT-cell receptor or a fragment thereof comprising a γCDR3 region and a δCDR3 region, wherein the γδT-cell receptor or fragment thereof comprises:
a soluble γT-cell receptor chain or fragment thereof comprising a γCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324, preferably selected from SEQ ID NO: 124, 126, 128, 129, 130, 131, 148, 150, 152, 153, 154, 155, 172, 174, 176, 177, 178, 179, 196, 198, 200, 201, 202, 203, 220, 222, 224, 225, 226, 227, 244, 246, 248, 249, 250, 251, 268, 270, 272, 273, 274, 275, 292, 294, 296, 297, 298, 299, 316, 318, 320, 321, 322, or 323, more preferably selected from SEQ ID NO: 124, 129, 130, 131, 148, 153, 154, 155, 172, 177, 178, 179, 196, 201, 202, 203, 220, 225, 226, 227, 244, 249, 250, 251, 268, 273, 274, 275, 292, 297, 298, 299, 316, 321, 322, or 323, most preferably selected from SEQ ID NO: 124, 148, 172, 196, 220, 244, 268, 292, or 316, and/or, preferably and;
a soluble δT-cell receptor chain or fragment thereof comprising a δCDR3 region comprising, consisting essentially of, or consisting of, preferably comprising, an amino acid sequence having at least 60%, at least 70%, at least 80%, at least 90%, at least 95%, or 100% sequence identity or similarity with an amino acid sequence selected from SEQ ID NO: 134-143, 158-167, 182-191, 206-215, 230-239, 254-263, 278-287, 302-311, or 326-335, preferably selected from SEQ ID NO: 136, 137, 138, 139, 141, 142, 160, 161, 162, 163, 165, 166, 184, 185, 186, 187, 189, 190, 208, 209, 210, 211, 213, 214, 232, 233, 234, 235, 237, 238, 256, 257, 258, 259, 261, 262, 280, 281, 282, 283, 285, 286, 304, 305, 306, 307, 309, 310, 328, 329, 330, 331, 333, or 334, more preferably selected from SEQ ID NO: 136, 137, 138, 160, 161, 162, 184, 185, 186, 208, 209, 210, 232, 233, 234, 256, 257, 258, 280, 281, 282, 304, 305, 306, 328, 329, or 330, most preferably selected from SEQ ID NO: 138, 162, 186, 210, 234, 258, 282, 306, or 330. In some embodiments, the soluble γδT-cell receptor or fragment thereof comprising a γCDR3 region and a δCDR3 region is comprised in a chimeric polypeptide.

In some embodiments, the nucleic acid molecule encodes a soluble γδT-cell receptor or a fragment thereof comprising a γCDR3 region and a δCDR3 region and comprises a nucleotide sequence having at least 60%, at least 70%, at least 80%, at least 90%, at least 95%, or 100% sequence identity with a nucleotide sequence selected from SEQ ID NO: 28, 29, 32, 33, 57, or 59, preferably selected from SEQ ID NO: 32, 33, or 59, and a nucleotide sequence having at least 60%, at least 70%, at least 80%, at least 90%, at least 95%, or 100% sequence identity with a nucleotide sequence selected from SEQ ID NO: 30, 31, 34, 35, 58 or 60, preferably selected from SEQ ID NO: 34, 35, or 60.

In preferred embodiments, the nucleic acid molecule encodes a soluble γδT-cell receptor or fragment thereof further comprising:
a γCDR1 region represented by an amino acid sequence comprising at least 60%, at least 70%, at least 80%, at least 85%, at least 90%, at least 95%, or 100%, sequence identity or similarity with SEQ ID NO: 375, and a γCDR2 region represented by an amino acid sequence comprising at least 70%, at least 80%, at least 85%, at least 90%, at least 95%, or 100%, sequence identity or similarity with SEQ ID NO: 376, and/or, preferably and;
a δCDR1 region represented by an amino acid sequence comprising at least 60%, at least 70%, at least 80%, at least 85%, at least 90%, at least 95%, or 100%, sequence identity or similarity with SEQ ID NO: 377, and a δCDR2 region represented by an amino acid sequence comprising at least 70%, at least 80%, at least 85%, at least 90%, at least 95%, or 100%, sequence identity or similarity with SEQ ID NO: 378.

In some embodiments, the encoded soluble γδT-cell receptor or fragment thereof further comprises:
a Cγ1 constant region, a Cγ2 constant region, or a fragment thereof, and/or, preferably and;
a Cδ constant region or fragment thereof, as described herein.

Suitable amino acid modifications for soluble γT-cell receptor chains, δT-cell receptor chains, or fragments thereof have been described earlier herein. Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

A nucleic acid molecule described herein may be comprised in a nucleic acid construct. A nucleic acid construct may be alternatively referred to herein as an “expression construct”. Accordingly, in a further aspect, there is provided a nucleic acid construct comprising a nucleic acid molecule as described herein. The skilled person understands that the nucleic acid molecule comprised in the nucleic acid constructs described herein may be operably linked to a regulatory sequence. A definition of “operably linked” is provided later herein. As used herein, a regulatory sequence refers to any genetic element that is known to the skilled person to drive or otherwise regulate expression of nucleic acids in a cell. Such sequences include without limitation promoters, transcription terminators, enhancers, repressors, silencers, kozak sequences, polyA sequences, and the like. A regulatory sequence can, for example, be inducible, non-inducible, constitutive, cell-cycle regulated, metabolically regulated, and the like. A regulatory sequence may be a promoter. More information on promoters is provided later herein. Accordingly, in some embodiments, the nucleic acid construct comprises a nucleic acid molecule operably linked to a promoter. Non-limiting examples of suitable promoters include EF1α, MSCV, EIF alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, MND, and derivatives (i.e. variants) thereof, of which the MSCV promoter is preferred. An example of an MSCV promoter comprises SEQ ID NO: 108.

A nucleic acid construct may comprise additional nucleic acid molecules, for example encoding a 2A-self cleaving peptide as described later herein.

A nucleic acid molecule or nucleic acid construct described herein may be comprised in a vector. Accordingly, in a further aspect, there is provided a vector comprising a nucleic acid molecule or nucleic acid construct as described herein. A preferred vector is a viral vector, preferably a retroviral or lentiviral vector. Suitable vectors are known to the skilled person and further information is provided later herein.

In some embodiments, the vector is a good manufacturing practices (GMP) compatible vector. For example, a GMP vector can be purer than a non-GMP vector. In some cases, purity can be measured by bioburden. For example, bioburden can be the presence or absence of aerobes, anaerobes, sporeformers, fungi, or combinations thereof in a vector composition. In some cases, a pure vector can be endotoxin low or endotoxin free. Purity can also be measured by double-stranded primer-walking sequencing. Plasmid identity can be a source of determining purity of a vector. A GMP vector of the disclosure can be from 10% to 99% more pure than a non-GMP vector. A GMP vector can be from 10%, 15%, 20%, 25%, 30%, 35%, 40%, 45%, 50%, 55%, 60%, 65%, 70%, 75%, 80%, 85%, 90%, 95%, 96%, 97%, 98%, or 99% more pure than a non-GMP vector as measured by the presence of bioburden, endotoxin, sequencing, or combinations thereof.

Each of the soluble δT-cell receptor chains, γT-cell receptor chains, γδT-cell receptors, or fragments thereof (such as e.g., extracellular domains or fragments thereof), and polypeptides comprising them (such as soluble polypeptides), described herein, or encoded by the nucleic acid molecules, constructs, and vectors described herein, are preferably able to mediate an anti-tumour activity/response and/or an anti-infective activity/response. Accordingly, they are preferably suitable for designing a medicament, such as for example for preventing, treating, regressing, curing and/or delaying a cancer or an infection in a subject, preferably in a human being.

TABLE 2
Exemplary sequences

| SEQ ID | | |
|---|---|---|
| NO: | Name | Sequence |
| 1 | TRG CDR3 E57 G1 | CATWDGFYYKKLF |
| 2 | TRD CDR3 E57 D3 | CAASSPIRGYTGSDKLIF |
| 4 | TRG full length E57 | MEWALAVLLAFLSPASQKSSNLEGRTKSVIRQTGSSAEITCDLAEG |
| | G1 | STGYIHWYLHQEGKAPQRLLYYDSYTSSVVLESGISPGKYDTYGST |
| | | RKNLRMILRNLIENDSGVYYCATWDGFYYKKLFGSGTTLVVTDKQL |
| | | DADVSPKPTIFLPSIAETKLQKAGTYLCLLEKFFPDVIKIHWEEKKSN |
| | | TILGSQEGNTMKTNDTYMKFSWLTVPEKSLDKEHRCIVRHENNKN |
| | | GVDQEIIFPPIKTDVITMDPKDNCSKDANDTLLLQLTNTSAYYMYLLL |
| | | LLKSVVYFAIITCCLLRRTAFCQNGEKS |
| 6 | TRD full length E57 | MAMLLGASVLILWLQPDWVNSQQKNDDQQVKQNSPSLSVQEGRI |
| | D3 | SILNCDYTNSMFDYFLWYKKYPAEGPTFLISISSIKDKNEDGRFTVFL |
| | | NKSAKHLSLHIVPSQPGDSAVYFCAASSPIRGYTGSDKLIFGKGTRV |
| | | TVEPRSQPHTKPSVFVMKNGTNVACLVKEFYPKDIRINLVSSKKITE |
| | | FDPAIVISPSGKYNAVKLGKYEDSNSVTCSVQHDNKTVHSTDFEVK |
| | | TDSTDHVKPKETENTKQPSKSCHKPKAIVHTEKVNMMSLTVLGLR |
| | | MLFAKTVAVNFLLTAKLFFL |
| 7 | TRG CDR3 E57 G2 | CATWDGYKKLF |
| 8 | TRG CDR3 E57 G3 | CATWDGYYKKLF |
| 9 | TRG CDR3 E57 G4 | CATWDGFYKKLF |
| 10 | TRG CDR3 E57 G5 | CATWDAFYYKKLF |
| 11 | TRG CDR3 E57 G6 | CATWEGFYYKKLF |
| 12 | TRG CDR3 E57 G7 | CATWEAFYYKKLF |
| 13 | TRG CDR3 E57 G8 | CATWDGFFYKKLF |
| 14 | TRG CDR3 E57 G9 | CATWDGYFYKKLF |
| 15 | TRG CDR3 E57 | CATWDGYYYKKLF |
| | G10 | |
| 16 | TRG CDR3 E57 | CATWDGAYYKKLF |
| | G11 | |
| 17 | TRG CDR3 E57 | CATWDGSYYKKLF |
| | G12 | |
| 18 | TRG CDR3 E57 | CATWDGHYYKKLF |
| | G13 | |
| 19 | TRD CDR3 E57 D1 | CAASSPIRGYTSGSDKLIF |
| 20 | TRD CDR3 E57 D2 | CAASSPIRAYTGSDKLIF |
| 21 | TRD CDR3 E57 D4 | CAASSPIRGFTGSDKLIF |
| 22 | TRD CDR3 E57 D5 | CAASSPIKGYTGSDKLIF |
| 23 | TRD CDR3 E57 D6 | CAASSPIKGFTGSDKLIF |
| 24 | TRD CDR3 E57 D7 | CAASSPLRGFTGSDKLIF |
| 25 | TRD CDR3 E57 D8 | CAASSPLRGYTGSDKLIF |
| 26 | TRD CDR3 E57 D10 | CAASSPLKGYTGSDKLIF |
| 27 | TRD CDR3 E57 D11 | CAASSPIRAFTGSDKLIF |
| 79 | TRG CDR3 E57 G1 | WDGFYYK |
| 80 | TRG CDR3 E57 G2 | WDGYK |
| 81 | TRG CDR3 E57 G3 | WDGYYK |
| 82 | TRG CDR3 E57 G4 | WDGFYK |
| 83 | TRG CDR3 E57 G5 | WDAFYYK |
| 84 | TRG CDR3 E57 G6 | WEGFYYK |
| 85 | TRG CDR3 E57 G7 | WEAFYYK |
| 86 | TRG CDR3 E57 G8 | WDGFFYK |
| 87 | TRG CDR3 E57 G9 | WDGYFYK |
| 88 | TRG CDR3 E57 | WDGYYYK |
| | G10 | |
| 89 | TRG CDR3 E57 | WDGAYYK |
| | G11 | |
| 90 | TRG CDR3 E57 | WDGSYYK |
| | G12 | |
| 91 | TRG CDR3 E57 | WDGHYYK |
| | G13 | |
| 92 | TRD CDR3 E57 D1 | IRGYTS |
| 93 | TRD CDR3 E57 D2 | IRAYTG |
| 94 | TRD CDR3 E57 D3 | IRGYTG |
| 95 | TRD CDR3 E57 D4 | IRGFTG |
| 96 | TRD CDR3 E57 D5 | IKGYTG |
| 97 | TRD CDR3 E57 D6 | IKGFTG |
| 98 | TRD CDR3 E57 D7 | LRGFTG |
| 99 | TRD CDR3 E57 D8 | LRGYTG |
| 100 | TRD CDR3 E57 D10 | LKGYTG |
| 101 | TRD CDR3 E57 D11 | IRAFTG |
| 102 | TRGC1 TM domain | YYMYLLLLLKSVVYFAIITCCLL |
| 103 | TRGC2 TM domain | YYTYLLLLLKSVVYFAIITCCLL |
| 104 | TRDC TM domain | LGLRMLFAKTVAVNFLLTAKLFF |
| 105 | OKT3 scFV | QVQLVQSGGGVVQPGRSLRLSCKASGYTFTRYTMHWVRQAPGK |
| | | GLEWIGYINPSRGYTNYNQKFKDRFTISRDNSKNTAFLQMDSLRPE |
| | | DTGVYFCARYYDDHYCLDYWGQGTPVTVSSGGGGSGGGGSGGG |
| | | GSDIQMTQSPSSLSASVGDRVTITCSASSSVSYMNWYQQTPGKAP |
| | | KRWIYDTSKLASGVPSRFSGSGSGTDYTFTISSLQPEDIATYYCQQ |
| | | WSSNPFTFGQGTKLQITRVDGS |
| 110 | TRD CDR3 E57 D9 | CAASSPLKGFTGSDKLIF |
| 111 | TRD CDR3 E57 D9 | LKGFTG |
| 312 | Extracellular E57 G1 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDGFYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 313 | Extracellular E57 G2 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDGYKKLFGSGTTLVVTDKQLDADVSPKPTIFLPSIAETKLQK |
| | | AGTYLCLLEKFFPDVIKIHWEEKKSNTILGSQEGNTMKTNDTYMKFS |
| | | WLTVPEKSLDKEHRCIVRHENNKNGVDQEIIFPPIKTDVITMDPKDN |
| | | CSKDANDTLLLQLTNTSAYYMY |
| 314 | Extracellular E57 G3 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDGYYKKLFGSGTTLVVTDKQLDADVSPKPTIFLPSIAETKLQ |
| | | KAGTYLCLLEKFFPDVIKIHWEEKKSNTILGSQEGNTMKTNDTYMKF |
| | | SWLTVPEKSLDKEHRCIVRHENNKNGVDQEIIFPPIKTDVITMDPKD |
| | | NCSKDANDTLLLQLTNTSAYYMY |
| 315 | Extracellular E57 G4 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDGFYKKLFGSGTTLVVTDKQLDADVSPKPTIFLPSIAETKLQ |
| | | KAGTYLCLLEKFFPDVIKIHWEEKKSNTILGSQEGNTMKTNDTYMKF |
| | | SWLTVPEKSLDKEHRCIVRHENNKNGVDQEIIFPPIKTDVITMDPKD |
| | | NCSKDANDTLLLQLTNTSAYYMY |
| 316 | Extracellular E57 G5 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDAFYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 317 | Extracellular E57 G6 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWEGFYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |

| | | |
|---|---|---|
| 318 | Extracellular E57 G7 | SSNLEGRTKSVIRQTGSSAEITCDLAEGSTGYIHWYLHQEGKAPQRLL |
| | | YYDSYTSSVVLESGISPGKYDTYGSTRKNLRMILRNLIENDSGVYYCA |
| | | TWEAFYYKKLFGSGTTLVVTDKQLDADVSPKPTIFLPSIAETKLQKAG |
| | | TYLCLLEKFFPDVIKIHWEEKKSNTILGSQEGNTMKTNDTYMKFSWL |
| | | TVPEKSLDKEHRCIVRHENNKNGVDQEIIFPPIKTDVITMDPKDNCSK |
| | | DANDTLLLQLTNTSAYYMY |
| 319 | Extracellular E57 G8 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDGFFYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 320 | Extracellular E57 G9 | SSNLEGRTKSVIRQTGSSAEITCDLAEGSTGYIHWYLHQEGKAPQR |
| | | LLYYDSYTSSVVLESGISPGKYDTYGSTRKNLRMILRNLIENDSGVY |
| | | YCATWDGYFYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 321 | Extracellular E57 | SSNLEGRTKSVIRQTGSSAEITCDLAEGSTGYIHWYLHQEGKAPQR |
| | G10 | LLYYDSYTSSVVLESGISPGKYDTYGSTRKNLRMILRNLIENDSGVY |
| | | YCATWDGYYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 322 | Extracellular E57 | SSNLEGRTKSVIRQTGSSAEITCDLAEGSTGYIHWYLHQEGKAPQR |
| | G11 | LLYYDSYTSSVVLESGISPGKYDTYGSTRKNLRMILRNLIENDSGVY |
| | | YCATWDGAYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 323 | Extracellular E57 | SSNLEGRTKSVIRQTGSSAEITCDLAEGSTGYIHWYLHQEGKAPQR |
| | G12 | LLYYDSYTSSVVLESGISPGKYDTYGSTRKNLRMILRNLIENDSGVY |
| | | YCATWDGSYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 324 | Extracellular E57 | SSNLEGRTKSVIRQTGSSAEITCDLAEGSTGYIHWYLHQEGKAPQR |
| | G13 | LLYYDSYTSSVVLESGISPGKYDTYGSTRKNLRMILRNLIENDSGVY |
| | | YCATWDGHYYKKLFGSGTTLVVTDKQLDADVSPKPTIFLPSIAETKL |
| | | QKAGTYLCLLEKFFPDVIKIHWEEKKSNTILGSQEGNTMKTNDTYM |
| | | KFSWLTVPEKSLDKEHRCIVRHENNKNGVDQEIIFPPIKTDVITMDP |
| | | KDNCSKDANDTLLLQLTNTSAYYMY |
| 325 | Extracellular E57 D1 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPIRGYTSGSDKLIFGKGTRVTVEPRSQPHTKPSVFVMKNGTNVAC |
| | | LVKEFYPKDIRINLVSSKKITEFDPAIVISPSGKYNAVKLGKYEDSNS |
| | | VTCSVQHDNKTVHSTDFEVKTDSTDHVKPKETENTKQPSKSCHKP |
| | | KAIVHTEKVNMMSLTV |
| 326 | Extracellular E57 D2 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPIRAYTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 327 | Extracellular E57 D3 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPIRGYTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 328 | Extracellular E57 D4 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPIRGFTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 329 | Extracellular E57 D5 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPIKGYTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 330 | Extracellular E57 D6 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPIKGFTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 331 | Extracellular E57 D7 | DQQVKQNSPSLSVQEGRISILNCDYTNSMFDYFLWYKKYPAEGPTFL |
| | | ISISSIKDKNEDGRFTVFLNKSAKHLSLHIVPSQPGDSAVYFCAASSPL |
| | | RGFTGSDKLIFGKGTRVTVEPRSQPHTKPSVFVMKNGTNVACLVKE |
| | | FYPKDIRINLVSSKKITEFDPAIVISPSGKYNAVKLGKYEDSNSVTCSVQ |
| | | HDNKTVHSTDFEVKTDSTDHVKPKETENTKQPSKSCHKPKAIVHTEK |
| | | VNMMSLTV |
| 332 | Extracellular E57 D8 | DQQVKQNSPSLSVQEGRISILNCDYTNSMFDYFLWYKKYPAEGPTFL |
| | | ISISSIKDKNEDGRFTVFLNKSAKHLSLHIVPSQPGDSAVYFCAASSPL |
| | | RGYTGSDKLIFGKGTRVTVEPRSQPHTKPSVFVMKNGTNVACLVKE |
| | | FYPKDIRINLVSSKKITEFDPAIVISPSGKYNAVKLGKYEDSNSVTCSVQ |
| | | HDNKTVHSTDFEVKTDSTDHVKPKETENTKQPSKSCHKPKAIVHTEK |
| | | VNMMSLTV |
| 333 | Extracellular E57 D9 | DQQVKQNSPSLSVQEGRISILNCDYTNSMFDYFLWYKKYPAEGPT |
| | | FLISISSIKDKNEDGRFTVFLNKSAKHLSLHIVPSQPGDSAVYFCAAS |
| | | SPLKGFTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 334 | Extracellular E57 | DQQVKQNSPSLSVQEGRISILNCDYTNSMFDYFLWYKKYPAEGPT |
| | D10 | FLISISSIKDKNEDGRFTVFLNKSAKHLSLHIVPSQPGDSAVYFCAAS |
| | | SPLKGYTGSDKLIFGKGTRVTVEPRSQPHTKPSVFVMKNGTNVAC |
| | | LVKEFYPKDIRINLVSSKKITEFDPAIVISPSGKYNAVKLGKYEDSNS |
| | | VTCSVQHDNKTVHSTDFEVKTDSTDHVKPKETENTKQPSKSCHKP |
| | | KAIVHTEKVNMMSLTV |
| 335 | Extracellular E57 | DQQVKQNSPSLSVQEGRISILNCDYTNSMFDYFLWYKKYPAEGPT |
| | D11 | FLISISSIKDKNEDGRFTVFLNKSAKHLSLHIVPSQPGDSAVYFCAAS |
| | | SPIRAFTGSDKLIFGKGTRVTVEPRSQPHTKPSVFVMKNGTNVACL |
| | | VKEFYPKDIRINLVSSKKITEFDPAIVISPSGKYNAVKLGKYEDSNSV |
| | | TCSVQHDNKTVHSTDFEVKTDSTDHVKPKETENTKQPSKSCHKPK |
| | | AIVHTEKVNMMSLTV |
| 362 | c-Jun dimerization | RIARLEEKVKTLKAQNSELASTANMLREQVAQLKQKVMNY |
| | motif | |
| 363 | c-Fos dimerization | LTDTLQAETDQLEDKKSALQTEIANLLKEKEKLEFILAA |
| | motif | |
| 369 | E57 G5 | DAFYY |
| 370 | E57 G7 | EAFYY |
| 371 | E57 G9 | DGYFY |
| 372 | E57 G10 | DGYYY |
| 373 | E57 G11 | DGAYY |
| 374 | E57 G12 | DGSYY |
| 375 | E57 gamma CDR1 | EGSTGY |
| 376 | E57 gamma CDR2 | YDSYTSSV |
| 377 | E57 delta CDR1 | NSMFDY |
| 378 | E57 delta CDR2 | ISSIKDK |

### Cells

In a further aspect, there is provided a cell expressing a soluble, such as a chimeric, polypeptide as described herein. In some embodiments, the cell is a mammalian cell, preferably a human cell. In some embodiments, the cell comprises a nucleic acid molecule, nucleic acid construct, or vector as described herein. Suitable examples of cells have been described earlier herein.

It is understood that in embodiments wherein a cell expresses a soluble γT-cell receptor chain or fragment thereof as described herein, the cell may also express a soluble δT-cell receptor chain or fragment thereof. Expression of any soluble δT-cell receptor chain or fragment thereof may be contemplated, as long as a functional soluble γδTCR which is able to mediate an anti-tumour or anti-infective response is obtained. It is also understood that in embodiments wherein a cell expresses a soluble δT-cell receptor chain or fragment thereof as described herein, the cell may also express a soluble γT-cell receptor chain or fragment thereof. Expression of any soluble γT-cell receptor chain or fragment thereof may be contemplated, as long as a functional soluble γδTCR which is able to mediate an anti-tumour or anti-infective response is obtained.

In some embodiments, bringing an immunoresponsive cell, preferably selected from a T-cell, an iPSC-derived T-cell, an αβT-cell, a γδT-cell, or an NK cell, more preferably selected from a γδT-cell or αβT-cell, most preferably an αβT-cell, into contact with a soluble γT-cell receptor chain, a δT-cell receptor chain, a γδT-cell receptor, or a fragment thereof, most preferably αβT-cell, may result in increased anti-tumour or anti-infective response, increased expansion, fitness and/or survival, and/or decreased exhaustion of the immunoresponsive cell relative to an otherwise comparable cell not brought into contact with the soluble γT-cell receptor chain, soluble δT-cell receptor chain, soluble γδT-cell receptor, or fragment thereof of the disclosure. Assays for measuring the anti-tumour or anti-infective response of immunoresponsive cells have been described earlier herein, and further examples are given in the experimental section.

In some embodiments, the anti-tumour or anti-infective response of an immunoresponsive cell brought into contact with a soluble γT-cell receptor chain, a soluble δT-cell receptor chain, a soluble γδT-cell receptor, or a fragment thereof, as described herein, is increased by at least 5%, at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, at least 10-fold, or more, relative to an otherwise comparable immunoresponsive cell not brought into contact with the soluble γT-cell receptor chain, δT-cell receptor chain, γδT-cell receptor, or fragment thereof.

The expansion (proliferative ability), fitness and/or survival (lifespan), and/or exhaustion of the immunoresponsive cell brought into contact with the soluble γT-cell receptor chain, soluble δT-cell receptor chain, soluble γδT-cell receptor, or fragment thereof, may be assessed using any technique known to the skilled person.

Fitness of T-cells may, for example, be monitored based on staining for various markers including, but not limited to CD4, CD8a, CD3, αβTCR, γδTCR, 4-1BB, OX40, PD-1, TIM-3, LAG-3, 4-1BBL, OX40L, CD86, Fab2, CD107a and CD69, for example staining with fluorescent-labeled antibodies targeting these markers in combination with flow cytometry.

In some embodiments, expansion of an immunoresponsive cell brought into contact with a soluble γT-cell receptor chain, a soluble δT-cell receptor chain, a soluble γδT-cell receptor, or a fragment thereof, as described herein, is increased by at least 5%, at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, at least 10-fold, or more, relative to an otherwise comparable immunoresponsive cell not brought into contact with the soluble γT-cell receptor chain, δT-cell receptor chain, γδT-cell receptor, or fragment thereof.

In some embodiments, fitness of an immunoresponsive cell brought into contact with a soluble γT-cell receptor chain, a soluble δT-cell receptor chain, a soluble γδT-cell receptor, or a fragment thereof, as described herein, is increased by at least 5%, at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, at least 10-fold, or more, relative to an otherwise comparable immunoresponsive cell not brought into contact with the soluble γT-cell receptor chain, δT-cell receptor chain, γδT-cell receptor, or fragment thereof.

Survival of immunoresponsive cells may, for example, be monitored based on any cell viability assay known to the skilled person, many of which are commercially available (see for example the assays offered by ThermoFisher Scientific, WA, MA, USA). Non-limiting examples of cell viability assays involve the use of dyes such as calcein AM, ethidium-homodimer-1, SYTOX Deep Red, DiOC 19(3), propidium iodide, SYBR 14, SYTO 10, green ethidium homodimer-2, SYTOX Green, C-12 resazurin, BOBO-3 iodide, DAPI, and others. By knowing a starting number of immunoresponsive cells, the skilled person may monitor their survival by measuring the number of viable cells overtime.

In some embodiments, survival of an immunoresponsive cell brought into contact with a soluble γT-cell receptor chain, a soluble δT-cell receptor chain, a soluble γδT-cell receptor, or a fragment thereof, as described herein, is increased by at least 5%, at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, at least 10-fold, or more, relative to an otherwise comparable immunoresponsive cell not brought into contact with the γT-cell receptor chain, δT-cell receptor chain, γδT-cell receptor, or fragment thereof. Survival may be measured over a defined period, for example over about 1 day, about 2 days, about 3 days, about 4 days, about 5 days, about 6 days, about 1 week, about 2 weeks, about 3 weeks, about 4 weeks, about 5 weeks, about 6 weks, about 7 weeks, about 8 weeks, about 9 weeks, or about 10 weeks.

In some embodiments, upon exposure of an immunoresponsive cell brought into contact with a soluble γT-cell receptor chain, a soluble δT-cell receptor chain, a soluble γδT-cell receptor, or a fragment thereof, as described herein, to target cells for at least 1 day, at least 2 days, at least 3 days, at least 4 days, at least 5 days, at least 6 days, at least 7 days, at least 8 days, at least 9 days, at least 10 days, at least 14 days, at least 21 days, or at least 28 days, expression of an exhaustion marker by the immunoresponsive cell is at least 5%, least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold lower compared to upon exposure of a corresponding control (comparable) immunoresponsive cell not brought into contact with the soluble γT-cell receptor chain, δT-cell receptor chain, γδT-cell receptor, or fragment thereof.

In some embodiments, the increased anti-tumour or anti-infective response, increased expansion, fitness and/or survival, and/or decreased exhaustion may persist over multiple stimulations of the immunoresponsive cells, such as at least two, at least three, at least four, at least five, at least six, at least seven, at least eight, at least nine stimulations or more, for example over multiple exposures to target antigens, epitopes, or target cells (serial stimulation). The persisting improvement may be at least 5%, at least 10%, at least 20%, at least 30%, at least 40%, at least 50%, at least 60%, at least 70%, at least 80%, at least 90%, at least 100% or more, relative to control (comparable) immunoresponsive cells not brought into contact with the soluble γT-cell receptor chain, δT-cell receptor chain, γδT-cell receptor, or fragment thereof. Stimulation of immunoresponsive cells has been described earlier herein and a further example is provided in the experimental section.

### Population of Cells

In a further aspect, there is provided a population of cells comprising the cell as defined earlier herein. In some embodiments, such a cell expresses a soluble γT-cell receptor chain, a soluble δT-cell receptor chain, a soluble γδT-cell receptor, or a fragment thereof as described herein. In some embodiments, such a cell comprises a nucleic acid molecule, construct, or vector encoding a soluble γT-cell receptor chain, a soluble δT-cell receptor chain, a soluble γδT-cell receptor, or a fragment thereof, as described herein.

In some embodiments, within the cell population, at least 1%, at least 2%, at least 3%, at least 4%, at least 5%, at least 10%, at least 20%, at least 30%, at least 40%, at least 50%, at least 60%, at least 70%, at least 80%, at least 90%, at least 95%, or 100% of the cells are expressing a soluble γT-cell receptor chain, soluble δT-cell receptor chain, soluble γδT-cell receptor, or fragment thereof, or are comprising a nucleic acid molecule, construct, or vector encoding a soluble γT-cell receptor chain, soluble δT-cell receptor chain, soluble γδT-cell receptor, or fragment thereof, as described herein.

The person skilled in the art is well capable of selecting and/or identifying cell populations, or cells within cell populations, characterized by expression of the soluble γT-cell receptor chain, soluble δT-cell receptor chain, soluble γδT-cell receptor, or fragment thereof using, e.g., flow cytometric methods such as FACS as explained earlier herein.

### Compositions

In a further aspect, there is provided a composition, preferably a pharmaceutical composition, comprising a soluble γT-cell receptor chain, a soluble δT-cell receptor chain, a soluble γδT-cell receptor, a fragment thereof, a conjugate, a nucleic acid molecule, a nucleic acid construct, a vector, or a cell, as described herein.

Pharmaceutical compositions of the present disclosure comprise an effective amount of one or more molecules (i.e., a soluble γT-cell receptor chain, a soluble δT-cell receptor chain, a soluble γδT-cell receptor, a fragment thereof, a conjugate, a nucleic acid molecule, a nucleic acid construct, a vector, or a cell, as described herein), optionally dissolved or dispersed in a pharmaceutically acceptable carrier.

The term “effective amount” as used herein is defined as the amount of the molecules of the present disclosure that are necessary to result in the desired physiological change in the cell or tissue to which it is administered. The term “therapeutically effective amount” as used herein is defined as the amount of the molecules of the present disclosure that achieves a desired effect with respect to cancer. In this context, a “desired effect” is synonymous with “an anti-tumour or anti-infective activity” as earlier defined herein. A skilled artisan readily recognizes that in many cases the molecules may not provide a cure but may provide a partial benefit, such as alleviation or improvement of at least one symptom or parameter. In some embodiments, a physiological change having some benefit is also considered therapeutically beneficial. Thus, in some embodiments, an amount of molecules that provides a physiological change is considered an “effective amount” or a “therapeutically effective amount.”

The phrases “pharmaceutically or pharmacologically acceptable” refers to molecular entities and compositions that do not produce or produce acceptable adverse, allergic or other untoward reaction when administered to an animal, such as, for example, a human, as appropriate. Whether certain adverse effects are acceptable is determined based on the severity of the disease. The preparation of a pharmaceutical composition that contains at least one active ingredient will be known to those of skill in the art in light of the present disclosure, as exemplified by Remington's Pharmaceutical Sciences, 18th Ed. Mack Printing Company, 1990, incorporated herein by reference in its entirety. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, supra). Except insofar as any conventional carrier is incompatible with the molecules described herein, its use in the therapeutic or pharmaceutical compositions is contemplated.

In certain embodiments, a pharmaceutical composition described herein further comprises a suitable amount of an antifungal agent. In some cases, a pharmaceutical composition described herein comprises an antifungal agent in an amount sufficient for the pharmaceutical composition to retain at least 20%, 30%, 40%, 50%, 60%, 70%, 80%, or 90% of its desired activity for a period of at least 1 month, 2 months, 3 months, 6 months, 1 year, 1.5 years, 2 years, 2.5 years or 3 years.

The actual dosage amount of a composition of the present disclosure administered to an animal or a patient (e.g., a human) can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### Therapy

The soluble γT-cell receptor chains, soluble δT-cell receptor chains, soluble γδTCRs, fragments thereof, conjugates, cells, populations of cells, and compositions (such as pharmaceutical compositions), all as described herein, are preferably able to mediate an anti-tumour activity/response and/or an anti-infective activity/response. The soluble γT-cell receptor chains, soluble δT-cell receptor chains, soluble γδTCRs, fragments thereof, conjugates, cells, populations of cells, and compositions (such as pharmaceutical compositions), all as described herein, are preferably suitable for use in therapy.

Accordingly, in a further aspect, there is provided a soluble γT-cell receptor chain or fragment thereof comprising a γCDR3 region, a soluble δT-cell receptor chain or fragment thereof comprising a δCDR3 region, a soluble γδT-cell receptor or fragment thereof comprising a γCDR3 and a δCDR3 region, a conjugate, a nucleic acid molecule, a nucleic acid construct, a vector, a cell, a population of cells, or a composition (such as a pharmaceutical composition), all as described herein, for use as a medicament. The medicament is preferably for the treatment, regression, curing, and/or delaying of cancer or an infection, more preferably of cancer, in a subject, preferably a human being.

In a further aspect, there is provided the use of a soluble γT-cell receptor chain or fragment thereof comprising a γCDR3 region, a soluble δT-cell receptor chain or fragment thereof comprising a δCDR3 region, a soluble γδT-cell receptor or fragment thereof comprising a γCDR3 and a δCDR3 region, a conjugate, a nucleic acid construct, a vector, a cell, a population of cells, or a composition (such as a pharmaceutical composition), all as described herein, for the manufacture of a medicament. The medicament is preferably for the treatment, regression, curing, and/or delaying of cancer or an infection, more preferably of cancer, in a subject, preferably a human being.

In a further aspect, there is provided a method of treatment, regression, curing, and/or delaying of a disease comprising providing a soluble γT-cell receptor chain or fragment thereof comprising a γCDR3 region, a soluble δT-cell receptor chain or fragment thereof comprising a δCDR3 region, a soluble γδT-cell receptor or fragment thereof comprising a γCDR3 and a δCDR3 region, a conjugate, a nucleic acid construct, a vector, a cell, a population of cells, or a composition (such as a pharmaceutical composition), all as described herein, to a subject in need thereof, preferably to a human. The method is preferably a method of treatment, regression, curing, and/or delaying of a cancer or infection.

In therapeutic uses, uses, and methods of treatment described herein, the soluble γT-cell receptor chain or fragment thereof comprising a γCDR3 region, soluble δT-cell receptor chain or fragment thereof comprising a δCDR3 region, soluble γδT-cell receptor or fragment thereof comprising a γCDR3 and a δCDR3 region, conjugate, nucleic acid construct, vector, cell, population of cells, or composition (such as pharmaceutical composition) are administer to a subject in need thereof, i.e., a subject that is afflicted by or is at risk of developing the disease or infection. A preferred subject is a human being. The amount administered is preferably a therapeutically effective amount, as described earlier herein.

In some embodiments, a cancer is a liquid cancer. In some embodiments, a cancer is a solid cancer. In some embodiments, a cancer is a colon cancer. In some embodiments, a cancer is a breast cancer.

In some embodiments, a cancer is a metastatic cancer. In some embodiments, a cancer is a relapsed or refractory cancer. In some embodiments, a cancer is a solid tumour or a hematologic malignancy. In some embodiments, a cancer is a solid tumour. In some embodiments, a cancer is a hematologic malignancy.

The following general part dedicated to the definitions provides more information as to the aspects and embodiments described herein.

### General Part Dedicated to Definitions

### Polypeptide/Nucleic Acid

A “wild type” protein/polypeptide amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide. In some embodiments, a reference sequence used herein is a wild type sequence. In some embodiments, a reference sequence used herein is isolated from a healthy individual. In some embodiments, a reference sequence used herein is isolated from a patient.

An amino acid sequence that is “derived from” a wild type sequence or reference sequence or another amino acid sequence disclosed herein can refer to an amino acid sequence that comprises an amino acid modification, for example an amino acid sequence that differs by one or more amino acids compared to the wild type or reference amino acid sequence, for example, containing one or more amino acid insertions, deletions, or substitutions as disclosed herein.

In the context of the disclosure, a polypeptide comprises an amino acid sequence. In the context of the disclosure, a nucleic acid molecule such as a nucleic acid molecule encoding a soluble γT-cell receptor chain, a soluble δT-cell receptor chain, a soluble γδTCR, or a fragment thereof comprises a nucleic acid or nucleotide sequence which encodes such a polypeptide. A nucleic acid molecule may comprise a regulatory region.

It is to be understood that each nucleic acid molecule or polypeptide or construct as identified herein by a given Sequence Identity Number (SEQ ID NO) is not limited to this specific sequence as disclosed. Throughout this application, each time one refers to a specific nucleotide sequence SEQ ID NO (take SEQ ID NO: X as example) encoding a given polypeptide, one may replace it by:
i. a nucleotide sequence comprising a nucleotide sequence that has at least 60% or at least 80% sequence identity with SEQ ID NO: X;
ii. a nucleotide sequences the complementary strand of which hybridizes to a nucleic acid molecule of sequence of (i);
iii. a nucleotide sequence the sequence of which differs from the sequence of a nucleic acid molecule of (i) or (ii) due to the degeneracy of the genetic code; or,
iv. a nucleotide sequence that encodes an amino acid sequence that has at least 60% or at least 80% amino acid identity or similarity with an amino acid sequence encoded by a nucleotide sequence SEQ ID NO: X.

Throughout this application, each time one refers to a specific amino acid sequence SEQ ID NO (take SEQ ID NO: Y as example), one may replace it by: a polypeptide comprising an amino acid sequence that has at least 60% sequence identity or similarity with amino acid sequence SEQ ID NO: Y.

In the context of the application, the minimum identity or similarity in relation with a γT-cell receptor chain or fragment thereof may mean an identity or a similarity of at least 60%.

In the context of the application, the minimum identity or similarity in relation with a δT-cell receptor chain or fragment thereof may mean an identity or a similarity of at least 60%.

Each nucleotide sequence or amino acid sequence described herein by virtue of its identity or similarity percentage (e.g. at least 60%) with a given nucleotide sequence or amino acid sequence respectively has in a further preferred embodiment an identity (or a similarity where applicable) of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% with the given nucleotide or amino acid sequence respectively. In a preferred embodiment, sequence identity or similarity is determined by comparing the whole length of the sequences as identified herein. Unless otherwise indicated herein, identity or similarity with a given SEQ ID NO means identity or similarity based on the full length of said sequence (i.e. over its whole length or as a whole).

### Sequence Identity

“Sequence identity” is herein defined as a relationship between two or more amino acid (polypeptide or protein) sequences or two or more nucleic acid (polynucleotide) sequences, as determined by comparing the sequences. The identity between two amino acid or two nucleic acid sequences may be defined by assessing their identity within a whole length SEQ ID NO as identified herein or part thereof. Part thereof in terms of comparing the identity or similarity of two or more sequences may mean at least 50% of the length of the SEQ ID NO, or at least 60%, or at least 70%, or at least 80%, or at least 90%. In a preferred embodiment, sequence identity or similarity is determined by comparing the whole length of sequences identified herein. In other words, sequence identity is preferably calculated based on the full length of two given sequences being compared (for example of a sequence represented by a SEQ ID NO herein and of another sequence it is being compared to).

In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference in its entirety.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water (EMBLI-EBI) using default parameters share at least a certain minimal percentage of sequence identity (as described herein).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for amino acid sequences the default scoring matrix is Blosum62 (Henikoff & Henikoff, 1992, PNAS 89, 915-919, incorporated herein by reference).

Alternatively percentage similarity or identity may be determined by searching against public databases, using algorithms such as FASTA, BLAST, etc. Thus, the nucleotide and amino acid sequences of some embodiments of the present disclosure can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference in its entirety. BLAST nucleotide searches can be performed with the NBLAST program, score=100, wordlength=12 to obtain nucleotide sequences having a certain identity with nucleic acid molecules of the disclosure. BLAST protein searches can be performed with the BLASTx program, score=50, wordlength=3 to obtain amino acid sequences having a certain identity or similarity with polypeptides of the disclosure. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called “conservative” amino acid substitutions, as will be clear to the skilled person. As used herein, “conservative” amino acid substitutions refer to the interchangeability of residues having similar side chains. “Similarity” between two amino acid sequences is determined by comparing the amino acid sequence and its conserved amino acid substitutes of one polypeptide to the sequence of a second polypeptide. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

| | |
|---|---|
| Alcohol group-containing residues | S and T |
| Aliphatic residues | I, L, V, and M |
| Cycloalkenyl-associated residues | F, H, W, and Y |
| Small residues | A, C, D, G, N, P, S, T, and V |
| Very small residues | A, G, and S |
| Residues involved in turn formation | A, C, D, E, G, H, K, N, Q, |
| | R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Other examples of conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Other examples of conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lie or Val; Lys to Arg; Gln or Glu; Met to Leu or lie; Phe to Met, Leu or Tyr; Ser to Thr; Thr to Ser; Trp to Tyr; Tyr to Trp or Phe; and, Val to lie or Leu.

In some embodiments described herein, an amino acid substitution is a substitution of a hydrophobic, a charged, or a polar amino acid. In some embodiments described herein, an amino acid modification is an insertion of a hydrophobic, charged, or polar amino acid. In some embodiments described herein, an amino acid modification is a deletion of a hydrophobic, charged, or polar amino acid.

Examples of hydrophobic, charged, and polar amino acids are given in the Table below:

| | |
|---|---|
| Hydrophobic amino acids | G, A, V, L, I, P, F, M, W |
| Charged amino acids | D, E, H, K, R |
| Polar amino acids | S, T, C, N, Q, Y |

### Antigen

A soluble γT-cell receptor chain, δT-cell receptor chain, γδTCR, or fragment thereof, as disclosed herein may, for example, bind to an antigen comprised in or displayed by a target cell. An “antigen” is a molecule or molecular structure that an antigen receptor or an antigen-binding protein can recognize (for example, bind to). An antigen can be or can comprise, for example, a peptide, a polypeptide, a carbohydrate, a chemical, a moiety, a non-peptide antigen, a phosphoantigen, a tumour-associated antigen, a neoantigen, a tumour microenvironment antigen, a microbial antigen, a viral antigen, a bacterial antigen, an autoantigen, a glycan-based antigen, a peptide-based antigen, a lipid-based antigen, or any combination thereof. In some embodiments, an antigen is capable of inducing an immune response. In some examples, an antigen binds to an antigen receptor or antigen-binding protein, or induces an immune response, when present in a complex e.g., presented by MHC. In some cases, an antigen adopts a certain conformation in order to bind to an antigen receptor or antigen-binding protein, and/or to induce an immune response, e.g., adopts a conformation in response to the presence or absence of one or more metabolites. Antigen can refer to a whole target molecule, a whole complex, a or a fragment of a target molecule or complex that binds to an antigen receptor or an antigen-binding protein. Antigen receptors that recognize antigens include γδTCR disclosed herein and other receptors, such as endogenous T-cell receptors. In some embodiments, the antigen is EPCR (Endothelial protein C receptor), preferably human EPCR.

### Gene or Coding Sequence

“Gene” or “coding sequence” or “coding nucleic acid” or “coding nucleic acid molecule” refers to a DNA or RNA region (the transcribed region) which “encodes” a particular polypeptide such as a soluble γT-cell receptor or a soluble δT-cell receptor or a soluble γδTCR or a fragment thereof described herein. A coding sequence is transcribed (DNA) and translated (RNA) into a polypeptide when placed under the control of an appropriate regulatory region, such as a promoter. A gene may optionally comprise several operably linked fragments, such as a promoter, a 5′ leader sequence, an intron, a coding sequence and a 3′ nontranslated sequence, comprising a polyadenylation site or a signal sequence. A chimeric or recombinant gene (such as the ones described herein) is a gene not normally found in nature, such as a gene in which for example the promoter is not associated in nature with part or all of the transcribed DNA region, or genes comprising nucleotide sequences encoding domains from multiple polypeptides. “Expression of a gene” refers to the process wherein a gene is transcribed into an RNA and/or translated into an active protein.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host cell. For example, to suit the codon preference of mammalian, insect, plant, or microbial cells, preferably human cells. Codon optimization also eliminates elements that potentially impact negatively RNA stability and/or translation (e.g. termination sequences, TATA boxes, splice sites, ribosomal entry sites, repetitive and/or GC rich sequences and RNA secondary structures or instability motifs). Codon optimization may be done according to standard methods available to skilled person.

### Promoter

As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is preferentially active in specific types of differentiated cells/tissues, such as preferably a T cell. A preferred promoter is the MSCV promoter. An example of an MSCV promoter comprises SEQ ID NO: 108.

### Operably Linked

“Operably linked” is defined herein as a configuration in which a control sequence such as a promoter sequence or regulating sequence is appropriately placed at a position relative to the nucleotide sequence of interest. For instance, a promoter is operably linked to a coding sequence if the promoter is able to initiate or regulate the transcription or expression of a coding sequence, in which case the coding sequence should be understood as being “under the control of” the promoter.

### Nucleic Acid Construct

An “expression construct” or “nucleic acid construct” comprises a nucleic acid molecule, such as the ones described herein, which may be expressed in a host cell. In some cases, such a construct is a viral expression construct. A viral expression construct comprises parts of a virus' genome, as further described later herein.

Within the context of the disclosure, a host cell may mean to encompass a cell used to make the construct or a cell wherein the construct will be administered. Alternatively, a nucleic acid construct is capable of integrating into a cell's genome, e.g., through homologous recombination or otherwise. A particularly preferred expression construct is one wherein a nucleotide sequence encoding a soluble γT-cell receptor chain, δT-cell receptor chain, γδTCR, or fragment thereof, as described herein, is operably linked to a promoter as defined herein wherein said promoter is capable of directing expression of said nucleotide sequence (i.e., coding sequence) in a cell, preferably a human cell. Such a preferred expression construct is said to comprise an expression cassette.

An expression construct may comprise two expression cassettes to allow the expression of two polypeptides such as a soluble γTCR and a soluble δTCR chain, or fragments thereof.

An expression construct may further comprise a sequence encoding a 2A-self cleaving peptide. These self-cleaving peptides are known to the skilled person and are further described, for example, in Xu Y., et al (2019), Cancer Immunology, Immunotherapy, 68: 1979-1993 and Pincha M., et al, (2011), Gene Therapy, 18: 750-764, both of which incorporated herein by reference in their entireties. Non-limiting examples of suitable 2A peptides are F2A (2A peptide derived from the foot-and-mouth disease virus), E2A (2A peptide derived from the equine rhinitis virus), P2A (2A peptide derived from the porcine teschovirus-1), or T2A (2A peptide derived from the Thosea asigna virus). In some embodiments, the 2A self-cleaving peptide is a F2A peptide. In some embodiments, the 2A self-cleaving peptide is an E2A peptide. In some embodiments, the 2A self-cleaving peptide is a P2A peptide. In some embodiments, the 2A self-cleaving peptide is a T2A peptide. The skilled person understands that an expression construct described herein may also comprise nucleotide sequences encoding different 2A self-cleaving peptides. In expression constructs encoding a soluble γδTCR or a fragment thereof, a sequence encoding a 2A self-cleaving peptide may in some cases be inserted between the sequence encoding the soluble γT-cell receptor chain or fragment thereof and the soluble δT-cell receptor chain or fragment thereof. An exemplary T2A self-cleaving peptide is represented by SEQ ID NO: 364.

Expression constructs disclosed herein could be prepared using recombinant techniques which result in nucleotide sequences being expressed in a suitable cell, e.g., cultured cells or cells of a multicellular organism.

Typically, a nucleic acid molecule or construct is used in a vector. A vector may alternatively be called an expression vector. The phrase “expression vector” generally refers to a nucleotide sequence that is capable of effecting expression of a gene in a host compatible with such sequences. These expression vectors typically include at least suitable promoter sequences and optionally, transcription termination signals. An additional factor necessary or helpful in effecting expression can also be used as described herein. An expression vector may optionally be suitable for replication in a prokaryotic host, such as bacteria, e.g., E. coli, or may be introduced into a cultured mammalian, plant, insect, (e.g., Sf9), yeast, fungi or other eukaryotic cell lines.

A nucleic acid molecule, construct, or vector, prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been defined herein. DNA signal sequences may be included. DNA for a signal sequence is operably linked to DNA encoding a polypeptide if it is expressed as a preprotein that participates in the secretion of a polypeptide. Generally, DNA sequences that are operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art. Additional examples have been provided earlier herein.

A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available. For example, an expression vector which includes the replication system and transcriptional and translational regulatory sequences together with the insertion site for the polypeptide encoding segment can be employed. In most cases, the replication system is only functional in the cell that is used to make the vector (bacterial cell as E. Coli). Most plasmids and vectors do not replicate in the cells infected with the vector. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media. A cell may be a cell line, e.g. a HEK293 or HEK293F cell line or a derivative thereof. In some cases, a host cell is a cell that is part of a multicellular organism such as a transgenic plant or animal, preferably a human. A vector as described herein may be selected from any genetic element known in the art which can facilitate transfer of nucleic acids between cells, such as, but not limited to, plasmids, transposons, cosmids, chromosomes, artificial chromosomes, viruses, virions, and the like. A vector may also be a chemical vector, such as a lipid complex or naked DNA. “Naked DNA” or “naked nucleic acid” refers to a nucleic acid molecule that is not contained in encapsulating means that facilitates delivery of a nucleic acid into the cytoplasm of a target host cell. Naked DNA may be circular or linear (linearized DNA sequence). Optionally, a naked nucleic acid can be associated with standard means used in the art for facilitating its delivery of the nucleic acid to the target host cell, for example to facilitate the transport of the nucleic acid through the cell membrane.

A preferred vector is a viral vector. Non-limiting examples of viral vectors are adenoviral vectors, adeno-associated viral vectors, retroviral vectors, and lentiviral vectors. Among viral vectors, retroviral and lentiviral vectors are preferred, with lentiviral vectors being further preferred. In an embodiment, a single bicistronic viral vector is used. In an embodiment, a single bicistronic lentiviral vector further comprising a 2A self-cleaving peptide sequence is used, as described earlier herein.

Adenoviral and adeno associated viral (AAV) vectors infect a wide number of dividing and non-dividing cell types including synovial cells and liver cells. The episomal nature of the adenoviral and AAV vectors after cell entry makes these vectors suited for therapeutic applications (Russell, 2000, J. Gen. Virol. 81: 2573-2604; Goncalves, 2005, Virol J. 2(1):43). AAV vectors are known to result in very stable long term expression of transgene expression. Preferred adenoviral vectors are modified to reduce the host response. Lentiviral vectors have the ability to infect and to stably integrate into the genome of dividing and non-dividing cells. Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Other suitable viral vectors include a herpes virus vector, a polyoma virus vector or a vaccinia virus vector. Transposon or other non-viral delivery systems may also be used in this context. All systems can be used in vitro or in vivo.

A viral vector may optionally comprise a further nucleotide sequence coding for a further polypeptide. A further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in standard handbooks.

In some embodiments, a viral vector may be formulated in a pharmaceutical composition as defined herein. In this context, a pharmaceutical composition may comprise a suitable pharmaceutical carrier as earlier defined herein.

An example of a suitable vector comprises SEQ ID NO: 109.

### Transgene

A “transgene” is herein defined as a gene or a nucleic acid molecule that has been newly introduced into a cell. The transgene may comprise sequences that are native to the cell, sequences that naturally do not occur in the cell and it may comprise combinations of both. A transgene may contain sequences coding for a soluble γTCR or δTCR chain or γδTCR or fragment thereof and/or additional domains as earlier identified herein that may be operably linked to appropriate regulatory sequences.

### Transduction

“Transduction” refers to the delivery of a soluble γTCR or soluble δTCR chain or soluble γδTCR or fragment thereof as earlier defined herein into a recipient host cell by a viral vector. For example, transduction of a cell with e.g., a retroviral or lentiviral vector as described herein leads to transfer of the genome contained in that vector into the transduced cell. In an embodiment, the vector is a retroviral or lentiviral vector. An example of transduction is provided in Pirona et al., 2020 (Biology Methods and Protocols, Volume 5, Issue 1, 2020, bpaa005). Additional examples are provided in the experimental section herein.

### Engineered Cells

The term “engineered cells” refers herein to cells having been engineered, e.g., by the introduction of an exogenous nucleic acid sequence as defined herein. Such a cell has been genetically modified for example by the introduction of for example one or more mutations, insertions and/or deletions in an endogenous gene and/or insertion of a nucleic acid construct in the genome. The modification may have been introduced using recombinant DNA technology. An engineered cell may refer to a cell in isolation or in culture. Engineered cells may be “transduced cells” wherein the cells have been infected with e.g. a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient, but may also be a stable transfection wherein cells can be selected that have the gene construct integrated in their genome. In some cases genetic engineering systems such as CRISPR or Argonaute may be utilized to design engineered cells that express a polypeptide described herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA. Genome editing tools as described above may also be used to introduce a genomic modification which results in the reduction or elimination of surface expression of an endogenous αβTCR in an αβT-cell as discussed earlier herein.

### TEG

A “TEG” as used herein refers to a T-cell engineered to express a defined γTCR chain, δTCR chain, γδTCR, or a fragment thereof.

As a non-limiting example, a TEG can be an αβT-cell that is engineered to express a defined γTCR chain, δTCR chain, γδTCR or fragment thereof. Examples of TEGs are shown in the experimental section

In some embodiments, the cells can be cultured for extended periods without stimulation or with stimulation. Stimulation may comprise contact with an anti-CD3 antibody or antigen binding fragment thereof immobilized on a surface. For co-stimulation of an accessory molecule on the surface of the T-cells, a ligand that binds the accessory molecule can be used. In some cases a population of T-cells can be CD3-CD28 co-stimulated, for example, contacted with an anti-CD3 antibody and an anti-CD28 antibody, under conditions that can stimulate proliferation of the T-cells.

Conditions appropriate for T-cell culture can include an appropriate media (e.g., Minimal Essential Media or RPMI Media 1640, TexMACS (Miltenyi Biotec, Bergisch Gladbach, Germany) or, X-vivo 5 (Lonza), that may contain factors necessary for proliferation and viability, including serum (such as e.g., human serum). In an aspect, cells can be maintained under conditions necessary to support growth; for example, an appropriate temperature (e.g., 37° C.) and atmosphere (e.g., air plus 5% CO₂).

Cells can be obtained from any suitable source for the generation of engineered cells. Cells can be primary cells. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumours. Cells can be derived from a healthy donor or from a patient diagnosed with cancer. Cells can also be obtained from a cell therapy bank. Cells can also be obtained from whole blood, apheresis, or a tumour sample of a subject. A cell can be a tumour infiltrating lymphocytes (TIL). In some cases an apheresis can be a leukapheresis.

A desirable cell population can also be selected prior to modification. A selection can include at least one of: magnetic separation, flow cytometric selection, antibiotic selection. The one or more cells can be any blood cells, such as peripheral blood mononuclear cell (PBMC), lymphocytes, monocytes or macrophages. The one or more cells can be any immune cells such as a lymphocyte, an alpha-beta T cell, a gamma-delta T cell, CD4+ T cell, CD8+ T cell, a T effector cell, a lymphocyte, a B cell, an NK cell, an NKT cell, a myeloid cell, a monocyte, a macrophage, or a neutrophil.

### General

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a method, respectively component as defined herein may comprise additional step(s), respectively component(s) than the ones specifically identified, said additional step(s), respectively component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” when used in association with an integer (about 10) preferably means that the value may be the given value of 10 more or less 1 of the value: about 10 preferably means from 9 to 11. The word “about” when used in association with a numerical value (about 10.6) preferably means that the value may be the given value of 10.6 more or less 1% of the value 10.6.

All patent and literature references cited in the present specification are hereby incorporated by reference in their entirety. The following examples are offered for illustrative purposes only and are not intended to limit the scope of the present invention in any way.

### DESCRIPTION OF THE FIGURES

FIG. 1. Illustration of γT-cell receptor chain, δT-cell receptor chain, and γδTCR library construction with an one step cloning strategy using Gibson assembly. In each case, four DNA fragments were assembled in a single reaction to generate a libraries with modified CDR3 regions. The two constant DNA fragments were: a lentiviral plasmid backbone containing the constant C-terminal coding region of the δTCR; and a small DNA fragment encoding the constant gamma TCR sequence followed by a T2A sequence. The two variable DNA fragments contained: the complete variable gamma TCR region including the reference sequence, or variants comprising modifications in the sequence of the gamma CDR3 and a constant TCR gamma overhang; the complete variable delta TCR region including the reference sequence or variants in the sequence of the delta CDR3 and a constant TCR delta overhang. Three randomized libraries were generated; 1) combination of a δT-cell receptor chain comprising a reference δCDR3 region from clone E57 (SEQ ID NO: 2, “D3”) with 12 variant γT-cell receptor chains comprising modifications in the γCDR3 sequences compared to a reference γCDR3 region from clone E57, (variants: SEQ ID NO: 7, 8, 9, 10, 11, 12, 13, 14, 15, 16, 17, 18, 19) (γ^{random}δ^{E57}); 2) combination of a γT-cell receptor chain comprising a reference γCDR3 region from clone E57 (SEQ ID NO: 1, “D3”) with 10 variant δT-cell receptor chains comprising modifications in the δCDR3 sequences compared to a reference δCDR3 region from clone E57 (variants: SEQ ID NO: 19, 20, 21, 22, 23, 24, 25, 26, 27, 110) (γ^{E57}δ^{random}); 3) combinations of the γT-cell receptor chains and the δT-cells receptor chains of the first and second libraries (including with the reference sequences, (γ^{random}δ^{random}). Plasmids were assembled using Gibson assembly and the complete random plasmid pool containing the γδTCRs with variant CDR3 regions were transformed in DH5α E. coli bacteria. Random LV-libraries were prepared from the random plasmid libraries, with each lentivirus containing two random ssRNA TCR copies per virion. Subsequently, αβT-cells were engineered (TEGs) with the random LV-libraries.

FIG. 2A-2D. Selection procedure of reactive γδTCR TEGs towards Luc-Tom HT-29 cells. TEGs expressing γδTCRs from three randomized γδTCR libraries (γ^{random}δ^{E57} TCR, γ^{E57}δr^{random} TCR, γ^{random}δr^{random} TCR) were incubated with Luc-Tom HT-29 cells (recognized by E57) during a 16 hours co-culture. Untransduced T-cells and TEGs expressing the reference E57 γδTCR (SEQ ID NO: 4, 6) were used as controls. TEGs were then stained for CD69 (CD69-APC) and CD107a (CD107a-BV421) expression, and analyzed by FACS flow cytometry. As non-specific TCR activation control, 1000× diluted TransAct was used and effector only was used as background control. CD69⁻CD107a⁻, CD69⁺CD107a⁻, CD69⁺CD107a⁺, populations (squares) were sorted. A representative density plot panel from 3 independent experiments is shown; FIG. 2A: γ^{random}δ^{E57} TCR library (γ-library-E57); FIG. 2B: γ^{E57}δ^{random} TCR library (δ-library-E57), FIG. 2C: γ^{random}δ^{random} TCR library (γδ-library); FIG. 2D: TEGs expressing reference E57 γδTCR.

FIG. 3A-3B. Amplification and barcoding of the lentiviral introduced γδTCR region from bulk sorted cells. DNA from sorted cell populations were extracted and the γδTCR region was PCR-amplified using a 5′ primer (SEQ ID NO: 41) containing a sequence overhang and 3′ primers (SEQ ID NO: 42-56) containing a sequence overhang and barcode (FIG. 3A). Bar-coded PCR fragments were pooled based on yield and specific adaptors for illumina sequencing were added to the PCR fragments in a second PCR reaction (FIG. 3B). PCR fragments were sequenced using illumina NGS sequencing.

FIG. 4A-4B. Variant CDR3 region distribution in random TEG libraries. The number of sequence reads of variant CDR3 regions per million reads was determined for the γ^{random}δ^{E57} TCR (FIG. 4A) and γ^{E57}δr^{random} TCR libraries (FIG. 4B). Data is shown in bars. The corresponding reference γCDR3 positions (WDGFYYK, SEQ ID NO: 79) and δCDR3 positions (IRGYTG, SEQ ID NO: 94) are shown.

FIG. 5. Distribution amongst CDR3 variants from the γ^{random}δr^{random} TCR TEG library (which includes the reference γCDR3 and δCDR3 regions; SEQ ID NO: 79, 94). The number of reads normalized per million reads is shown. Paired γδTCR reads where 1 nucleotide mismatch was observed in either the γ- or δ-TCR were deleted. Data is shown as table format.

FIG. 6A-6B. Relative enrichment of CDR3 variants in the CD69⁺/CD107a⁺ over the CD69⁻/CD107a⁻ population as sorted by FACS flow cytometry. The enrichment of each CDR3 variant for the γ^{random}δ^{E57} TCR and γ^{E57}δ^{random} TCR libraries was determined as a percentage from two independent co-cultures. In the case of the γ^{random}δ^{E57} library (FIG. 6A) the γ^{G5}δ^{D3} (G5/D3; SEQ ID NOs: 10, 2), γ^{G10}δ^{D3} (G10/D3; SEQ ID NOs: 15, 2) TCR CDR3 variants were enriched in the CD69⁺/CD107a⁺ population, whilst the CDR3 variants γ^{G2}δ^{D3} (G2/D3; SEQ ID NOs: 7, 2) and γ^{G3}δ^{D3} (G3/D3; SEQ ID NOs: 8, 2) were negatively correlated with the upregulation of these markers. In the case of the γ^{E57}δ^{random} library (FIG. 6B) the γ^{G1}δ^{D4} (G1/D4; SEQ ID NOs: 1, 21), γ^{G1}δ^{D5} (G1/D5; SEQ ID NOs: 1, 22), γ^{G1}δ^{D6} (G1/D6; SEQ ID NOs: 1, 23), γ^{G1}δ^{D9} (G1/D9; SEQ ID NOs: 1, 110) CDR3 variants were enriched in the CD69⁺/CD107a⁺ population, whilst the CDR3 variants γ^{G1}δ^{D1} (G1/D1; SEQ ID NOs: 1, 19), γ^{G1}δ^{D2}(G1/D2; SEQ ID NOs: 1, 20) were negatively correlated with the upregulation of these markers.

FIG. 7A-7C. The cytotoxicity profile of TEGs expressing γδTCRs with CDR3 region variants correlate with negative or positive enrichment obtained from the selection screening. Luc-Tom HT-29 cells were co-cultured for 72 hours with TEGs expressing γδTCRs comprising the E57 reference CDR3 regions (G1/D3; SEQ ID NOs: 1, 2), G2/D3 (SEQ ID NOs: 7, 2), G8/D3 (SEQ ID NOs: 13, 2) CDR3 variants (FIG. 7A), with TEGs expressing γδTCRs comprising the E57 reference CDR3 regions (G1/D3; SEQ ID NOs: 1, 2), G5/D3 (SEQ ID NOs: 10, 2), or G10/D3 (SEQ ID NOs: 15, 2) CDR3 variants (FIG. 7B), or with TEGs expressing γδTCRs comprising the E57 reference CDR3 regions (G1/D3; SEQ ID NOs: 1, 2), G1/D1 (SEQ ID NOs: 1, 19), G1/D2 (SEQ ID NOs: 1, 20), G1/D4 (SEQ ID NOs: 1, 21), G1/D5 (SEQ ID NOs: 1, 22), or G1/D6 (SEQ ID NOs: 1, 23) CDR3 variants (FIG. 7C), or untransduced matched αβT-cells (untransduced), at effector to target (E:T) ratios of 2:1, 1:1, 1:2, 1:4 and 1:8. Cytotoxicity was measured by decreased Luciferase activity relative to target cells cultured alone. Data is plotted as percentage of cytolysis relative to maximum cytolysis induced by treatment of the target cells with the detergent Triton-X-100. Bars represent mean±SD of triplicates in a single experiment. Statistical differences is only shown for the 1:4 E:T ratio. n.s: not significant; *P<0.05; **P<0.01; ***P<0.001 ****P<0.0001, multiple t-test.

FIG. 8A-8B. Pairing single chain γ-, or δ-CDR3 variants with improved cytotoxicity profile further augments the cytotoxicity profile towards target cells. TEGs expressing γδTCRs comprising the E57 reference CDR3 regions (G1/D3; SEQ ID NOs: 1, 2) or the G1/D5 (SEQ ID NOs: 1, 22), G1/D6 (SEQ ID NOs: 1, 23), G5/D3 (SEQ ID NOs: 10, 2), G10/D3 (SEQ ID NOs: 15, 2), G5/D6 (SEQ ID NOs: 10, 23), G10/D6 (SEQ ID NOs: 15, 23) CDR3 variants were co-cultured with Luc-Tom HT-29 cells for 72 hours with an E:T ratio of 1:1, 1:2, 1:4 and 1:8. Untransduced matched αβT-cells (untransduced) were used as control. Luciferase activity relative to target cells cultured alone was determined and plotted as % of cytotoxicity (FIG. 8A). IFNγ levels (FIG. 8B) were measured by ELISA. Bars represent mean±SD of triplicates in a single experiment. Statistical differences for cytolyses is only shown for the 1:4 E:T ratio. Statistical differences for IFN release is only shown for the 1:1 E:T ratio. n.s: not significant; *P<0.05; **P<0.01; ***P<0.001, ****P<0.0001 multiple t-test.

FIG. 9. Relative enrichment of CD69⁺/CD107a⁺ over the CD69⁻/CD107a⁻ TEG population expressing γδTCR-CDR3 variants from the γ^{random}δr^{random} TCR library. The enrichment of each CDR3 variant for the γ^{random}δ^{random} TCR library was determined as a percentage. Paired CDR3 mutants with an enrichment above 170% are highlighted.

FIG. 10. Correlation between the percentage enrichment for CD69⁺/CD107a⁺ TEGs expressing γδTCRs from the paired γ^{random}δ^{random} CDR3 library and tested cytotoxicity. Relative enriched γ- or δ-, or γδ-CDR3 variants with decreased or increased cytotoxicity in a TEG format towards target cells versus the reference γδ-E57-TCR (G1/D3; SEQ ID NOs: 1, 2) at an E:T ratio of 1:4 are shown in a correlation plot as percentage.

FIG. 11A-11B. TEGs expressing a γδTCR comprising the G11/D5 CDR3 variant region (SEQ ID NOs: 16, 22) or the G12/D6 CDR3 variant region (SEQ ID NOs: 17, 23) showed enhanced cytotoxicity compared to TEGs expressing the reference γδTCR from E57 (G1/D3; SEQ ID NOs: 1, 2). Luc-Tom HT-29 cells were co-cultured with TEGs for 72 h at an E:T ratio of 1:1, 1:2, 1:4, or 1:8 and cytolysis was measured with luciferase assay (FIG. 11A). IFNγ release was measured with ELISA (FIG. 11B). **P<0.01, ****P<0.0001 multiple t-test.

FIG. 12. More persistent cytolyses by TEGs expressing γδTCR comprising the G5/D6 CDR3 variant regions versus the reference E57 γδTCR. Luc-Tom HT-29 cells were co-cultured with TEGs expressing either the G5/D6 γδTCR-CDR3 region variant (SEQ ID NOs: 10, 23) or reference γδTCR from E57 (G1/D3; SEQ ID NOs: 1, 2). The weekly serial cytolyses profile, depicted as percentage, of TEGs from two donors at a E:T ratio of 1:1 was monitored, for up to 9 stimulations.

FIG. 13A-13L. TEGs expressing the γδTCR comprising the G5/D6 CDR3 variant regions (SEQ ID NOs: 10, 23) showed improved reactivity towards tumour CRC cell lines expressing the target antigen compared to TEGs expressing the reference γδTCR from E57 (G1/D3; SEQ ID NOs: 1, 2). Twelve tumour cell lines (FIG. 13A: HT29; FIG. 13B: RKO; FIG. 13C: T84; FIG. 13D: LS174T; FIG. 13E: SW480; FIG. 13F: KM12; FIG. 13G: LS180; FIG. 13H: HT55; FIG. 13I: MDST-8; FIG. 13J: MDA-MB-231; FIG. 13K: HT-29 with target knocked out; FIG. 13L: OUMS23) were co-cultured for 72 hours with TEGs or untransduced donor matched αβT-cells (negative control), at effector to target (E:T) ratio of 1:1. Cytotoxicity was measured by xCELLigence and plotted as percentage of cytolysis relative to maximum cytolysis induced by treatment of the target cells with the detergent Triton-X-100. Bars represent mean±SD of triplicates in a single experiment. Graphs describe one representative TEG batch.

FIG. 14A-14M. TEGs expressing the γδTCR comprising the G5/D6 CDR3 variant regions (SEQ ID NOs: 10, 23) showed improved IFNγ release compared to TEGs expressing the reference γδTCR from E57 (G1/D3; SEQ ID NOs: 1, 2). Twelve tumour cell lines (FIG. 14A: HT29; FIG. 14B: RKO; FIG. 14C: T84; FIG. 14D: LS174T; FIG. 14E: SW480; FIG. 14F: KM12; FIG. 14G: LS180; FIG. 14H: HT55; FIG. 14I: MDST-8; FIG. 14J: MDA-MB-231; FIG. 14K: HT-29 with target knocked out; FIG. 14L: OUMS23, FIG. 14M: TEGs without target cells) were co-cultured for 72 hours with TEGs or untransduced donor matched αβT-cells (negative control), at effector to target (E:T) ratio of 1:1. The levels of IFN-γ released into the supernatants were measured by ELISA. Bars represent mean±SD of triplicates in a single experiment. Graphs describe one representative TEG batch

FIG. 15A-15B. Soluble γδTCR comprising the G5/D6 variant CDR3 regions (SEQ ID NO: 10, 23) with or without the original cysteine bond in the connecting peptide region bind HT-29 expressing the recognised target (EPCR) specifically. Different concentrations of sE57 G5/D6 (SEQ ID NOs: 220, 234) and sE57cys G5/D6 (SEQ ID NOs: 244, 258) were added to HT-29 cells knocked out (KO) for target antigen expression or with enhanced target antigen expression, and binding was determined by FACS flow cytometry. Target cells mock treated with PBS served as control binding (FIG. 15A). In FIG. 15B, the binding of 10 μg sE57 G1/D3 (SEQ ID NOs: 216, 231), sE57 G2/D1 (SEQ ID NOs: 217, 229), or sE57 G5/D6 (SEQ ID NOs: 220, 234) to HT-29 (wild-type), HT-29 with enhanced expression, or HT-29 KO for recognised target was determined. Mock treated and unstained target cells served as controls. Data was analysed with flowjo and shown as histograms.

FIG. 16A-16D. Soluble γδTCR-CD3 bispecific engagers comprising the G5/D6 CDR3 variant regions induce potent cytotoxicity towards HT-29 tumour cells. Soluble γδTCR-CD3 bispecific engagers (5 μg) comprising the reference E57 CDR3 regions (G1/D3; SEQ ID NOs: 288, 303), the G5/D6 CDR3 variant regions (SEQ ID NOs: 292, 306), or the G2/D1 variant regions (SEQ ID NOs: 289, 301) were added to HT-29 cells (wild-type), HT-29 cells with enhanced EPCR expression, or KO for the recognised target and binding was determined by FACS flow cytometry (FIG. 16A). PBS-mock treated (secondary only) or unstained target cells served as controls. Data was analysed with flowjo and shown as histograms.

In FIG. 16B-16D, serially diluted soluble γδTCR-CD3 bispecific engagers with indicated concentrations were added to Luc-Tom-HT-29 (FIG. 16B), HT-29 KO (FIG. 16C), and HT-29 cells with ectopic expression (FIG. 16D) for the recognised target antigen (EPCR) and co-incubated with αβT cells. Cytolyses was determined after 72 hours and data was plotted as measured relative light units (RLU).

FIG. 17A-17B. TEGs expressing a γδTCR comprising the G5/D6 CDR3 variant regions (SEQ ID NOs: 10, 23) showed enhanced cytotoxicity compared to TEGs expressing the reference γδTCR from E57 (G1/D3; SEQ ID NOs: 1, 2) or TEGs expressing a γδTCR comprising the CDR3 region combinations of SEQ ID NO: 379/SEQ ID NO: 2 or SEQ ID NO: 1/SEQ ID NO: 380. Luc-Tom HT-29 cells (FIG. 15A) were co-cultured with TEGs at an E:T ratio of 1:2, 1:4, 1:8, or 1:16, and Luc-Tom RKO cells (FIG. 15B) were co-cultured with TEGs at an E:T ratio of 1:1, 1:2, 1:4, or 1:8. The co-culture lasted 72 hours, and cytolysis was measured using luciferase assay. Bars represent mean±SD of triplicates in a single experiment. Graphs describe one representative TEG batch.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

Unless specified, reagents employed in the examples are commercially available or can be prepared using commercially available instrumentation, methods, or reagents known in the art. The examples illustrate various aspects of the invention and practice of the methods of the invention. The examples are not intended to provide an exhaustive description of the many different embodiments of the invention. Thus, although the invention has been described in some detail by way of illustration and example for purposes of clarity of understanding, those of ordinary skill in the art will realize readily that many changes and modifications can be made thereto without departing from the spirit or scope of the appended claims.

### Material & Methods Pertaining to the Examples

### γδTCR DNA Subcloning and Lentivirus Preparation

Single human codon optimized DNA fragments encoding respectively the variable γ- or δ-chain region with modifications in the CDR3 region compared to a reference sequence and with an overlapping part in the constant region were designed. The γCDR3 (SEQ ID NO: 1, “G1”) and δCDR3 (SEQ ID NO: 2, “D3”) regions of clone E57 were used as reference sequences. In total, 12 γ-CDR3 (SEQ ID NOs: 7-18, “G2-G13”), and 10 δ-CDR3 (SEQ ID NO: 19-27, 110; “D1, D2, D4-D11”) variants, including the reference CDR3 regions were synthesized by IDT (Integrated DNA Technologies, IA, USA). The synthesized DNA fragments were assembled into the pLenti 6.3 lentiviral bicistronic vector (SEQ ID NO: 109) via Gibson assembly together with a DNA fragment encoding the constant γ-region followed by a T2A self-cleaving peptide (SEQ ID NO: 364) to create a random plasmid pool encoding γδTCRs with variant CDR3 regions. The assembled plasmid pool was transformed into DH5α E. coli bacteria and bacteria were grown in the presence of carbenicillin. Plasmid DNA from bacteria was extracted with NucleoBond xtra Midi kit (Macherey-Nagel, PA, USA) according to manufacturer instructions. Transcription of the obtained bicistronic expression was driven by an MSCV promoter (SEQ ID NO: 108). Viral genome packaging and transgene expression enhancement are achieved by LTR/Y and WPRE regulatory elements, respectively. Lentiviral particles were produced using the LV-Max system from Thermo Fisher Scientific (MA, USA). LV-MAX producer cells (A35827) were transfected with pLenti 6.3 γδTCR transfer construct and packaging mix (pLP1, pLP2, pLP-VSVG). Lentiviral titers were assessed in αβTCR-deficient Jurkat-76 cells by flow cytometry analysis, measuring the percentage of CD3/γδTCR among live cells.

sTCR Construction

For generation of the tested soluble γδTCRs, codon optimized DNA sequences encoding the reference E57 and affinity enhanced sequences without or with a cysteine bridge were utilized. Two soluble reference TCRs with or without a cysteine were generated by combining the sE57 gamma chain (G1) (without or with a cysteine) (SEQ ID NOs: 216, 240 respectively) with the sE57 delta chain (D3) (without or with a cysteine) (SEQ ID NOs: 231, 255 respectively). An affinity enhanced soluble TCR was generated by combining the sE57 G5 (without or with cysteine) variant (SEQ ID NOs: 220, 244 respectively) with the sE57 D6 (without or with a cysteine) variant (SEQ ID NOs: 234, 258 respectively). An affinity decreased soluble TCR was generated by combining the sE57 G2 (without or with cysteine) variant (SEQ ID NOs: 217, 241 respectively) with the sE57 D1 (with or without a cysteine) variant (SEQ ID NOs: 229, 253 respectively). All tested γ-subunits contained a part of the TRGC1 region (Cγ1 constant region) and a c-terminal c-Fos-dimerization motif (SEQ ID NO: 363). All tested δ-subunits contained a c-Jun-dimerization motif (SEQ ID NO: 362) followed by an Avi-tag (SEQ ID NO: 365) and His-tag (suitable for purification purposes; SEQ ID NO: 366). The nucleotide sequences encoding the above were subcloned into pHCAG-L2EOP vector (SEQ ID NO: 113) by gBlock gene assembly (Addgene, MA, USA).

### γδTCR-CD3 Bispecific Engager Construction

For generation of the tested soluble γδTCR-CD3 bispecific engagers, codon optimized DNA sequences encoding soluble γT-cell receptor chains of the reference E57 (SEQ ID NO: 288, “G1”) or variant sequences (SEQ ID NO: 292, “G5”; SEQ ID NO: 289, “G2”) were paired with the δT-cell receptor chain of the reference E57 (SEQ ID NO: 303, “D3”) or variant sequences (SEQ ID NO: 306, “D6”; SEQ ID NO: 301, “D1”). Each γT-cell receptor chain-encoding sequence was paired with the a δT-cell receptor-chain-encoding sequence. The γT-cell receptor chains were connected to an anti-CD3 scFv derived from OKT3 antibody clone (SEQ ID NO: 105) via a linker (SEQ ID NO: 78), followed by a second linker (Gly-Ser-Gly), an Avi-tag (suitable for biotinylation purposes; SEQ ID NO: 365) and His-tag (suitable for purification purposes; SEQ ID NO: 367). The nucleotide sequences encoding the above were subcloned into pHCAG-L2EOP vector (SEQ ID NO: 113) by gBlock gene assembly (Addgene, MA, USA).

Production of sTCRs and γδTCR-CD3 Bispecific Engagers

Soluble γδTCRs and γδTCR-CD3 bispecific engagers were produced by co-transfecting the γ-chain and δ-chain encoding plasmids into HEK293F cells in the presence or absence of the BirA plasmid (SEQ ID NO: 368) with a 1:1:8 ratio, using the transfection reagent PEImax (Polysciences Inc., PA, USA) at PEImax 1 mg/ml:DNA 1 μg/μl ratio of 3:1 v/v. The transfection was carried out using Optimem solution (Thermo Fisher Scientific) and in the presence of Biotin (final concentration 25 μg/ml, Thermo Fisher Scientific). 7 days post-transfection, supernatants were collected and soluble γδTCRs or γδTCR-CD3 bispecific engagers were purified via His-Tag using Histrap columns (Sigma-Aldrich, MO, USA) following the manufacturer's protocol. Purity was assessed by SDS-PAGE gel (in denaturating/non denaturating conditions) and Coomassie staining.

Staining (Binding) of HT-29 Target Cells with sTCR or TCR-CD3 Bispecific Engagers

Trypsinized HT-29 target cells seeded in 96-well round bottom-plates were incubated for 2 hours at 37° C. to recover cell surface expression molecules. Cells were co-incubated with 0.155 μg, 0.625 μg, 2.5 μg, or 10 μg of sTCR, or 5 μg TCR-CD3 bispecific engager for 1 hour at 4° C. Unbound sTCR/TCR-CD3 bispecific engager was washed. Bound sTCR or TCR-CD3 bispecific engager was stained respectively with streptavidin-labelled A647 (BioLegend, CA, USA) or anti-His-APC (BioLegend). Staining was determined with FACS flow cytometry was results were analyzed with Flowjo software.

### TEG Production

TEGs were manufactured starting from αβT-cells enriched by MACS separation (Miltenyi Biotec, Bergisch Gladbach, Germany) from healthy donor leukapheresis material, according to manufacturer instructions. Purified αβT-cells were cultured in TEXMACS medium supplemented with 2.5% human serum (Sanquin, Amsterdam, NL), rhIL-7 (20-2000 IU/mL) and rh IL15 (20-200 IU/mL) (both from Miltenyi Biotec), and 1% Penicillin/Streptomycin, and activated using TransAct (Miltenyi Biotec) per manufacturer's recommendations. Cells were transduced with γδTCR LV particles (MOI 3) and then expanded for 12 days in TEXMACS medium, 2.5% human serum, rhIL-7 (20-2000 IU/mL) and rh IL15 (20-200 IU/mL), 1% Penicillin/Streptomycin. At the end of the production, transduction efficiency (% γδTCR, >40% in all cases), T-cell purity (>90% in all cases), and relative expression of T-cell markers CD4 and CD8 were measured by flow cytometry. Cells were then cryopreserved in 1 volume of NaCl 0.9%/5% human serum albumin and 1 volume of Cryostor CS10 (Sigma-Aldrich).

xCELLigence Cytotoxicity Assay

TEG anti-tumour activity towards several tumour cell lines was evaluated in vitro by measuring the killing of tumour target cells in a xCELLigence co-culture assay (Agilent, CA, USA). First, cell lines were harvested, counted and seeded to the appropriate number of cells per well in triplicate in 96 well E-plates, and then placed in the xCELLigence cradles. Target cell adhesion and proliferation was measured for 24 hours. TEG or negative control untransduced αβT-cells were then harvested, counted, resuspended in IMDM medium, 5% human serum, and 1% Penicillin/Streptomycin, and added to the tumour target cells at Effector/Target ratio of 1:1. Loss of target cell adherence, as a readout for cytotoxicity, was measured for 72 hours. Cytotoxicity was calculated as percentage of cytolysis relative to maximum cytolysis induced by treatment of the target cells with the detergent Triton-X-100. Supernatant depleted from TEGs by centrifugation force was used for IFN-γ ELISA.

### Luciferase-Based (Serial) Cytotoxicity Assay

Luc-Tom HT-29 or Luc-Tom RKO tumour cells were harvested, counted and seeded to the appropriate number of cells per well, in triplicate in 96-well E-plates, and cultured in McCoy's 5a Medium (Luc-Tom HT-29) or EMEM (Luc-Tom RKO), 10% fetal bovine serum and 1% Penicillin/Streptomycin (ThermoFisher Scientific) for 24 hours at 37° C.

TEGs expressing a γδT-cells receptor comprising the E57 reference CDR3 region sequences (G1/D3, SEQ ID NO: 1, 2), or comprising the CDR3 region sequences SEQ ID NO: 379 (paired with SEQ ID NO: 2) or SEQ ID NO: 380 (paired with SEQ ID NO: 1), or variant sequences such as G5/D6 (SEQ ID NO: 10, 23) were then harvested, counted, resuspended in IMDM medium, 5% human serum, and 1% Penicillin/Streptomycin, and added to the tumour target cells at E:T ratio of 2:1, 1:1, 1:2, 1:4, 1:8, or 1:16. The resulting co-culture was maintained at 37° C. for 72 hour or one week (for serial cytotoxicity assays). At the end of the co-culture, TEGs were harvested and transferred to a new cell culture plate containing fresh Luc-Tom HT-29 or Luc-Tom RKO tumour cells, in cases where a serial cytotoxicity assay was performed, for a new round of target exposure/stimulation (weekly). Otherwise, supernatant depleted from TEGs by centrifugation force was used for IFN-γ ELISA. Luciferase activity of Luc-Tom HT-29 or Luc-Tom RKO tumour cells from the co-culture plate was determined by the addition of D-luciferine substrate (ThermoFisher Scientific) and reading the luminescence in endpoint mode using Glomax luminometer according to the manufacturer's instructions (Promega, Madison, WI, USA). Cytolysis/cytotoxicity was calculated using the following formula: 100×[1−(Luminescence from target cells in co-culture with effector T-cells/Luminescence from target cells cultured alone)]. In cases where multiple stimulation rounds were employed, the co-culture assay was repeated for up to 9 consecutive stimulation rounds.

### IFN-γ ELISA Assay

Cell culture supernatants from xCELLigence or Luciferase-based cytotoxicity assays were harvested at the end of the co-culture to measure IFN-γ secretion using a commercial Human IFN-gamma DuoSet ELISA assay (cat nr. DY2858-05, R&D Systems, Minneapolis, MN, US), according to manufacturer's instructions. This is a standard sandwich ELISA using a plate-bound capture antibody and a detection antibody both specific for IFN-γ. The detection antibody is linked to an enzyme which can convert a substrate into an absorbance signal which is measured with a plate reader. The internal standard curve allows absorbance values to be calculated into the IFN-γ concentration (pg/mL) released into the supernatants.

### γδTCR-CD3 Bispecific Engager Cytolyses

Luc-Tom HT-29 tumour cells were seeded for 24 hours at 37° C. and incubated for 1 hour with 10 μg of serially diluted TCR-CD3 bispecific engagers concentrations. αβT cells were added to the co-culture with an E:T ratio of 1:1 for 72 hours and cytotoxicity was determined according to the luciferase-based cytotoxicity assay as described above.

### Screening of Random CDR3 TCR Libraries

Luc-Tom HT-29 tumour cells were seeded for 24 hours at 37° C. and incubated for 16 hours with CDR3 TEG libraries with an E:T of 1:3. TEGs were harvested and stained for CD69-APC (Clone REA824, Miltenyi Biotec) and CD107a-BV421 (H4A3, Biolegend). Populations marked for CD69+/CD107a+, CD69+/CD107a−, CD69−/CD107a− were sorted by FACS flow cytometry using the BD FACSMelody™ sorter.

### Example 1

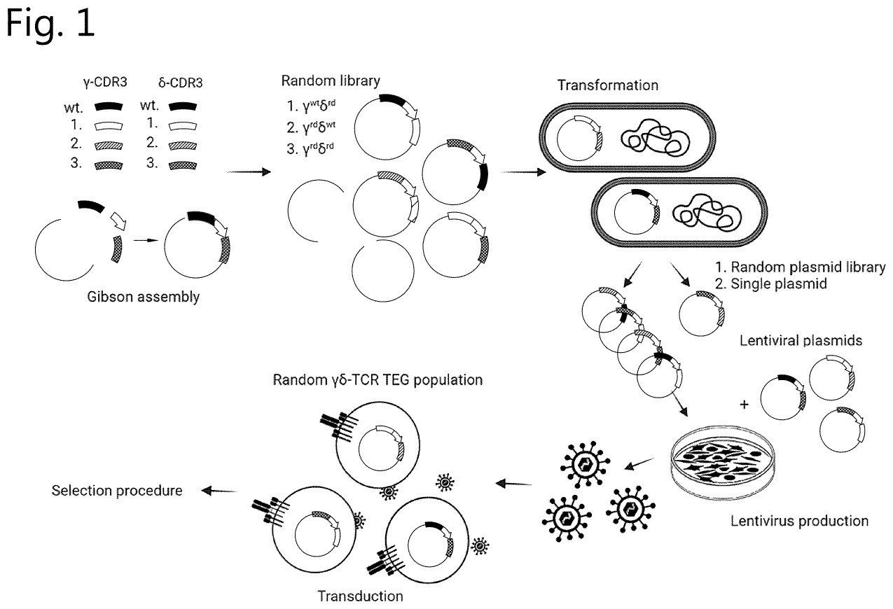

In this Example, three bicistronic lentiviral vector γδTCR libraries, and TEG libraries expressing the γδTCRs, were constructed according to the procedure described in the Materials and Methods. An illustration of γδTCR library construction is given in FIG. 1.

The vectors of the first library (γ^{random}δ^{E57}) encoded a δT-cell receptor chain comprising a reference δCDR3 region from clone E57 (SEQ ID NO: 2, “D3”) in combination with 12 variant γT-cell receptor chains comprising modifications in the γCDR3 region sequences compared to the reference sequence SEQ ID NO: 1 (SEQ ID NOs: 7 (“G2”), 8 (“G3”), 9 (“G4”), 10 (“G5”), 11 (“G6”), 12 (“G7”), 13 (“G8”), 14 (“G9”), 15 (“G10”), 16 (“G11”), 17 (“G12”), or 18 (“G13”)). For the assembly of the vectors, a 1:1:1:1 stoichiometry between the vector backbone, variant γCDR3 chains, γ-constant-t2A, and reference E57 δCDR3 chains was used. A random lentivirus library was generated from the assembled γ^{random}δ^{E57} plasmid pool and TEGs were produced containing all 12 TCR-encoding variants as described in the Materials and Methods.

The vectors of the second library (γ^{E57}δ^{random}) encoded a γT-cell receptor chain comprising a reference γCDR3 region from clone E57 (SEQ ID NO: 1, “G1”) in combination with 10 variant δT-cell receptor chains comprising modifications in the δCDR3 region sequences compared to the reference sequence SEQ ID NO: 2 (SEQ ID NOs: 19 (“D1”), 20 (“D2”), 21 (“D4”), 22 (“D5”), 23 (“D6”), 24 (“D7”), 25 (“D8”), 110 (“D9”), 26 (“D10”), or 27 (“D11”). For the assembly of the vectors, a 1:1:1:1 stoichiometry between the vector backbone, variant δCDR3 chains, 5-constant-t2A and reference E57 γCDR3 chains was used. A random lentivirus library was generated from the assembled γ^{E57}δ^{random} plasmid pool and TEGs were produced containing all 10 TCR-encoding variants as described in the Materials and Methods.

The vectors of the third library (γ^{random}δ^{random}) encoded combinations of all γCDR3 variant chains and all δCDR3 variant chains, including both the γ and δ reference (E57) CDR3 sequences. For the assembly of the vectors, a 1:1:1:1 stoichiometry between the vector backbone, all γCDR3 chains, γ-constant-t2A and all δCDR3 chains were used. A random lentivirus library was generated from the assembled γ^{random}δ^{random} plasmid pool and TEGs were produced containing all 143 γδTCR-encoding variants as described in the Materials and Methods.

TEGs transduced with the three random γδTCR libraries were co-cultured Luc-Tom HT-29 cells (recognized by E57) at 37° C. for 16 hours as described in the Luciferase based cytotoxicity assay. 1000× diluted TransAct (Miltenyi Biotec) was used as positive control and TEGs cultured in media without Luc-Tom HT-29 cells served as negative control. After 16 hours, TEGs were harvested and stained for CD69 (CD69-APC) and CD107a (CD107a-BV421) expression to determine their degranulation and activation status by flow cytometry. Relative enriched TEGs expressing γδTCRs with an activated and degranulated profile were compared to the non-activated and non-degranulated TEG population. The number of individual reads normalized per million reads of each activated/degranulated sorted population over non-activated/non-degranulated population was determined and was shown as percentages.

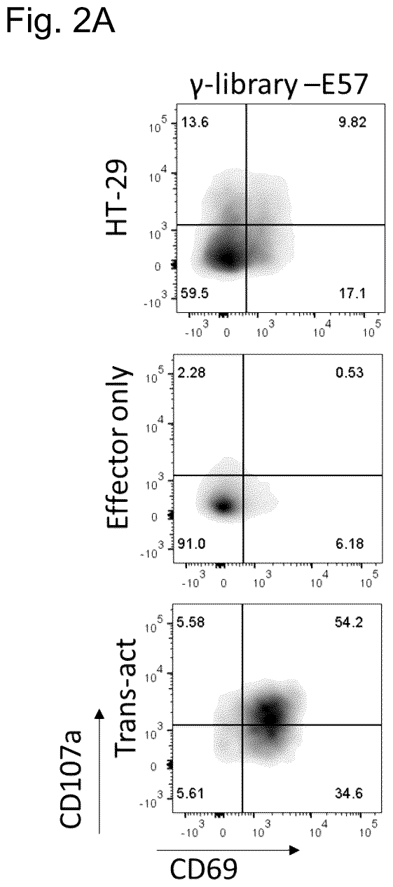

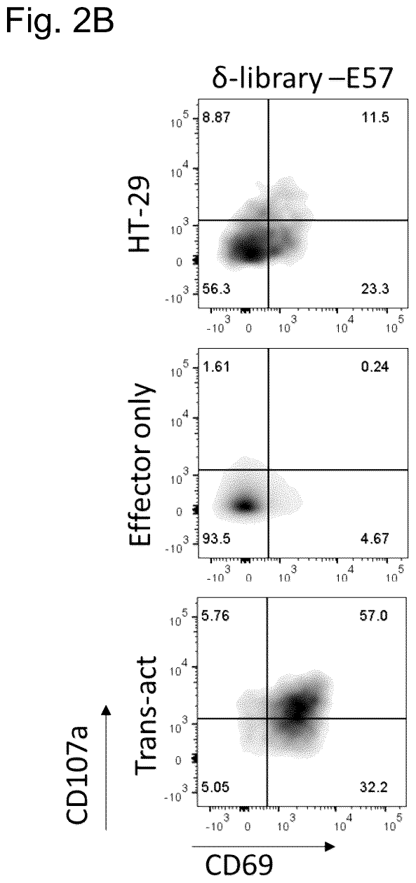

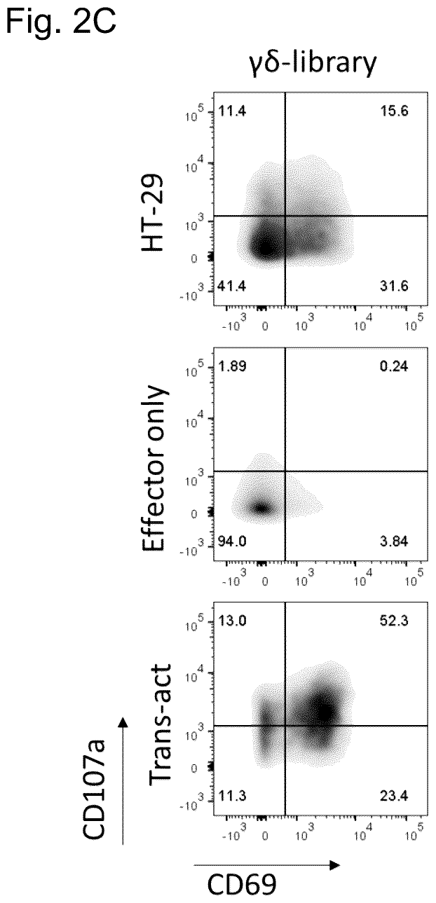

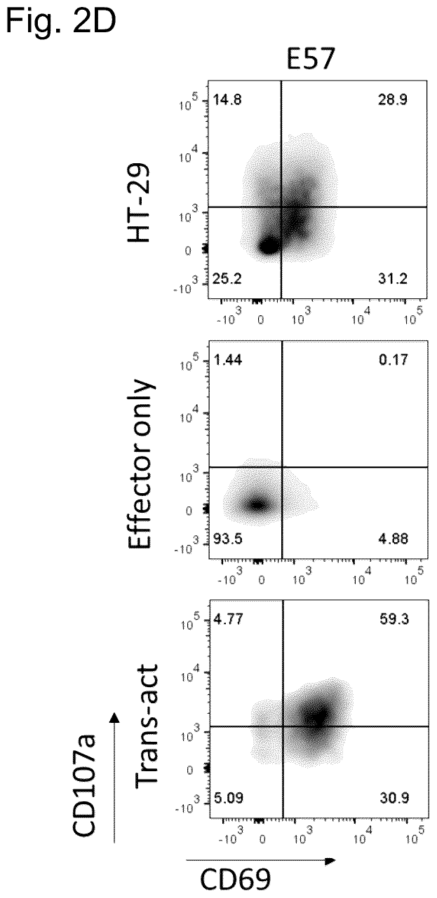

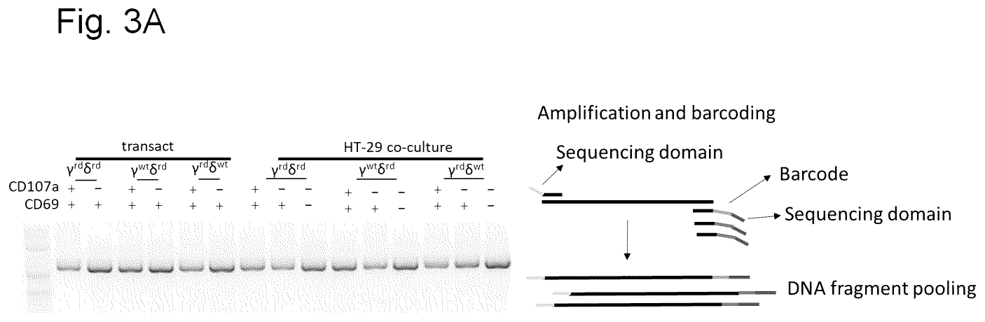

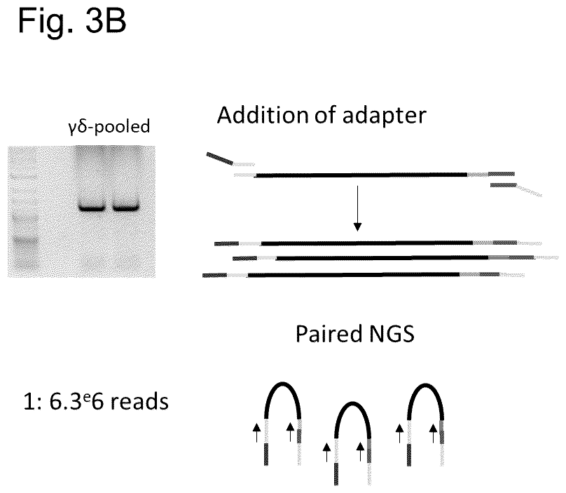

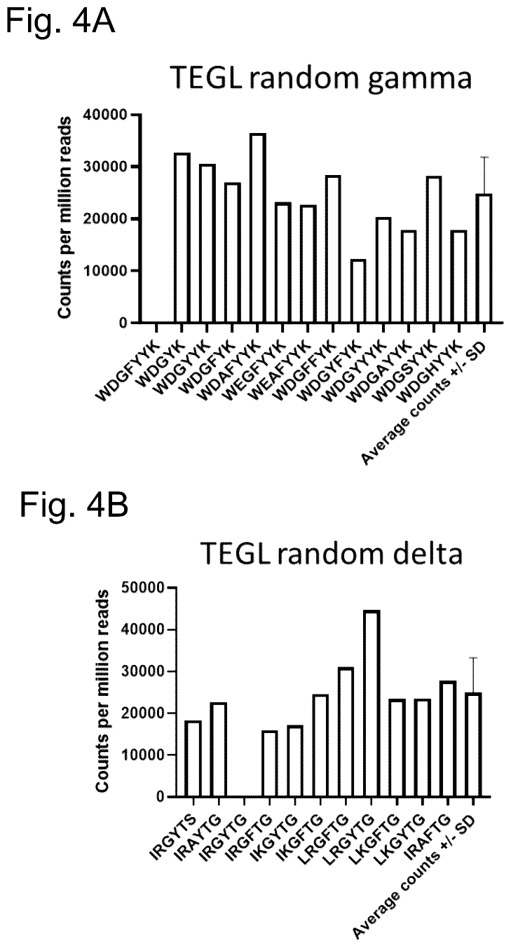

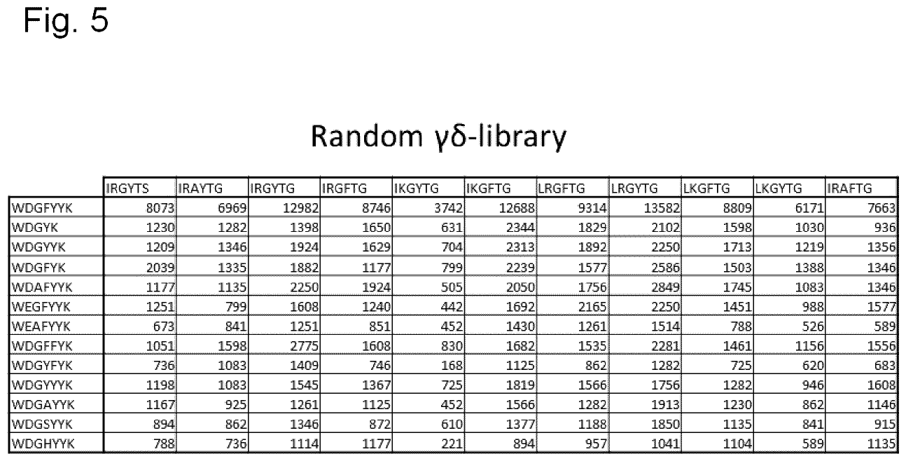

The activation and degranulation status of TEGs transduced with the first (γ^{random}δ^{E57}), second (γ^{E57}δ^{random}) and third (γ^{random}δ^{random}) TCR library is depicted in FIGS. 2A, 2B and 2C respectively. The activation and degranulation status of TEGs expressing the reference γδTCR E57 (G1/D3) is depicted in FIG. 2D. TEGs gated in the CD69⁻CD107a⁻, CD69⁺CD107a⁻ and CD69⁺CD107a⁺ quadrants were sorted in bulk by FACS Melody (BD biosciences) and DNA from sorted TEG populations was extracted. The viral integrated TCR region for each sorted TEG population was PCR amplified with primers (SEQ ID NOs: 41-56) containing a specific barcode and sequence identifier (FIG. 3A). Specific adaptors were attached to the 5′- and 3′-ends of the TCR-amplified fragment by PCR and the sequence of each fragment was determined via illumina NGS (Next Generation Sequencing) (Novogene, Cambridge, UK, FIG. 38). The number of sequences normalized per million reads for each CRD3 γδTCR combination for the complete first, second and third γδTCR TEG library was determined. The stoichiometry of the different CDR3 variant regions were equally present following a normal poisson distribution (FIG. 4A-4B, FIG. 5).

### Example 2

In this example, a screening method was developed on a random γδCDR3 library to determine whether γδTCRs with increased or decreased reactivity towards tumour target cells could be selected based on TCR specific activation followed by assessment of expression of CD69 and CD107a as selection markers.

With this method it is possible to screen for increased or decreased reactivity of a γδTCR with a variation in only the γTCR chain (γ^{random}δ^{reference}TCR) or δTCR chain (γ^{reference}δ^{random}TCR) and synergize the reactivity by combining a desired γTCR chain with a desired δTCR chain. It is also possible to screen for increased or decreased reactivity of a γδTCR with a variation in both the γ- and δ-TCR chains (γ^{random}δ^{random} TCR).

Here, we show application of this method using three random γδTCR libraries comprising variant CDR3 regions: γ^{random}δ^{E57}TCR, γ^{E57}δ^{random}TCR, and γ^{random}δ^{random}TCR, obtained as shown in Example 1.

TEGs expressing γδTCRs from each γδTCR CDR3 library were co-cultured for 16 hours with HT-29 tumour target cells and TEG populations marked for CD69⁻CD107a⁻, CD69⁺CD107a⁻ or CD69⁺CD107a⁺ were sorted in bulk. The number sequence reads per million reads for each CDR3 variant was determined for the sorted populations as described in Example 1. The normalized total reads corresponding to each CDR3 variant in the CD69⁺/CD107a⁺ population was divided by the number of reads corresponding to the same CDR3 variant in the CD69⁻/CD107a⁻ population and the data was shown as percentage. With this selection procedure it is possible to determine the reactivity of a specific TCR chain within a pool variant TCR chains against any tumour target cell within a certain time frame.

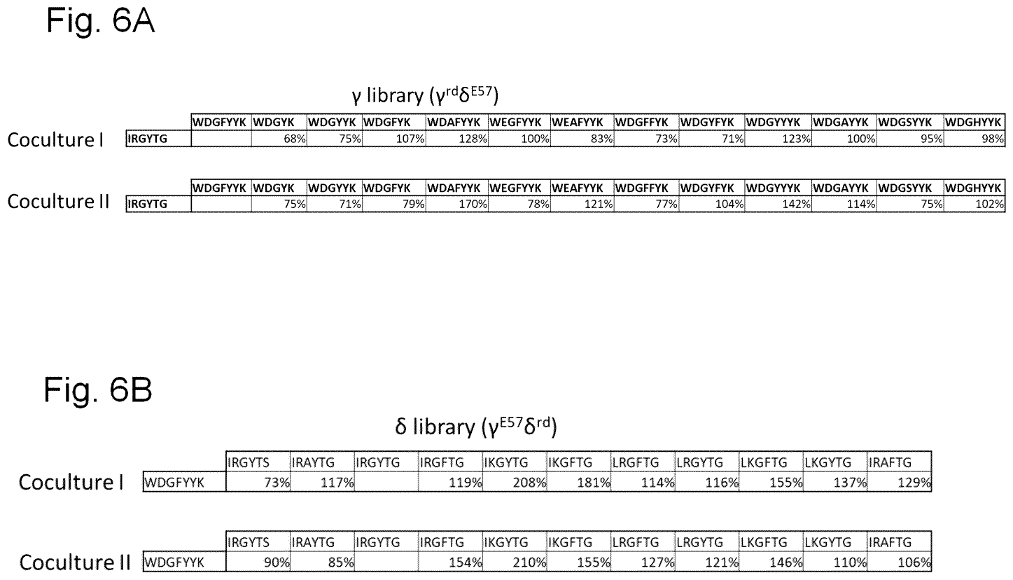

For the γ^{random}δ^{E57}TCR library (FIG. 6A), TEGs expressing γδTCRs comprising the G5/D3 (SEQ ID NO: 10, 2), or G10/D3 (SEQ ID NO: 15, 2) CDR3 region variants were enriched for the activation and degranulation marker CD69 and CD107a respectively, whilst the TEGs expressing γδTCRs comprising the G2/D3 (SEQ ID NO: 7, 2), or G3/D3 (SEQ ID NO: 8, 2) variants were negatively correlated with these markers.

For the γ^{E57}δ^{random}TCR library (FIG. 6B), TEGs expressing γδTCRs comprising the G1/D4 (SEQ ID NOs: 1, 21), G1/D5 (SEQ ID NO: 1, 22), G1/D6 (SEQ NO: 1, 23), or G1/D9 (SEQ ID NOs: 1, 110) CDR3 region variants were enriched for CD69 and CD107a, whilst TEGs expressing γδTCRs comprising the G1/D1 (SEQ ID NO: 1, 19) or G1/D2 (SEQ ID NO: 1, 20) variants showed an inverse enrichment for these markers.

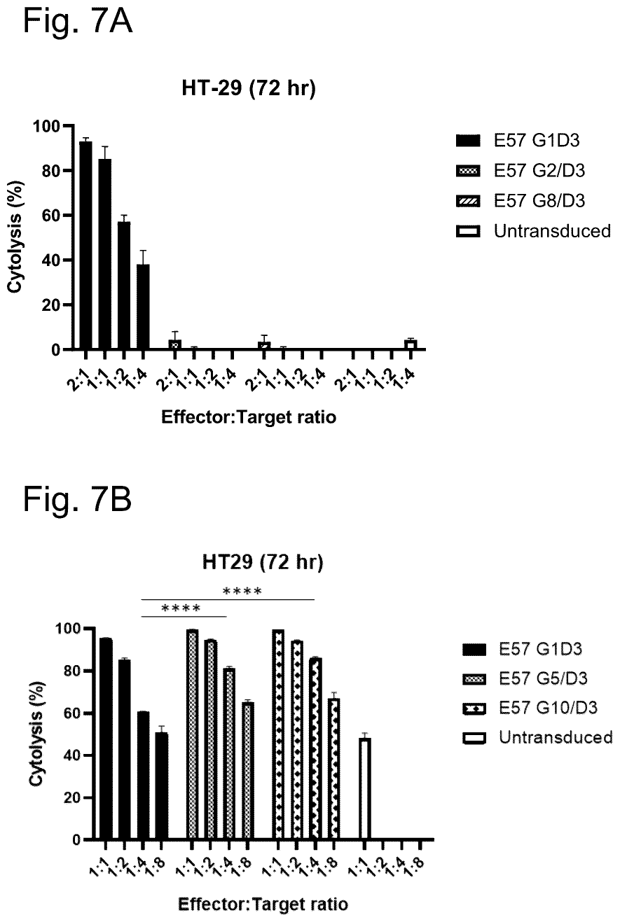

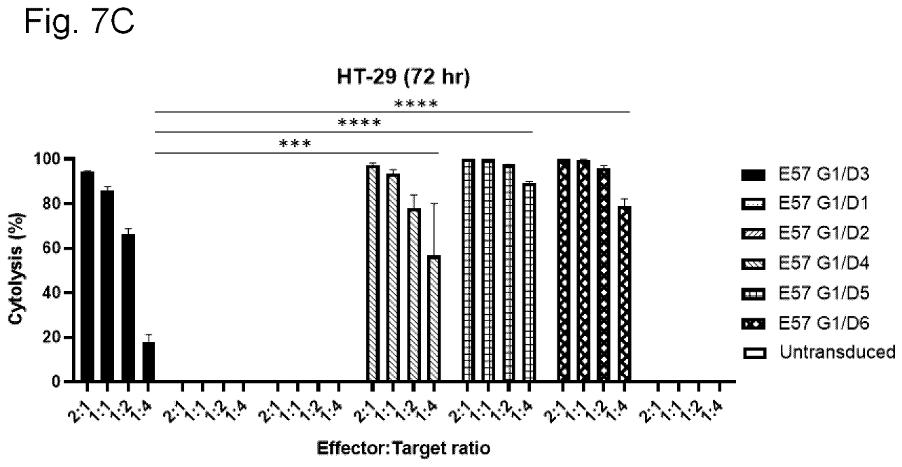

From the γ^{random}δ^{E57}TCR and γ^{E57}δ^{random}TCR library screen a selected number of TEGs, expressing γδTCRs comprising CDR3 region variants G1/D1, G1/D2, G1/D4, G1/D5, G1/D6, G2/D3, G8/D3, G5/D3, or G10/D3, were compared to the TEGs expressing the reference E57 γδTCR (G1/D3) and tested for reactivity against the tumour Luc-Tom HT-29 cell line as measured with cytolyses (FIG. 7A-7C).

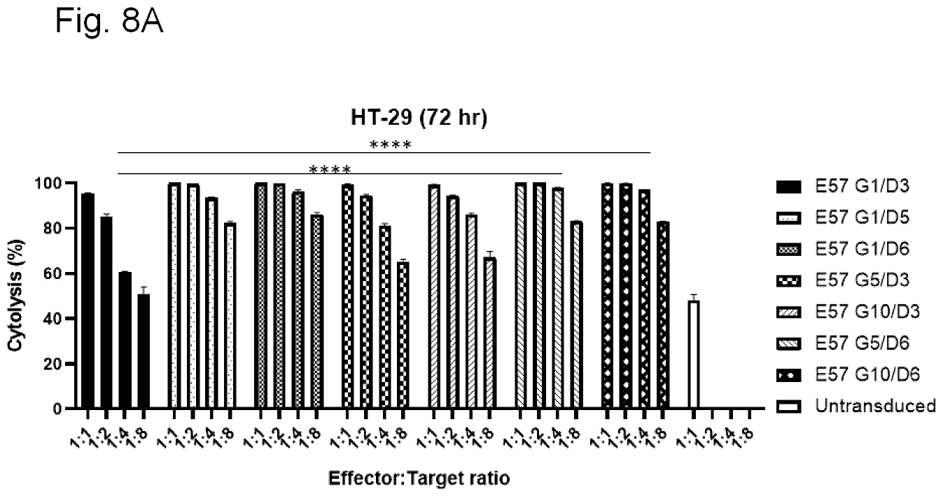

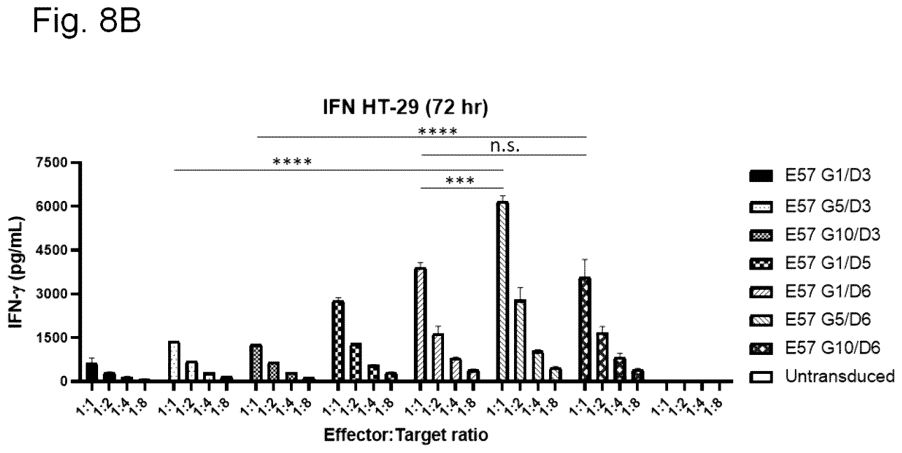

Pairing of the more reactive γTCR variants (G5 and G10) with the more reactive δTCR variant (D6) in TEGs showed a synergistic reactivity towards the Luc-Tom HT-29 tumour cell line as determined by cytolyses and release of IFNγ release in the supernatant (FIG. 8A-8B).

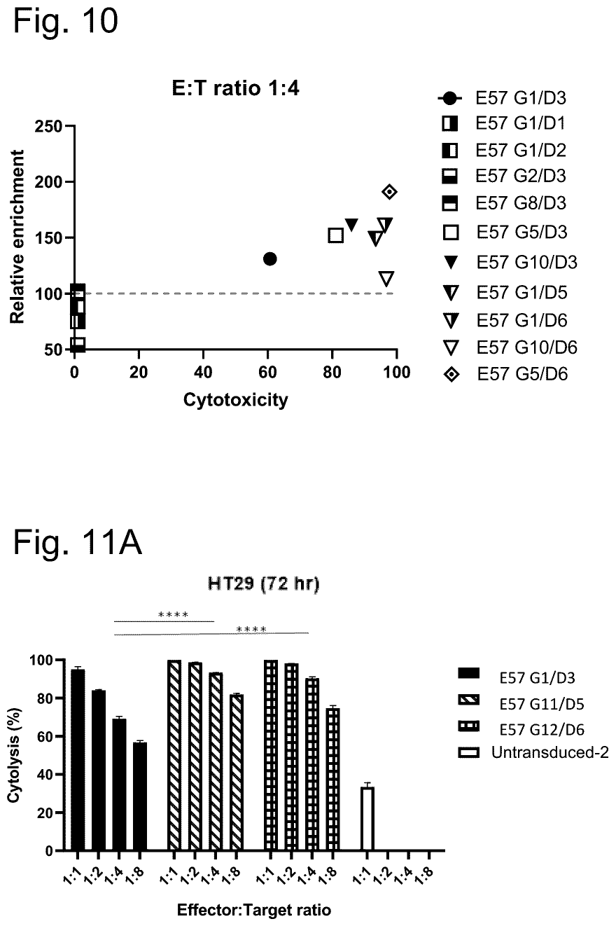

The reactivity of TEGs expressing all tested γδTCR-CDR3 variants correlated with the obtained outcome of the selection screening method as displayed in the correlation plot (FIG. 10).

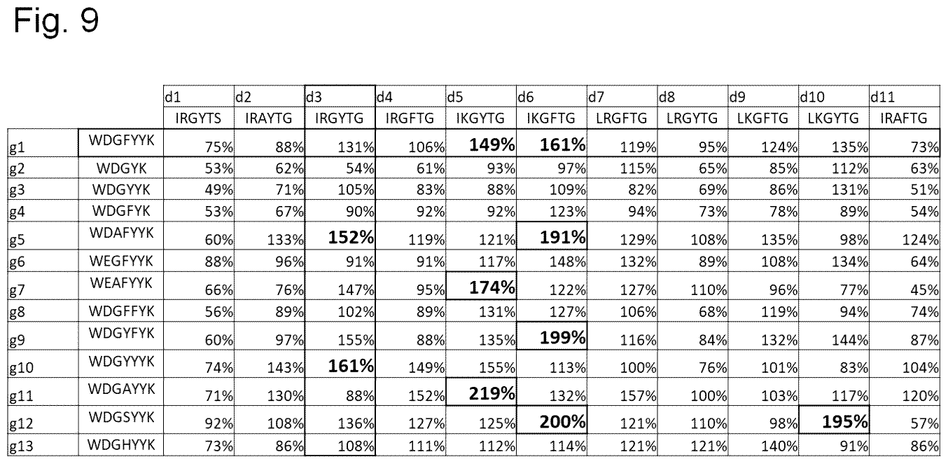

For the γ^{random}δ^{random}TCR library (FIG. 9), TEGs expressing γδTCR-CDR3 variants comprising one random and one reference TCR chain, like G5/D3 (SEQ ID NO: 10, 2), G10/D3 (SEQ ID NO: 15, 2), G1/D5 (SEQ ID NO: 1, 22), G1/D6 (SEQ NO: 1, 23) were enriched for CD69 and CD107a.

TEGs expressing a number of randomly paired γδTCR-CDR3 variant chains (G7/D5 (SEQ ID NO: 12, 22), G11/D5 (SEQ ID NO: 16, 22), G5/D6 (SEQ ID NO: 10, 23), G9/D6 (SEQ ID NO: 14, 23) G12/D6 (SEQ ID NO: 17, 23) and G12/D10 (SEQ ID NO: 17, 26), showed an enrichment percentage above 170%.

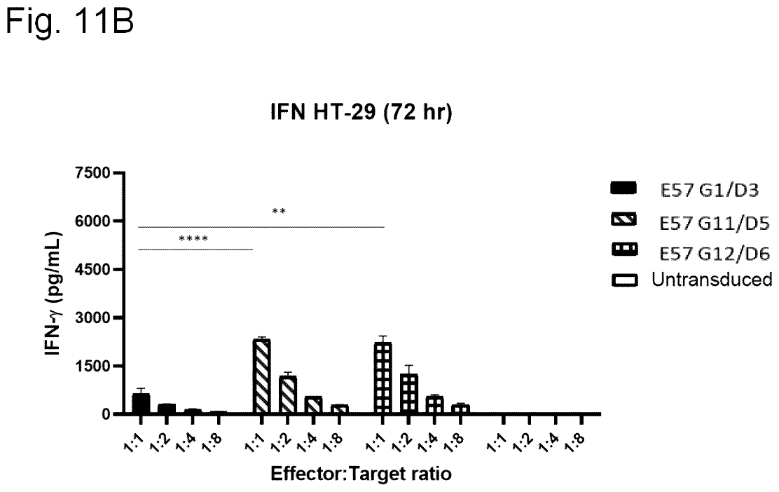

TEGs expressing the G11/D5 (SEQ ID NO: 16, 22) or G12/D6 (SEQ ID NO: 17, 23) variants showed an augmented reactivity towards Luc-Tom HT-29 tumour cells in comparison to TEGs expressing the reference E57 γδTCR (G1/D3; SEQ ID NO: 1, 2) (FIG. 11A-11B).

### Example 3

In this example, we compared the persistence of TEGs from two different donors expressing a γδTCR comprising the reference CDR3 regions from E57 (G1/D3, SEQ ID NOs: 1, 2) or the G5/D6 variant (SEQ ID NOs: 10, 23) to cytolyse Luc-Tom HT-29 tumour cells following serial weekly stimulations.

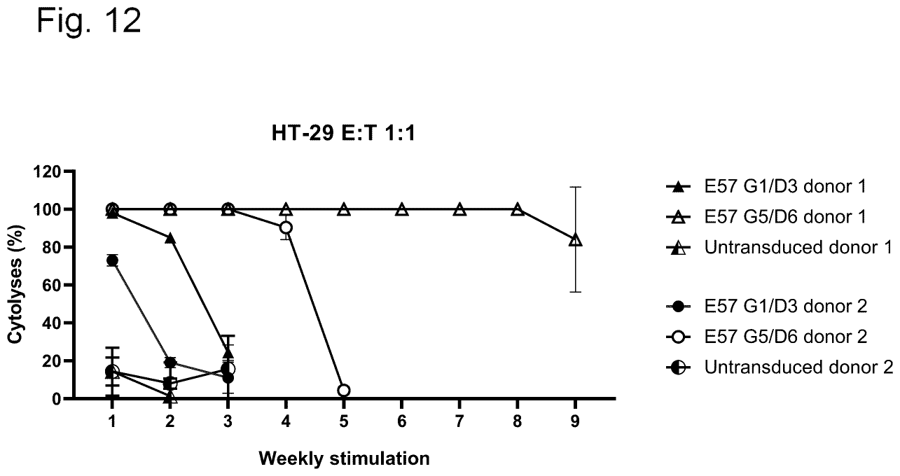

Serial stimulation was performed as described in the Materials and methods, for a total of 3, 5, or 9 stimulation rounds. Cytotoxicity was measured using a luciferase-based cytotoxicity assay, as described in the Materials and Methods. As depicted in FIG. 12, TEGs expressing the G5/D6 variant were more persistent in killing the tumour Luc-Tom HT-29 cells in subsequent stimulations compared to the TEGs expressing the reference E57 TCR (G1/D3).

### Example 4

In this example, we compared the cytolyses profiles and release of IFNγ of TEGs expressing a γδTCR comprising the reference CDR3 regions from E57 (G1/D3, SEQ ID NOs: 1, 2) or the G5/D6 variant (SEQ ID NOs: 10, 23) towards a panel of colorectal tumour cells and the MDA-MB-231 triple negative breast cell line. Tumour cell lines (OUMS23 and HT-29 KO) lacking the recognised target by E57 (EPCR) were used as a negative cell line. MDST-8 cells were used as a control of γTCR target specificity as these cells highly express the target, but are not recognized by E57. A tumour breast cell line positive for the recognized target that is not recognized by E57 was used as another control to determine cell type specificity.

Cytolytic activity comparisons were made using xCELLigence as described in the Materials & Methods, using HT-29, HT-29 target knock out (EPCR KO), RKO, T84, LS174T, SW480, LS80, HT55, KM12, MDST-8, OUMS23 colon carcinoma cells and the MDA-MB-231 triple negative breast cell line.

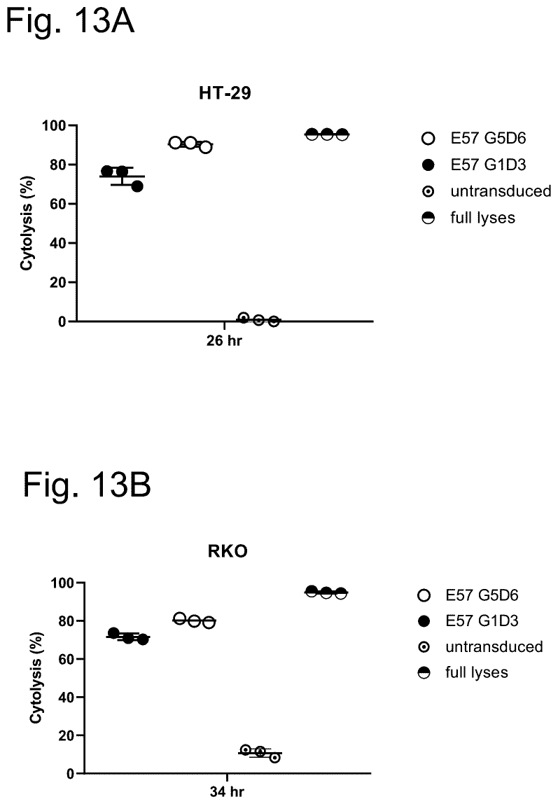

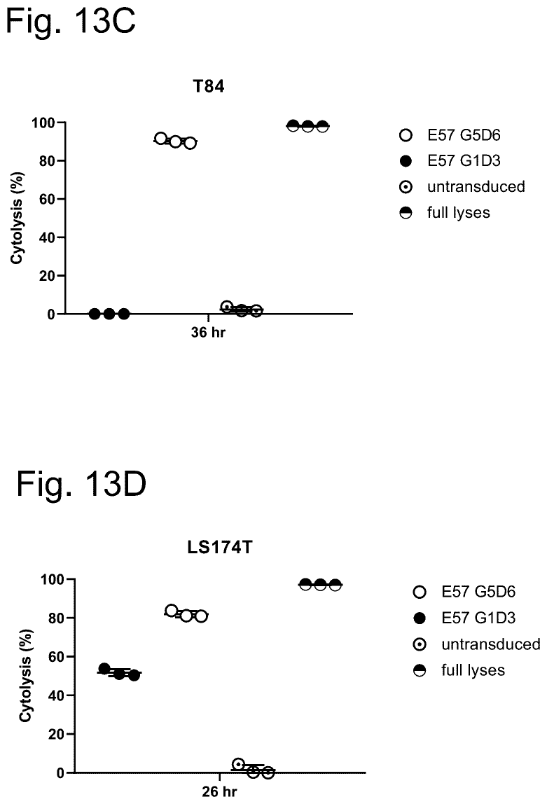

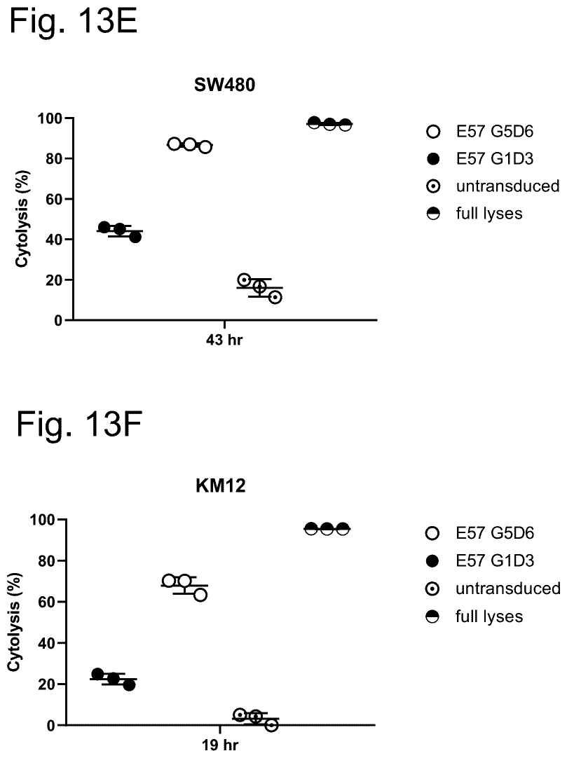

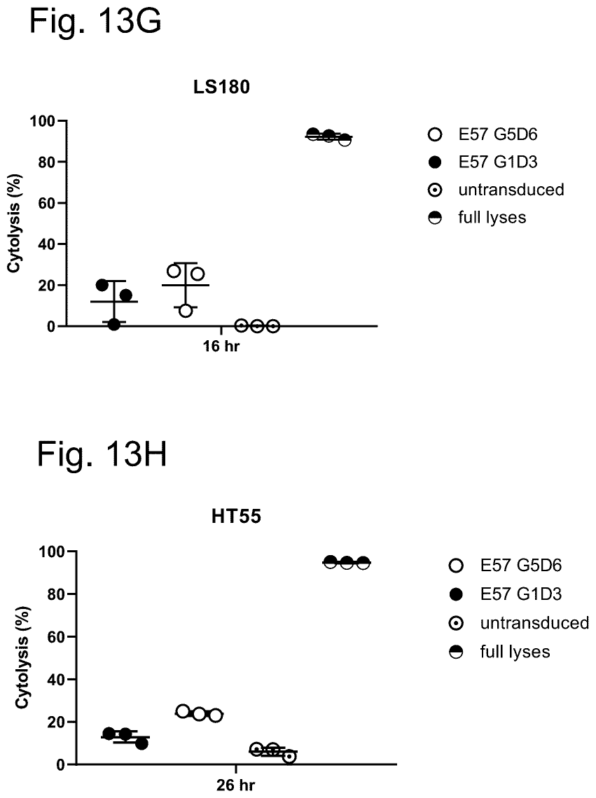

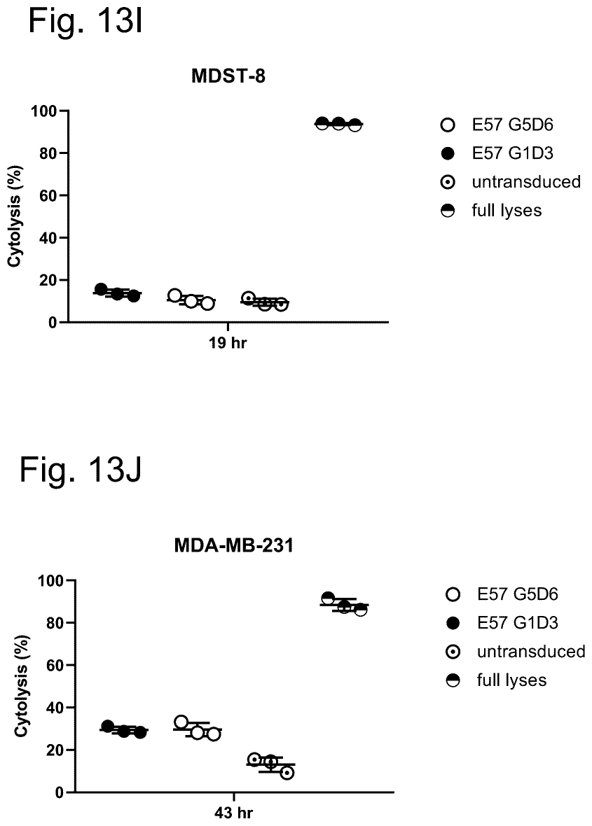

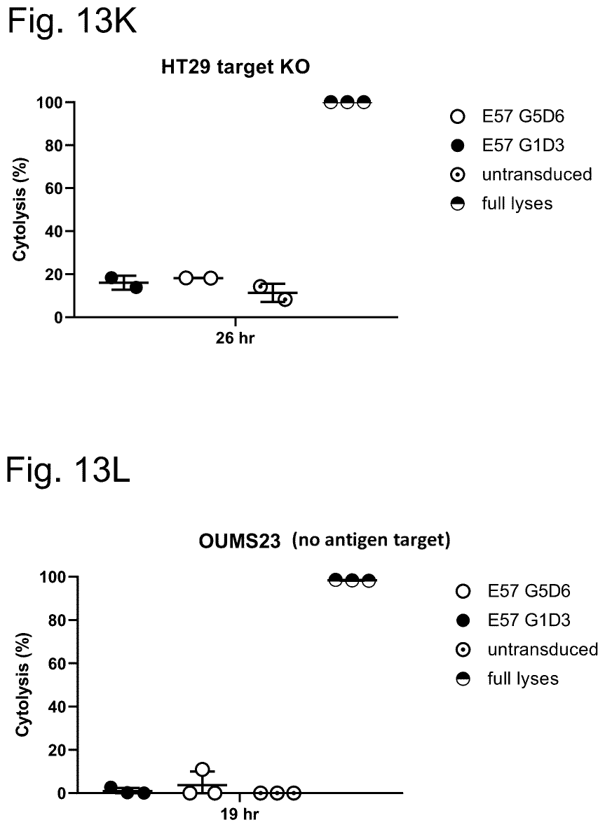

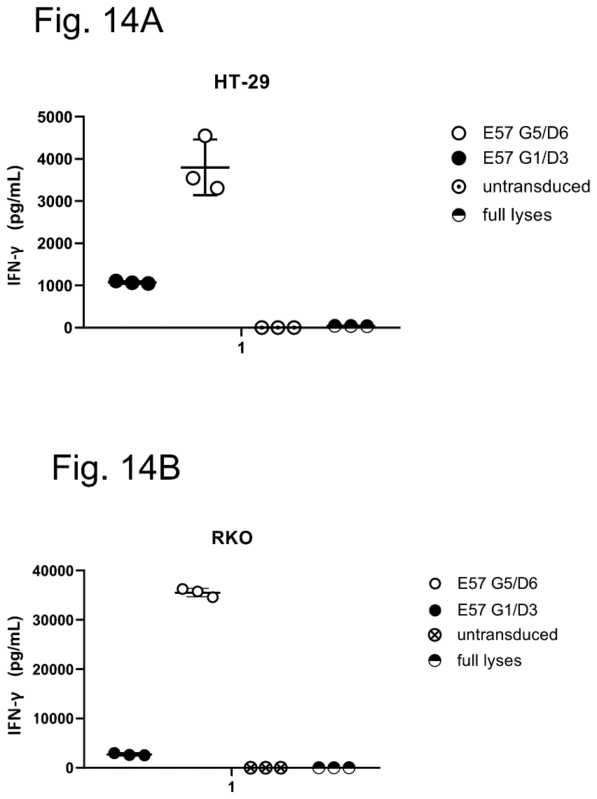

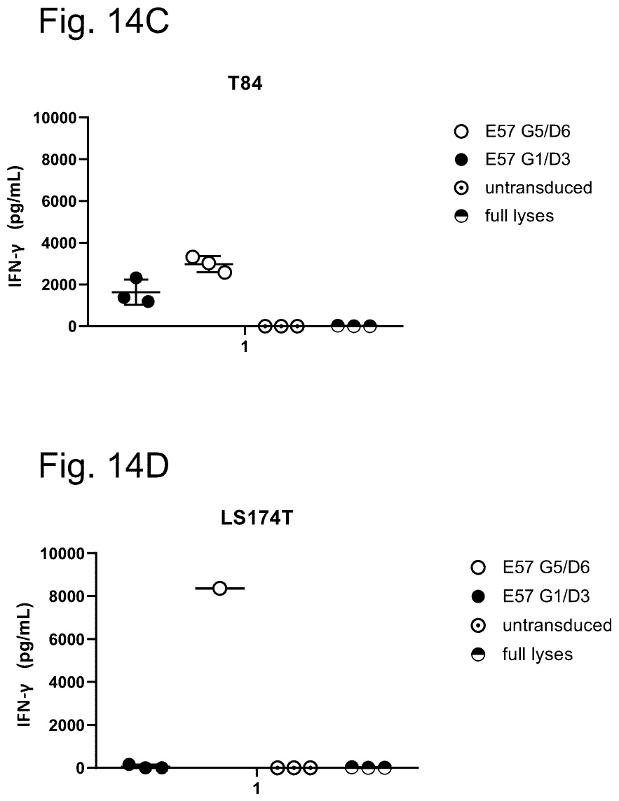

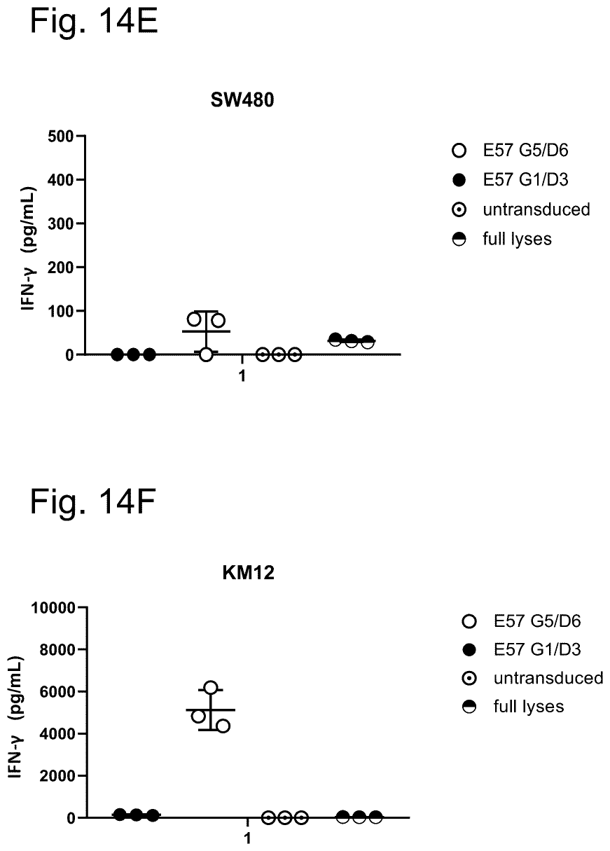

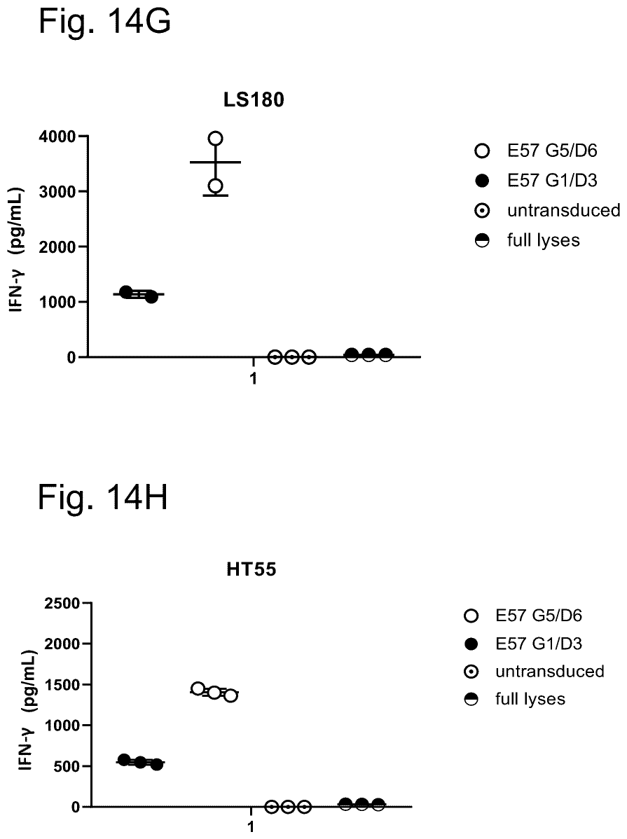

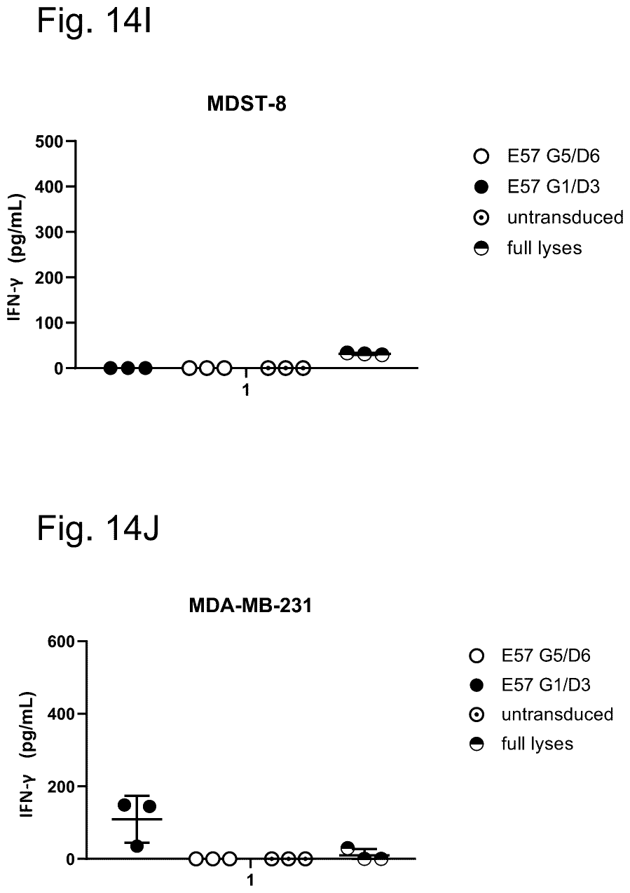

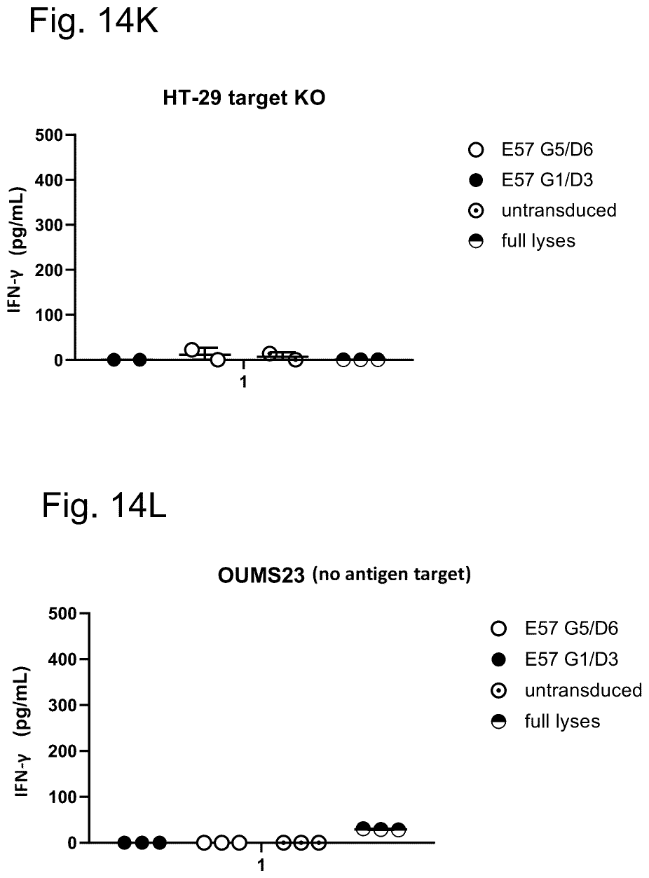

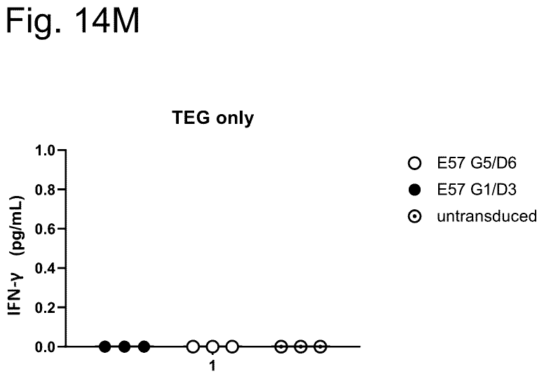

As depicted in FIG. 13A-13L and FIG. 14A-14M, an increase in cytolyses and IFNγ production was shown by TEGs expressing the γδTCR comprising the G5/D6 variant as compared to TEGs expressing the reference E57 γδTCR (G1/D3). TCR selectivity for cell type, or target specificity did not change between reference and G5/D6 variant.

### Example 5

In this example, we engineered soluble γδTCRs comprising the γδCDR3 regions from reference E57 (G1/D3, SEQ ID NOs: 1, 2), the G5/D6 variant (SEQ ID NOs: 10, 23), or the G2/D1 variant (SEQ ID NOs: 7, 19) CDR3 regions, with or without a natural cysteine bond formed between the constant 1 γTCR region (Cγ1) and the constant δ-connecting peptide region (in the case of G5/D6), as shown in the Materials and Methods. The sTCRs were truncated in the connecting peptide region and lacked the transmembrane domain. The encoding c-Jun/c-Fos region, Avi-tag and His-tag were attached to the C-terminus of the γδTCRs.

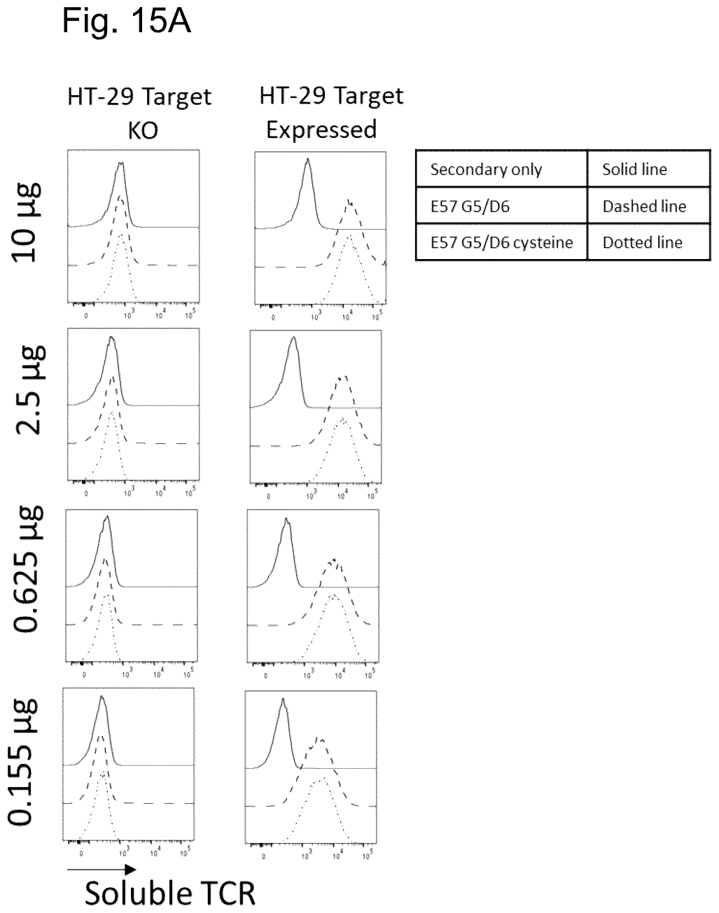

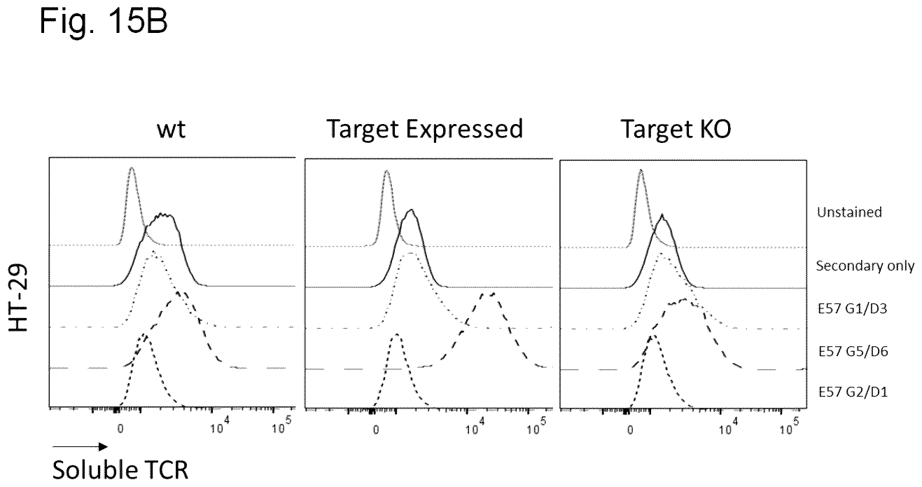

The soluble γδTCRs were used to assess specific binding to HT-29 cells, HT-29 knocked out (KO) for the expressed target (EPCR) or HT-29 cells with ectopic target expression. Bound soluble γδTCRs were targeted with a secondary streptavidin A647-labelled antibody that could be detected with FACS flow cytometry. As depicted in FIG. 15A specific target binding was observed for both the G5/D6 variant (SEQ ID NOs: 10, 23) with (SEQ ID NOs: 244, 258) or without cysteine (SEQ ID NOs: 220, 234). Effective binding was observed even when small amounts of soluble G5/D6 γδTCR were used (FIG. 15A). On the contrary, the soluble reference E57 γδTCR (G1/D3; SEQ ID NOs: 216, 231) and the G2/D1 (SEQ ID NOs: 217, 229) variant did not effectively bind the target (FIG. 15B), whilst the G5/D6 variant did. The target specificity of the soluble G5/D6 CDR3 variant γδTCR remained intact and the variant showed superior target binding in comparison to the reference E57 γδTCR.

This example shows that γδTCR-CDR3 variants with improved reactivity towards the target molecules as obtained from the selection screening method have an increased affinity for their target also in soluble form, as the specific binding to the target with a soluble γδTCR having the same γ- and δ-CDR3 is increased.

These results were consistent with the results obtained in the experiments utilizing TEG expression.

### Example 6

In this example, we engineered γδTCR-CD3 bispecific engagers comprising a γTCR chain that was truncated in the connecting peptide region and lacked the transmembrane domain, and connected to the OKT3 scFv followed by an Avi-Tag and His-tag, paired with a δTCR chain that was truncated at the connecting peptide region.

Three γδTCR-CD3 bispecific engagers were generated, comprising the reference E57 CDR3 regions (G1/D3; SEQ ID NOs: 288, 303), G5/D6 (SEQ ID NOs: 292, 306), or G2/D1 variant (SEQ ID NOs: 289, 301).

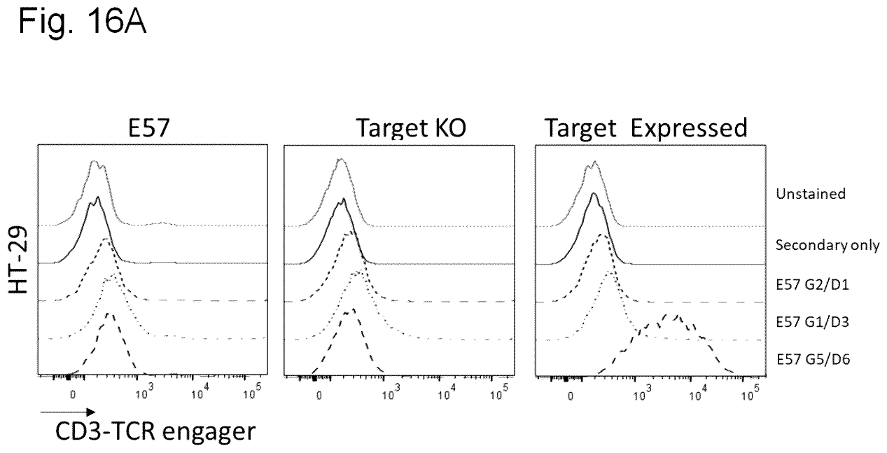

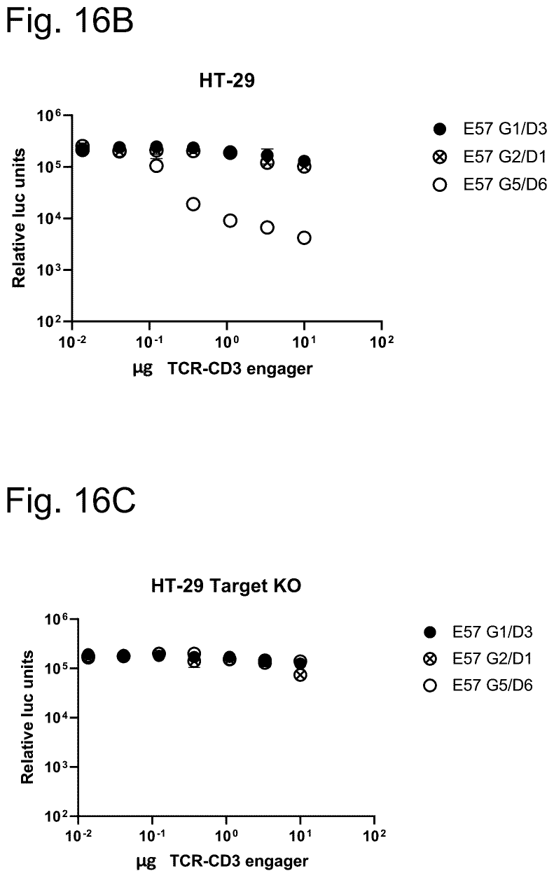

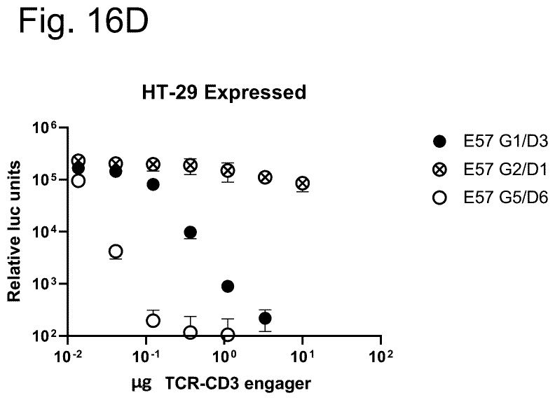

The γδTCR-CD3 bispecific engagers were used in binding experiments utilizing HT-29 cells, HT-29 with the target antigen (EPCR) knocked out (KO) or with ectopic expression of the target. Bound γδTCR-CD3 bispecific engagers were targeted with an anti-His-APC-labelled secondary antibody that could be detected with FACS flow cytometry. As depicted in FIG. 16A, the soluble reference E57 and G2/D1 γδTCR-CD3 bispecific engagers did not bind to the target cells, whereas the G5/D6 did.

γδTCR-CD3 bispecific engagers can be used as soluble biologicals directed to target tumour cells, as they are able to interact with CD3-TCR expressing cells, such as αβ or γδ T-cells, via the CD3-specific scFv and bring them into contact with the tumour cells that are bound via the γδTCR. This bridging of the tumour cells with the CD3-TCR expressing cells can trigger the cytolysis of the tumour cells. Here, we targeted Luc-Tom HT-29 cells, Luc-Tom HT-29 KO (EPCR) cells, and Luc-Tom HT-29 with EPCR ectopically expressed cells with serial diluted reference E57, G5/D6, and G2/D1 γδTCR-CD3 bispecific engagers. Subsequently, αβT-cells were added at a 1:1 E:T ratio and cytolyses was determined using a luciferase-based cytotoxicity assay as described in the materials and methods.

As shown in FIG. 16B, the G5/D6 γδTCR-CD3 bispecific engager was able to mediate the cytolysis of Luc-Tom HT-29 tumour cells, whilst the reference E57 and G2D1 γδTCR-CD3 bispecific engager were not (FIG. 16B). Luc-Tom HT-29 KO cells lacking the E57 recognised target (EPCR) were not subjected to cytolysis (FIG. 16C). The Luc-Tom HT-29 “EPCR ectopically expressed” cell line was lysed upon engaging αβT-cells mediated via the reference E57 γδTCR-CD3 bispecific engager, but a significant improved cytolysis profile was observed with the G5/D6 γδTCR-CD3 bispecific engager (FIG. 16D).

### Example 7

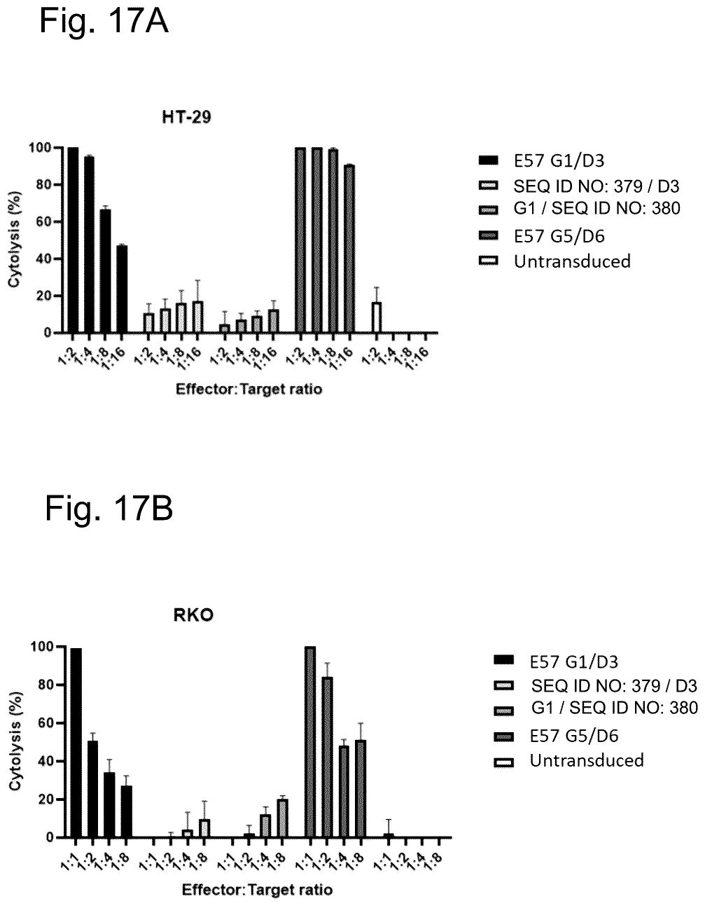

In this example, we compared the cytolyses profiles of TEGs expressing a γδTCR comprising the reference CDR3 regions from E57 (G1/D3, SEQ ID NOs: 1, 2), or the control CDR3 regions represented by SEQ ID NO: 379 (paired with SEQ ID NO: 2) or SEQ ID NO: 380 (paired with SEQ ID NO: 1), with TEGs expressing a γδTCR comprising the CDR3 regions from the G5/D6 variant (SEQ ID NOs: 10, 23) against Luc-Tom HT-29 cells (FIG. 17A) or Luc-Tom RKO cells (FIG. 17B). Cytolytic activity comparisons were made using a luciferase-based (serial) cytotoxicity assay as described in the materials and methods. An E:T ratio of 1:1, 1:2, 1:4, 1:8, or 1:16 was used. As depicted in FIG. 17A and FIG. 17B, an increase in cytolyses was shown by TEGs expressing the γδTCR comprising the G5/D6 variant as compared to TEGs expressing the reference E57 γTCR or γTCRs comprising the control CDR3 regions.

1. A soluble γT-cell receptor chain or a fragment thereof comprising a γCDR3 region, wherein said γCDR3 region is represented by an amino acid sequence comprising at least 60% sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 4-10 or one or more of positions 5-9 of SEQ ID NO: 1.

2-3. (canceled)

4. The soluble γT-cell receptor chain or fragment thereof according to claim 1, wherein said modification in the γCDR3 region is a substitution of an aspartic acid by a glutamic acid at position 5 of SEQ ID NO: 1, a substitution of a glycine by an alanine at position 6 of SEQ ID NO:1, a substitution of a phenylalanine by an alanine, serine, or tyrosine at position 7 of SEQ ID NO:1, a substitution of a tyrosine by a phenylalanine at position 8 of SEQ ID NO:1, and/or a substitution of the amino acid at position 9 of SEQ ID NO:1.

5-8. (canceled)

9. The soluble γT-cell receptor chain or fragment thereof according to claim 1, wherein said γCDR3 region comprises an amino acid sequence selected from the group consisting of DAFYY (SEQ ID NO: 369), EAFYY (SEQ ID NO: 370), DGYFY (SEQ ID NO: 371), DGYYY (SEQ ID NO: 372), DGAYY (SEQ ID NO: 373), and DGSYY (SEQ ID NO: 374) at the amino acid positions corresponding to positions 5-9 of SEQ ID NO: 1.

10. (canceled)

11. The soluble γT-cell receptor chain or fragment thereof according to claim 1, wherein said receptor chain or fragment thereof further comprises a γCDR1 region represented by an amino acid sequence comprising at least 70% sequence identity or similarity with SEQ ID NO: 375, and a γCDR2 region represented by an amino acid sequence comprising at least 70% sequence identity or similarity with SEQ ID NO: 376.

12-13. (canceled)

14. The soluble γT-cell receptor chain or fragment thereof according to claim 1, wherein said receptor chain or fragment thereof comprises an amino acid sequence comprising at least 70% identity or similarity with an amino acid sequence selected from SEQ ID NO: 120 or 122-132, SEQ ID NO: 144 or 146-156, SEQ ID NO: 168 or 170-180, SEQ ID NO: 192 or 194-204, SEQ ID NO: 216 or 218-228, SEQ ID NO: 240 or 242-252, SEQ ID NO: 264 or 266-276, SEQ ID NO: 288 or 290-300, or SEQ ID NO: 312 or 314-324.

15. The soluble γT-cell receptor chain or fragment thereof according to claim 1, wherein said receptor chain or fragment thereof comprises an amino acid sequence comprising at least 70% identity or similarity with an amino acid sequence selected from SEQ ID NO: 124, 148, 172, 196, 220, 244, 268, 292, or 316.

16. (canceled)

17. The soluble γT-cell receptor chain or fragment thereof according to claim 1, wherein said receptor chain or fragment thereof mediates an anti-tumor or anti-infective response, preferably against a target cell expressing endothelial protein C receptor (EPCR).

18. A soluble δT-cell receptor chain or a fragment thereof comprising a δCDR3 region, wherein said δCDR3 region is represented by an amino acid sequence comprising at least 60% sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 7-12 of SEQ ID NO: 2.

19. (canceled)

20. The soluble δT-cell receptor chain or fragment thereof according to claim 18, wherein said modification in the δCDR3 region is a substitution of an isoleucine by a leucine at position 7 of SEQ ID NO: 2, a substitution of an arginine by a lysine at position 8 of SEQ ID NO:2, a substitution at position 9 of SEQ ID NO: 2, a substitution of a tyrosine by a phenylalanine at position 10 of SEQ ID NO: 2, a substitution at position 11 of SEQ ID NO:2 and/or a substitution at position 12 of SEQ ID NO: 2.

21.-25. (canceled)

26. The soluble δT-cell receptor chain or fragment thereof according to claim 18, wherein said δCDR3 region comprises an amino acid sequence selected from the group consisting of IRGFTG (SEQ ID NO: 95), IKGYTG (SEQ ID NO: 96), IKGFTG (SEQ ID NO: 97), LRGFTG (SEQ ID NO: 98), LKGFTG (SEQ ID NO: 111), and LKGYTG (SEQ ID NO: 100) at the amino acid positions corresponding to positions 7-12 of SEQ ID NO: 2.

27. The soluble δT-cell receptor chain or fragment thereof according to claim 18, wherein said receptor chain or fragment thereof further comprises a δCDR1 region represented by an amino acid sequence comprising at least 70% sequence identity or similarity with SEQ ID NO: 377, and a δCDR2 region represented by an amino acid sequence comprising at least 70% sequence identity or similarity with SEQ ID NO: 378.

28-29. (canceled)

30. The soluble δT-cell receptor chain or fragment thereof according to claim 18, wherein said receptor chain or fragment thereof comprises an amino acid sequence comprising at least 70% identity or similarity with an amino acid sequence selected from SEQ ID NO: 134-143, 158-167, 182-191, 206-215, 230-239, 254-263, 278-287, 302-311, or 326-335.

31. The soluble δT-cell receptor chain or fragment thereof according to claim 18, wherein said receptor chain or fragment thereof comprises an amino acid sequence comprising at least 70% identity or similarity with an amino acid sequence selected from SEQ ID NO: 138, 162, 186, 210, 234, 258, 282, 306, or 330.

32. (canceled)

33. The soluble δT-cell receptor chain or fragment thereof according to claim 18, wherein said receptor chain or fragment thereof mediates an anti-tumor or anti-infective response, preferably against a target cell expressing endothelial protein C receptor (EPCR).

34. A soluble γδT-cell receptor or fragment thereof comprising a γCDR3 and a δCDR3 region, wherein the γδT-cell receptor or fragment thereof comprises:
a soluble γT-cell receptor chain or fragment thereof comprising a γCDR3 region, wherein said γCDR3 region is represented by an amino acid sequence comprising at least 60% sequence identity or similarity with SEQ ID NO: 1, and wherein said receptor chain or fragment thereof comprises a modification in the γCDR3 region relative to SEQ ID NO: 1 at an amino acid position corresponding to a position selected from one or more of positions 4-10 or one or more of positions 5-9 of SEQ ID NO: 1; and/or
a soluble δT-cell receptor chain or fragment thereof comprising a δCDR3 region, wherein said δCDR3 region is represented by an amino acid sequence comprising at least 60% sequence identity or similarity with SEQ ID NO: 2, and wherein said receptor chain or fragment thereof comprises a modification in the δCDR3 region relative to SEQ ID NO: 2 at an amino acid position corresponding to a position selected from one or more of positions 7-12 of SEQ ID NO: 2.

35. The soluble γδT-cell receptor or fragment thereof according to claim 34, wherein the γδT-cell receptor or fragment thereof comprises:
a soluble γT-cell receptor chain or fragment thereof comprising a γCDR3 region comprising the amino acid sequence DAFYY (SEQ ID NO: 369) at the amino acid positions corresponding to positions 5-9 of SEQ ID NO: 1 and/or,
a soluble δT-cell receptor chain or fragment thereof comprising a δCDR3 region comprising the amino acid sequence IKGFTG (SEQ ID NO: 97) at the amino acid positions corresponding to positions 7-12 of SEQ ID NO: 2.

36. A soluble γδT-cell receptor or fragment thereof according to claim 34, wherein the γδT-cell receptor or fragment thereof further comprises a T-cell- and/or NK-cell binding domain.

37-42. (canceled)

43. The soluble γδT-cell receptor or fragment thereof according to claim 36, wherein said T-cell binding domain is an anti-CD3 domain.

44. A nucleic acid molecule encoding the soluble γδT-cell receptor or fragment thereof as defined in claim 34.

45. A nucleic acid construct comprising the nucleic acid molecule as defined in claim 44.

46. A cell expressing the soluble γδT-cell receptor or fragment thereof as defined in claim 34.

47. A cell comprising the nucleic acid molecule as defined in claim 44.

48. A composition comprising the soluble γδT-cell receptor or fragment thereof as defined in claim 34.

49. A method of treatment of a cancer or an infection comprising administering an effective amount of a soluble γδT-cell receptor or fragment thereof as defined in claim 15, a cell expressing the soluble γδT-cell receptor or fragment thereof and/or a nucleic acid encoding the soluble γδT-cell receptor or fragment thereof, to a subject in need thereof.

50. A method of treatment according to claim 49, wherein the cancer is an EPCR-expressing cancer.
